SEQUENCE LISTING (927 sequences)

SEQ ID NO: 1 - DNA, 60 bp, Mus musculus
atgagcctagaactcacatgcattttcctgacttctatcattagaataagttcatcaaga

SEQ ID NO: 2 - DNA, 60 bp, Mus musculus
cctattgttgagtgtcaaacatcaccactaagtggatggttatgtagtccattatccaaa

SEQ ID NO: 3 - DNA, 60 bp, Mus musculus
tacctgaaccactctctactgttgttgtcacaaggcaaaagtggcattccttcctccaag

SEQ ID NO: 4 - DNA, 60 bp, Mus musculus
ccctttgctgtgtgggcagtactctgaagcaggcaaatgggtcttaggatccctcccaga

SEQ ID NO: 5 - DNA, 60 bp, Mus musculus
tccaaagataaaatgagcaaccgcactggcttagccatagatgactgacagtgattggaa

SEQ ID NO: 6 - DNA, 60 bp, Mus musculus
tgccttggagggcaacaaggagcagatacagaagatcattgagacactgttcacagcagc

SEQ ID NO: 7 - DNA, 60 bp, Mus musculus
catgaattccaaaccagttattattaacatgaacctgaacctgaacaattatgactgtgc

SEQ ID NO: 8 - DNA, 60 bp, Mus musculus
tttctgtcactgctcaggccaaggtctatgaacgttgtgagtttgccagaactctgaaaa

SEQ ID NO: 9 - DNA, 60 bp, Mus musculus
ttcataccaaggaacctgacctctctgacaattgcattttgaacattgttgtccccaaag

SEQ ID NO: 10 - DNA, 60 bp, Mus musculus
cattggaaacagacacgtttgtaggcatttgcgtattcttgaagagactgttttatgaat

SEQ ID NO: 11 - DNA, 60 bp, Mus musculus
gtaatggagaatgtatctgaacccatatcaagccatctctcttccttaacatgttaagca

SEQ ID NO: 12 - DNA, 60 bp, Mus musculus
acacctctaactcccaagaagacggagtgaatgtcctctcctttacttgtgaaatcattt

SEQ ID NO: 13 - DNA, 60 bp, Mus musculus
gtgagattcggcagcataaattgcggaaactgaacccacccgatgagagtggtcctggct

SEQ ID NO: 14 - DNA, 60 bp, Mus musculus
tcataagggctaaattcatgggttccccagaaatcaacgagaccaccttataccagcgtt

SEQ ID NO: 15 - DNA, 60 bp, Mus musculus
aaagactgagaggagtcatgaaccagggtaaaacttattggtgctttgagacttccagca

SEQ ID NO: 16 - DNA, 60 bp, Mus musculus
gcagcatcgcttccttggtttattctttgtgtttgttccttcagtaaacatttattgagc

SEQ ID NO: 17 - DNA, 60 bp, Mus musculus
ttttaacggagcctgaatatagcaggtttaaaatttaaacaggtataaaatgaaaaataa

SEQ ID NO: 18 - DNA, 60 bp, Mus musculus
tagcatgaaccaccatgtttggcaatactgtattttagaaagaattaatggactggagag

SEQ ID NO: 19 - DNA, 60 bp, Mus musculus
cctgagctcactgtttctcatgctgtcttgagacaaagtatccatatggaacctaggtta

SEQ ID NO: 20 - DNA, 60 bp, Mus musculus
gctggtgtttgtgtcaagaaaatggctgaagcttgtttccaggctgtaggaatgttgaac

SEQ ID NO: 21 - DNA, 60 bp, Mus musculus
acttaagttatctgcatagaggcaatcctcctgggtttgctttatgtctcgaaaatctaa

SEQ ID NO: 22 - DNA, 60 bp, Mus musculus
gggcaaaggtactttctgacaaactgagtacctgagatcaacccccaagaagggaaaaaa

SEQ ID NO: 23 - DNA, 60 bp, Mus musculus
actatgcaattggacagatggattaccaaggagactaaaaatatattctttgactttggg

SEQ ID NO: 24 - DNA, 60 bp, Mus musculus
tcactgacctcaacccctcctgcagagaagcctgaagaccccaaaagctgccagtccaaa

SEQ ID NO: 25 - DNA, 60 bp, Mus musculus
gatataatgtgataaagttccaaaaggatctctctggctgaaggagatactggatggaac

SEQ ID NO: 26 - DNA, 60 bp, Mus musculus
ctgaaccccaattaatagcaaaggatatatctctcttcaaaaacggatagatttctgaag

SEQ ID NO: 27 - DNA, 60 bp, Mus musculus
ttttgttctctccatctgttagccgttctgaggactgaatgcagattgtcagctcaaaaa

SEQ ID NO: 28 - DNA, 60 bp, Mus musculus
gccaatctcagaacccacatagaagggtctgcagtattattcctgtttcatgtgtgcaca

SEQ ID NO: 29 - DNA, 60 bp, Mus musculus
agtgcaaaatttggtttgttggtgtgcttttctggtttaggagcctgaaacaagcacact

SEQ ID NO: 30 - DNA, 60 bp, Mus musculus
catgagtaagttgtgaaggctggacccacatcttgatacttgttttctgcatcttgggca

SEQ ID NO: 31 - DNA, 60 bp, Mus musculus
tagacgttgtaaaaaggagccaagtttatcattttgttccttaaatccgtcatatgtggg

SEQ ID NO: 32 - DNA, 60 bp, Mus musculus
actgtggtgacagcttcctaacgtgtttgtgtctaaaataaactatccttagcatccttc

SEQ ID NO: 33 - DNA, 60 bp, Mus musculus
tataaatagaaagtgaacctgtaacctaccacggtatctatcataacactagactttcag

SEQ ID NO: 34 - DNA, 60 bp, Mus musculus
catcctacaaagaggataagcactttgggtacacttcctacagcgtgtctaacagtgtga

SEQ ID NO: 35 - DNA, 60 bp, Mus musculus
cctgaaaatctgtcatgtccaccttggagcctgagtaactttgaacagctggtaactagt

SEQ ID NO: 36 - DNA, 60 bp, Mus musculus
agtcaaggagcctaaagattattatgtcagagagaccagctttagatacacccctgagca

SEQ ID NO: 37 - DNA, 60 bp, Mus musculus
ttatgctgcagtttcacttggaaaagggacaaggagccttctattgtcccctgtttgtag

SEQ ID NO: 38 - DNA, 60 bp, Mus musculus
gtaaccaagagccctgaataaggaattcattgtagtagtgaaagggaaactaatgctctt

SEQ ID NO: 39 - DNA, 60 bp, Mus musculus
tcccatgccttcccagagggaattttaacaatgtaacaataaatgcttggccttgaagct

SEQ ID NO: 40 - DNA, 60 bp, Mus musculus
aggacatcttcccagatctcaaaagaagaagagagcctgtaaccacctccatgacctaaa

SEQ ID NO: 41 - DNA, 60 bp, Mus musculus
tcctgtgggagatcccataaatcctgaacctcacgtagtgttacttttccaggtcattct

SEQ ID NO: 42 - DNA, 60 bp, Mus musculus
cgacgacgagttcgaagacgacctgctcgacctgaaccccagctcaaactttgagagcat

SEQ ID NO: 43 - DNA, 60 bp, Mus musculus
gaagagatggaagatggtagtgccttgaacacagccacccaagcaaagttgaagaacagg

SEQ ID NO: 44 - DNA, 60 bp, Mus musculus
gcctgcaggagtttgtgttggtagcctccaaggagctggaatgtgctgaagatccaggct

SEQ ID NO: 45 - DNA, 60 bp, Mus musculus
ctgtcttctaattccaaagggttggttggtaaagctccacccccttttcctttgcctaaa

SEQ ID NO: 46 - DNA, 60 bp, Mus musculus
ttcacagggttcctggtgttgcatgcagagcctgaacaaaagactcaggtggacctggaa

SEQ ID NO: 47 - DNA, 60 bp, Mus musculus
tctacaaggaagcattcaaccaccaagaggagcttggaccacgttcactctgtattcttt

SEQ ID NO: 48 - DNA, 60 bp, Mus musculus
gggcctgaactatggcttaatttacattaattagttaacattaatcacacagtaaggagc

SEQ ID NO: 49 - DNA, 60 bp, Mus musculus
tgtgttgtgatttcaactcccaagacgccctttatgtccattctggaaaaatacaataaa

SEQ ID NO: 50 - DNA, 60 bp, Mus musculus
actgatgtttctgcacactgcccagtggtttctttaagcactttctggaataaacgatcc

SEQ ID NO: 51 - DNA, 60 bp, Mus musculus
tcacagatgtatgtggaggggttgttttctgagtactagactaccctctgtggttataaa

SEQ ID NO: 52 - DNA, 60 bp, Mus musculus
tcggggatggagctgagatgttccaccacaacccaagatctaagagtattgttttgaaga

SEQ ID NO: 53 - DNA, 60 bp, Mus musculus
ggagactgaagcttttattgtttaatgttgaagatattgatctacaaggtgggaatggtg

SEQ ID NO: 54 - DNA, 60 bp, Mus musculus
aactgtgggtataattgtaagagcctgaaacttccagaactggagaaactgtcactggga

SEQ ID NO: 55 - DNA, 60 bp, Mus musculus
gtgttgtgattgtcgtccctgcttaatgaacccacctgagggacagttagtgtcttaccc

SEQ ID NO: 56 - DNA, 60 bp, Mus musculus
ctatatgaactgagaaacaacacgtatgctgaaccccaattctacaacaaagtctacgcc

SEQ ID NO: 57 - DNA, 60 bp, Mus musculus
ggaatatattatgtagactattctggcctgaaccttgtggttgactgatgctctgcctcc

SEQ ID NO: 58 - DNA, 60 bp, Mus musculus
ttgggtgatccatatttttcaaacccatactcccaaaaggagacctacttaaatttctct

SEQ ID NO: 59 - DNA, 60 bp, Mus musculus
gttcctgaagctcttgatattttaggacaaaacccaccacgacaaaatgagaaggaattt

SEQ ID NO: 60 - DNA, 60 bp, Mus musculus
tgacttcaaatgtcccatcccacccaaagagcctgtgataacagatgtctctggctatat

SEQ ID NO: 61 - DNA, 60 bp, Mus musculus
tgggtaggttcctaggtctccctgatatctaagctacagttatactgtagctgtgtgaca

SEQ ID NO: 62 - DNA, 60 bp, Mus musculus
cctgtctcagaactcaaagaataaatccagtgtatcttcagagtcactttgtaaccctac

SEQ ID NO: 63 - DNA, 60 bp, Mus musculus
tactccctggagactagaaccgtggctatagcggagcatgctccagagcacaggactgat

SEQ ID NO: 64 - DNA, 60 bp, Mus musculus
gggacaccagaagtcaaccagaccaccttataccagcgttatgagatcaagatgaccaag

SEQ ID NO: 65 - DNA, 60 bp, Mus musculus
gaaaaccaaaactcttggtcagagacaatatgcaaaacagagatgtcaagtactatgtcc

SEQ ID NO: 66 - DNA, 60 bp, Mus musculus
tcaaggagactgtagacttaaaggcagaaccccgtaacaaagggctcacaggtcatcctc

SEQ ID NO: 67 - DNA, 60 bp, Mus musculus
caccacggactacaaccagttcgccatggtatttttccgaaagacttctgaaaacaagca

SEQ ID NO: 68 - DNA, 60 bp, Mus musculus
gtaccctctgactgtatatttcaatcggcctttcctgataatgatctttgacacagaaac

SEQ ID NO: 69 - DNA, 60 bp, Mus musculus
aagaactactgatacagaaccacttcagttgttcagttagaatctttttaagactctctc

SEQ ID NO: 70 - DNA, 60 bp, Mus musculus
cttgacctttagatggaaattgtacctagagacgagaaggagccaaactaaggtctgtca

SEQ ID NO: 71 - DNA, 60 bp, Mus musculus
ggaacggacaacgtggctttgtccctgggtcgtacttggagaagctctgaggaaaggcta

SEQ ID NO: 72 - DNA, 60 bp, Mus musculus
ttcgaatgcacatcattgacaagtttctcttattgcctttccactctggatgggaccctg

SEQ ID NO: 73 - DNA, 60 bp, Mus musculus
gcctggagactgaaggcagttttacaaaggaaaacttagatttctattcatttgcttttg

SEQ ID NO: 74 - DNA, 60 bp, Mus musculus
ctggatgaagaaacagagcatgattaccagaaccacatttagtctcccttggcattggga

SEQ ID NO: 75 - DNA, 60 bp, Mus musculus
ttaatattgtcaatgtcagggggttccctgtctcagagcattatgtgtactaactgtagc

SEQ ID NO: 76 - DNA, 60 bp, Mus musculus
ccagagttttttccatcatgttttgccccaaagacctcggtttgtagaagcccaaggaaa

SEQ ID NO: 77 - DNA, 60 bp, Mus musculus
gacagggtcaatgtttattatacatactgcactgatgagaacaatatcatatgtgaagag

SEQ ID NO: 78 - DNA, 60 bp, Mus musculus
actctcagcttcctgttggcaacagtggcagtgggaatttatgccatgtaaatgcaatac

SEQ ID NO: 79 - DNA, 60 bp, Mus musculus
gacagggactccatatggaagtaaggacgtttacctcattactaagtctcgtcaaaagaa

SEQ ID NO: 80 - DNA, 60 bp, Mus musculus
ctcggatcttcatgttcttcagtaagaatctctctgtggatttggaacaatcgtaaataa

SEQ ID NO: 81 - DNA, 60 bp, Mus musculus
ctaagacacctgtgatttggcaactggtcaattcatgcttgttacattcagaactcagga

SEQ ID NO: 82 - DNA, 60 bp, Mus musculus
tccctctctgtgaatccagattcaacactttcaatgtatgagagatgaattttgtaaaga

SEQ ID NO: 83 - DNA, 60 bp, Mus musculus
ttctcagttcagtggatatatgtatgtagagaaagagaggtaatattttgggctcttagc

SEQ ID NO: 84 - DNA, 60 bp, Mus musculus
ctgaccaaggtggctgactccagcccttttgcctctgaactgctaattccagatgactgc

SEQ ID NO: 85 - DNA, 60 bp, Mus musculus
gatacctggcttatcttttatcaacagcaaattatgcagtggtggaaatgtcatcacaga

SEQ ID NO: 86 - DNA, 60 bp, Mus musculus
gtttgagaagagacattatttataaaacccagatccttaatactgtttattacagccccg

SEQ ID NO: 87 - DNA, 60 bp, Mus musculus
ctctgatactgaataaacctgatgtgatgtacttatagtccttaagtcttgagagttaga

SEQ ID NO: 88 - DNA, 60 bp, Mus musculus
ggcaactacgactttgtagaggccatgattgtgaacaatcacacttcacttgatgtagaa

SEQ ID NO: 89 - DNA, 60 bp, Mus musculus
acttcataggattcacaatggagagggctaggaagatactggacaattttcagcagtgtg

SEQ ID NO: 90 - DNA, 60 bp, Mus musculus
cacctcttgtctccagccatgcccaggatcaattctagaatcagaggctacccctgcctg

SEQ ID NO: 91 - DNA, 60 bp, Mus musculus
cgtcagtgacccactcaatactgtggtgggaagtaagatgatgccaaatctataacctgt

SEQ ID NO: 92 - DNA, 60 bp, Mus musculus
cgaatgagaatgcatcttccaagaccatgaagagttccttgggtttgcttttgggaaagc

SEQ ID NO: 93 - DNA, 60 bp, Mus musculus
ccggcgggccctagtttctatgtatttagaatgaactcgtgtacatatgtaaagatcttt

SEQ ID NO: 94 - DNA, 60 bp, Mus musculus
caagctggttggagcctccagccttcaaaattctgaatctaataaacattaatgcacact

SEQ ID NO: 95 - DNA, 60 bp, Mus musculus
caatcctagaacaactacttgagtgttgtgagtgttcagatactcattaatatatatggg

SEQ ID NO: 96 - DNA, 60 bp, Mus musculus
tcccacctctctgatgagttatagccaagaagccttaggagtctccataaggcatattca

SEQ ID NO: 97 - DNA, 60 bp, Mus musculus
aagaaatattcccacttcagagtgtgtaagcaatatttaaacccagataaagatgcatgc

SEQ ID NO: 98 - DNA, 60 bp, Mus musculus
tttgggagtgggcttcatgaatgcgctcttaccaaaggagccatgtttccattgtatcaa

SEQ ID NO: 99 - DNA, 60 bp, Mus musculus
tttcattaaactaatatttattgggagaccactaagtgtcaaccactgtgctagtagaag

SEQ ID NO: 100 - DNA, 60 bp, Mus musculus
aagtgactccattttcatatgtacttaaacacagagttcctgtggcctctgtaagctcag

SEQ ID NO: 101 - DNA, 60 bp, Mus musculus
caaggtgaagagcctggaaactgagaacaggagactggagagcaaaatccgggaacatct

SEQ ID NO: 102 - DNA, 60 bp, Mus musculus
gcatgtgattgattcatgatttccccttagagagcaagtgttaccaaagttctgttgagc

SEQ ID NO: 103 - DNA, 60 bp, Mus musculus
tgctccagatgtgaaacttatagacgtagactaccctgaagtgaatttctatacaggaag

SEQ ID NO: 104 - DNA, 60 bp, Mus musculus
tgtacaactgaactcacctcttgtgaagaattatgattgtcttacttgtaaagaaagcac

SEQ ID NO: 105 - DNA, 60 bp, Mus musculus
ttttgcaggggtcgagtgtgatgcattgaaggttaaaactgaaatttgaaagagttccat

SEQ ID NO: 106 - DNA, 60 bp, Mus musculus
caaacagaaaacagggagatgtaaaacagtttcaactccatcagttatgaaaccatagct

SEQ ID NO: 107 - DNA, 60 bp, Mus musculus
tcagcaaattggcgatttcggaatcctatgacacctacatcaataggagtttccaggtga

SEQ ID NO: 108 - DNA, 60 bp, Mus musculus
catgtgcaacctcatgggaaaaatagtaacttgaatcttcagtggttagaaattaaagac

SEQ ID NO: 109 - DNA, 60 bp, Mus musculus
gtctcaaggatctgggaccagaactgggaaagaaaaggaatgaccaagacaagatcatac

SEQ ID NO: 110 - DNA, 60 bp, Mus musculus
tgaatcagagaaaagagagttggtgtttaaagaatatgggcaagagtatgctcaggtgac

SEQ ID NO: 111 - DNA, 60 bp, Mus musculus
aaaggaaatcatatcaggataagatttgtatctgatgagtattttccatctgaacccgga

SEQ ID NO: 112 - DNA, 60 bp, Mus musculus
cagtcctcttgaaaggtctcagaagctggtgagcaattacttggagggacatgactaatt

SEQ ID NO: 113 - DNA, 60 bp, Mus musculus
agaggagtctccttatattaatggcaggcattatagtaaaattatcatttcccctgagga

SEQ ID NO: 114 - DNA, 60 bp, Mus musculus
gcatgagtgtataggtgaaggtttcactttaagatgctgtcttcagttctcttgcctatg

SEQ ID NO: 115 - DNA, 60 bp, Mus musculus
atcgtctctgattatgacaagggctatgtggtgtggcaggaggtatttgataataaagtg

SEQ ID NO: 116 - DNA, 60 bp, Mus musculus
ctgttcgtgttgggttttgttcatgtcagatacgtggttcattctcaggaccaagggaaa

SEQ ID NO: 117 - DNA, 60 bp, Mus musculus
gtgcaatagaaatatatgatttcaaacacatttctgaactgccagggcaagaaagtatag

SEQ ID NO: 118 - DNA, 60 bp, Mus musculus
cttgtcgtttttgggggttgtaatatctaagggtgaaaaaattaatttccaaagccaaga

SEQ ID NO: 119 - DNA, 60 bp, Mus musculus
caactgtttacctggaaatgtagtccagaccatatttatataaggtatttatgggcatct

SEQ ID NO: 120 - DNA, 60 bp, Mus musculus
ccttccagagctttgccaaatttggaaaatttggagatgacctgtactccggatggttgg

SEQ ID NO: 121 - DNA, 60 bp, Mus musculus
taggtgagttaggaatctgccataaggtcgtttataggatctgtttatatgaagtaatgg

SEQ ID NO: 122 - DNA, 60 bp, Mus musculus
atgactttctctgcttggttggagaagaagaatctttactattcagcttcttttcttttt

SEQ ID NO: 123 - DNA, 60 bp, Mus musculus
ccggggtgggaagttgttttttcctgggggttttttccccttatttgttttggggcccct

SEQ ID NO: 124 - DNA, 60 bp, Mus musculus
ggaagatgggtaaatagtagactgtggtgtatttggaacaaggtagctttaaagacacaa

SEQ ID NO: 125 - DNA, 60 bp, Mus musculus
ccaggttcagagcggactgctaataataatgtgtgtattgatcgaggaaaaagtgcggag

SEQ ID NO: 126 - DNA, 60 bp, Mus musculus
tgcatgggaaatttctacgtggctcacttcaccaaggcttattgcactgggaaaagaaga

SEQ ID NO: 127 - DNA, 60 bp, Mus musculus
ttaacctaaaggtgcaaccttttaatgtgacaaaaggacagtattctacagctcaagact

SEQ ID NO: 128 - DNA, 60 bp, Mus musculus
tccccactactataaggccaaggagctagaagatccccatgctaagaaaatgcccaaaaa

SEQ ID NO: 129 - DNA, 60 bp, Mus musculus
ataggtactccccgattcccaaggagcagctagtggaaccctggagttttgggtagtaga

SEQ ID NO: 130 - DNA, 60 bp, Mus musculus
agtagtatttccagtattctttataaattccccttgacatgaccatcttgagctacagcc

SEQ ID NO: 131 - DNA, 60 bp, Mus musculus
accgctacttggagcctgttcactgtgtttattgcaaaatcctttcgaaataaacagtct

SEQ ID NO: 132 - DNA, 60 bp, Mus musculus
tgaactctgaccttttgcaacttctcatcaacagggaagtctcttcgttatgacttaaca

SEQ ID NO: 133 - DNA, 60 bp, Mus musculus
gtctgttcttgggaatggtttaagtaattgggactctagctcatcttgacctagggtcac

SEQ ID NO: 134 - DNA, 60 bp, Mus musculus
ccagcctgaccagattttagttaccttttaaggaagagagatttattctaatgccataaa

SEQ ID NO: 135 - DNA, 60 bp, Mus musculus
cacctctgtgctttgaaggttggctgaccttattcccataatgatgctaggtaggcttta

SEQ ID NO: 136 - DNA, 60 bp, Mus musculus
ctgagctcaggctgagcccacgcacctccaaaggactttccagtaaggaaatggcaacgt

SEQ ID NO: 137 - DNA, 60 bp, Mus musculus
agaacagcagttagttcctggttctgagaaccacttgtcccagtatgacacctcttacta

SEQ ID NO: 138 - DNA, 60 bp, Mus musculus
atgtgtgtactcaggacagaatccagagatttcttttttatatagcttgatataaaacag

SEQ ID NO: 139 - DNA, 60 bp, Mus musculus
acgtttcacacagtggtatttcggcgcctactctatcgctgcaggtgtgctcatctgtct

SEQ ID NO: 140 - DNA, 60 bp, Mus musculus
ttttttaattctgcaaattgtctcacagtggaatgaggaaatgagttagagatcacagcc

SEQ ID NO: 141 - DNA, 60 bp, Mus musculus
gtgctatctttactcactcccaagacatacacaggagcctttaatctcattaaagagaca

SEQ ID NO: 142 - DNA, 60 bp, Mus musculus
gaggtccaagtttaaatgttagtctcctaacaactgtcaaatcaatttctagcctctaaa

SEQ ID NO: 143 - DNA, 60 bp, Mus musculus
cttctagatccttctgcagaaatcatcgtcctaaaggagcctccaactattcgaccgaat

SEQ ID NO: 144 - DNA, 60 bp, Mus musculus
acttattcatccttgcctatacccaccccccaaaaacaggttttattaataaaaaatgtg

SEQ ID NO: 145 - DNA, 60 bp, Mus musculus
tacagtaacaagcaagctatcatccatttttacaataaagttgtcagcattcatgtcagc

SEQ ID NO: 146 - DNA, 60 bp, Mus musculus
ttatttactttatcttagtatgtaaccttagctgacctgaaacccactggtagactagac

SEQ ID NO: 147 - DNA, 60 bp, Mus musculus
cctgtcctgagttcatggccaaaacttaaataagagaaggaggagagggtcagatggata

SEQ ID NO: 148 - DNA, 60 bp, Mus musculus
aaaggggcctgagtatacgctgttgcaagctgtatacttcatttccttcggctggtttat

SEQ ID NO: 149 - DNA, 60 bp, Mus musculus
tatccggacagtctatgtgaaataggaccaaggtcgaaagccggaaagacatcaacagaa

SEQ ID NO: 150 - DNA, 60 bp, Mus musculus
ctgcttttccctgacatggatgcgtaatcacggggtcaaattacacctatccaacaccat

SEQ ID NO: 151 - DNA, 60 bp, Mus musculus
aacaaagaggacagtatgaatttgaatagctcccactagataagcaatttccacgagaac

SEQ ID NO: 152 - DNA, 60 bp, Mus musculus
ctgactgtgaatgtcgtgactcagagcaaagacagagaatatatttaattcatgttgtac

SEQ ID NO: 153 - DNA, 60 bp, Mus musculus
gcctgaagaacatgacagaactcttctcaatattcgttgggctttcagaatcataaacat

SEQ ID NO: 154 - DNA, 60 bp, Mus musculus
cctgtgtgaataaaaatacaagaactgcttataggagaccagttgatcttgggaaacagc

SEQ ID NO: 155 - DNA, 60 bp, Mus musculus
gtaagaaatattagactgattggagttaaagtagcactctacatttaccatggtgtttgg

SEQ ID NO: 156 - DNA, 60 bp, Mus musculus
tgtgaaagattgtgcatctgcattcaactaccctgaacccttagggaagaaatggattcc

SEQ ID NO: 157 - DNA, 60 bp, Mus musculus
agctgcctactagcagtttaacaaggagccttgctgtctcagacaggtgaaagaaaatgt

SEQ ID NO: 158 - DNA, 60 bp, Mus musculus
ccatgtttgaaagtatgtaatgaagaggagcctattaaccatatgaaagacaggaatact

SEQ ID NO: 159 - DNA, 60 bp, Mus musculus
gtgaattggatgcatagcatgttttgtatgtaaatgttccttaaaagtgtcaccatgaac

SEQ ID NO: 160 - DNA, 60 bp, Mus musculus
acccactgactaggataactggaaaggagtctgacctgaatgacgcattaaactcctgca

SEQ ID NO: 161 - DNA, 60 bp, Mus musculus
cccgcttcaatgagaacaacaggagagtcattgtgtgtaacacgaagcaggacaataact

SEQ ID NO: 162 - DNA, 60 bp, Mus musculus
atattaactctataaaataaggctgtctctaaaatggaacttcctttctaagggtcccac

SEQ ID NO: 163 - DNA, 60 bp, Mus musculus
tgtgggttttttgaagaattaatgagcatgtacatagaaatagtgactgcttgaatcctg

SEQ ID NO: 164 - DNA, 60 bp, Mus musculus
ctactcttaatgatgttatcttaacactgaaattgcctgaaacccatttacttaggactg

SEQ ID NO: 165 - DNA, 60 bp, Mus musculus
tcgaccatttctaggcacagtgttctgggctatggcgctgtatggacatatcctatttat

SEQ ID NO: 166 - DNA, 60 bp, Mus musculus
tctgaatctgggcactgaagggatgcataaaataatgttaatgttttcagtaatgtcttc

SEQ ID NO: 167 - DNA, 60 bp, Mus musculus
gatccttaggtctccataggatgatttttgaggtagttaatcagtgtaaactcttacaca

SEQ ID NO: 168 - DNA, 60 bp, Mus musculus
ctcagcagtaacagagaaaagatgaatgaagccactgaggcttcgtgaatgaatgaatct

SEQ ID NO: 169 - DNA, 60 bp, Mus musculus
ctttgttcctacccagccaccaaagccacctacataacaatccactcatgtactagcaaa

SEQ ID NO: 170 - DNA, 60 bp, Mus musculus
aaattgtctacgcatccttatgggggagctgtctaaccaccacgatcaccatgatgaatt

SEQ ID NO: 171 - DNA, 60 bp, Mus musculus
acatgatgtgaaagaatcattgaagatcacagttgtctaccgagttcagatttccttaca

SEQ ID NO: 172 - DNA, 60 bp, Mus musculus
caccccccagaaaatgagactattgaacattttcctttgtggtaagatcactggacagga

SEQ ID NO: 173 - DNA, 60 bp, Mus musculus
agtgatggggaccatgacgagctgtagcctgaacctcaaggcctgcaaccagtctactga

SEQ ID NO: 174 - DNA, 60 bp, Mus musculus
aaaggtcccaggtttcgatctgtttggagtttggagtctaatggttgcatagataaacag

SEQ ID NO: 175 - DNA, 60 bp, Mus musculus
tctatgtgcattagggggtgacccagggaaatccaaagggaacagtatttgatttctcac

SEQ ID NO: 176 - DNA, 60 bp, Mus musculus
ctacacatgtactttaggattctaggtttctccctgagccctgctttcgatgtaacactg

SEQ ID NO: 177 - DNA, 60 bp, Mus musculus
aagtctaaagggaatggcttactcaatggcctttgttctgggaaatgataagataaataa

SEQ ID NO: 178 - DNA, 60 bp, Mus musculus
ggaagaaaaagacctcaggaaaaaatttaagtacgacggtgaaattcgagttctatattc

SEQ ID NO: 179 - DNA, 60 bp, Mus musculus
ggatgaagaaactgagtttgtcccttctgagatcttcatgcaccaagcaatccacaccat

SEQ ID NO: 180 - DNA, 60 bp, Mus musculus
ctgttcaggctcaaacaatgggttcctccttggggacattctacatcattccaaggaaaa

SEQ ID NO: 181 - DNA, 60 bp, Mus musculus
aggagttcccagttttgacacatgtatttatatttggaaagagaccaacactgagctcag

SEQ ID NO: 182 - DNA, 60 bp, Mus musculus
ctcaataaaagctctaaggagacatcacaacccagtcttaagggttcatgaggttttaat

SEQ ID NO: 183 - DNA, 60 bp, Mus musculus
acttaaaatgtagactgttcatacagtgggtaccagtatgagttgaatgtgtgtattact

SEQ ID NO: 184 - DNA, 60 bp, Mus musculus
tttcataatagaaccgtctaccagtgacctcttgattatgatttgatttgactgcaaaac

SEQ ID NO: 185 - DNA, 60 bp, Mus musculus
atccatgtggcatcaattcaattatgtataataatgactttacaagggccccttaaaacc

SEQ ID NO: 186 - DNA, 60 bp, Mus musculus
cacaaaagtcaaatgtggatatcgtacgctgcatcacgtcatagacaagtctaaagaaga

SEQ ID NO: 187 - DNA, 60 bp, Mus musculus
ctatcaggatagtgataagaacgtcattctccgacattatgaagacatggtagtcgatga

SEQ ID NO: 188 - DNA, 60 bp, Mus musculus
ggagatcatcactcttgtatgaaatatactaactccaaacctttttagagcagattaggc

SEQ ID NO: 189 - DNA, 60 bp, Mus musculus
actattaagcactcaggagaatgtaggaaagatttcctttgctacagtttttgttcagta

SEQ ID NO: 190 - DNA, 60 bp, Mus musculus
aaagagaaaatatgtcagatggtgataccagtgcaactgaaagtggtgatgaagttcctg

SEQ ID NO: 191 - DNA, 60 bp, Mus musculus
gagagaggaatggggcccagagaaaagaaaggatttttaccaaagcatcaacacaaccag

SEQ ID NO: 192 - DNA, 60 bp, Mus musculus
gttgtactactggaaagattttgctgggacatacaatatgtgtgagaaaaatagagttgt

SEQ ID NO: 193 - DNA, 60 bp, Mus musculus
agaccaaagacacggacattgtggatgaagccatctactacttcaaggccaatgtcttct

SEQ ID NO: 194 - DNA, 60 bp, Mus musculus
cagagcagggggcttttatttttattttttaatggaaaataatcaataaagacttttgta

SEQ ID NO: 195 - DNA, 60 bp, Mus musculus
cttggcagctctccttacttctgggacatttgccactgtggtactgccaggaaggaatct

SEQ ID NO: 196 - DNA, 60 bp, Mus musculus
acttatagaaaaggacaggttgaagcctaagaagaaagagaagaaagatccgagcgcgct

SEQ ID NO: 197 - DNA, 60 bp, Mus musculus
tagttcagtgaacaagtatctgtcaatgagtgagctgtgtcaaaatcaagttatatgttc

SEQ ID NO: 198 - DNA, 60 bp, Mus musculus
catgaatgtcaaaacctaattacaaagcatcggtctctttgttgtgaggtatcagaaccc

SEQ ID NO: 199 - DNA, 60 bp, Mus musculus
cctgtctcatgggagatttgaatcataaggagaatcactttttgtaactttattgaggaa

SEQ ID NO: 200 - DNA, 60 bp, Mus musculus
aagtaaatatgcaaaggagagaagttagagaaactcctctcataagaaaaatgtcttccc

SEQ ID NO: 201 - DNA, 60 bp, Mus musculus
tcggaactgtcccttaaggagggtgatatcatcaagatcctcaataagaagggacagcaa

SEQ ID NO: 202 - DNA, 60 bp, Mus musculus
ttagtgggctgaacctatcggttttaactggttgtcttaattaaccataaacttggagaa

SEQ ID NO: 203 - DNA, 60 bp, Mus musculus
ttttgtacaaccctgactcgttctccacaactttttctataaagcatgtaactgacaata

SEQ ID NO: 204 - DNA, 60 bp, Mus musculus
agacttggaaaaggcttgggtacaattaagaaaaaccctacatcccaccctcctcttgac

SEQ ID NO: 205 - DNA, 60 bp, Mus musculus
taataaagaaactgtggaaatacttggatttctactgaagacaaaagacttctaggctgg

SEQ ID NO: 206 - DNA, 60 bp, Mus musculus
aggttaaacatatattcttggaaacatgaaatcacaactctcaaaaaccgtgaaccacca

SEQ ID NO: 207 - DNA, 60 bp, Mus musculus
cctcgtgttgtcttctttggacctcagtttttccatgaaccagaagagaattggaacaag

SEQ ID NO: 208 - DNA, 60 bp, Mus musculus
aatagcaatgtatcaaacaatggatgtgaaaaagatgcgctctatcatcatgaaaatgcc

SEQ ID NO: 209 - DNA, 60 bp, Mus musculus
tctctggagaaatcagtaactgcaaaaggaagagagggtctttaaagcacatgtagtaat

SEQ ID NO: 210 - DNA, 60 bp, Mus musculus
tggaatgttgaagaatgaaatctcgagggaattagaggttgaggtcatctggatattcag

SEQ ID NO: 211 - DNA, 60 bp, Mus musculus
atagaaccaatgtaggaaaatcaggcaaaataaaatgatgatcagtccatgtcatcatgg

SEQ ID NO: 212 - DNA, 60 bp, Mus musculus
agatgggaaaaagtactgtaggttcctgaactctggatctcaagcagaaatgtactgtct

SEQ ID NO: 213 - DNA, 60 bp, Mus musculus
aggaaaaccccggtagttaggacatctgaattctcaattattggattgccaaaagtgaaa

SEQ ID NO: 214 - DNA, 60 bp, Mus musculus
gtttttggaatttggacctgaaaattgtacctcatggattaagtttgcagaattagagac

SEQ ID NO: 215 - DNA, 60 bp, Mus musculus
tgggacctgtgaagcgactgaagaaaatgtttgaaacaacaagattgcttgcaacaatta

SEQ ID NO: 216 - DNA, 60 bp, Mus musculus
tccattattacatacaacaatcaagaaaaagacagaaaactacccttagagagatcaggg

SEQ ID NO: 217 - DNA, 60 bp, Mus musculus
attcaacagcattctaggaaaatggcaagaaagtaaattatcatccatttcaggtctgtg

SEQ ID NO: 218 - DNA, 60 bp, Mus musculus
ccatatgatcacagtcgtgttaaactgcaaagtactgaaaatgattatattaatgccagc

SEQ ID NO: 219 - DNA, 60 bp, Mus musculus
gggccatattttaaagataaggagagagaaactagcatacagaattttcctcatattgag

SEQ ID NO: 220 - DNA, 60 bp, Mus musculus
gaaaggcgtttattcagaaaatgatggtaagattcagactttaaagcacagttagaccca

SEQ ID NO: 221 - DNA, 60 bp, Mus musculus
taaggtgttttctccagttaagttcagttcctgaatagtagtgattgccccagttgcaac

SEQ ID NO: 222 - DNA, 60 bp, Mus musculus
ccaccataaaggaaaaaggacatgtgtatgagtaggtgttcatctatgtgcataattggc

SEQ ID NO: 223 - DNA, 60 bp, Mus musculus
gcacaagatggagtcattaaaattaaggcatcatcattttcagcatataacatagcagag

SEQ ID NO: 224 - DNA, 60 bp, Mus musculus
gattaaaaacattagggatgagaaataataagggcttgcaactgtgtagaagctagagcc

SEQ ID NO: 225 - DNA, 60 bp, Mus musculus
tgaagtacactctctaaatgaaaatgggctataaatatgtttgagtaggataggaggaag

SEQ ID NO: 226 - DNA, 60 bp, Mus musculus
gtgtaagaaaagatgggactgacaataaaaatgaaggtcaggtaagaagtaccagactcc

SEQ ID NO: 227 - DNA, 60 bp, Mus musculus
gggaaatatgcagcgttctatgtttccataagtgattttagcagaatgaggtattatgtg

SEQ ID NO: 228 - DNA, 60 bp, Mus musculus
gtaggactgtagaactgtagaggaagaaactgaacattccagaatgtgtggtaaattgaa

SEQ ID NO: 229 - DNA, 60 bp, Mus musculus
tcataggtctccatttagttcaagtgttttatggacaatcagcaagtttaggctcatagg

SEQ ID NO: 230 - DNA, 60 bp, Mus musculus
ttggaatatatgaatgacaaagaaatgggaaaaactgctgaacccgagtctctgaatgtc

SEQ ID NO: 231 - DNA, 60 bp, Mus musculus
ctatcttgaattgctagattaaagagaaagaaaatgttagagcaaaataggaacctggcc

SEQ ID NO: 232 - DNA, 60 bp, Mus musculus
aatccctagagaaaatgggaatagaaataagctgcatacaaactcaaagacacagatact

SEQ ID NO: 233 - DNA, 60 bp, Mus musculus
agactgaagaaaaccttaaaatacccaaaattcaggggagacatagcaactgagtctcat

SEQ ID NO: 234 - DNA, 60 bp, Mus musculus
agaggacttcctgtctgtatcagatattattgactacttcaggaaaatgacgctgttgct

SEQ ID NO: 235 - DNA, 60 bp, Mus musculus
atggagatgtgtaaacagtaggacatttcgataactatgtcaggtcagttcttagttcag

SEQ ID NO: 236 - DNA, 60 bp, Mus musculus
gaggctattataaataacctgaaatgcatatgagaactgaacgtgtaataattcagctcc

SEQ ID NO: 237 - DNA, 60 bp, Mus musculus
aagtcggaatatgtcttagtgttcttctcacttagctcagtgtaagatggtagctcaagt

SEQ ID NO: 238 - DNA, 60 bp, Mus musculus
cacttttctatgaagaaagccgtgtgtaaagtttccgtgacagtagtaatggaaatatct

SEQ ID NO: 239 - DNA, 60 bp, Mus musculus
tgtaagaatacaaggtaaaacaaaatagagaaatacaggcatcatatctgcaaatcgccg

SEQ ID NO: 240 - DNA, 60 bp, Mus musculus
cagaaacagtagtatggggttaaatcacaatgagggaaattatagggatatgcagccaag

SEQ ID NO: 241 - DNA, 60 bp, Mus musculus
actgaaagttggggagatacatgtaatttaataggatagggtacttaggtccagacaacc

SEQ ID NO: 242 - DNA, 60 bp, Mus musculus
aagctgttgaatatggacgtaactgtaaatcccagagtgttttattttgagatgagagtt

SEQ ID NO: 243 - DNA, 60 bp, Mus musculus
tttatcaaacatggaaacatctagagactatgggagagaaaatgggtttttagatatggg

SEQ ID NO: 244 - DNA, 60 bp, Mus musculus
ggaagttaatagaactgttcaaaatgtgaaagtggaaatagcgtcaataaggaaagcccc

SEQ ID NO: 245 - DNA, 60 bp, Mus musculus
agtgtagttttcagtggacagatttgttagcataagtctcgagtagaatgtagctgtgaa

SEQ ID NO: 246 - DNA, 60 bp, Mus musculus
gaaagtggggaatgaaaagtataacaaagtaaaaagagaatttctaggccctttaggccc

SEQ ID NO: 247 - DNA, 60 bp, Mus musculus
ggttttctcttgttttatcatgattctttttatgaagcaataaatccatttccctgttgg

SEQ ID NO: 248 - DNA, 60 bp, Mus musculus
ctttttgaggtttatttttccacagttttcatttgttcattaggcattttcccttttact

SEQ ID NO: 249 - DNA, 60 bp, Mus musculus
agtgtttttctttaattcttgaggttgttattgtaatatttacatatagtgcaagaatgt

SEQ ID NO: 250 - DNA, 60 bp, Mus musculus
taaagtatccactgaagtcactatggaaaacagccttttgatttatggactatttagctc

SEQ ID NO: 251 - DNA, 60 bp, Mus musculus
gcctagttttttcagcatcaattttggaaaaccttagaccacaggcatatttcgtcaagt

SEQ ID NO: 252 - DNA, 60 bp, Mus musculus
tcatttttcaagtcgtcaaggggatgtttctcattttccgtgacgacttgaaaaatgacg

SEQ ID NO: 253 - DNA, 60 bp, Mus musculus
ctgaaaatcacggaaaatgagaaatacacactttaggacgtgaaatatgtcgaggaaaac

SEQ ID NO: 254 - DNA, 60 bp, Mus musculus
gcgagaaaactgaaaatcacggaaaatgagaaatacacactttaggacgtgaaatatggc

SEQ ID NO: 255 - DNA, 60 bp, Mus musculus
agaaagctatggactggataggaggagaatgtaaatatttcagctccacattatttatag

SEQ ID NO: 256 - DNA, 60 bp, Mus musculus
acaaaaaggttacctatgaagacagtgaaataagagagaaatgtttagtacctcaggttg

SEQ ID NO: 257 - DNA, 60 bp, Mus musculus
ctaagggaggaaatgttggtataaaatgtttaaaagaacttggaggcaaacttggagtgg

SEQ ID NO: 258 - DNA, 60 bp, Mus musculus
ccacatcattggaaagaaatacacttatcttaattgccatggaataggagcatgaaagtc

SEQ ID NO: 259 - DNA, 60 bp, Mus musculus
atgagaaatacacactttaggacgtgaaatatggcgaggaaaactgaaaaaggtctattc

SEQ ID NO: 260 - DNA, 60 bp, Mus musculus
cctgtgaactgaaaatgcagatgatccacaggctaaatgggaaacctggagagtagatga

SEQ ID NO: 261 - DNA, 60 bp, Mus musculus
gcgagaaaactgaaaatcacggaaaatgagaaatacacactttaggaccagaaatatggc

SEQ ID NO: 262 - DNA, 60 bp, Mus musculus
tggaggaaattgattgaaaaacgattggtcaaatcgaaaatggagaaaactcatgttcac

SEQ ID NO: 263 - DNA, 60 bp, Mus musculus
cttcatcctggttttcacggcaataataatgatgaaaagacaaggtaaatcaaatcactg

SEQ ID NO: 264 - DNA, 60 bp, Mus musculus
actgaaaatcatggaaaatgagaaacatccacttgacgacttgaaaaatgacgaaatcac

SEQ ID NO: 265 - DNA, 60 bp, Mus musculus
caagcactgtgctgcaaaatgtcggtggaatatgataagttcctagaatctggacgaaaa

SEQ ID NO: 266 - DNA, 60 bp, Mus musculus
tttgagaagaaaggcatacacttgaaataaaggcaaaaacattatactgtctaccgagac

SEQ ID NO: 267 - DNA, 60 bp, Mus musculus
gaagaaaacgaggtgaagagcactttagaacacttggggattacagacgaacatatccgg

SEQ ID NO: 268 - DNA, 60 bp, Mus musculus
atcataaaaactgtggaaatccatattgcccttttaaaagaaaactatggggatggagag

SEQ ID NO: 269 - DNA, 60 bp, Mus musculus
aaatggcagaagaaagggttaatggctggaaaaatggatcagtagtcttgcagaggaacc

SEQ ID NO: 270 - DNA, 60 bp, Mus musculus
attttagggggctttattgttacttgacgtggaatttgaaaactaaaaagatgagtctgg

SEQ ID NO: 271 - DNA, 60 bp, Mus musculus
gtggaaatcagagatctaagtacgtttatgcataggagtaggaatgaggggttattaaag

SEQ ID NO: 272 - DNA, 60 bp, Mus musculus
aaaccccccaagtagcccaaaggcccgcttcccaccaaaatgttttttatgttttaagga

SEQ ID NO: 273 - DNA, 60 bp, Mus musculus
attatgatgcctgtaacacacagaagtatctgactgtgaacgaatcaacctcatggatga

SEQ ID NO: 274 - DNA, 60 bp, Mus musculus
agaagagatactgagccaatgaaccctttcgtataggattcatgacaaaaccaaactcag

SEQ ID NO: 275 - DNA, 60 bp, Mus musculus
ctgccttcccataaaaataaaaggcatgcaaaaccaatttttggccaggcccagttaaga

SEQ ID NO: 276 - DNA, 60 bp, Mus musculus
acaagccctgggcctctgagaccacccgacacaccatcctaccaagaagcctctaagtat

SEQ ID NO: 277 - DNA, 60 bp, Mus musculus
caagtcagcaagaagccaaccttggtgaaataattctggttgtttgaaagctaggtcttg

SEQ ID NO: 278 - DNA, 60 bp, Mus musculus
ggtcaagagagtgccaactagctttgtttaaaaaatcctagtcctgaatccacaagcctg

SEQ ID NO: 279 - DNA, 60 bp, Mus musculus
agtggaagccttataagcattgaacccaggatgagtcgctcgtatttccaccttactcat

SEQ ID NO: 280 - DNA, 60 bp, Mus musculus
cttcccacaaccccaccgtaccttgtctatgtatgcatgtttttgtaaaaaagaaaaaag

SEQ ID NO: 281 - DNA, 60 bp, Mus musculus
tgcctgactccaagaaaagaagccagaactcggaaccatagtcatctttaaagatcttct

SEQ ID NO: 282 - DNA, 60 bp, Mus musculus
gttaatattattaactgagcctgcccataccccccgtggtcattggtgttgggtgcagtg

SEQ ID NO: 283 - DNA, 60 bp, Mus musculus
ggaggacgacatcctcatggacctcatctgaacccaacacccaataaagttccttttaac

SEQ ID NO: 284 - DNA, 60 bp, Mus musculus
tctgaacctcaacccatcaccaaccccgtgtcttcaacattactttccaaaaaagtctgg

SEQ ID NO: 285 - DNA, 60 bp, Mus musculus
aggagcctgtgtccttatagagttggaattaacttcagccctctatctcacttcctctgt

SEQ ID NO: 286 - DNA, 60 bp, Mus musculus
gaaaaaagatgagatctcctccatgacaagagcctgcatacaacatttgagtacccttct

SEQ ID NO: 287 - DNA, 60 bp, Mus musculus
tttgattttagcagaaaccaccaccaaaattgtgccttagctgtatttctgtttagggga

SEQ ID NO: 288 - DNA, 60 bp, Mus musculus
agatactatggtactgtcatgaaatgcagtgggactctattcaaacaaccctccaaaatg

SEQ ID NO: 289 - DNA, 60 bp, Mus musculus
agagaacccacactcctttcatcaagacttgcagagcatcccacaaccaagatgctattt

SEQ ID NO: 290 - DNA, 60 bp, Mus musculus
tatgagcctgacccacactctctgtaaggtgtgactttataaatagacttctccgggtgt

SEQ ID NO: 291 - DNA, 60 bp, Mus musculus
ataccccaccacaacctctcaaaagagggctcttaacttggaaggataaaataaatcagg

SEQ ID NO: 292 - DNA, 60 bp, Mus musculus
tatcctcccacaaagatgagaggagcccatccagtgttactgttagaagtcacagtgaaa

SEQ ID NO: 293 - DNA, 60 bp, Mus musculus
tattgtccaatgaaacccacaaactaccctctatctggagttggaacatttatctgcatt

SEQ ID NO: 294 - DNA, 60 bp, Mus musculus
taaggagactgccctacaaaactacgatactactatcactttaaaaattagtgtaaaggg

SEQ ID NO: 295 - DNA, 60 bp, Mus musculus
tcaaggccaagtttctgcaagaagcaaggatcctgaaacagtacaaccaccccaacattg

SEQ ID NO: 296 - DNA, 60 bp, Mus musculus
gattgccagagacttacacttaatagagtcataaagcccatagagcctgagtgagagcca

SEQ ID NO: 297 - DNA, 60 bp, Mus musculus
ttattcctgaagcccccgctacagatgtttccacaaccgaagaagcggtctccaaagagc

SEQ ID NO: 298 - DNA, 60 bp, Mus musculus
agctccacatgaactcacagaagaaccaggctaagtacccaaggaccgagctcaaggaca

SEQ ID NO: 299 - DNA, 60 bp, Mus musculus
accattattcttttaaaaaacccaaaaaccaccagcaagggggcctttggttggcctcaa

SEQ ID NO: 300 - DNA, 60 bp, Mus musculus
cttcatcttaaaactccagaacaactcccttcctaacctggaacccagcagctttcagtt

SEQ ID NO: 301 - DNA, 60 bp, Mus musculus
ctgcacgccccaggagcctgggtgaagcatcacagcactaagtcatgttaaaaggagtct

SEQ ID NO: 302 - DNA, 60 bp, Mus musculus
cactggagcactgaacatgatgtacaagtatcacacagaaaagcagcactggactgtact

SEQ ID NO: 303 - DNA, 60 bp, Mus musculus
ataagaacttataggaaccccaactccccatgaaaaatataagacctcaaggcctgggga

SEQ ID NO: 304 - DNA, 60 bp, Mus musculus
gcccaccaactctaatttgtgctacttatatatattcctgggagtaggactgtcctcctg

SEQ ID NO: 305 - DNA, 60 bp, Mus musculus
cagtcaggtcttccagaacaattacaaccccgaggagaacctcaatgacgtgcttctcct

SEQ ID NO: 306 - DNA, 60 bp, Mus musculus
cgtagctcgctggtagaaagcctgaccaccatgcatacgatcctgggtttcaacaaggaa

SEQ ID NO: 307 - DNA, 60 bp, Mus musculus
gagcctgagatctacgagcccaatttcatcttcttcaagaggatttttgaggctttcaag

SEQ ID NO: 308 - DNA, 60 bp, Mus musculus
gagtctgtgggtattcgcctgaacaagcataagcccaacatctatttcaagcccaagaaa

SEQ ID NO: 309 - DNA, 60 bp, Mus musculus
agcatcaaacaaagcacataaactcgtacataagcaagggatgtccttattggtcaaaca

SEQ ID NO: 310 - DNA, 60 bp, Mus musculus
gggaaaaaatagcaaaaccccaaactccacaaccacaaaaacctgttaattatggtggca

SEQ ID NO: 311 - DNA, 60 bp, Mus musculus
acacagagccagaaaacccaggcctgaagacatcccctagtcctgctgagagaccacagt

SEQ ID NO: 312 - DNA, 60 bp, Mus musculus
cgaccaatctgcctgggaaacaacaccccacagaacggggcttcagaaacacgtgagtga

SEQ ID NO: 313 - DNA, 60 bp, Mus musculus
gtttaggtgagtttccattgtatcttataacagagaaacccattaggcagtagttagttc

SEQ ID NO: 314 - DNA, 60 bp, Mus musculus
tcgaaacacctaccaaataccaataataagtccaataacattacaaagatgggcatttcc

SEQ ID NO: 315 - DNA, 60 bp, Mus musculus
tgctaccctccaggaccaacgatggatgcaccacggagtcccaagagctgaaaagcagaa

SEQ ID NO: 316 - DNA, 60 bp, Mus musculus
cggagctcttcagaaccccaactctctctggctggctacccccagaactcctaggtttat

SEQ ID NO: 317 - DNA, 60 bp, Mus musculus
ataaagagaattcccaccaccctgggcgaaggaattaccagcaataaaacctattccttc

SEQ ID NO: 318 - DNA, 60 bp, Mus musculus
actttcaagtctgaatcctatgagcctgaagtgagatcttatttagaaacagaaccccaa

SEQ ID NO: 319 - DNA, 60 bp, Mus musculus
gacaagcccttagggagccagaaaaagagcaggaagaagttaaaatgtttaattttttaa

SEQ ID NO: 320 - DNA, 60 bp, Mus musculus
gcccaagagctagaaaacctactctatgtgtagagatacttcctattaaaataatagtac

SEQ ID NO: 321 - DNA, 60 bp, Mus musculus
ctccacttttaaagtctgtaggaataggagccgattagacaactctcggtctcatgctca

SEQ ID NO: 322 - DNA, 60 bp, Mus musculus
tttctgggatcccactgcaccgccatttcttcccagatttatgtgtataacttaaactgg

SEQ ID NO: 323 - DNA, 60 bp, Mus musculus
atacagtagatgctgaacacacttgagtccatcatgagggggtaataagtctcaccagca

SEQ ID NO: 324 - DNA, 60 bp, Mus musculus
tcttatactttcaacaaagctgaaccctaacattacactaaccagcagctcaacacgagt

SEQ ID NO: 325 - DNA, 60 bp, Mus musculus
ctgaatgtatacacacccacaggagactgtggctgagcgttcatccaaataaatttgaat

SEQ ID NO: 326 - DNA, 60 bp, Mus musculus
gttcctgttcagagtgcctgaaaacccaaagtgtctgagagtctgaaggaattcaactgt

SEQ ID NO: 327 - DNA, 60 bp, Mus musculus
aaacacccacacttgaaacttccatgaacccactcaaattcatttctatccccctttgga

SEQ ID NO: 328 - DNA, 60 bp, Mus musculus
tcatggagatataactatagagataaagagcgacaccctgtctgaagcaatcagcgtccg

SEQ ID NO: 329 - DNA, 60 bp, Mus musculus
ggacactgtgaacactgtgtggacagagcccacaacttctccatttgtgtctggcagcaa

SEQ ID NO: 330 - DNA, 60 bp, Mus musculus
aggaaagaaaggggttagaatctctcaggagattaaagtttctgcctaacaagaggtgtt

SEQ ID NO: 331 - DNA, 60 bp, Mus musculus
ctcaagactttgccaacatgttccgtttcttacaccctgaaccctgatcggaacattcat

SEQ ID NO: 332 - DNA, 60 bp, Mus musculus
tctgtacatggccgaaaatcagagtccaccatattcttttgaatatccagggttctctga

SEQ ID NO: 333 - DNA, 60 bp, Mus musculus
ttctggctccttatttcagttctctttaaaaccagttcaacaccagtgtgttaaaaagaa

SEQ ID NO: 334 - DNA, 60 bp, Mus musculus
gcagatttaacaactagcaactctgtcatctttttctaaaaatgaccaactgctgattac

SEQ ID NO: 335 - DNA, 60 bp, Mus musculus
cttaaaaagggagatacagttttactctgatccagcaaatctagttaagacactagaatg

SEQ ID NO: 336 - DNA, 60 bp, Mus musculus
cttcctgaaccattaccagatggaaaacccaaatggcccgtacccatatactctgaagtt

SEQ ID NO: 337 - DNA, 60 bp, Mus musculus
gtaacggagcctgggggttgaaggttatctttacatatatgtacaaactgttgtcaagag

SEQ ID NO: 338 - DNA, 60 bp, Mus musculus
tccccaccactcatggggatcttcaagaagcatcaccattcactgaaaggtcctaaaaaa

SEQ ID NO: 339 - DNA, 60 bp, Mus musculus
gcgcagaggcaaaccaacgtggagccagacattggtgaacccaacctatccacaccttca

SEQ ID NO: 340 - DNA, 60 bp, Mus musculus
cttattttagacagatccaaagttctcacaagccccctttctttgctctgcctatcatcg

SEQ ID NO: 341 - DNA, 60 bp, Mus musculus
aacctctgaacctaatcactgtggattcccaccaacaccatatatgaaaatgcaggccga

SEQ ID NO: 342 - DNA, 60 bp, Mus musculus
tgcggaaggaggggattcaaaccagaaaacggaagcccaagaacctgaataaatctaaga

SEQ ID NO: 343 - DNA, 60 bp, Mus musculus
ccctagtccgttttctgatcagtcagaacccacaataactactagtagtcctgtggcttt

SEQ ID NO: 344 - DNA, 60 bp, Mus musculus
gtagccaccaagccacaagtaacaaatgatctctgtgaatgccatatggaaacttttatt

SEQ ID NO: 345 - DNA, 60 bp, Mus musculus
ggctccatttctgaactctgtgttaagctaataagattttaaataaacgctgatgaaagc

SEQ ID NO: 346 - DNA, 60 bp, Mus musculus
tgctgggggcctagaaccctgagacatagaccatggataaatggcaaccggggtggcaaa

SEQ ID NO: 347 - DNA, 60 bp, Mus musculus
aacgcaaagagcaagaaccaaacaaagacaggaacaactcgcagaagaaatcccgcctgg

SEQ ID NO: 348 - DNA, 60 bp, Mus musculus
tgttttctgatgaccaaagcaatgacaaggagcagaaagaagaactgaacgaattgatca

SEQ ID NO: 349 - DNA, 60 bp, Mus musculus
cccaccactgaatatagaccatactgtgagaggaccataattaggtcctgaatttttaat

SEQ ID NO: 350 - DNA, 60 bp, Mus musculus
gtatgacttccaaccagaaaaaggctctaaaagctgaacacactaaccggctgaaaaacg

SEQ ID NO: 351 - DNA, 60 bp, Mus musculus
cttctggctcccttacatgaaggactgatttaagaaaccagaccattcctttactttgaa

SEQ ID NO: 352 - DNA, 60 bp, Mus musculus
gcagggtgcttactttctcagagcctgaagttacttccattgttttggcactgaataaca

SEQ ID NO: 353 - DNA, 60 bp, Mus musculus
ttagcacaagagaaaagctgagaacgtgggttttgcctccttcagaaatatgtctggctc

SEQ ID NO: 354 - DNA, 60 bp, Mus musculus
acacagcacccacaactaatcttgggacacccctatctggttggaagagagtaaactaat

SEQ ID NO: 355 - DNA, 60 bp, Mus musculus
caatggcctattctgtcagatgggtgtcctttcaagggtgacaactacagaacacaagta

SEQ ID NO: 356 - DNA, 60 bp, Mus musculus
aaagtaggttcacacagtaaagggataataccatctggaacaatgatcagtgtagagtta

SEQ ID NO: 357 - DNA, 60 bp, Mus musculus
cacctgggtctacagctactctgattctacaaagacagggtcaagcatctctaacaaagt

SEQ ID NO: 358 - DNA, 60 bp, Mus musculus
tattaaacccaggagatacaaggagtctgccattaacctctctgtaactcaagagtagtt

SEQ ID NO: 359 - DNA, 60 bp, Mus musculus
ttcctcccaaaatggagtttcctcttcaaaccacagctcccccaagatctatcctgatat

SEQ ID NO: 360 - DNA, 60 bp, Mus musculus
tatgtcttgatactggacccacactactggggcactccaaaaaaccgttgtgaactacaa

SEQ ID NO: 361 - DNA, 60 bp, Mus musculus
agtaaagggcaccggaaatgttaaatccttgtttaggatatgaaaggaattaggggatgg

SEQ ID NO: 362 - DNA, 60 bp, Mus musculus
gaatgtctgatacatgacccatcagttaggaaccactgaactagaggagtagctaaactc

SEQ ID NO: 363 - DNA, 60 bp, Mus musculus
gcttctactggctcttgtatgcatatgtgcacttatccagactgaggattttacaaagca

SEQ ID NO: 364 - DNA, 60 bp, Mus musculus
ctgtctaagcgctgaaccacttagcagaaatgacacccatatgagagcttgtgccaaata

SEQ ID NO: 365 - DNA, 60 bp, Mus musculus
aaaggagactgcatcaggtattctgatagagagctgaggaagagattgaggtatgggatt

SEQ ID NO: 366 - DNA, 60 bp, Mus musculus
tgactggaatcaccacccttgcctgagtttgcgatctcacagttggaactgagagtttcc

SEQ ID NO: 367 - DNA, 60 bp, Mus musculus
ggatcagatgatgcaccattgctttccattgctacatttaaaatcttttactagtcaacc

SEQ ID NO: 368 - DNA, 60 bp, Mus musculus
ttgagaccttaaagaaataacaaactcaaggaagattagggtccagtgtttaagtcatgg

SEQ ID NO: 369 - DNA, 60 bp, Mus musculus
gtctcctttgtgttattgccttcccaacacttctaagtcccagctcaacagctacttcta

SEQ ID NO: 370 - DNA, 60 bp, Mus musculus
cacagctgcttgtagtcatcattccagtgaggagtaagaagaattttatgtgtgtctcta

SEQ ID NO: 371 - DNA, 60 bp, Mus musculus
aacttaaacagtctcccaccacctaccccaaaagatactggttgtattttttgttttggt

SEQ ID NO: 372 - DNA, 60 bp, Mus musculus
cagcagaaaaggctcccaccaagaaggccaacagcacaaccacagccagcaggatgtgtt

SEQ ID NO: 373 - DNA, 60 bp, Mus musculus
ggcttcacatctaagtggggactattttaacttatttacaggtatatggtgtggaaataa

SEQ ID NO: 374 - DNA, 60 bp, Mus musculus
cgctcagttgtagaaagcaacaaggacacaaacttgattgcccaaagtcactgccagtta

SEQ ID NO: 375 - DNA, 60 bp, Mus musculus
gtctgaacacactattatgtatccatccaatctcaactgaataaagggagatgccttttg

SEQ ID NO: 376 - DNA, 60 bp, Mus musculus
aaagaatttcaagaacgaagcataggtggttatgtagtttgattacagaaaagagatgcc

SEQ ID NO: 377 - DNA, 60 bp, Mus musculus
aaaccaccttcagtgtgaggagcccacgtcagttgtagtatctctgttcataccaacaat

SEQ ID NO: 378 - DNA, 60 bp, Mus musculus
gcactccagcctgattctttgagactttggggtacacatattgaaagtactttgaatttg

SEQ ID NO: 379 - DNA, 60 bp, Mus musculus
actgtatcggttccatgtaagtctgaccagtcaaaggcaagaggtatcaaggtggagaaa

SEQ ID NO: 380 - DNA, 60 bp, Mus musculus
gtgtttgaattaaaacccccaccctcggaggcctttaaagaaatggtttttgtccgttgt

SEQ ID NO: 381 - DNA, 60 bp, Mus musculus
ctctcgacaaaatataaatggacagtaccaaactaagagggatataagtgggagcaaagg

SEQ ID NO: 382 - DNA, 60 bp, Mus musculus
tatggtacgagtttagggcttagtcagtttacaatggggattgaattttgtgtcaaaacc

SEQ ID NO: 383 - DNA, 60 bp, Mus musculus
ctggctcctactggcaacaggcatacttgtggtttaatacagagaaacaaaacattcata

SEQ ID NO: 384 - DNA, 60 bp, Mus musculus
tttgacctaatgaaatacccatttcatctgtgacaacacatagcccagtaaacatcactg

SEQ ID NO: 385 - DNA, 60 bp, Mus musculus
cctgttcctagtatcctggcgtccacatatacccaaagttaggcatactaaccaagagat

SEQ ID NO: 386 - DNA, 60 bp, Mus musculus
ctggaactcagcactgcccaccacacttggtccgaaatgccaggtttgcccctcttaagt

SEQ ID NO: 387 - DNA, 60 bp, Mus musculus
cctggaggtctccacctgaagttccctgatgcagggtcagtccagccttggtaagggcca

SEQ ID NO: 388 - DNA, 60 bp, Mus musculus
aaatgagaaccagattaccaaaattaccactaccaccaaaataacccctctgattccttg

SEQ ID NO: 389 - DNA, 60 bp, Mus musculus
cagatagatgacagcaggaaattttctttatttcctgaaagaaaataccagactctcaac

SEQ ID NO: 390 - DNA, 60 bp, Mus musculus
ggtgccaaatgcggccatggtgctgaacaatttatcgtcagaggggaagaacagttgacc

SEQ ID NO: 391 - DNA, 60 bp, Mus musculus
ccaaaacagagccaacaccaccgacaacaaccccacagcaaacccggagagaaacccaaa

SEQ ID NO: 392 - DNA, 60 bp, Mus musculus
tttcaacccgcccattatttccagatttatccgcatcattcctaaaacatggaaccagag

SEQ ID NO: 393 - DNA, 60 bp, Mus musculus
tggagactgagttcgacaatcccatctacgagactggcgaaacaagagagtatgaagttt

SEQ ID NO: 394 - DNA, 60 bp, Mus musculus
gatacaacagcatctgttttccaaggagaaatcatttgaggaacaaaacctatcaagaga

SEQ ID NO: 395 - DNA, 60 bp, Mus musculus
aactagaaaacatagatgcacaggactcggatccatgatatttacactgggaaatgttct

SEQ ID NO: 396 - DNA, 60 bp, Mus musculus
atctcaagatttctatccaagtggaaacaaactgaatcatgcacacgacttatctgtgtg

SEQ ID NO: 397 - DNA, 60 bp, Mus musculus
agaggagccacacttgatgtgaattaaactcataaacattatgccactaacagcttttat

SEQ ID NO: 398 - DNA, 60 bp, Mus musculus
ctgccgcctgtacaaaggaaactgaacctttttcatattctaataaatcaatgtgagttt

SEQ ID NO: 399 - DNA, 60 bp, Mus musculus
aagctgagattaaacggctacacaataccatcatagatatcaacaaccgaaaactcaagg

SEQ ID NO: 400 - DNA, 60 bp, Mus musculus
gacttgggaaaacaatgcaactcccataaaccaaaactccaattccatgcctaacttgct

SEQ ID NO: 401 - DNA, 60 bp, Mus musculus
agcagggaacaatttgagtgctgacctataacacattcctaaaggatgggcagtccagaa

SEQ ID NO: 402 - DNA, 60 bp, Mus musculus
agctccaactcaacagatggctacacaggcagtgggaacactcctggggaggaccatgaa

SEQ ID NO: 403 - DNA, 60 bp, Mus musculus
actagctgcattgtaaagaaacaaatcgaaactgagtcttttcacatattgtgacggaca

SEQ ID NO: 404 - DNA, 60 bp, Mus musculus
gtagggtcatcatacacccagactaccgccaagatgaacctaacaattttgaaggagaca

SEQ ID NO: 405 - DNA, 60 bp, Mus musculus
tccccaccacgaattatcgtggctagtggatgaaggccactaatacaggttcaaattgtt

SEQ ID NO: 406 - DNA, 60 bp, Mus musculus
tatgtgcataggctggagttttggttatacatggtacacttttgggccaatataatagga

SEQ ID NO: 407 - DNA, 60 bp, Mus musculus
ccacactccctggagacaatgtctgccatttttgcatcacttgtcaaaccactaacttct

SEQ ID NO: 408 - DNA, 60 bp, Mus musculus
tcggttgacctgattccaccaaggagaaggagatcaaggaagagtaaactgtaagagcat

SEQ ID NO: 409 - DNA, 60 bp, Mus musculus
gagtgctttgatggttgttagggaccgtaagaatagtcctgtgtcagacagcagattcta

SEQ ID NO: 410 - DNA, 60 bp, Mus musculus
aactgtcataaaatccaacgtgccttcatgatcaaagttcgatagtcagtagtactagaa

SEQ ID NO: 411 - DNA, 60 bp, Mus musculus
actctcatctgtaaagccttcccatctcattattccttgcactaaccacagccactagga

SEQ ID NO: 412 - DNA, 60 bp, Mus musculus
cagactgaaaggaaattccaaagaaaacaaaaacctttcaatctatgaactcaatggctg

SEQ ID NO: 413 - DNA, 60 bp, Mus musculus
ctgagaataacctactaccacctctcttttcccaccaacatccaagtgccagcggtggtt

SEQ ID NO: 414 - DNA, 60 bp, Mus musculus
agcgacatgcaaccaaataccactcaaaacaaaaatccagcaaaactgagttgtgaggga

SEQ ID NO: 415 - DNA, 60 bp, Mus musculus
gtttgtacatgtaaaagattgaccagtgaagccatcctatttgtttctggggaacaatga

SEQ ID NO: 416 - DNA, 60 bp, Mus musculus
acttagaccacaacagcatctaagcatcattaccttaagtactaaagcaaaaatctagtc

SEQ ID NO: 417 - DNA, 60 bp, Mus musculus
taaaccactcttaaactgctggctccagtgtttttagaatgatatgaagtcattttggag

SEQ ID NO: 418 - DNA, 60 bp, Mus musculus
agtaagtgccattatccacccaactaccaaccaatgcctaagcagattctatatcttagc

SEQ ID NO: 419 - DNA, 60 bp, Mus musculus
gcttctggcagagatctgtttagcatagtgtggtattaattatagcaaatgttaaggtag

SEQ ID NO: 420 - DNA, 60 bp, Mus musculus
gttgtctgaataatagcacccaagaaaaagtgtggagatcagtaggtattcattaagcat

SEQ ID NO: 421 - DNA, 60 bp, Mus musculus
taaaggagctttccacatgaactcacaattttcttgaaataaacttcttaaccaactgcc

SEQ ID NO: 422 - DNA, 60 bp, Mus musculus
gtcacttggatggtgtatttatgcacaaaagggctcagagactaaagttcctgtgtgaac

SEQ ID NO: 423 - DNA, 60 bp, Mus musculus
gtcatgaacccaatacactgtggaaatgtgtgattctttatattaaacgtctgctgttca

SEQ ID NO: 424 - DNA, 60 bp, Mus musculus
tgtcgataccatctaaagaccacaacttctagccatagggtatttcatatatgtccattt

SEQ ID NO: 425 - DNA, 60 bp, Mus musculus
atgcaaacctaaaaagcacccaaaaaattcacattggactgaagaagagtgatccaagca

SEQ ID NO: 426 - DNA, 60 bp, Mus musculus
tttgagaccctttcataagcccaattatacagatatccaatattactgcaatcattggag

SEQ ID NO: 427 - DNA, 60 bp, Mus musculus
acctaaatttccacaggcaacttactttgttattaaatttggggatcatatcctgtgccc

SEQ ID NO: 428 - DNA, 60 bp, Mus musculus
ttttttcagacttaagaacagctaaacaaaaccttcctctagctttttcatcacatccag

SEQ ID NO: 429 - DNA, 60 bp, Mus musculus
ataatgatgatgataacaacaagaaaacagactcgaacctaaagacgctggtctcagata

SEQ ID NO: 430 - DNA, 60 bp, Mus musculus
cgcaaacataccctgtataagaaggctcctaacgagagatttattaacaacactatatat

SEQ ID NO: 431 - DNA, 60 bp, Mus musculus
tttgactgggaccagcccagccattctcagcctctcgacatgtaatttcatttcttttac

SEQ ID NO: 432 - DNA, 60 bp, Mus musculus
aggactcatagacttacagaatgatgccgaatggaatgttttgtgcatgaccttttaacc

SEQ ID NO: 433 - DNA, 60 bp, Mus musculus
ccacctcgcccaagtctccttttactgaaataaaatttgaggggaagagaaaaaatttac

SEQ ID NO: 434 - DNA, 60 bp, Mus musculus
gatgttcttctgtaaaagttactaatatatctgtaagactattacagtattgctatttat

SEQ ID NO: 435 - DNA, 60 bp, Mus musculus
cttaagattcaggaaaatggttctttctgcccttcctagcgtttacagaacagactccga

SEQ ID NO: 436 - DNA, 60 bp, Mus musculus
tatattgacatccataacaccaaaaactgtctttttagctaaaatcgacccaagactgtc

SEQ ID NO: 437 - DNA, 60 bp, Mus musculus
tctttagtgctgcatttaagtggcatacaaaatacaatcccatatgtatgaactgttgtg

SEQ ID NO: 438 - DNA, 60 bp, Mus musculus
aatctatgccagatactgtatattctaccatggtgctaatatcagagctaaatgatactc

SEQ ID NO: 439 - DNA, 60 bp, Mus musculus
aatttacacatgtggtagtagtaggtccagattcctaagttacagtgtgctgaaaaataa

SEQ ID NO: 440 - DNA, 60 bp, Mus musculus
atgaggctaaatttgaagatgatgtcaactattggctaaacagaaatcgaaacggccatg

SEQ ID NO: 441 - DNA, 60 bp, Mus musculus
tctactactttgcttatcatgttcactgcaagggaggcaacgtatgggttgctctcttca

SEQ ID NO: 442 - DNA, 60 bp, Mus musculus
gtactgaactcacaagcgtatctcctattttatgagagaatactgtgataacaaaaagtg

SEQ ID NO: 443 - DNA, 60 bp, Mus musculus
ttggcccacccccaaagggccaagattataagtaaataattgtctgtatagcctgtgctt

SEQ ID NO: 444 - DNA, 60 bp, Mus musculus
ctgggaaccacctaatggtattattcctgtggccatttatcaataccttatgagactatt

SEQ ID NO: 445 - DNA, 60 bp, Mus musculus
tcctctggggtaaatgagcttgaccttgtgcaaatggagagaccaaaagcctctgatttt

SEQ ID NO: 446 - DNA, 60 bp, Mus musculus
gccgcaacgcaacagaaattgtttttaatttcatgtaaaataagggatcaatttcaaccc

SEQ ID NO: 447 - DNA, 60 bp, Mus musculus
acttttgggtctttagaactgagcccacctactgagtctcagtttctgttggtgtgacct

SEQ ID NO: 448 - DNA, 60 bp, Mus musculus
tgcttactaagaagccagtttgggtgggtaaagctctctggaagaaggaactttgcttct

SEQ ID NO: 449 - DNA, 60 bp, Mus musculus
tcccaatgtgtagaattcaactatgtaacgcaatggtacattctcactggatgagataga

SEQ ID NO: 450 - DNA, 60 bp, Mus musculus
cttatggacactatgtccaaaggaattcagcttaaaactgaccaaacccttattgagtca

SEQ ID NO: 451 - DNA, 60 bp, Mus musculus
gccatatgatgaacagaatttcaagaatgctgttttatgccttttaacctccaaagcagt

SEQ ID NO: 452 - DNA, 60 bp, Mus musculus
tcattttcctgtctaggctaaagctaaacttaaactatggctttacgtaaattaagctcc

SEQ ID NO: 453 - DNA, 60 bp, Mus musculus
caacatctaacgctttacataaatgcccttttagcttctctatttcgacacaactgtgat

SEQ ID NO: 454 - DNA, 60 bp, Mus musculus
ttacccaaataagcattttttaaatataccctgtactgtaggatagtgatgaacgcctag

SEQ ID NO: 455 - DNA, 60 bp, Mus musculus
ataagccgtatctgggtcttggactactttggtggacctaaagtagtgacacctgaagaa

SEQ ID NO: 456 - DNA, 60 bp, Mus musculus
aagtggaatggagccggccaagctgagcctgacttttttcaataaaacattgtgtacttc

SEQ ID NO: 457 - DNA, 60 bp, Mus musculus
cttaaaactactgttgtgtctaaaaagtcggtgttgtacatagcataaaaatcctttgcc

SEQ ID NO: 458 - DNA, 60 bp, Mus musculus
cagctgcctaacccgcaacatttgcattatgttcagactgtaacctgcttactgatggta

SEQ ID NO: 459 - DNA, 60 bp, Mus musculus
ctgtggtaccaaggagttattttggatgattagaagcacagaatgatcaggcctttagag

SEQ ID NO: 460 - DNA, 60 bp, Mus musculus
ttgtttttgtttttaacctagaagaaccaaatctggacgccaaaacgtaggcttagtttg

SEQ ID NO: 461 - DNA, 60 bp, Mus musculus
tgcctgaaaacacttaacactgattgtctaagagatgaaagtcctccaaagatgacacag

SEQ ID NO: 462 - DNA, 60 bp, Mus musculus
acttcagttaatgggtttataaagtcaagcactggcattggtcagttttgtatgatagga

SEQ ID NO: 463 - DNA, 60 bp, Mus musculus
tcccctatgcggtacgacctttactgtcagaaatatatttaagaaaatgttctaaacggt

SEQ ID NO: 464 - DNA, 60 bp, Mus musculus
gatccagccttctatgaagaatgcaaactggagtatctcaaggaaagggaagaattcaga

SEQ ID NO: 465 - DNA, 60 bp, Mus musculus
catgactgttgagttctctttatcacaaacactttacatggaccttcatgtcaaacttgg

SEQ ID NO: 466 - DNA, 60 bp, Mus musculus
cttgtaatcagacacgtgttttcctaaaataaagggtatagacaaaatttaagcccatgg

SEQ ID NO: 467 - DNA, 60 bp, Mus musculus
tgtctgaagatgcttgaaaaactcaaccaaatcccagttcaactcagactttgcacatat

SEQ ID NO: 468 - DNA, 60 bp, Mus musculus
tactcccattactatttgctggtaatagtgtaacgccacagtaatactgttctgattcaa

SEQ ID NO: 469 - DNA, 60 bp, Mus musculus
cagccgatgctttttcaataggatttttatgctttgtgtacctcaaccaagtatgaagag

SEQ ID NO: 470 - DNA, 60 bp, Mus musculus
gggacacttaatttacatgtactttaaccccatgaaagagtctagatagagagaagacac

SEQ ID NO: 471 - DNA, 60 bp, Mus musculus
gcctgccagtaaccccaggaagagtctagcttcaaaaacccacaaactcattatttttaa

SEQ ID NO: 472 - DNA, 60 bp, Mus musculus
aatctagatgttagaaatcaatgtgtatgatgtattgtatttagaccatacccgtgaccg

SEQ ID NO: 473 - DNA, 60 bp, Mus musculus
acgatgagcagtgtttgaaagctttccagtgagaactataatccggaaaaatgaatgttt

SEQ ID NO: 474 - DNA, 60 bp, Mus musculus
gatgcgtgaaatgttcctccaggaaaagccattcaagcctgattatttttctaagtaact

SEQ ID NO: 475 - DNA, 60 bp, Mus musculus
catcttagatctcagagacttgaaccttgaagctgttcctagtacccagatgtggatgga

SEQ ID NO: 476 - DNA, 60 bp, Mus musculus
cgtgtcctacacaatggtgctattctgtgtcaaacacctctgtattttttaaaacatcaa

SEQ ID NO: 477 - DNA, 60 bp, Mus musculus
aaggagccacgataatacttgacctctgtgaccaactattggattgagaaactgacaagc

SEQ ID NO: 478 - DNA, 60 bp, Mus musculus
gtttataggtagacctaagagataaaactgcagggtatcacattaacgttggttaaaaga

SEQ ID NO: 479 - DNA, 60 bp, Mus musculus
aaacttgagacattttgtaggacgcctgacaaagcgtagcctttttcttgtgtcaggatg

SEQ ID NO: 480 - DNA, 60 bp, Mus musculus
ctcataccaaagaaatacttgacactgctttgaaggagatagatgaagttggggatctgc

SEQ ID NO: 481 - DNA, 60 bp, Mus musculus
aaatccagcctttaaaagctcagtttcttcctctaagtgaatgtcattactctggtatac

SEQ ID NO: 482 - DNA, 60 bp, Mus musculus
accaggaactctggtaacatttgagggcatgcagataaaataataaagaatgagaacatt

SEQ ID NO: 483 - DNA, 60 bp, Mus musculus
tcaacatctatgacctttttatggtttcagcactctcagagttaatagagactggcttag

SEQ ID NO: 484 - DNA, 60 bp, Mus musculus
gaccgagagccaccacaaggccaagggaaaataagaccagccgttcactcacccgaaaag

SEQ ID NO: 485 - DNA, 60 bp, Mus musculus
ttctacctcactaactccactgacatggtgtaaatggtacatctcagtggtggtgatgca

SEQ ID NO: 486 - DNA, 60 bp, Mus musculus
ttggagaaattaggagttgtaagcaggacctaggcctgcttgattctttcccacctaagt

SEQ ID NO: 487 - DNA, 60 bp, Mus musculus
ttattgaaaagtttgaagttagaacttaggctgttggaatttacgcataaagcagactgc

SEQ ID NO: 488 - DNA, 60 bp, Mus musculus
caccatttccaacttgctgtctcactaatgggtctgcattagttgcaacaataaatgttt

SEQ ID NO: 489 - DNA, 60 bp, Mus musculus
aacaagagatcctgtggatgagggggtctgtataagttatactccaataaagctttacct

SEQ ID NO: 490 - DNA, 60 bp, Mus musculus
ttttgaccagttgaacccattttgttttcctagcgaacactagcataatattggaaaagc

SEQ ID NO: 491 - DNA, 60 bp, Mus musculus
gtgaggattggaattagaacattcataagaaaatatgacccaacatttcttagcatgacc

SEQ ID NO: 492 - DNA, 60 bp, Mus musculus
cgccctggagcctctgtcaagtcttggaccaagtaaaaataaagctttttgagacagcaa

SEQ ID NO: 493 - DNA, 60 bp, Mus musculus
aagatggagagttgtccaaacaagatcccaagtctaaatagagcaagggattctgaggtg

SEQ ID NO: 494 - DNA, 60 bp, Mus musculus
gttttaaaaggtgccaggggtacatttttgcactgaaacctaaagatgttttaaaaacac

SEQ ID NO: 495 - DNA, 60 bp, Mus musculus
tctgaggtattaaaatatctagactgaattttgccaaatgtaagagggagaaagttcctg

SEQ ID NO: 496 - DNA, 60 bp, Mus musculus
aagtattgctagactgaaaccacttgaacttctcagagaggttagactgacagaaggtgt

SEQ ID NO: 497 - DNA, 60 bp, Mus musculus
acatttttgtcatcatcatgtaaatcccacgatttcaaactgtaaacatctgttcagtgg

SEQ ID NO: 498 - DNA, 60 bp, Mus musculus
ctggggaaattgatctttaaattttgaaacagtataaggaaaatctggttggtgtctcac

SEQ ID NO: 499 - DNA, 60 bp, Mus musculus
aggactcaaaactatattaatctgctctgagataatgttccaaaagctccaaagaaagcc

SEQ ID NO: 500 - DNA, 60 bp, Mus musculus
gctccaacatgccatgtattgtatagacttttactacaattcaaataacgtgtacagctt

SEQ ID NO: 501 - DNA, 60 bp, Mus musculus
cagctgaatgggttttggtttgcaggaaaacagtccagagctttgaaaaggctcctaaga

SEQ ID NO: 502 - DNA, 60 bp, Mus musculus
tgtttttattgtgtttggtggagaagaataatacacttcttgcctaaatccagaagcccc

SEQ ID NO: 503 - DNA, 60 bp, Mus musculus
tccagttcccgaagaagctgataggaattgcccttgtgcatatactacacaagcatgcta

SEQ ID NO: 504 - DNA, 60 bp, Mus musculus
cataaagacatagtggaggttctgtttactcagccgaatgtggagctgaaccagcagaat

SEQ ID NO: 505 - DNA, 60 bp, Mus musculus
ggattcggctcgatgaatgaagcactttatggactgcggggatcagttactgccacaccc

SEQ ID NO: 506 - DNA, 60 bp, Mus musculus
tgcttttaccatgttctcgaggttcctgaacaaagagccttactgatagttccgctgcaa

SEQ ID NO: 507 - DNA, 60 bp, Mus musculus
tgaagcaaaaaacataaaacctcaccactgcctgctgaacctagaaccttttgttggggc

SEQ ID NO: 508 - DNA, 60 bp, Mus musculus
gaatccttagatgaagttatggattactttgttaacaacacgcctctcaactggctggta

SEQ ID NO: 509 - DNA, 60 bp, Mus musculus
gatattagtagtatatcataaaacttgagaaataaagatgcgctcaccccctatctgttg

SEQ ID NO: 510 - DNA, 60 bp, Mus musculus
tgtgataaagttgtgacatacgtattagttggcacatatttaagctccaaatcagtttgc

SEQ ID NO: 511 - DNA, 60 bp, Mus musculus
taaaagttaaagtaagcgaagaaaggaagctgtatctacactgctttccagtttaatcag

SEQ ID NO: 512 - DNA, 60 bp, Mus musculus
ggagatttttctcttcagggtgtctacataccttacacacacttgtgtcttaataagcaa

SEQ ID NO: 513 - DNA, 60 bp, Mus musculus
aatccatgggaggggggaacaagtccagactgcttaagaaatgagtaaaatatctggctt

SEQ ID NO: 514 - DNA, 60 bp, Mus musculus
aatgtggagtgtggagaagggcatttctgccatgataaccagacctgttgtaaagacagt

SEQ ID NO: 515 - DNA, 60 bp, Mus musculus
tgacatgaatgaaatcaaagtattttaccagaagaagtatggaatctctctttgccaagc

SEQ ID NO: 516 - DNA, 60 bp, Mus musculus
actggatactgtaactatgagaataaaatatagaagtgacagacgtctacagcattccag

SEQ ID NO: 517 - DNA, 60 bp, Mus musculus
atacaagcaagctgttaaagatcttggatcccattctatagtgtgtatacctaaatcaac

SEQ ID NO: 518 - DNA, 60 bp, Mus musculus
agcatcaactgtcctgtcaagcacaaaaaatgaagaagaaaataattacccaaaagatgg

SEQ ID NO: 519 - DNA, 60 bp, Mus musculus
cctctgttctgaggaacattctagcatagaaaatggaatatgctgcaaacatttctagat

SEQ ID NO: 520 - DNA, 60 bp, Mus musculus
gtgtagaagcctattgaaatatcagtcctataaagaccatctcttaattctaggaaatgg

SEQ ID NO: 521 - DNA, 60 bp, Mus musculus
ctgatcccgcctcatctcgctgctccgtgctgccctagcatccaaagtcaaagttggttt

SEQ ID NO: 522 - DNA, 60 bp, Mus musculus
tgtagaaaatgtggcctctcgttataaatgaaaataaatgtttaatttaatgggagtttc

SEQ ID NO: 523 - DNA, 60 bp, Mus musculus
ggtgccacagagaagagcccagttggaagctatacccgatttaattccagaattagtcaa

SEQ ID NO: 524 - DNA, 60 bp, Mus musculus
cagtgttgtttaagagaatcaaaagttcttatggtttggtctgggatcaatagggaaaca

SEQ ID NO: 525 - DNA, 60 bp, Mus musculus
ataactatatatacttagagtctgtcatacactttgccacttgaattggtcttgccagca

SEQ ID NO: 526 - DNA, 60 bp, Mus musculus
ccttgggacatttttgtggagtagtttgcagtgagataacagtgcaataaagatacagca

SEQ ID NO: 527 - DNA, 60 bp, Mus musculus
tctatacctggataaaaagaaacctacacttcactgtaaaacttcatgtttcaaggcaag

SEQ ID NO: 528 - DNA, 60 bp, Mus musculus
cctgtttactaaacccccgttttctaccgagtacgtgaataataaaagcctgtttgagtc

SEQ ID NO: 529 - DNA, 60 bp, Mus musculus
accgtgtagacactcatattttgcatgacatgatctaccattcggtgtaaacatttgtgt

SEQ ID NO: 530 - DNA, 60 bp, Mus musculus
gccaaaggaaaatgtttcagatgtctatttgtataattacttgatctacccagtgaggaa

SEQ ID NO: 531 - DNA, 60 bp, Mus musculus
tccagaagctgcattgccaacatcacaccccaaaattgtcctgacatcgctgcccgcatt

SEQ ID NO: 532 - DNA, 60 bp, Mus musculus
aaggactctgaggccatccgtagtcagtatgctcattactttgacctctctttggtgaat

SEQ ID NO: 533 - DNA, 60 bp, Mus musculus
atctcccaaggcaaagaactgaaactcagagctgtctggattgaagaaatgtgtgttgtt

SEQ ID NO: 534 - DNA, 60 bp, Mus musculus
atgaaggtaggataattaattacaagtccacatcatgagacaaactgaagtaacttaggc

SEQ ID NO: 535 - DNA, 60 bp, Mus musculus
ggtgtagccatacaatacacaaatacaatagatattctctctacaatctttatggtgtgg

SEQ ID NO: 536 - DNA, 60 bp, Mus musculus
ggagaagcagattatctgtgtggcttcctctttctgttctaatactggtaatcagtggac

SEQ ID NO: 537 - DNA, 60 bp, Mus musculus
gtgaacaccagaatttaatttccatacttgtacaggtaggactattcttcagctctctac

SEQ ID NO: 538 - DNA, 60 bp, Mus musculus
ggcttcacacatgtggagataagccccaaagaaatgaccatcatatatgtggaagcctct

SEQ ID NO: 539 - DNA, 60 bp, Mus musculus
gtttgtaaagttggtgattatattttttgggggctttctttttattttttaaatgtaaag

SEQ ID NO: 540 - DNA, 60 bp, Mus musculus
atggaattctgttagagtaaaaaagagaaaagcagatactattggctggccttggaggtc

SEQ ID NO: 541 - DNA, 60 bp, Mus musculus
aatagtgctgaatttgtctaaacagaattgagaggtcatagaaatccttaacagggtaac

SEQ ID NO: 542 - DNA, 60 bp, Mus musculus
tatgaagatttgggaaagaacagctatctgacacctggaaggctcagccagagtaacagt

SEQ ID NO: 543 - DNA, 60 bp, Mus musculus
gaggcaacattccttattcaccaactagtctcaaaagattgtcttaagccctgacgatgg

SEQ ID NO: 544 - DNA, 60 bp, Mus musculus
taatgaaggatgtataattgatgccaaataagcttgttctttagtcacgatgacgtcttg

SEQ ID NO: 545 - DNA, 60 bp, Mus musculus
cagtttgcgaagtagaattttgtttctaaaagtaaaagctaagttgaagtcctcacagag

SEQ ID NO: 546 - DNA, 60 bp, Mus musculus
tagaaaagatcaccaacagccggcctccctgtgtcatcctgtgactaagaaatgattctt

SEQ ID NO: 547 - DNA, 60 bp, Mus musculus
tatctaagagccaagtctatggcattagctgtgagaagtagttaccactgtaattcacct

SEQ ID NO: 548 - DNA, 60 bp, Mus musculus
aaattatcacttggatacggaggaacatgactaggcacattttatgaatactccaaatcc

SEQ ID NO: 549 - DNA, 60 bp, Mus musculus
aactattggtggtatatttttgaacacaggttaactgtggaggttatctgctaatagcaa

SEQ ID NO: 550 - DNA, 60 bp, Mus musculus
acctctggaacaggcattggaggactgccatggtcacacaaagaaacagaacttttacat

SEQ ID NO: 551 - DNA, 60 bp, Mus musculus
ccagtatacctacaaaatgacccacaagtaacccgcatgagtccaagttgtcagccatat

SEQ ID NO: 552 - DNA, 60 bp, Mus musculus
gtaaagggaccattactaagtgtatttctctagcatattatgtttaagggactgttcaag

SEQ ID NO: 553 - DNA, 60 bp, Mus musculus
ctctaagtcattcattttgtaaaattattatagagaaatctctacttatacagatgcaat

SEQ ID NO: 554 - DNA, 60 bp, Mus musculus
tctaatctcagggccttaacctgttcaggagaagtagaggaaatgccaaatactcttctt

SEQ ID NO: 555 - DNA, 60 bp, Mus musculus
attcagatcaggaaaggttgaaatggtcttcgttaccaggaggtctacatttattaattt

SEQ ID NO: 556 - DNA, 60 bp, Mus musculus
cagttatgggcttccattttcaaatatcttttcaactgtaatgactatgacaggaactga

SEQ ID NO: 557 - DNA, 60 bp, Mus musculus
gctttctatgcacgtattgtacaaattgtgctttgtgccacaggtcatgatcgtggatga

SEQ ID NO: 558 - DNA, 60 bp, Mus musculus
tggctagatttaattgaggataaggtttctgcaaaccagaattgaaaagccacagtgtcg

SEQ ID NO: 559 - DNA, 60 bp, Mus musculus
agaggaccattatgaagaagctgttctctttccggtcagggaagcatacctagactgaaa

SEQ ID NO: 560 - DNA, 60 bp, Mus musculus
agaaaagaaaaaagcagagaaaaagttcattgacatagcagctgctaaagaagtcctctc

SEQ ID NO: 561 - DNA, 60 bp, Mus musculus
atatttgcttatttaagcgtacgttcctttggtttatagagaacacccccaaatcacctg

SEQ ID NO: 562 - DNA, 60 bp, Mus musculus
gactctccaacttacagacttttatcagatatggagaagataatgttaagagacttcaca

SEQ ID NO: 563 - DNA, 60 bp, Mus musculus
taaaatcccattgaaagtggactcagttgtaagaataacaatgtgtaccattctggaatg

SEQ ID NO: 564 - DNA, 60 bp, Mus musculus
ccaatgaaccgacagtgtcaaaacttaactgtgtccaataccaaaatgcttcagtatttg

SEQ ID NO: 565 - DNA, 60 bp, Mus musculus
tcaaatcagtttcaactttcataaaatggattctttaatggatggagacttactcgtcgg

SEQ ID NO: 566 - DNA, 60 bp, Mus musculus
ctatacacaagatatgctaggagatgtgaaagataatggagactttccagtaagcacttt

SEQ ID NO: 567 - DNA, 60 bp, Mus musculus
ctgagatttttcaaatctttggcaactgagatgggatggatccatttaattagagaacgg

SEQ ID NO: 568 - DNA, 60 bp, Mus musculus
aaatgtctttccaacagtaatggtactatgtctatcccctaataaaacttcacttcagcc

SEQ ID NO: 569 - DNA, 60 bp, Mus musculus
tgaacattcacaggatttctaactatactgatataaacccagtgttttctggactcaggg

SEQ ID NO: 570 - DNA, 60 bp, Mus musculus
caacaaagttgatttacatgtataatccacacccttaaagatgaacagttagagtagcac

SEQ ID NO: 571 - DNA, 60 bp, Mus musculus
tggacacagttcactaaattcctgatttagtcaaagtaactagactgaaagaacctaaac

SEQ ID NO: 572 - DNA, 60 bp, Mus musculus
ttgttgtggcttcacacttaaattgttagaagaaacttaaaacacctaagtgactaccac

SEQ ID NO: 573 - DNA, 60 bp, Mus musculus
tgaacacatcaagtattctggagcttcagcggcagttaaatgccagtgacgaacatggaa

SEQ ID NO: 574 - DNA, 60 bp, Mus musculus
aaggtccaaaatacagacatttttgctagggcctagaaatcgaccataaaacacactgca

SEQ ID NO: 575 - DNA, 60 bp, Mus musculus
gactgaaatgaaagttccactaacggtatttgctctagtgatatgtggacattgtgatat

SEQ ID NO: 576 - DNA, 60 bp, Mus musculus
tcaaataaaaaacccttaatcaggctgtaaatcaaatgacactatgcgatgtcactacag

SEQ ID NO: 577 - DNA, 60 bp, Mus musculus
gcactataaatttcatcttttgaaggttgttgactacaagggtacaaaaatgatacaggc

SEQ ID NO: 578 - DNA, 60 bp, Mus musculus
cttgcatgagtgcgtgtttaagttctcggaatttcctgagaggatggagtgccattgtta

SEQ ID NO: 579 - DNA, 60 bp, Mus musculus
agtgttagctgcaaagctacaaagctctggaatggttacattatgattctggaacgttcg

SEQ ID NO: 580 - DNA, 60 bp, Mus musculus
tccagacttctcagagacaaggatcttgccttattttcaaatggtgctaaatttaaattc

SEQ ID NO: 581 - DNA, 60 bp, Mus musculus
agtgacttccaccttttaatgtcattaaaagcaggagcttaaactaaaagcagcattcca

SEQ ID NO: 582 - DNA, 60 bp, Mus musculus
acatacatttcatcaccaatatgttttatcttaccccatctctcagagtgttccctgcaa

SEQ ID NO: 583 - DNA, 60 bp, Mus musculus
ttttttgtattattgtgttttgtgctactgtagttttggtgtggcactattataattaaa

SEQ ID NO: 584 - DNA, 60 bp, Mus musculus
cttagggagactactaacatggagagaatgccgtgtatacctcacgtactgtgtgcttta

SEQ ID NO: 585 - DNA, 60 bp, Mus musculus
catacatagaagcaaaatactttaactgctgtaaaccttcaaaagttagtagacgtgagg

SEQ ID NO: 586 - DNA, 60 bp, Mus musculus
acttcctgcaatacatcccagtaggtacacctagtttacaatttaaactagtttgtgaaa

SEQ ID NO: 587 - DNA, 60 bp, Mus musculus
ggaggcacataattccaagcaatacaggctgttaaaatataaataatgggaactgtgatt

SEQ ID NO: 588 - DNA, 60 bp, Mus musculus
aagcgttaggaaggaaatttcctggaaggataggttgtcttcctagcagcctcgtcaata

SEQ ID NO: 589 - DNA, 60 bp, Mus musculus
ttttttaacttcactcatgacaacagaggaagaaaggaattgaggtttaggtaagttctc

SEQ ID NO: 590 - DNA, 60 bp, Mus musculus
aggcatatctcatagagccttaagttagaatcttactcttatggaaggagttatttccta

SEQ ID NO: 591 - DNA, 60 bp, Mus musculus
gatcacctcattcctcgactgtgagatgagtttatgaaaagaattaaaagtgagcacttg

SEQ ID NO: 592 - DNA, 60 bp, Mus musculus
taaaggtaactccatcaagatgagaagccttccgagactttgtaattaaatgaaccaaaa

SEQ ID NO: 593 - DNA, 60 bp, Mus musculus
ggccaggtatatgtgtaccagtgctcttcaaagggagaaccattaaaaccaacatggaat

SEQ ID NO: 594 - DNA, 60 bp, Mus musculus
ccaagagattatttaacattttatttaattaaggggtaggaaaatgaatgggctggtccc

SEQ ID NO: 595 - DNA, 60 bp, Mus musculus
agtgaacgaaaaagacaccttaacatgtttcatctactcagtgaggaacgacaagaacaa

SEQ ID NO: 596 - DNA, 60 bp, Mus musculus
gatatttattgagtgtcaaataaaaaggtgccataatcttcagtagcgtacacagtagag

SEQ ID NO: 597 - DNA, 60 bp, Mus musculus
gtgttaccagaagaagtctctaaggataaccctaagtttatggacacagtggcggagaag

SEQ ID NO: 598 - DNA, 60 bp, Mus musculus
tgtctttattttaatgccaaaaggaagtgattatgcagctgtgtgtagagtttcagagca

SEQ ID NO: 599 - DNA, 60 bp, Mus musculus
agaacaaactggaattttattctgaagcttgctttaaagacactgatgtgcctaaacgct

SEQ ID NO: 600 - DNA, 60 bp, Mus musculus
tatggtctttccaaggaaactagtcacagtgtcatcttaatcttactgatccaataaaac

SEQ ID NO: 601 - DNA, 60 bp, Mus musculus
atcctcctgattggtctgaatgcatttccaatgatgtcagggagtctgccttcctcagcc

SEQ ID NO: 602 - DNA, 60 bp, Mus musculus
taagccctgtcttctgggaaatatcagttttaaagagaacttttgtgcaattccaaatga

SEQ ID NO: 603 - DNA, 60 bp, Mus musculus
ggaagattaattttccagggattgtatcaatcaggaccatttttgtggggcacttgggac

SEQ ID NO: 604 - DNA, 60 bp, Mus musculus
atgtgatctacagtggtgtgacaacttgccttgtatctgatggactgtccagatttatgg

SEQ ID NO: 605 - DNA, 60 bp, Mus musculus
aaacgaagtgactttccatgaatgcctttaacattcttgtgtcaacatttggtactaaac

SEQ ID NO: 606 - DNA, 60 bp, Mus musculus
aatactcattatgctgtgtgggaatttcctgattactagaagctgacctctgctatcctg

SEQ ID NO: 607 - DNA, 60 bp, Mus musculus
gaattattataaacaataatgtgttacagaagctgatgctgaccttgtgttactgagcac

SEQ ID NO: 608 - DNA, 60 bp, Mus musculus
ttcttgaggtttaaggacgacaactttatggaccctgaatggaaactgaggaatcacaag

SEQ ID NO: 609 - DNA, 60 bp, Mus musculus
gtcacatgccaataaaaacaggaaactctgaaaataatatgaatgtacagtatcagaccg

SEQ ID NO: 610 - DNA, 60 bp, Mus musculus
ccctattgcaaattgatttgttttcccttaaccctgttcccttttaaccccggctttttt

SEQ ID NO: 611 - DNA, 60 bp, Mus musculus
cattgcatcgttttccaacatacttttagatttacaaagtaaaaccaaccatggatctgc

SEQ ID NO: 612 - DNA, 60 bp, Mus musculus
ttgagaaattaaaaacaaatatccaaaatcgacttttcctcaaggctatgtgcttcgtcc

SEQ ID NO: 613 - DNA, 60 bp, Mus musculus
acgactcttgttaatgtgcgtttctcatggagtaattttcagagcctgaacttgtagcac

SEQ ID NO: 614 - DNA, 60 bp, Mus musculus
gttggtgtgtcctgaaagggatggagttatggcagaagtgcttttgtgatcaactggttt

SEQ ID NO: 615 - DNA, 60 bp, Mus musculus
cagaaaactcaagtcatggactatgcgagtcaagaattaaaatacaactgtattatgtgc

SEQ ID NO: 616 - DNA, 60 bp, Mus musculus
aaatttctcatttaattttccagtctcgattgcagtaacaaagtcaaccacacagtcaga

SEQ ID NO: 617 - DNA, 60 bp, Mus musculus
ggaggaagacaactgaacatttgtataaaacgtaaaaagtttactgattggggtgggaca

SEQ ID NO: 618 - DNA, 60 bp, Mus musculus
cagcagcttacaaacactgaagttaggcgactagagaaaaacgttaaagaggtattagaa

SEQ ID NO: 619 - DNA, 60 bp, Mus musculus
gagaaatgttagtaaaatggtaaaagggaatcacgtgacattcagggtaggaagagcttg

SEQ ID NO: 620 - DNA, 60 bp, Mus musculus
tcaggaaaaatgtcataagccatctggtaagttttcttaaaggatgttgttaagaagtcc

SEQ ID NO: 621 - DNA, 60 bp, Mus musculus
caaaacaaatacatattataaaataaaagaaaaggcgtgataaatggatgtgacaaaatt

SEQ ID NO: 622 - DNA, 60 bp, Mus musculus
gtagggaaaatatgtccataggttttaggaaacacttagcctttaatatactggttgtag

SEQ ID NO: 623 - DNA, 60 bp, Mus musculus
gtatacagatggtagttagaaatactggatgaactgatcagttattgtgtgtagaaagtg

SEQ ID NO: 624 - DNA, 60 bp, Mus musculus
ttgtatcccaaagggaaacgggaatcaagatacggacctatgcttttcatatgaaaccgt

SEQ ID NO: 625 - DNA, 60 bp, Mus musculus
tgcagctaaggtacatttgtagaaaagacatttccgacagacttttgtagataagaggaa

SEQ ID NO: 626 - DNA, 60 bp, Mus musculus
ggcaatggaaaatgttgaaatccattcagtttccatgttagctaaattactgtaagatcc

SEQ ID NO: 627 - DNA, 60 bp, Mus musculus
ccccaaagaaaactggaaaaattgttttccactcctgaaatttcttggatgggccccctg

SEQ ID NO: 628 - DNA, 60 bp, Mus musculus
ccagacagtgtattcttcggacaaatggtgtgaaagtgaaataagaattcataatgtaac

SEQ ID NO: 629 - DNA, 60 bp, Mus musculus
agcaaaagtatgtatattttagcttgtcatgaaatgtcaacgaaggacactgagaaagag

SEQ ID NO: 630 - DNA, 60 bp, Mus musculus
tagaatgggaattttctgtctcatagtgacatattgctatgtttaacagtgaacactcac

SEQ ID NO: 631 - DNA, 60 bp, Mus musculus
tgacggtatatttgcaaaaagagaaagaaaaatctggtatttgcaatgatctgtgccttc

SEQ ID NO: 632 - DNA, 60 bp, Mus musculus
gaaatatcatttgtagctttaaggctagaaaatgaaaaagaatccaagccagtagaaggc

SEQ ID NO: 633 - DNA, 60 bp, Mus musculus
ataccaggaaaataaaagtaccagtaaggaagcatcaaatcaagatgtcatagtcagtgg

SEQ ID NO: 634 - DNA, 60 bp, Mus musculus
cagtgtaaatatagcatatggttaggtggtgagaaaatgatcttgagactgataagaatc

SEQ ID NO: 635 - DNA, 60 bp, Mus musculus
atcctttagatgttagtacagtgtttatgagaaaactgttactagaagctgaagaacagc

SEQ ID NO: 636 - DNA, 60 bp, Mus musculus
agtgttctatatgtgtaaattagtattttcaactggaaaatgttggctggtgcaaaaggc

SEQ ID NO: 637 - DNA, 60 bp, Mus musculus
gtctgggctagtgcccgtttttaaccctacccattgatcatttcaagaaacctctggtta

SEQ ID NO: 638 - DNA, 60 bp, Mus musculus
tgtaagaccatttctaaattgctggtaatagaaactcatggcagtaaaaatgtaacctcg

SEQ ID NO: 639 - DNA, 60 bp, Mus musculus
actggaataggaatgtgatgggcgtcgcaccctctgtaaatgtgggaatgtttgtaactt

SEQ ID NO: 640 - DNA, 60 bp, Mus musculus
tctactagaagggttaaaagccatatgaatgcaagaaatcatttgaggcttaaaatgctg

SEQ ID NO: 641 - DNA, 60 bp, Mus musculus
ggacaccatttttcatgttaaatagattttaacctcgtatctatgcataggctaaggtgg

SEQ ID NO: 642 - DNA, 60 bp, Mus musculus
tagataaagcccgtatgagaagagaaaaccaaattaatccacttcagcaaaaagaaagcc

SEQ ID NO: 643 - DNA, 60 bp, Mus musculus
caatgtcagactgccatgttcaagttttaatttcctcatagagtgtatttacagatgccc

SEQ ID NO: 644 - DNA, 60 bp, Mus musculus
ctttggggggggttttggaaaaccggttttttcgggggggtttccttttggggggttttt

SEQ ID NO: 645 - DNA, 60 bp, Mus musculus
gccatacagcttatatttgtactggtatgtccagaaatcatggaggaaagaaaagtaaaa

SEQ ID NO: 646 - DNA, 60 bp, Mus musculus
tggtgttttgattacagtgagacatcacaggttatctaaaagcccttcgttataaccagc

SEQ ID NO: 647 - DNA, 60 bp, Mus musculus
tatttggtggtaaagaatatggttgaaaattgtcatccacatgcatgcatcaagtaacac

SEQ ID NO: 648 - DNA, 60 bp, Mus musculus
cgaggagttattagggagaatcatggagccacataagaaaatcttgggcaagaaaagagg

SEQ ID NO: 649 - DNA, 60 bp, Mus musculus
tggtgacaggattacgtgaaaatctctgacattgtgataaactcgataaaggcttaagag

SEQ ID NO: 650 - DNA, 60 bp, Mus musculus
accctttgcttaaatagtgggaaaacgtgaatgtttagcataatataaaaacatgcaggc

SEQ ID NO: 651 - DNA, 60 bp, Mus musculus
gttggactctaatacaactgaccattgaaaaatgaacaacggcttattgttttgtaacag

SEQ ID NO: 652 - DNA, 60 bp, Mus musculus
ttcctacaaagtgtgtttctataggattactagagtagcggttttgtactgtgaggaaac

SEQ ID NO: 653 - DNA, 60 bp, Mus musculus
tagataacagtgactattgacgattttagtaaaagaaagttgacatgcgtaccgctacct

SEQ ID NO: 654 - DNA, 60 bp, Mus musculus
ggggggacagttaatatcgtttgttagataccataagtggtggaaataaagtgactaaag

SEQ ID NO: 655 - DNA, 60 bp, Mus musculus
aaagaggaaactgtcctatttctcaactgataagtactcctggtaagatgtaatatttgc

SEQ ID NO: 656 - DNA, 60 bp, Mus musculus
caaatgtactgagaaacaaaatcatgaacgaccttgaaatcaccttcttatttcagctcc

SEQ ID NO: 657 - DNA, 60 bp, Mus musculus
aacataaatcaaaatatacttaggaatatttacaattaaacatgatgttttaaacttagt

SEQ ID NO: 658 - DNA, 60 bp, Mus musculus
gactatttattagattagaaagtcatgtttcactcgtcaactgagccaaatgtctctgtg

SEQ ID NO: 659 - DNA, 60 bp, Mus musculus
acaaacacatgaaaaaatcaagtaggaactggagaaacgtctcacagttaagaatgtttg

SEQ ID NO: 660 - DNA, 60 bp, Mus musculus
aattcacagatggcttacatttatgtaaagaattcctgtaaggcactcatgtttgacatc

SEQ ID NO: 661 - DNA, 60 bp, Mus musculus
tataccaaactgaaaacgtttaaatctcaaatgaagtaagcaaggttttgttctccctgc

SEQ ID NO: 662 - DNA, 60 bp, Mus musculus
tagccatttaggagatgtcccttcaaagtgacgtgatgatggacttgcacttgggaatca

SEQ ID NO: 663 - DNA, 60 bp, Mus musculus
gctcagcttaggctagactttgaccaggtaagcagaagaaatgagaaacaaaactcagca

SEQ ID NO: 664 - DNA, 60 bp, Mus musculus
tatcactggaatattgaaaggttgtatgtagtatgggagatcaactttcttccctaaggt

SEQ ID NO: 665 - DNA, 60 bp, Mus musculus
actgctgagaaaaacaaaattcactacatacctcaatagttatttaccatgagattggcg

SEQ ID NO: 666 - DNA, 60 bp, Mus musculus
gaaggaaatgcaaacacctttgaacttcaattctttcagtaggaaaacaagaattgtccc

SEQ ID NO: 667 - DNA, 60 bp, Mus musculus
agaaaaacactaaactccaaattagtataataacgagcactacagtggtgaaaaagctcc

SEQ ID NO: 668 - DNA, 60 bp, Mus musculus
aaaggaatcttaagagtgtacatttggaggtggaaagattgttcagtttaccctaaagac

SEQ ID NO: 669 - DNA, 60 bp, Mus musculus
gaaatggattttgaggctttgaaaatgaaaatggctagtatctcaaagatgtcagtatcc

SEQ ID NO: 670 - DNA, 60 bp, Mus musculus
actatttcttgtcaatagtttggcaaaagacgactaattgcactgtatattgccagtgta

SEQ ID NO: 671 - DNA, 60 bp, Mus musculus
tcctctaaagatgtgtcttatatacatgattgtcattggtgggctcaaacaataagggtg

SEQ ID NO: 672 - DNA, 60 bp, Mus musculus
ttggaaactacaagtaaccctcagacggcctaattcttataatccggaaaaacaccccaa

SEQ ID NO: 673 - DNA, 60 bp, Mus musculus
gtgtgataatcttttcatgttttctagagcaaagacaaagcagttactcttctatcgcaa

SEQ ID NO: 674 - DNA, 60 bp, Mus musculus
ggctttagagaaaacttcggtcttcaaagaactcttctaattagttccttcttggaaaaa

SEQ ID NO: 675 - DNA, 60 bp, Mus musculus
aaagtaggagatgagatttacatttccccaatattttcttcaactcagaagacgagactg

SEQ ID NO: 676 - DNA, 60 bp, Mus musculus
agtcctctgcatgtttccaaaatttcctttacatgaaggctatattggatcagagcttac

SEQ ID NO: 677 - DNA, 60 bp, Mus musculus
aagaataaatcacttgaaatcatactgtttttggaaatccaaactgtttaaagaaaactt

SEQ ID NO: 678 - DNA, 60 bp, Mus musculus
gttagatgccattgaaggggaaataactttggctaatagcttggaaaactcagtactaag

SEQ ID NO: 679 - DNA, 60 bp, Mus musculus
agcagatatgtgacttctcatatacacagttacgctaactcaggtgtatgatgaatacag

SEQ ID NO: 680 - DNA, 60 bp, Mus musculus
tgtctatgggagaagtaatagcctgaaataagataaggctcaaacaaacactacttactt

SEQ ID NO: 681 - DNA, 60 bp, Mus musculus
gggaagaaaaagaattggtcggaagatgttcaggtttttcgagttttttctagatttaca

SEQ ID NO: 682 - DNA, 60 bp, Mus musculus
cttgaagaaaagtatatcacgtaggcatagatgagaaagccgtttgatcaagtctggtta

SEQ ID NO: 683 - DNA, 60 bp, Mus musculus
tccttcagtcagatatctgtcccagagaaaggaaaataaggagcatggtaagaaatgagt

SEQ ID NO: 684 - DNA, 60 bp, Mus musculus
tatggaatggagaaataaatacatctgtgttgaagaaccttttgatggaactaataccgc

SEQ ID NO: 685 - DNA, 60 bp, Mus musculus
aggtcaatgttaagttttctgagtttaatatatagttagggtgaaagacttagcacacgg

SEQ ID NO: 686 - DNA, 60 bp, Mus musculus
aatgcttaactttgagtcacactgtttacccttcctatgaggttgcattttgacaacaac

SEQ ID NO: 687 - DNA, 60 bp, Mus musculus
taaagggaacccccatttctgacccattagtagtcttgaatgtggggctctgagataaag

SEQ ID NO: 688 - DNA, 60 bp, Mus musculus
cccctttttgtaactgggatataaatccttgaaagaaaggagaatttagagttttgcccc

SEQ ID NO: 689 - DNA, 60 bp, Mus musculus
gtcagtgagttggtttcctttccatcaggaaaaatggattctgtaaagagtcagggcgtt

SEQ ID NO: 690 - DNA, 60 bp, Mus musculus
gaaagccgtcagcgaaagttttctcgtgacccgttgaatctgatccaaaccaggaaatat

SEQ ID NO: 691 - DNA, 60 bp, Mus musculus
gaaatatgttaactaagagcagcccaaaaatactggatatgcttatccaatcgcttagtt

SEQ ID NO: 692 - DNA, 60 bp, Mus musculus
gtatacaatgctatttttaggttaaggcctaaacttctgaagatcttggtaacagcagag

SEQ ID NO: 693 - DNA, 60 bp, Mus musculus
ggatgaagtggaagattactggcaggtccaaaaacctgattttctagtacatttcactct

SEQ ID NO: 694 - DNA, 60 bp, Mus musculus
ttcaatcaagaaagtagatgtaagttcttcaacatctgtttctattcagaactttctcag

SEQ ID NO: 695 - DNA, 60 bp, Mus musculus
aaattttcttaaagctatgaactctgacttttgattttgtgtttccatttagtagaaact

SEQ ID NO: 696 - DNA, 60 bp, Mus musculus
agaatctcactactaaagtcaagtatagaaataactgttcttatgttttcctccaaggcc

SEQ ID NO: 697 - DNA, 60 bp, Mus musculus
atctttggctatattttcctggtagcatatgacaaatgtttctacagtgagaagctgaga

SEQ ID NO: 698 - DNA, 60 bp, Mus musculus
gggttataatgcactgagatccagaagttgggaaaactcaataaatgtacaaaggaaagc

SEQ ID NO: 699 - DNA, 60 bp, Mus musculus
tacttgtgtgacaagctagagaagttacagaagagaaatgacgaactagaagagcaatgc

SEQ ID NO: 700 - DNA, 60 bp, Mus musculus
taaataatcccttcccatgagcccactgctctgaatggacaagctgtccttatcttcaat

SEQ ID NO: 701 - DNA, 60 bp, Mus musculus
aaatagttgtttttaaggttgaaggaagagacattccgatagttcacagagtaatcaagg

SEQ ID NO: 702 - DNA, 60 bp, Mus musculus
tgaatctacaggcaactcttcatctctgtaatgctacctgacttctcttgtgaggagctg

SEQ ID NO: 703 - DNA, 60 bp, Mus musculus
tggcaaagagtagatgagaaaatgttggatttaaatcagcagactcatttcatactttgc

SEQ ID NO: 704 - DNA, 60 bp, Mus musculus
accacgtttaaatgaccagtctcaggataaagagttttacagaaaatttaaaatgcctgg

SEQ ID NO: 705 - DNA, 60 bp, Mus musculus
gacatcgttttctctctaaattcagtagcagtttcatcgacagtgccattgaactatggg

SEQ ID NO: 706 - DNA, 60 bp, Mus musculus
tctgtggggttctcatgccagtgtctgaaatctcacctcactagagatgtttctcgaatt

SEQ ID NO: 707 - DNA, 60 bp, Mus musculus
ttccagttctcatgtcttgagatttcaagtaaagatgtgttagtgtaagctcagatccga

SEQ ID NO: 708 - DNA, 60 bp, Mus musculus
aaccattgggaaaatgcaatacagataaactagagattcgtataatgccacgtgttagct

SEQ ID NO: 709 - DNA, 60 bp, Mus musculus
gtgaatggagtgtttactgtatgtaagaaagaagaaaagtggaactacatttgctatgag

SEQ ID NO: 710 - DNA, 60 bp, Mus musculus
ttcacaatttagacacaagatttggaagattgaaactgacatgaaagtcttcttcctgag

SEQ ID NO: 711 - DNA, 60 bp, Mus musculus
gaagattttttgatgtataaaagtggcgtctactccagtaaatcctgtcataaaactcca

SEQ ID NO: 712 - DNA, 60 bp, Mus musculus
agaatgaaccagaatggagaaaacgtaaaatttgaagaatctcgttgaagagctatttgc

SEQ ID NO: 713 - DNA, 60 bp, Mus musculus
tcgacaagaggtaatccgagaaatggagcagaaaacctccttgcacttcagtgatataca

SEQ ID NO: 714 - DNA, 60 bp, Mus musculus
tatatgcaacttcatagatcctctgcaatatgtacttagctacctaagcatgaaatagac

SEQ ID NO: 715 - DNA, 60 bp, Mus musculus
cgtcatatatcctatttgtaatcaagaggaaagactacattaagaagatagggtgcatag

SEQ ID NO: 716 - DNA, 60 bp, Mus musculus
ctcagatcagttctttagaaagagctggtatagaaatgggtgatgtaaaacttgagaagc

SEQ ID NO: 717 - DNA, 60 bp, Mus musculus
aatgaaaatctgcgtctaacttttgaaagtaagtgttaacttacttgaatgctggttccc

SEQ ID NO: 718 - DNA, 60 bp, Mus musculus
aatcttcgaccagacattggatatttgaactatcctgaaacattttagaaatatccaggc

SEQ ID NO: 719 - DNA, 60 bp, Mus musculus
taccccattaaaggcatcaaatccgggtttagatcagtccctctgaagaatgggtacagt

SEQ ID NO: 720 - DNA, 60 bp, Mus musculus
ttttttctcttgccaatgtatttttgtaaggctcgtaaataaattattttgaacaaaaca

SEQ ID NO: 721 - DNA, 60 bp, Mus musculus
cacaccctctgatgttccaaaagctccaggaccagatcttcaatctcatgaagtatgaca

SEQ ID NO: 722 - DNA, 60 bp, Mus musculus
cccaggtatttctaagcatgctaggtttgaggtcatttaccatgttcaaataaaagacgg

SEQ ID NO: 723 - DNA, 60 bp, Mus musculus
ggagcaaaacttgaataatgtcctttatcctgatttgaaataatcacgtcatctttctgc

SEQ ID NO: 724 - DNA, 60 bp, Mus musculus
tggaataagaaagaatctgtggtagaaataatagacttgctacatagggttagctaaggc

SEQ ID NO: 725 - DNA, 60 bp, Mus musculus
accacagtttatcagcatttgaagatttccttgatgatccatacttgtcttgggataggg

SEQ ID NO: 726 - DNA, 60 bp, Mus musculus
agggtcagcgccgaatcttgtggacacactgacaaggatgtctaatccaaatagatgtat

SEQ ID NO: 727 - DNA, 60 bp, Mus musculus
agtggagtattcagtctggagtttcaggattttgtgaataaatgcttaataaagaaccct

SEQ ID NO: 728 - DNA, 60 bp, Mus musculus
tttgggcccttaaaaacatatttcagttttgcccaagtgaggccttaaaaattgcccatg

SEQ ID NO: 729 - DNA, 60 bp, Mus musculus
aaaggaaaataaagtggatctgaaagtagactctgcttctgcgcatgtgtgagtggtgcc

SEQ ID NO: 730 - DNA, 60 bp, Mus musculus
ttcactcctggactgtgattttcagtgggagatggaaatttttcagagaactgaactgtg

SEQ ID NO: 731 - DNA, 60 bp, Mus musculus
caccatccttccagaatatggtatgaaaaatctatgcaaactgtgtaagcttttgctcat

SEQ ID NO: 732 - DNA, 60 bp, Mus musculus
ttgtggagtgtgaaataaaggataattgcctacctctagcaagtggatcttattatgttg

SEQ ID NO: 733 - DNA, 60 bp, Mus musculus
accagaaaggacagtctggacttcagccaacaggactcctgagctgagatgaagtaacaa

SEQ ID NO: 734 - DNA, 60 bp, Mus musculus
gatactgccggctttgaaaatgaagaacagaagctaaaattcctgaagcttatgggtggc

SEQ ID NO: 735 - DNA, 60 bp, Mus musculus
ccatttgagcctcactgcaatgttagtgcagaggagaaaacaatttttaatgtaatcttg

SEQ ID NO: 736 - DNA, 60 bp, Mus musculus
ggcaacttgtaaagtgtgttcattctaactgttaaactgagaaaacttgagaacatactg

SEQ ID NO: 737 - DNA, 60 bp, Mus musculus
cagaagagattctgaaaatgttagttgtggtgactctaatgtagatccataactgaaaag

SEQ ID NO: 738 - DNA, 60 bp, Mus musculus
tatcgtaagttgcacctattgttaagtggaaaatgctctgattacactcaggaagctggg

SEQ ID NO: 739 - DNA, 60 bp, Mus musculus
tgttttgtccctaaatcaccaccactcactatttctcccagggtctgataatgcctttac

SEQ ID NO: 740 - DNA, 60 bp, Mus musculus
agccactttaactctaaactcgaatttcaaagccttgagtgaagtcctctagaatgttta

SEQ ID NO: 741 - DNA, 60 bp, Mus musculus
gctttgtttaaatggtcagactcccaaacattggagccttttgaatgtgttctgagacct

SEQ ID NO: 742 - DNA, 60 bp, Mus musculus
ccttagaaagatggtaattcactttaggtaaaagtactatttcacgccattatgaaaccc

SEQ ID NO: 743 - DNA, 60 bp, Mus musculus
taaaatgaggcttttggaaagaaagatgaaaacgtagaatgtagtgctaagaacgtttcc

SEQ ID NO: 744 - DNA, 60 bp, Mus musculus
gcagttactcatctttggtctatcacaacataagtgacatactttccttttggtaaagca

SEQ ID NO: 745 - DNA, 60 bp, Mus musculus
tgcttagaactacatagaatcagaagcaaaatggatgccttagcactgaggaaaggtttc

SEQ ID NO: 746 - DNA, 60 bp, Mus musculus
ggttttcgaaccacgtacctttatgcctcgtgattgtgaaacattgacttttgtaaaccc

SEQ ID NO: 747 - DNA, 60 bp, Mus musculus
gttcactgtagaaatttgtgataagaaagacacacagacgtagaaaatgagaatacttgc

SEQ ID NO: 748 - DNA, 60 bp, Mus musculus
aaagacttttttggacttaatactgattctgtgaaaactgaagaagtgtagatgtctccc

SEQ ID NO: 749 - DNA, 60 bp, Mus musculus
ctggtgtgggatattttccacactttagaatttgtataagaaactggtccatgtaagtac

SEQ ID NO: 750 - DNA, 60 bp, Mus musculus
taaaggttttagtgtcctaactccccaggatcaggagattatcccaactatttctggggt

SEQ ID NO: 751 - DNA, 60 bp, Mus musculus
ctgaattttgatcacttgtggtttctcatggtgacctccatttgcaacaaaaagatgtct

SEQ ID NO: 752 - DNA, 60 bp, Mus musculus
tgtgctttaccaaaatgggaaataattctgctttagaggatactatcaagacaaccttac

SEQ ID NO: 753 - DNA, 60 bp, Mus musculus
tctgtgagatgttgtagacattccgtaagagaatccagaatgatagcaggatcaggaaag

SEQ ID NO: 754 - DNA, 60 bp, Mus musculus
cttacatgatctcctaaaaggatgggcccctccttccttttgcgggttgaaagtaatgaa

SEQ ID NO: 755 - DNA, 60 bp, Mus musculus
ctgtttaaaaaatgaaatcaggaagcttgaagaagacgatcagacgaaagacatttgagc

SEQ ID NO: 756 - DNA, 60 bp, Mus musculus
tgaatatagtagggccatgagtatataaaatctatccagtcaaaatggctagaattgtgc

SEQ ID NO: 757 - DNA, 60 bp, Mus musculus
gggggaaattctatatgagcttcgttttctaatgacttacatggatagtatggaaacttc

SEQ ID NO: 758 - DNA, 60 bp, Mus musculus
aaacttgaaaacacagacattgaaggaatcataggtatttttgctttatgctctctggca

SEQ ID NO: 759 - DNA, 60 bp, Mus musculus
aataagcaggaagaatttgacttggaaaactaatacacgcatgttaggcattctcaaggc

SEQ ID NO: 760 - DNA, 60 bp, Mus musculus
tcccactgtttacagatgtagttcttgtgcacaggtgccactagctggtaccctaggcct

SEQ ID NO: 761 - DNA, 60 bp, Mus musculus
tatttttgtcattgcctctagtgatttttgtaaatgggaatggaaaagtacaaggcaacc

SEQ ID NO: 762 - DNA, 60 bp, Mus musculus
ttaactggcctgtcaaactggtcttgaagcgtctctaagtgaagagccagaagaaaccct

SEQ ID NO: 763 - DNA, 60 bp, Mus musculus
caatgtgatttttcaatggtattagttcaaattgacgtggattcatgccacatggaaatc

SEQ ID NO: 764 - DNA, 60 bp, Mus musculus
aactgaataaagttgaccagaaagtgaaagtctttaacatggatggaaaagacttcatcc

SEQ ID NO: 765 - DNA, 60 bp, Mus musculus
ggatataaagtgtatttctttcagtgatttctcagtgcataagaagtgcataagtctcag

SEQ ID NO: 766 - DNA, 60 bp, Mus musculus
tagctttttaaaagaagtttttctacctacagtgaccattgttaaaggaatccatcccac

SEQ ID NO: 767 - DNA, 60 bp, Mus musculus
atttgcaaggtcagaaactagccaaggtccttctcaggcatctatccttaacttggtctc

SEQ ID NO: 768 - DNA, 60 bp, Mus musculus
ttggaatttgaggaggagaaatgaaaaaacagtgtgtccctggtgtcaccctggcatcat

SEQ ID NO: 769 - DNA, 60 bp, Mus musculus
tcttatgatttaagtgattggtggataaatgtataggaattttacactccagcagcatgg

SEQ ID NO: 770 - DNA, 60 bp, Mus musculus
gcctcaaatggaaccacaagtggtgtgtgttttcatcctaataaaaagtcaggtgttttg

SEQ ID NO: 771 - DNA, 60 bp, Mus musculus
ccgtacacaaaagtgaagatttcagcgaaatgccaaggaagtgccatctatctggcttct

SEQ ID NO: 772 - DNA, 60 bp, Mus musculus
aagaaaatgctgtatgatgttagaagacattgtaattatcatcccgtgtctttgctgtac

SEQ ID NO: 773 - DNA, 60 bp, Mus musculus
ggcatttcagtttatcttgggtttgtaattagttaaaacaaaaaccaacctaggtctgtg

SEQ ID NO: 774 - DNA, 60 bp, Mus musculus
attagccaaggagtccggacataatatttatccagatctctaagcagttagctttaaatt

SEQ ID NO: 775 - DNA, 60 bp, Mus musculus
tacattagctaatactaaccacatagaatatcagacttagatacgtgaatagggatcctg

SEQ ID NO: 776 - DNA, 60 bp, Mus musculus
aagattttctagtcactgcataaaggaaacgcctaagagttgccgtattgctttctgaga

SEQ ID NO: 777 - DNA, 60 bp, Mus musculus
acaagaattcattcttaacatttgaacgagtgtatttgcttaggtcgatgaaagtgttgc

SEQ ID NO: 778 - DNA, 60 bp, Mus musculus
aggattttctcatgaagaaccagatgacatgtggtaataacattagctgtctagtttctc

SEQ ID NO: 779 - DNA, 60 bp, Mus musculus
tagagtcatgaagaacagaaattcaaggtcattttcaattacagagtgaggttagagcca

SEQ ID NO: 780 - DNA, 60 bp, Mus musculus
tctaaaacatgccaaatgacttatgtcacaaagaataggtcctaatatactgtatacccc

SEQ ID NO: 781 - DNA, 60 bp, Mus musculus
gtgtttcttcccatttgtaaatgtcctgaaccataaattactatcaggattaactgacag

SEQ ID NO: 782 - DNA, 60 bp, Mus musculus
gaagctggaagcatttgtttttgaagttgtacatattgataagtcagcgtatgtgtcaga

SEQ ID NO: 783 - DNA, 60 bp, Mus musculus
ttacatggcaaatctgaaaggaagacttaagcagggtaaagttaattgaaaggaggagct

SEQ ID NO: 784 - DNA, 60 bp, Mus musculus
agcaatctttgtatcaattatatcacactaatggatgaactgtgtaaggtaaggacaagc

SEQ ID NO: 785 - DNA, 60 bp, Mus musculus
ggtgtatggaaataaagtttagtcaatgttgaaaatctctcctggttgaatgacttgctc

SEQ ID NO: 786 - DNA, 60 bp, Mus musculus
ctttcagtctccttctgtgtctcgaaccttgaacaggatgtgataacttttctagaccac

SEQ ID NO: 787 - DNA, 60 bp, Mus musculus
gactgtttctgggaaaataagtatgtgaagtgatgcagaaaatccatctagacagttgag

SEQ ID NO: 788 - DNA, 60 bp, Mus musculus
tggtggcttgattgatttgatctgagagcagtttataacataatggagaactgtttgcag

SEQ ID NO: 789 - DNA, 60 bp, Mus musculus
agaagtctacctttaagatgacctatattggagagatattcactaagattctgttgcttc

SEQ ID NO: 790 - DNA, 60 bp, Mus musculus
actctctggtcatgatggttttccgaaatcaggttcctgacctgaaaatttgggttaatc

SEQ ID NO: 791 - DNA, 60 bp, Mus musculus
gttttcatgctttggaagtcttttctttgaaaaggcaaactgctgtatgaggagaaaata

SEQ ID NO: 792 - DNA, 60 bp, Mus musculus
gtgtgtaggaaaatgtaattaagtacaaggcttgtttatgggtggctatggaatgcagtc

SEQ ID NO: 793 - DNA, 60 bp, Mus musculus
gtttcctcatcaggtgtaatggcgtgtcctaatgaagctatttcttatgtataacagaga

SEQ ID NO: 794 - DNA, 60 bp, Mus musculus
tgaaaaaatgaaaagaatcagagatgaaataggagcgctcagaagtttttatgttctccc

SEQ ID NO: 795 - DNA, 60 bp, Mus musculus
aaagaaatgaaaaccgtcatttgcgattttcagggtacgtttctaatgtatccagaagtc

SEQ ID NO: 796 - DNA, 60 bp, Mus musculus
tttccagtgttctagttacattaatgagaacagaaacataaactatgacctaggggtttc

SEQ ID NO: 797 - DNA, 60 bp, Mus musculus
ttttgactcagttgactgtctcagactgtaagacctgaatgtctctgctccgaattcctg

SEQ ID NO: 798 - DNA, 60 bp, Mus musculus
cccgagttactaacaacattcttttgctatatgtagatcaagattaacagttcctcattc

SEQ ID NO: 799 - DNA, 60 bp, Mus musculus
gttttggtgcaaaagtcgtcctgtgtctcttgttcccttcattagaaaacatgctagagg

SEQ ID NO: 800 - DNA, 60 bp, Mus musculus
aggaaggaaaataggctttgttgtatgtacataagtggaattaacaagagtctttagtcc

SEQ ID NO: 801 - DNA, 60 bp, Mus musculus
tacagggaatggtctaagcataccatttcattcactgtattagtagacataactgttgag

SEQ ID NO: 802 - DNA, 60 bp, Mus musculus
gaaacgggctttgttgtaaaggtaatgaataggaaactcctcagattcaatggttaagaa

SEQ ID NO: 803 - DNA, 60 bp, Mus musculus
aagttaaggaaatactgagaatcggtcagttaacactctgaaaagctattcaaagcatag

SEQ ID NO: 804 - DNA, 60 bp, Mus musculus
aaatacatgcatttgtacagtgggccctgttcttgtgaagtccatctccatggtcattag

SEQ ID NO: 805 - DNA, 60 bp, Mus musculus
ccgttttattgattggaaatgtaagactcaaagaactcaggtttactggccaagatggca

SEQ ID NO: 806 - DNA, 60 bp, Mus musculus
ggaaagagagatcaaactaggaacctacaagatagttcactagcctaagatctttacttg

SEQ ID NO: 807 - DNA, 60 bp, Mus musculus
ttgattggtgtttctgagcattcagactccgcaccctcatttctaataaatgcaacattg

SEQ ID NO: 808 - DNA, 60 bp, Mus musculus
ctagtgaaatttatgtcagaatgacatatctgaactctgaattcatctctagtttccacg

SEQ ID NO: 809 - DNA, 60 bp, Mus musculus
tagttaatacttctctgaaatacatggtaacaactagtaagcaagagataccgcagattg

SEQ ID NO: 810 - DNA, 60 bp, Mus musculus
tggattattcccgccaaagcacccaagtcggcctgtttaattggagaaagatggaattaa

SEQ ID NO: 811 - DNA, 60 bp, Mus musculus
gatccaggcaacctctgtttaccctggggcctacaatgcctttcagatccgttctggaaa

SEQ ID NO: 812 - DNA, 60 bp, Mus musculus
gttccatctgacttaaacaaaaaccgtagtttccagctcagaatcatcctaacatagaaa

SEQ ID NO: 813 - DNA, 60 bp, Mus musculus
gtaggggaataactaaccaaagtagagggaattctaagtttagtagtaaatgtggcttgg

SEQ ID NO: 814 - DNA, 60 bp, Mus musculus
ggtgtgggacttatggggtctacacaaaggtaaagaattacgtggactggatcctgaaaa

SEQ ID NO: 815 - DNA, 60 bp, Mus musculus
aggtatgacattttacatccttgaatcttacttactatgtgctaaacaattggcagaagg

SEQ ID NO: 816 - DNA, 60 bp, Mus musculus
tgcttgtgtgaactacctcaggatgaagggtaatgtttaacattccatacatgcctactg

SEQ ID NO: 817 - DNA, 60 bp, Mus musculus
cgatggacccaagataccgacatgagagtagtgttgaggatcaacagtgcccattattat

SEQ ID NO: 818 - DNA, 60 bp, Mus musculus
gcagccaaaatggaaatgtttaaattaactgtgttgtacaaatgtacccaacacaaaacc

SEQ ID NO: 819 - DNA, 60 bp, Mus musculus
ttgacatgatacattacgcctttgcagtgagctaataagctaacatttgtgcacagataa

SEQ ID NO: 820 - DNA, 60 bp, Mus musculus
tctcaactcatctcagattaggaagtatttggcagtattagccatcatgtgtccctgtga

SEQ ID NO: 821 - DNA, 60 bp, Mus musculus
attttcatgccgaatattccagcagctattataaaatgctaaattcactcatcctgtacg

SEQ ID NO: 822 - DNA, 60 bp, Mus musculus
gagaattaatcataaacggaagtttaaatgaggatttggactttggtaattgtccctgag

SEQ ID NO: 823 - DNA, 60 bp, Mus musculus
catgagcaaagcccaccctcccgagctgaagaagtttatggacaagaagttatcattgaa

SEQ ID NO: 824 - DNA, 60 bp, Mus musculus
ctctgtaaagtcaagttgcattgcatttacagttaattatggaaaagtcctaaatctggc

SEQ ID NO: 825 - DNA, 60 bp, Mus musculus
ttttcagggctataaaagtattatgtggaaatgaggcatcagaccaccggacgttaccac

SEQ ID NO: 826 - DNA, 60 bp, Mus musculus
aagaagctgaggaaaaacaggagagtgagaaaccgcttttggaactatgagttctgctct

SEQ ID NO: 827 - DNA, 60 bp, Mus musculus
cctgatggagtctgtgttactcaggaggcagcagttattgtggattctcaaacaaggaaa

SEQ ID NO: 828 - DNA, 60 bp, Mus musculus
agcaaatgggcattttacaagaagtacgaatcttatttttcctgtcctgcccctgggggt

SEQ ID NO: 829 - DNA, 60 bp, Mus musculus
ctgcacttgaatggactgaaaacttgctggattatctagaacaacaagatgacatgctac

SEQ ID NO: 830 - DNA, 60 bp, Mus musculus
agatttcaccgtactttctgatggtgtttttaaaaggccaagtgttgcaaaagtttgcac

SEQ ID NO: 831 - DNA, 60 bp, Mus musculus
ataaaaccacaaactagtatcatgcttataagtgcacagtagaagtatagaactgatggg

SEQ ID NO: 832 - DNA, 60 bp, Mus musculus
acctaaatgttcatgacttgagactattctgcagctataaaatttgaacctttgatgtgc

SEQ ID NO: 833 - DNA, 60 bp, Mus musculus
tttatagttctaggtttacaccagagaggagttaatttatcaacagcctaaaactgttgc

SEQ ID NO: 834 - DNA, 60 bp, Mus musculus
ttcttccacgaacagatattatgtcattttatccaatgccgataaaggagaaacaacttg

SEQ ID NO: 835 - DNA, 60 bp, Mus musculus
tacgtggtctggggacctgatgttggaatcctattgttgttaataaaactgagtaaagga

SEQ ID NO: 836 - DNA, 60 bp, Mus musculus
accaacttctgtcaaagaacagtaaagaacttgagatacatccatctttgtcaaatagtc

SEQ ID NO: 837 - DNA, 60 bp, Mus musculus
tgacacaaatagaggggtcaataaatttttagccaaaagcttcaaattctttcaggaagc

SEQ ID NO: 838 - DNA, 60 bp, Mus musculus
atcaccattgttagtgtcatcatcattgttcttaacgctcaaaaccttcacacttaatag

SEQ ID NO: 839 - DNA, 60 bp, Mus musculus
gccgcttttttgtaacctaaaaggccccatgaataagggcccatgttttgggcatttgta

SEQ ID NO: 840 - DNA, 60 bp, Mus musculus
ccaagaacaagtataaacttaagctctgtagaactgaaattctttcaagtcctttcgatc

SEQ ID NO: 841 - DNA, 60 bp, Mus musculus
aggacatcttgcaacttctatgcaataataaggatttccatctgacaaataagacaagtg

SEQ ID NO: 842 - DNA, 60 bp, Mus musculus
ggggagttctaataatagtaccattcatatcagcaagaacctaaaaatggttctgacttt

SEQ ID NO: 843 - DNA, 60 bp, Mus musculus
tgccactagttctgacttggggaatatggtcccttaaacatgccaaagtgagctttttaa

SEQ ID NO: 844 - DNA, 60 bp, Mus musculus
catcaatcctttgatggaacctcaaagtcctatagtcctaagtgacgctaacctccccta

SEQ ID NO: 845 - DNA, 60 bp, Mus musculus
cagttggaaaaatggatgaagctcaatgtagaagagggattatacagcagaactctggca

SEQ ID NO: 846 - DNA, 60 bp, Mus musculus
tcagtcaaatgtgcataactgtaaatcaacactaagagctctggaaggttaaaaaggtca

SEQ ID NO: 847 - DNA, 60 bp, Mus musculus
agcaggtgtttcggacttgcaatgagcaatgcaattttttctaaatatgaggatatttac

SEQ ID NO: 848 - DNA, 60 bp, Mus musculus
cttgcttctttagcaaaatattctggtttctagaagaggaagtctgtccaacaaggcccc

SEQ ID NO: 849 - DNA, 60 bp, Mus musculus
tctcaattttcaaggtgtatttcctatcaggaaacttgaagataatatggtctgaaccca

SEQ ID NO: 850 - DNA, 60 bp, Mus musculus
actggacaaagtattatgactttcaacaccaggaggtctccaaatacctgcacagacagc

SEQ ID NO: 851 - DNA, 60 bp, Mus musculus
ggctgttgagtgtaaaatgtgctttgtgtttgcttacaacatcagcttttagacacacag

SEQ ID NO: 852 - DNA, 60 bp, Mus musculus
tgagtgcaatgtgtcagatttcaccaagagatctccaaggtttgtaggtaatttgtggtt

SEQ ID NO: 853 - DNA, 60 bp, Mus musculus
gtcattgtccaaggtgacaggaggaactcagtcgttaaaatgacgagccttatttcatga

SEQ ID NO: 854 - DNA, 60 bp, Mus musculus
tcttagaatctggaattgagtgccatattttctgttctccaatgatacctggagaaatcc

SEQ ID NO: 855 - DNA, 60 bp, Mus musculus
tgctttcttattctttaaagatatttatttttcttctcattaaaataaaaccaaagtatt

SEQ ID NO: 856 - DNA, 60 bp, Mus musculus
ctgcatgttataactttatatgatggtgtagtgcatataagctatgagaatcagttatac

SEQ ID NO: 857 - DNA, 60 bp, Mus musculus
cgtgctggaggacgagagattccagaagcttctgaagcaagcagagaagcaggctgaaca

SEQ ID NO: 858 - DNA, 60 bp, Mus musculus
tggaggctttgtacccaaaacttttcaagcctgaaggaaaagcagaactgcgggattaca

SEQ ID NO: 859 - DNA, 60 bp, Mus musculus
tggaggatctgtgtgaaaaagaagtcaccctcacaaaccgccgtgcctaaggactctgtc

SEQ ID NO: 860 - DNA, 60 bp, Mus musculus
ctattttgtgtagacatcgtcttgcctgaatagactgtgggtgaatccaaatttggtcca

SEQ ID NO: 861 - DNA, 60 bp, Mus musculus
taattatctacattggggtaattgaagtagaaagatccatcttaactacggtaatctccg

SEQ ID NO: 862 - DNA, 60 bp, Mus musculus
ttgggtatcgtttatgtttccatcataacacatgcaataacatctaggaaatctttaccg

SEQ ID NO: 863 - DNA, 60 bp, Mus musculus
tctgatgtggaagtgcggtcattcctggtttaactcacagcaacttttaattggtctaag

SEQ ID NO: 864 - DNA, 60 bp, Mus musculus
atctcctgttaatgtatttgggtcaaatgcaaggccttaataaagaaatctggggcagaa

SEQ ID NO: 865 - DNA, 60 bp, Mus musculus
gcagcaagagaaaagagcaagagagccaaaggcaagaaatctctctgtcactccctttta

SEQ ID NO: 866 - DNA, 60 bp, Mus musculus
tgaggaaaagccccatgtgaaaccttatttctctaagaccatccgtgatctggaagtcgt

SEQ ID NO: 867 - DNA, 60 bp, Mus musculus
accggctgtacccaaatagaacgtcattttgatatgaaggatttcagcccctgaagattt

SEQ ID NO: 868 - DNA, 60 bp, Mus musculus
atggtttcttccagcaatttagcattgcctgaggggtctaaaagaataagttggttcttg

SEQ ID NO: 869 - DNA, 60 bp, Mus musculus
acaatctctgtcagcgaaaagttctacaacagctgtgctgcaaaacatgtacattccaag

SEQ ID NO: 870 - DNA, 60 bp, Mus musculus
aactgttactggattgaaattcccatcccctttccctaaaaattgtgccttagaaaaccc

SEQ ID NO: 871 - DNA, 60 bp, Mus musculus
cgactgaggttatgacatccttagactttgttgtatgctgcttcgaatgaaccagagata

SEQ ID NO: 872 - DNA, 60 bp, Mus musculus
tgcctcttcatcgccagtggtccaaagggcgcagagagcgcactagcagtcaatagtgtt

SEQ ID NO: 873 - DNA, 60 bp, Mus musculus
ccactaatatttagccagccttcatgtagaagacacatggaaacacagaagtaaactttt

SEQ ID NO: 874 - DNA, 60 bp, Mus musculus
agaaatgaacatacattgtcagcatttagaagtaagttgtgaagacagggacattaagtg

SEQ ID NO: 875 - DNA, 60 bp, Mus musculus
caaacgggatcctgtcttcttcttttctaatagaattttgtaaaggaaatgaatgtagcc

SEQ ID NO: 876 - DNA, 60 bp, Mus musculus
accgttctatcactgtggatggagaagaagcgtcactattggtctatgacatttgggaag

SEQ ID NO: 877 - DNA, 60 bp, Mus musculus
ctatttttgggagatgtctattgcggagtacagtaatatatacccagagtatgtctatag

SEQ ID NO: 878 - DNA, 60 bp, Mus musculus
acccaactccagtgctctctgtcttttagtacaggattttcacccatgtgcatgaaaaat

SEQ ID NO: 879 - DNA, 60 bp, Mus musculus
ttaccatttttggttaaatggccaaattcagaaaataactccatttgaatctccagcagg

SEQ ID NO: 880 - DNA, 60 bp, Mus musculus
tcaccatactttgaaagtgtaaactaccacatattaacatgtgtgatttaagaccctcag

SEQ ID NO: 881 - DNA, 60 bp, Mus musculus
tgttgccctcagatatgtcagatcaacttggaaggaaagaccttctactccaagaaggac

SEQ ID NO: 882 - DNA, 60 bp, Mus musculus
tctaacaagtgtatttgtgttatctttaaaatagaacaattgtatcttgaaatggtaaat

SEQ ID NO: 883 - DNA, 60 bp, Mus musculus
cgacactgggtggccctgcgacaggtagatggcatctactataatctggactcaaagctc

SEQ ID NO: 884 - DNA, 60 bp, Mus musculus
tctcagaggtgttgaagatttatcatcttgaatcctccacaaatacagatacagtcccaa

SEQ ID NO: 885 - DNA, 60 bp, Mus musculus
tcttttcacctcgatcagcatcatgagtcatcacagatcatgtaattagtttctgggcca

SEQ ID NO: 886 - DNA, 60 bp, Mus musculus
tgggaattgcatttaggatagaattgtatctgatttgcaaaatccataagctctcatgcc

SEQ ID NO: 887 - DNA, 60 bp, Mus musculus
tactcccacagttgtatagaagtcgaatagtgaaggagctgggagaaaactgcttcagct

SEQ ID NO: 888 - DNA, 60 bp, Mus musculus
ccgcacttagcctagcacctttcttacatgatctcaagttgaaccgacttccttaactct

SEQ ID NO: 889 - DNA, 60 bp, Mus musculus
gctttggaattaaagaggaggatatagatgaaaaccccctcttttgaattaagatttgag

SEQ ID NO: 890 - DNA, 60 bp, Mus musculus
aaatcagatatgcaggtcatctgataaatgagttaatgtttgatattcggggtatctcac

SEQ ID NO: 891 - DNA, 60 bp, Mus musculus
gaaccatatgctggaatgaaacataagagttttcaacagttatcctctcacctctgtatg

SEQ ID NO: 892 - DNA, 60 bp, Mus musculus
gtatcgtcaatcccagtcagtaagataagttgaaacaagattatcctcaagtgtagattt

SEQ ID NO: 893 - DNA, 60 bp, Mus musculus
gtcaaaaacgccttcaggaagccttagagcgtcagaaggagtttgatccgaccataacag

SEQ ID NO: 894 - DNA, 60 bp, Mus musculus
aatagaatcttttcacttaggaatggagaacaagccagttcagaggaccccaaagtctag

SEQ ID NO: 895 - DNA, 60 bp, Mus musculus
cgtggaggatgggctagcctgagctctgggactaatctttattacatacttgttaatgag

SEQ ID NO: 896 - DNA, 60 bp, Mus musculus
cttatagggagaatgttctattcctcaatccatactcattcctacagtatgcgctctgga

SEQ ID NO: 897 - DNA, 60 bp, Mus musculus
agcagggggattatgttaagtcaaatgcgtgtgtctcaaaagtgacatgtttaactgctc

SEQ ID NO: 898 - DNA, 60 bp, Mus musculus
actctgtaccctactggaaccactctgtaaagagacaaagctgtatgtgccacttcagta

SEQ ID NO: 899 - DNA, 60 bp, Mus musculus
ttacaggtcactgtttgtcacttttgtgtaccagcttccccattagaattcaaccgatac

SEQ ID NO: 900 - DNA, 60 bp, Mus musculus
atggaagcgaggtcattctgcgaacattggagatcttttattacaagtctgcttgttaat

SEQ ID NO: 901 - DNA, 60 bp, Mus musculus
taaaattagtgtcctgggagagatgaccattttaacttctatgcttatttcacatgggaa

SEQ ID NO: 902 - DNA, 60 bp, Mus musculus
tcgacgtcaatcttacctctctaggcaacatgttatccccggatgatcagaaattcccaa

SEQ ID NO: 903 - DNA, 60 bp, Mus musculus
acctgtgttttgtttttgttttaagaaaccaaagtgcaccaagatagcatgctcttgaga

SEQ ID NO: 904 - DNA, 60 bp, Mus musculus
ctgcaggtaactctcattggaagaaaaagaaactacaagagcaaacagaagccatgggaa

SEQ ID NO: 905 - DNA, 60 bp, Mus musculus
aaagatttcatccacgtctggcgtagtggaaaacccgaagggaatatgtaatgatctttc

SEQ ID NO: 906 - DNA, 60 bp, Mus musculus
gtgttgtaccctaatttgaatttaaagtaggcagtaggtagggttaattggtagactatc

SEQ ID NO: 907 - DNA, 60 bp, Mus musculus
cttgggtttgagcactcagaacacatggctgcaatcatcaagacagttcacagttagctt

SEQ ID NO: 908 - DNA, 60 bp, Mus musculus
ccctaagacaatgaaactcagaactctgtgattcctgtggaaatatttaaaactgaaatg

SEQ ID NO: 909 - DNA, 60 bp, Mus musculus
atttatagaggtatccttaacatgctgacttcagtaactgcccttgtttctaaggaagtc

SEQ ID NO: 910 - DNA, 60 bp, Mus musculus
acctgtagcttcactgtgaacttgtgggcttggctggtcttaggaacttgtacctataaa

SEQ ID NO: 911 - DNA, 60 bp, Mus musculus
taatccctggcaaagtcaagactgtgggaaactagaactggttactcactactgctggta

SEQ ID NO: 912 - DNA, 60 bp, Mus musculus
ttagcttcatgaccccaaggttaaggttctgccaacaagcattctgcctgacatctactt

SEQ ID NO: 913 - DNA, 60 bp, Mus musculus
aataaaggccccttagaagctactgtaaagctcttcaaagttttcatgtaatcataggca

SEQ ID NO: 914 - DNA, 60 bp, Mus musculus
agagatggagactacactgggtagattctagtttttagttcttattaatgtgggggagta

SEQ ID NO: 915 - DNA, 60 bp, Mus musculus
tatggccatttggtttcagcatgtcaggagatttctaatgatttgtggcaatatcagcaa

SEQ ID NO: 916 - DNA, 60 bp, Mus musculus
tgtgtcaagataatcctgagtcaacctggacacttaatccctttggacctctatctggag

SEQ ID NO: 917 - DNA, 60 bp, Mus musculus
ccacccattaaaatgacagtacaagtagaccacagtttaaagtagttagtctaattctac

SEQ ID NO: 918 - DNA, 60 bp, Mus musculus
catagtggaaatatgctcatcttttatgctatatgtattaaacctcgacttagccctgaa

SEQ ID NO: 919 - DNA, 60 bp, Mus musculus
gttgaggctgacgacctcccagaggcaatctctggatctggaactttgggcatcatcgga

SEQ ID NO: 920 - DNA, 60 bp, Mus musculus
accaaccagggactagtttgatgctatctttgcctgtctcttggctcttaacaatgccta

SEQ ID NO: 921 - DNA, 60 bp, Mus musculus
ccagggaaggaacgatccattcagtggttttaaaatatctcttcctcaacagaaaaagat

SEQ ID NO: 922 - DNA, 60 bp, Mus musculus
ggtgcaagctagtactcacactgtcacacctttacgcatgcgaaaggtaatgtgctaaat

SEQ ID NO: 923 - DNA, 60 bp, Mus musculus
agatcagtgctctggacagtaagatccatgagacgattgagtccataaaccagctcaaga

SEQ ID NO: 924 - DNA, 60 bp, Mus musculus
atatccctgctaacttaacagcagttagtttccttgttatgaataaaaatgacagtctgg

SEQ ID NO: 925 - DNA, 60 bp, Mus musculus
aaagcaaatgttagtaaaaagctggtgtgcatagtcttgttacattgatgcagtttttcc

SEQ ID NO: 926 - DNA, 60 bp, Mus musculus
caacttgctgaataatgacttccattgagtaaacatttggctctggttatcttcagggat

SEQ ID NO: 927 - DNA, 60 bp, Mus musculus
aggaattagtaacgtttcatccaagtaaccttgttacagtgaacaagtgtcaagtgctca

(19) United States
(12) Patent Application Publication
(10) Pub. No.: US 2007/0092886 A1
(43) Pub. Date: Apr. 26, 2007

(54) Methods and compositions for diagnosis, monitoring and development of therapeutics for treatment of atherosclerotic disease

(76) Inventors: Raymond Tabibiazar, Menlo Park, CA (US); Thomas Quertermous, Stanford, CA (US)

Correspondence Address: MORRISON & FOERSTER LLP, 755 PAGE MILL RD, PALO ALTO, CA, 94304-1018, US

(21) Appl. No.: 11/387,484

(22) Filed: Mar. 22, 2006

Related U.S. Application Data
(60) Provisional application No. 60/664,550, filed on Mar. 22, 2005.

(51) Int. Cl. C12Q 1/68; C12M 3/00

(52) U.S. Cl. 435/6; 435/287.2

(57) ABSTRACT

Polynucleotide sequences are provided that correspond to genes that are differentially expressed in atherosclerotic disease conditions. Methods for using these sequences to detect gene expression and/or for transcriptional profiling in mammals are also provided. The polynucleotide sequences of the invention may be used, for example, to diagnose atherosclerotic disease, to monitor extent of progression or efficacy of treatment or to assess prognosis of atherosclerotic disease, and/or to identify compounds effective to treat an atherosclerotic disease condition.

### CROSS-REFERENCE TO RELATED APPLICATIONS

This application claims the benefit of U.S. Provisional Application No. 60/664,550, filed Mar. 22, 2005, which is incorporated by reference herein in its entirety.

### FIELD OF THE INVENTION

This application is in the field of atherosclerotic disease. In particular, this invention relates to methods and compositions for diagnosing, monitoring, and development of therapeutics for atherosclerotic disease.

### BACKGROUND OF THE INVENTION

Atherosclerosis is the primary cause of heart disease and stroke (Kannel and Belanger (1991) Am. Heart J 121:951-57), and is the most common cause of morbidity and mortality in the United States (NHLBI Morbidity and Mortality Chartbook, National Heart, Lung, and Blood Institute, Bethesda, MD, May, 2002; NHLBI Fact Book, Fiscal Year 2003, pp. 35-53, National Heart, Lung, and Blood Institute, Bethesda, MD, February, 2004). Atherosclerosis is currently conceptualized as a chronic inflammatory disease of the arterial vessel wall that develops due to complex interactions between the environment and the genetic makeup of an individual (Ross (1999) N Engl J Med 340:115-26). Development of an atherosclerotic plaque occurs in stages, beginning with simple fatty streak formation and culminating in complex calcified lesions containing abnormal accumulation of smooth muscle cells, inflammatory cells, lipids, and necrotic debris. It is likely that the various stages of atherosclerotic disease are governed by a set of genes that are expressed by a variety of cell types present in the vessel wall.

The propensity for developing atherosclerosis is dependent on underlying genetic risk, and varies as a function of age and exposure to environmental risk factors. However, despite the chronic nature of atherosclerotic disease, knowledge regarding temporal gene expression during the course of disease progression is very limited. The prolonged, chronic, and unpredictable nature of the disease in humans, by virtue of heterogeneous genetic and environment factors, has limited systematic temporal gene expression studies in humans.

The roles of a limited number of genes that are differentially expressed in vascular disease have been identified, and a few of these genes linked through mechanistic studies to disease processes (Glass and Witztum (2001) Cell 104:503-16; Breslow (1996) Science 272:685-88; Lusis (2000) Nature 407:233-41). Recent efforts to identify disease related gene expression patterns have employed transcriptional profiling with DNA microarrays. However, these studies have included relatively small arrays (Wuttge et al. (2001) Mol Med 7:383-392) as well as limited time points, with the primary comparison between normal and late stage diseased tissue (Archacki et al. (2003) Physiol Genomics 15:65-74; Faber et al. (2002) Curr Opin Lipidol 13:545-552; McCaffrey et al. (2000) J Clin Invest 105:653-662; Randi et al. (2003) J Throm Haemost 1:829-835; Seo et al. (2004) Arterioscler Thromb Vasc Biol 24:1922-1927; Zohlnhofer et al. (2001) Mol Cell 7:1059-1069. Utilizing microarrays in animal models, where a disease process can be studied over time, the impact of individual risk factors and perturbations on the expression of individual genes during disease development can be studied systematically without a priori knowledge of gene identity. The temporal expression patterns of the genes can then be correlated with the well-described disease stages.

There is a need for a comprehensive list of atherosclerosis-related genes that are predictive of atherosclerotic disease conditions, for use as diagnostic markers and for discovery of biochemical pathways involved in development of atherosclerotic disease and discovery and/or testing of new therapeutics.

### BRIEF SUMMARY OF THE INVENTION

This invention provides compositions, methods, and kits for detection of gene expression, diagnosis, monitoring, and development of therapeutics with respect to atherosclerotic disease.

In one aspect, the invention provides a system for detecting gene expression, comprising at least two isolated polynucleotide molecules, wherein each isolated polynucleotide molecule detects an expressed gene product from a gene that is differentially expressed in atherosclerotic disease in a mammal. In one embodiment, the differentially expressed gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the differentially expressed gene is selected from the group of genes corresponding to the polynucleotide sequences depicted in SEQ ID NOs: 1-927. In various embodiments, a system for detecting gene expression comprises any of at least 3, 5, 10, 15, 20, 25, 30, 35, 40, 50, 60, 70, 80, 90, or 100 of the isolated polynucleotide molecules described herein or their polynucleotide complements, or human homologs or orthologs thereof. In one embodiment, the gene expression system comprises at least two isolated polynucleotide molecules, wherein each isolated polynucleotide molecule detects an expressed gene product, wherein the gene is selected from the group of genes corresponding to the polynucleotide sequences depicted in SEQ ID NOs: 1-927, wherein the gene is differentially expressed in atherosclerotic disease in a mammal, and wherein the gene expression system comprises at least 1, 3, 5, 10, 15, 20, 25, or 30 isolated polynucleotide molecules that detect genes corresponding to the polynucleotide sequences selected from the group consisting of SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

In some embodiments, the isolated polynucleotide molecules are immobilized on an array, which may be selected from the group consisting of a chip array, a plate array, a bead array, a pin array, a membrane array, a solid surface array, a liquid array, an oligonucleotide array, a polynucleotide array, a cDNA array, a microtiter plate, a membrane, and a chip. The isolated polynucleotide molecules may be selected from the group consisting of synthetic DNA, genomic DNA, cDNA, RNA, or PNA. A gene corresponding to an isolated polynucleotide molecules described herein may be differentially expressed in any blood vessel or portion thereof which has developed an atherosclerotic or inflammatory disease, for example, the aorta, a coronary artery, the carotid artery, or a blood vessel of the peripheral vasculature.

In another aspect, the invention provides a kit comprising a system for detecting gene expression as described above. In one embodiment, the kit comprises an array comprising a system for detecting gene expression as described above.

In another aspect, the invention provides a method of detecting gene expression, comprising contacting products of gene expression with the system for detecting gene expression as described above. In one embodiment, the method comprises isolating mRNA, for example from a sample from individual who has or who is suspected of having an atherosclerotic disease, and hybridizing the RNA to the polynucleotide molecules from the system for detecting gene expression. In another embodiment, the method comprises isolating mRNA, converting the RNA to nucleic acid derived from the RNA, e.g., cDNA, and hybridizing the nucleic acid derived from the RNA to the polynucleotide molecules of the system for detecting gene expression. Optionally, the RNA may be amplified prior to hybridization to the system for gene expression. Optionally, the RNA is detectably labeled, and determination of presence, absence, or amount of an RNA molecule corresponding to a gene detected by a polynucleotide molecule of the system for detecting gene expression comprises detection of the label.

In another embodiment, the method for detecting gene expression comprises isolating proteins from an individual who has or who is suspected of having an atherosclerotic disease, and detecting the presence, absence, or amount of one or more proteins corresponding to the gene expression product of a gene that is differentially expressed in atherosclerotic disease and corresponds to a polynucleotide molecule of the system for detecting gene expression as described above. Detection may be via an antibody that recognizes the protein, for example, by contacting the isolated proteins with an antibody array.

In another aspect, the invention provides a method for diagnosing an atherosclerotic disease in an individual, comprising contacting polynucleotides derived from a sample from the individual with a system for detecting gene expression as described above. In one embodiment, the method comprises detecting hybridization complexes formed, if any, wherein presence, absence or amount of hybridization complexes formed from at least one of the polynucleotides from the individual is indicative of presence or absence of the atherosclerotic disease. In another embodiment, the method comprises comparing levels of expression of the genes with a molecular signature indicative of the presence or absence of the atherosclerotic disease.

In another aspect, the invention provides a method for assessing extent of progression of atherosclerotic disease in an individual, comprising contacting polynucleotides derived from a sample from the individual with a system for detecting gene expression as described above. In one embodiment, the method comprises detecting hybridization complexes formed, if any, wherein presence, absence or amount of hybridization complexes formed from at least one of the polynucleotides from the individual is indicative of extent of progression of the atherosclerotic disease. In another embodiment, the method comprises detecting hybridization complexes formed, if any, and comparing levels of expression of the genes with a molecular signature indicative of extent of progression of the atherosclerotic disease.

In another aspect, the invention provides a method of assessing efficacy of treatment of atherosclerotic disease in an individual, comprising contacting polynucleotides derived from a sample from the individual with a system for detecting gene expression as described above. In one embodiment, the method comprises detecting hybridization complexes formed, if any, wherein presence, absence or amount of hybridization complexes formed from at least one of the polynucleotides from the individual is indicative of extent of progression of the atherosclerotic disease. In another embodiment, the method comprises comparing levels of expression of the genes with a molecular signature indicative of extent of progression of the atherosclerotic disease.

In another aspect, the invention provides a method for determining prognosis of atherosclerotic disease in an individual, comprising contacting polynucleotides derived from a sample from the individual with a system for detecting gene expression as described above. In one embodiment, the method comprises detecting hybridization complexes formed, if any, wherein presence, absence or amount of hybridization complexes formed from at least one of the polynucleotides from the individual is indicative of prognosis of the atherosclerotic disease. In another embodiment, the method comprises comparing levels of expression of the genes with a molecular signature indicative of prognosis of the atherosclerotic disease.

In another aspect, the invention provides a method for identifying a compound effective to treat an atherosclerotic disease, comprising administering a test compound to a mammal with an atherosclerotic disease condition and contacting polynucleotides derived from a sample from the mammal with a system for detecting gene expression as described above. In one embodiment, the method comprises detecting hybridization complexes formed, if any, wherein presence, absence or amount of hybridization complexes formed from at least one of the polynucleotides from the individual is indicative of treatment of the disease. In another embodiment, the invention comprises detecting hybridization complexes formed, if any, and comparing levels of expression of the genes with a molecular signature indicative of treatment of the disease.

In another aspect, the invention provides a method of monitoring atherosclerotic disease in a mammal, comprising detecting the expression level of at least one, at least two, at least ten, at least one hundred, or more genes selected from the group of genes corresponding to the polynucleotide sequences depicted in SEQ ID NOs: 1-927. In some embodiments, at least one of the genes for which expression level is detected is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In one embodiment, the atherosclerotic disease comprises coronary artery disease. In one embodiment, the atherosclerotic disease comprises carotid atherosclerosis. In one embodiment, the atherosclerotic disease comprises peripheral vascular disease. In some embodiments, the expression level of said gene(s) is detected by measuring the RNA expression level. In one embodiment, RNA is isolated from the individual prior to detection of the RNA expression level. Measurement of RNA expression level may comprise amplifying RNA from an individual, for example, by polymerase chain reaction (PCR), using a primer that is complementary to a polynucleotide sequence corresponding to a gene to be detected, wherein the gene corresponds to a polynucleotide sequence selected from the group of genes depicted in SEQ ID NOs: 1-927. In some embodiments, a primer is used that is complementary to a polynucleotide sequence corresponding to a gene to be detected, wherein the gene corresponds to a polynucleotide sequence selected from the group of genes depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. Measurement of RNA expression level may comprise hybridization of RNA from the individual to a polynucleotide corresponding to a gene to be detected, wherein the gene corresponds to a polynucleotide sequence selected from the group of genes depicted in SEQ ID NOs: 1-927. In some embodiments, RNA from the individual is hybridized to a polynucleotide corresponding to a gene to be detected, wherein the gene to be detected is selected from the group of genes depicted in 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In some embodiments, gene expression level is detected by measuring the expressed protein level. In some embodiments, the method further comprises selecting an appropriate therapy for treatment or prevention of the atherosclerotic disease. In some embodiments, gene expression level, for example, RNA or protein level, is detected in serum from an individual.

In another aspect, the invention provides a method of monitoring atherosclerotic disease in an individual, comprising detecting RNA expressed from at least one gene selected from the group of genes corresponding to at least one polynucleotide sequence depicted in SEQ ID NOs: 1-927. In one embodiment, the at least one gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In one embodiment, the method comprises measuring the expressed RNA in serum from the individual.

In another aspect, the invention provides a method of monitoring atherosclerotic disease in an individual, comprising detecting protein expressed from at least one gene selected from the group of genes corresponding to at least one polynucleotide sequence depicted in SEQ ID NOs:1-927. In one embodiment, the at least one gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In one embodiment, the method comprises measuring the expressed protein in serum from the individual.

### BRIEF DESCRIPTION OF THE FIGURES

FIG. 1 depicts the experimental design of the experiments described in Example 1. ApoE deficient mice (C57BL/6J-Apoe^{5mlUnc}), were fed non-cholate-containing high-fat diet from 4 weeks of age for a maximum period of 40 weeks. Aortas were obtained for transcriptional profiling at pre-determined time intervals corresponding to various stages of atherosclerotic plaque formation. For each time point, aortas from 15 mice were combined into 3 pools for microarray replicate studies. To eliminate gene expression differences due to aging, diet, and genetic differences, a number of control groups were also used at each time point, including apoE deficient mice on normal chow, aw well as C57Bl/6 and C3H/HeJ wild type mice on both normal and atherogenic diets.

FIG. 2 depicts quantification of atherosclerotic disease in the experiments described in Example 1. Percent lesion area was determined by calculating the ratio of atherosclerotic area versus total surface area of the aorta. ApoE-deficient mice (n=7) on high-fat diet were compared to other control mice (n=5-7 for each mouse/diet combination). Representative time intervals were used for analysis, including baseline (TOO) measurements in mice prior to initiation of diet at 4 weeks of age and end point measurements corresponding to 40 weeks (T40) on either high-fat or normal diet. At T00, three were no statistically significant differences in lesion area among the various conditions. At 40 weeks on high-fat diet, the controls did not develop any lesions. In contrast to the control mice, the ApoE-deficient mice on normal chow and on high-fat diet had significantly larger atherosclerotic area (14.00% +/−3.92%, p<0.0001, and 37.98% +/−6.3%, p<0.0001, respectively.)

FIG. 3 depicts atherosclerosis genes identified in the experiments described in Example 1. Employing a newly-developed statistical algorithm which relies on permutation analysis and generalized regression, atherosclerosis-related genes were identified. Selecting the genes on the basis of their false detection rate (FDR<0.05) and depicting their expression with a heatmap (ordered by hierarchical clustering), demonstrates profiles which closely correlate with disease progression. The heatmap is a graphic representation of expression patterns of 6 parallel time course studies with time progressing from left to right for each of the 6 sets of strain-diet combination. Each set of the strain-diet combination therefore contains 15 columns (3 for each of 5 time points). Each row represents the row normalized expression pattern of a single gene. The dominant temporal pattern of expression is one that increases linearly with time (667 genes). Fewer genes (64) reveal an opposite pattern. HF: high-fat diet; NC: normal chow.

FIG. 4 depicts time-related patterns of gene expression in atherosclerosis observed in the experiments described in Example 1. Using AUC analysis, a number of distinct time-related patterns of gene expression in ApoE-deficient mice on high-fat diet were observed. Eight different time-related patterns are depicted, with the y-axis representing normalized gene expression values and the x-axis representing 6 different time points from time 0 to 40 weeks. The genes in each pattern were clustered based on positive correlation values. The mean distance of genes from the center of each cluster is noted in parentheses for each pattern. Using enrichment analysis for each cluster of genes, specific pathways were found to be associated with these patterns that reflect particular biological processes.

FIG. 5 depicts the identification and validation of mouse atherosclerotic disease classifier genes as determined in the experiments described in Example 1. FIG. 5A depicts identification of the classification gene set. The SVM algorithm described in Example 1 was employed to rank genes based on their abilities to accurately discriminate between 5 time points in ApoE-deficient mice on high-fat diet. An optimal set of 38 genes was identified to classify the experiments at a minimal error rate of 15%. The optimal 15% error rate was determined with a 1000 step cross-validation method with 25% of the experiments employed as the test group and the rest as the training group. FIG. 5B depicts classification of an independent mouse atherosclerosis data set. Aortas of ApoE-deficient mice aged 16 weeks were used for gene expression profiling utilizing a different microarray and labeling protocol than in the experiment depicted in FIG. 5A. Using the SVM algorithm, where known experiments were the five time points in the original experimental design and the independent set of experiments was the test set, these mice most closely classified with the 24 week time point. SVM scores for each experiment based on one-versus-all comparisons are represented graphically in a heatmap.

FIG. 6 depicts expression of atherosclerosis-related genes in human coronary artery disease, as described in Example 1. To investigate the expression profile of differently regulated mouse genes in human coronary artery atherosclerosis, 40 coronary artery samples with and without atherosclerotic lesions were used for transcriptional profiling. Atherosclerosis-associated mouse genes were matched to human orthologs/homologs by gene symbol and by known homology, and their expression was compared in human atherosclerotic plaques classified as lesion versus no lesion (SAM FDR<0.025). The expression of the top genes is represented graphically as a heatmap, where rows represent row normalized expression of each gene and the columns represent coronary artery samples. Calculated SAM FDR<0.009 for d-score 4.25-2.45, FDR<0.015 for d-score 2.41-2.357, FDR<0.025 for d-score 2.33-2.05.

FIG. 7 depicts the experimental design of the experiments described in Example 2. FIG. 7A: Four-week-old female C3H/HeJ (C3H) and C57B16 (C57) mice were fed normal chow vs. high-fat diet for the maximum period of 40 weeks. Triplicate microarray experiments were performed for each time point using 3 pools of 5 aortas at 0, 4, 10, 24, and 40 weeks on either diet (total of 15 mice per time point). FIG. 7B: Data analysis overview. Of the 20,283 genes present on the array, 311 genes were found to be significantly differentially expressed between C3H and C57 mice at baseline (SAM FDR 10% and >1.5-fold change). Differential gene expression during aging was determined by comparing C57 vs. C3H time-course differences on normal and atherogenic high-fat diets using AUC analysis.

FIG. 8 depicts differential gene expression between C3H and C57 mice at baseline. The SAM analysis shown was associated with an FDR of 10%, and a total of 311 probes were identified as differentially regulated at this level of confidence. Lists represent a select group of genes (expressed sequence tags excluded) with higher expression in C3H (top 20 ranking genes) and C57 (top 45 ranking genes). The heatmap reflects normalized gene expression ratios and is organized with individual hybridizations for each of the 3 replicates for each mouse strain arranged along the x axis.

FIG. 9 depicts differential gene expression between C3H and C57 mice in response to normal aging. FIG. 9A: Response to aging was determined by comparing C57 vs. C3H time-course differences on normal diet (AUC analysis F statistic>10). FIG. 9B: Functional annotation of the 413 differentially expressed genes reveals differences in various biological processes, including growth and differentiation. The probability rates provided area based on Fisher exact test (P<0.02). FIG. 9C: K-means clustering of the 413 genes reveals several profiles of gene expression. Clusters 1, 4, and 9 reveal increased gene expression in C3H vs. C57 mice, whereas clusters 2, 6, and 14 reveal the opposite pattern.

FIG. 10 depicts differential gene expression between C3H and C57 mice in response to high-fat diet. FIG. 10A: Response to atherogenic stimulus was determined by comparing C57 vs. C3H time-course differences on high -fat diet (AUC analysis F statistic>10). FIG. 1OB: Functional annotation of the 509 differentially expressed genes reveals differences in various biological processes and cellular components. The probability rates provided are based on Fisher exact test (P<0.02). FIG. 1OC: K-means clustering of the 509 differentially expressed genes revealed several patterns of gene expression with clusters 3 and 9 exhibiting increased gene expression in C3H vs. C57 mice and clusters 8 and 10 with the opposite pattern.

FIG. 11 shows the results of evaluation in the apoe knockout model of genes identified as differentially expressed between C3H and C57 strains. FIG. 11A: ApoE knockout mice (C57BL/6J-Apoe^{™lUnc}) were fed normal chow versus high-fat diet for the maximum period of 40 weeks. Triplicate microarray experiments were preformed for each time point using 3 pools of 5 aortas at 0, 4, 10, 24, and 40 weeks for regular and high-fat diet groups (total of 15 mice per time point). SOMs were used to visualize patterns of expression of genes of interest. Genes which were differentially regulated by aging (FIG. 9, K-means clusters 1, 4, and 9 with higher expression in C3H and clusters 4, 6, and 14 with higher expression in C57) and genes identified with atherogenic stimuli (FIG. 10, K-means clusters 3 and 9 with higher expression in C3H and clusters 8 and 10 with opposite pattern) as well as genes which were differentially expressed at the baseline time point (FIG. 8), were grouped and their expression was studied using SOM analysis. SOM analysis reveals diverse patterns of expression of these genes throughout the development of atherosclerosis in apoe knockout mice. Cluster 8 contains genes that are consistently increasing in expression with progression of atherosclerosis. Pie charts reflect the analysis group from which the genes populating each cluster were derived. The relative size of sectors of the pie chart indicates the relative number of genes that are derived from the various staging groups. FIG. 11B lists genes with higher expression in C57 mice at baseline and in C3H mice at baseline or on a high fat diet.

### DETAILED DESCRIPTION OF THE INVENTION

The invention provides polynucleotide sequences that correspond to genes that are differentially expressed in atherosclerotic disease conditions, and methods for using these sequences to detect gene expression and/or for transcriptional profiling in mammals. The polynucleotide sequences provided herein may be used, for example, to diagnose, assess extent of progression, assess efficacy of treatment of, to determine prognosis of, and/or to identify compounds effective to treat an atherosclerotic disease condition. The polynucleotide sequences herein may also be used in methods for elucidation of biochemical pathways that are involved in development and/or maintenance of atherosclerotic disease conditions.

General Techniques

The practice of the present invention will employ, unless otherwise indicated, conventional techniques of molecular biology (including recombinant techniques), microbiology, cell biology, and biochemistry, which are within the skill of the art. Such techniques are explained fully in the literature, such as: Molecular Cloning: A Laboratory Manual, vol. 1-3, third edition (Sambrook et al., 2001); Oligonucleotide Synthesis (M.J. Gait, ed., 1984); Methods in Enzymology (Academic Press, Inc.); Current Protocols in Molecular Biology (F.M. Ausubel et al., eds., 1987); PCR Cloning Protocols, (Yuan and Janes, eds., 2002, Humana Press).

In addition to the above references, protocols for in vitro amplification techniques, such as the polymerase chain reaction (PCR), the ligase chain reaction (LCR), Qβ-replicase amplification, and other RNA polymerase mediated techniques (e.g., NASBA), useful, e.g., for amplifying oligonucleotide probes of the invention, are found in Mullis et al., U.S. Pat. No. (1987) 4,683,202; PCR Protocols: A Guide to Methods and Applications (Innis et al., eds.) Academic Press, Inc., San Diego, CA (1990); Amnheim and Levinson (1990) C&EN 36; The Journal of NIH Research (1991) 3:81; Kwoh et al. (1989) Proc Natl Acad Sci USA 86:1173; Guatelli et al. (1990) Proc Natl Acad Sci USA 87:1874; Lomell et al. (1989) J Clin Chem 35:1826; Landegren et al. (1988) Science 241:1077; Van Brunt (1990) Biotechnology 8:291; Wu and Wallace (1989) Gene 4:560; Barringer et al. (1990) Gene 89:117; Sooknanan and Malek (1995) Biotechnology 13:563. Additional methods, useful for cloning nucleic acids, include Wallace et al., U.S. Patent No. 5,426,039. Improved methods of amplifying large nucleic acids by PCR are summarized in Cheng et al. (1994) Nature 369:684, and the references therein.

Definitions

Unless defined otherwise, all scientific and technical terms are understood to have the same meaning as commonly used in the art to which they pertain. For the purpose of the present invention, the following terms are defined below.

As used herein, the term “gene expression system” or “system for detecting gene expression” refers to any system, device or means to detect gene expression and includes candidate libraries, oligonucleotide sets or probe sets.

The term “diagnostic oligonucleotide set” generally refers to a set of two or more oligonucleotides that, when evaluated for differential expression of their products, collectively yields predictive data. Such predictive data typically relates to diagnosis, prognosis, monitoring of therapeutic outcomes, and the like. In general, the components of a diagnostic oligonucleotide set are distinguished from nucleotide sequences that are evaluated by analysis of the DNA to directly determine the genotype of an individual as it correlates with a specified trait or phenotype, such as a disease, in that it is the pattern of expression of the components of the diagnostic nucleotide set, rather than mutation or polymorphism of the DNA sequence that provides predictive value. It will be understood that a particular component (or member) of a diagnostic nucleotide set can, in some cases, also present one or more mutations, or polymorphisms that are amenable to direct genotyping by any of a variety of well known analysis methods, e.g., Southern blotting, RFLP, AFLP, SSCP, SNP, and the like.

A “disease specific target oligonucleotide sequence” is a gene or other oligonucleotide that encodes a polypeptide, most typically a protein, or a subunit of a multi-subunit protein, that is a therapeutic target for a disease, or group of diseases.

A “candidate library” or a “candidate oligonucleotide library” refers to a collection of oligonucleotide sequences (or gene sequences) that by one or more criteria have an increased probability of being associated with a particular disease or group of diseases. The criteria can be, for example, a differential expression pattern in a disease state, tissue specific expression as reported in a sequence database, differential expression in a tissue or cell type of interest, or the like. Typically, a candidate library has at least 2 members or components; more typically, the library has in excess of about 10, or about 100, or about 500, or even more, members or components.

The term “disease criterion” is used herein to designate an indicator of a disease, such as a diagnostic factor, a prognostic factor, a factor indicated by a medical or family history, a genetic factor, or a symptom, as well as an overt or confirmed diagnosis of a disease associated with several indicators. A disease criterion includes data describing a patient's health status, including retrospective or prospective health data, e.g., in the form of the patient's medical history, laboratory test results, diagnostic test results, clinical events, medications, lists, response(s) to treatment and risk factors, etc.

The terms “molecular signature” or “expression profile” refers to the collection of expression values for a plurality (e.g., at least 2, but frequently at least about 10, about 30, about 100, about 500, or more) of members of a candidate library. In many cases, the molecular signature represents the expression pattern for all of the nucleotide sequences in a library or array of candidate or diagnostic nucleotide sequences or genes. Alternatively, the molecular signature represents the expression pattern for one or more subsets of the candidate library.

The terms “oligonucleotide” and “polynucleotide” and “nucleic acid,” used interchangeably herein, refer to a polymeric form of two or more nucleotides of any length and any three-dimensional structure (e.g., single-stranded, double-stranded, triple-helical, etc.), which contain deoxyribonucleotides, ribonucleotides, and/or analogs or modified forms of deoxyribonucleotides or ribonucleotides. Nucleotides may be DNA or RNA, and may be naturally occurring, or synthetic, or non-naturally occurring. A nucleic acid of the present invention may contain phosphodiester bonds or an alternate backbone, comprising, for example, phosphoramide, phosphorothioate, phosphorodithioate, O-methylphosphoroamidite linkages, and peptide nucleic acid backbones and linkages. The term polynucleotide includes peptide nucleic acids (PNA).

The terms “polypeptide,”“peptide,” and “protein” are used interchangeably herein to refer to a polymer of amino acid residues. The terms apply to amino acid polymers in which one or more amino acid residue is an analogue of a corresponding naturally occurring amino acid, as well as to naturally occurring amino acid polymers. The term also includes variants on the traditional peptide linkage joining the amino acids making up the polypeptide.

An “isolated” or “purified” polynucleotide or polypeptide is one that is substantially free of the materials with which it is associated in nature. By substantially free is meant at least 50%, preferably at least 70%, more preferably at least 80%, and even more preferably at least 90% free of the materials with which it is associated in nature.

As used herein, “individual” refers to a vertebrate, typically a mammal, such as a human, a nonhuman primate, an experimental animal, such as a mouse or rat, a pet animal, such as a cat or dog, or a farm animal, such as a horse, sheep, cow, or pig.

The term “healthy individual,” as used herein, is relative to a specified disease or disease criterion, e.g., the individual does not exhibit the specified disease criterion or is not diagnosed with the specified disease. It will be understood that the individual in question can exhibit symptoms, or possess various indicator factors, for another disease.

Similarly, an “individual diagnosed with a disease” refers to an individual diagnosed with a specified disease (or disease criterion). Such an individual may, or may not, also exhibit a disease criterion associated with, or be diagnosed with another (related or unrelated) disease.

An “array” is a spatially or logically organized collection, e.g., of oligonucleotide sequences or nucleotide sequence products such as RNA or proteins encoded by an oligonucleotide sequence. In some embodiments, an array includes antibodies or other binding reagents specific for products of a candidate library.

When referring to a pattern of expression, a “qualitative” difference in gene expression refers to a difference that is not assigned a relative value. That is, such a difference is designated by an “all or nothing” valuation. Such an all or nothing variation can be, for example, expression above or below a threshold of detection (an on/off pattern of expression). Alternatively, a qualitative difference can refer to expression of different types of expression products, e.g., different alleles (e.g., a mutant or polymorphic allele), variants (including sequence variants as well as post-translationally modified variants), etc.

In contrast, a “quantitative” difference, when referring to a pattern of gene expression, refers to a difference in expression that can be assigned a numerical value, such as a value on a graduated scale, (e.g., a 0-5 or 1-10 scale, a +-+++ scale, a grade 1-grade 5 scale, or the like; it will be understood that the numbers selected for illustration are entirely arbitrary and in no-way are meant to be interpreted to limit the invention).

The term “monitoring” is used herein to describe the use of gene sets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

System for Detecting Gene Expression

The invention provides a system for detecting expression of genes that are differentially expressed in atherosclerotic disease. In one embodiment, the system for detecting gene expression detects at least two expressed gene products of genes selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the system for detecting gene expression detects at least two expressed gene products of genes selected from the group of genes corresponding to the polynucleotide sequences depicted in SEQ ID NOs: 1-927. The term “corresponding” as used herein in the context of a gene corresponding to a polynucleotide sequence depicted in the Sequence Listing refers to a gene that is detectable by interaction of a product of expression of the gene (e.g., mRNA, protein) or a product derived from a product of expression of the gene (e.g., cDNA) with the system for detecting gene expression. The polynucleotide sequences represented by Sequence Identification Nos. 1-927 and accompanying identifying information are depicted in Table 1 below. These sequences have been shown to be differentially expressed in atherosclerosis in mice (see Example 1). The 60 mer sequences represented in Table I are encompassed within the genes indicated therein. The gene sequences are obtainable from publicly available databases such as GenBank, and at http://www.ncbi.nlm.nih.gov or http://source.stanford.edu/cgi-bin/source/sourceSearch, using the identifying information provided in Table 1.

In one embodiment, the system for detecting gene expression includes at least two isolated polynucleotide molecules, each of which detects an expressed gene product of a gene that is differentially expressed in atherosclerotic disease in a mammal. The gene expression system includes at least two isolated polynucleotides that each comprise at least a portion of a sequence depicted in the Sequence Listing or its complement (i.e., a polynucleotide sequence capable of hybridizing to a sequence depicted in the sequence listing). A system for detecting gene expression in accordance with the invention may include any of at least 2, 3, 5, 10, 15, 20, 25, 30, 40, 50, 60, 70, 80, 90, or 100 polynucleotides each comprising at least a portion of a polynucleotide depicted in the Sequence Listing or a polynucleotide complement thereof.

It is understood that the polynucleotides of the invention may have slightly different sequences than those identified herein. Such sequence variations are understood to those of ordinary skill in the art to be variations in the sequence that do not significantly affect the ability of the sequences to detect gene expression. For example, homologs and variants of the polynucleotides disclosed herein may be used in the present invention. Homologs and variants of these polynucleotide molecules possess a relatively high degree of sequence identity when aligned using standard methods. Polynucleotide sequences encompassed by the invention have at least 40-50, 50-60, 70-80, 80-85, 85-90, 90-95 or 95-100% sequence identity to the sequences disclosed herein.

It is understood that for expression profiling, variations in the disclosed polynucleotide sequences will still permit detection of gene expression. The degree of sequence identity required to detect gene expression varies depending on the length of an oligonucleotide. For example, for a 60mer (i.e., an oligonucleotide with 60 nucleotides), 6-8 random mutations or 6-8 random deletions do not affect gene expression detection. Hughes, T. R., et al. (2001) Nature Biotechnology 19:343-347. As the length of the polynucleotide sequence is increased, the number of mutations or deletions permitted while still allowing gene expression detection is increased.

As will be appreciated by those skilled in the art, the sequences of the present invention may contain sequencing errors. For example, there may be incorrect nucleotides, frameshifts, unknown nucleotides, or other types of sequencing errors in any of the sequences; however, the correct sequences will fall within the homology and stringency definitions herein.

In some embodiments, polynucleotide molecules are less than about any of the following lengths (in bases or base pairs): 10,000; 5000; 2500; 2000; 1500; 1250; 1000; 750; 500; 300; 250; 200; 175; 150; 125; 100; 75; 50; 25; 10. In some embodiments, polynucleotide molecules are greater than about any of the following lengths (in bases or base pairs): 10; 15; 20; 25; 30; 40; 50; 60; 75; 100; 125; 150; 175; 200; 250; 300; 350; 400; 500; 750; 1000; 2000; 5000; 7500; 10,000; 20,000; 50,000. Alternately, a polynucleotide molecule can be any of a range of sizes having an upper limit of 10,000; 5000; 2500; 2000; 1500; 1250; 1000; 750; 500; 300; 250; 200; 175; 150; 125; 100; 75; 50; 25; or 10 and an independently selected lower limit of 10; 15; 20; 25; 30; 40; 50; 60; 75; 100; 125; 150; 175; 200; 250; 300; 350; 400; 500; 750; 1000; 2000; 5000; or 7500, wherein the lower limit is less than the upper limit.

The isolated polynucleotides of the system for detecting gene expression may include DNA or RNA or a combination thereof, and/or modified forms thereof, and/or may also include a modified polynucleotide backbone. In some embodiments, the isolated polynucleotides are selected from the group consisting of synthetic oligonucleotides, genomic DNA, cDNA, RNA, or PNA.

In one embodiment, the system for detecting gene expression comprises two antibody molecules or antigen binding fragments thereof, each of which detects an expressed gene product (e.g., a polypeptide) of a gene that is differentially expressed in atherosclerotic disease in a mammal.

As used herein, “atherosclerotic disease” refers to a vascular inflammatory disease characterized by the deposition of atheromatous plaques containing cholesterol, lipids, and inflammatory cells within the walls of large and medium-sized blood vessels, which can lead to hardening of blood vessels, stenosis, and thrombotic and embolic events. Atherosclerosis includes coronary vascular disease, cerebral vascular disease, and peripheral vascular disease. The term “atherosclerotic disease” as used herein includes any condition associated with atherosclerosis in a mammal in which differential gene expression may be detected by a system for detecting gene expression as described herein. Examples of such atherosclerotic disease conditions include, but are not limited to, coronary artery disease (e.g., stable angina, unstable angina, exertional angina, myocardial infarction, congestive heart failure, sudden cardiac death, atrial fibrillation), cerebral vascular disease (e.g., stroke, cerebrovascular accident (CVA), transient ischemic attack (TIA), cerebral infarction, cerebral intermittent claudication), peripheral vascular disease (e.g., claudications), extracranial carotid disease, carotid plaque, and carotid bruit.

Arrays

In some embodiments, a system for detecting gene expression in accordance with the invention is in the form of an array. “Microarray” and “array,” as used interchangeably herein, comprise a surface with an array, preferably ordered array, of putative binding (e.g., by hybridization) sites for a biochemical sample (target) which often has undetermined characteristics. In one embodiment, a microarray refers to an assembly of distinct polynucleotide or oligonucleotide probes immobilized at defined positions on a substrate. Arrays may be formed on substrates fabricated with materials such as paper, glass, plastic (e.g., polypropylene, nylon, polystyrene), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

For example, microarrays, in which either defined cDNAs or oligonucleotides are immobilized at discrete locations on, for example, solid or semi-solid substrates, or on defined particles, enable the detection and/or quantification of the expression of a multitude of genes in a given specimen.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into the amplified nucleic acids. The amplified product is then contacted with a solid substrate, such as a glass slide, which is coated with an aldehyde or another reactive group which will form a covalent link with the reactive group that is on the amplified product and become covalently attached to the glass slide. Microarrays comprising the amplified products can be fabricated using a Biodot (BioDot, Inc. Irvine, Calif.) spotting apparatus and aldehyde-coated glass slides (CEL Associates, Houston, Tex.). Amplification products can be spotted onto the aldehyde-coated slides, and processed according to published procedures (Schena et al., Proc. Natl. Acad. Sci. U.S.A. (1995) 93:10614-10619). Arrays can also be printed by robotics onto glass, nylon (Ramsay, G., Nature Biotechnol. (1998), 16:40-44), polypropylene (Matson, et al., Anal Biochem. (1995), 224(l):110-6), and silicone slides (Marshall, A. and Hodgson, J., Nature Biotechnol. (1998), 16:27-31). Other approaches to array assembly include fine micropipetting within electric fields (Marshall and Hodgson, supra), and spotting the polynucleotides directly onto positively coated plates. Methods such as those using amino propyl silicon surface chemistry are also known in the art, as disclosed at www.cmt.corning.com and http://cmgm.stanford.edu/pbrown/.

One method for making microarrays is by making high-density polynucleotide arrays. Techniques are known for rapid deposition of polynucleotides (Blanchard et al., Biosensors & Bioelectronics, 11:687-690). Other methods for making microarrays, e.g., by masking (Maskos and Southern, Nuc. Acids. Res. (1992), 20:1679-1684), may also be used. In principle, and as noted above, any type of array, for example, dot blots on a nylon hybridization membrane, could be used. However, as will be recognized by those skilled in the art, very small arrays will frequently be preferred because hybridization volumes will be smaller.

In one embodiment, the invention provides an array comprising at least two isolated polynucleotide molecules, wherein each isolated polynucleotide molecule detects an expressed gene product of a gene selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927, and wherein the gene is differentially expressed in atherosclerotic disease in a mammal. In one embodiment, the invention provides an array comprising at least two isolated polynucleotide molecules, wherein each isolated polynucleotide molecule detects an expressed gene product of a gene selected from the group of genes corresponding to the polynucleotide sequences depicted in SEQ ID NOs:1-927, and wherein the gene is differentially expressed in atherosclerotic disease in a mammal. In various embodiments, an array in accordance with the invention comprises any of at least 2, 3, 5, 10, 15, 20, 25, 30, 40, 50, 60, 70, 80, 90, or 100 polynucleotides each comprising at least a portion of a polynucleotide depicted in the Sequence Listing or a polynucleotide complement thereof.

In another embodiment, the invention provides an array comprising at least two antibody molecules or antigen binding fragments thereof, wherein each antibody molecule or antigen binding fragment thereof detects an expressed gene product of a gene selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927, and wherein the gene is differentially expressed in atherosclerotic disease in a mammal. In another embodiment, the invention provides an array comprising at least two antibody molecules or antigen binding fragments thereof, wherein each antibody molecule or antigen binding fragment thereof detects an expressed gene product of a gene selected from the group of genes corresponding to the polynucleotide sequences depicted in SEQ ID NOs:1-927, and wherein the gene is differentially expressed in atherosclerotic disease in a mammal. In various embodiments, an antibody array in accordance with the invention comprises any of at least 2, 3, 5, 10, 15, 20, 25, 30, 40, 50, 60, 70, 80, 90, or 100 antibodies or antigen binding fragments thereof each recognizing an expression product (e.g., a polypeptide) of a gene corresponding to a polynucleotide sequence depicted in the Sequence Listing.

Methods of the Invention

Methods for Detecting Gene Expression

The invention provides methods for detecting gene expression, comprising contacting products of gene expression (e.g., mRNA, protein) in a sample with a system for detecting gene expression as described above, and detecting interaction between the products of gene expression in the sample and the system for detecting gene expression. The methods for detecting gene expression described herein may be used to detect or quantify differential expression and/or for expression profiling of a sample. As used herein, “differential expression” refers to increased (upregulated) or decreased (downregulated) production of an expressed product of a gene (e.g., mRNA, protein). Differential expression may be assessed qualitatively (presence or absence of a gene product) and/or quantitatively (change in relative amount, i.e., increase or decrease, of a gene product).

In one embodiment, MRNA from a sample is contacted with a system for detecting gene expression comprising isolated polynucleotide molecules as described above, and hybridization complexes formed, if any, between the mRNA in the sample and the polynucleotide sequences of the system for detecting gene expression, are detected. In other embodiments, the mRNA is converted to nucleic acid derived from the mRNA, for example, cDNA, and/or amplified, prior to contact with the system for detecting gene expression.

In another embodiment, polypeptides from a sample are contacted with a system for detecting gene expression comprising antibodies or antigen fragments thereof that bind to polypeptide expression products of genes corresponding to the polynucleotide sequences described herein, and binding between the antibodies and polypeptides in the sample, if any, is detected.

Methods for Expression Profiling

An “expression profile” or “molecular signature” is a representation of gene expression in a sample, for example, evaluation of presence, absence, or amount of a plurality of gene expression products, such as mRNA transcripts, or polypeptide translation products of mRNA transcripts. Expression patterns constitute a set of relative or absolute expression values for a number of RNA or protein products corresponding to the plurality of genes evaluated, referred to as the subject's “expression profile” for those nucleotide sequences. In various embodiments, expression patterns corresponding to at least about 2, 5, 10, 20, 30, 50, 100, 200, or 500, or more nucleotide sequences are obtained. The expression pattern for each differentially expressed component member of the expression profile may provide a specificity and sensitivity with respect to predictive value, e.g., for diagnosis, prognosis, monitoring treatment, etc. In some embodiments, a molecular signature is determined by a statistical algorithm that determines the optimal relation between patterns of expression for various genes.

In some embodiments, an expression profile from an individual is compared with a reference expression profile to determine, for example, presence or absence of a disease condition, symptom, or criterion, extent of progression of disease, effectiveness of treatment of disease, or prognosis for prophylaxis, therapy, or cure of disease.

As used herein, the term “subject” refers to an individual regardless of health and/or disease status. For example, a subject may be a patient, a study participant, a control subject, a screening subject, or any other class of individual from whom a sample is obtained and assessed in the context of the invention. Accordingly, a subject may be diagnosed with a disease, can present with one or more symptom of a disease, or may have a predisposing factor, such as a genetic or medical history factor, for a disease. Alternatively, a subject may be healthy with respect to any of the aforementioned disease factors or criteria. It will be appreciated that the term “healthy” as used herein, is relative to a specified disease condition, factor, or criterion. Thus, an individual described as healthy with reference to any specified disease or disease criterion, can be diagnosed with any other one or more disease, or may exhibit any other one or more disease criterion.

Methods for Obtaining Expression Data

Numerous methods for obtaining expression data are known, and any one or more of these techniques, singly or in combination, are suitable for determining expression profiles in the context of the present invention. For example, expression patterns can be evaluated by northern analysis, PCR, RT-PCR, Taq Man analysis, FRET detection, monitoring one or more molecular beacon, hybridization to an oligonucleotide array, hybridization to a CDNA array, hybridization to a polynucleotide array, hybridization to a liquid microarray, hybridization to a microelectric array, molecular beacons, cDNA sequencing, clone hybridization, cDNA fragment fingerprinting, serial analysis of gene expression (SAGE), subtractive hybridization, differential display and/or differential screening (see, e.g., Lockhart and Winzeler (2000) Nature 405:827-836, and references cited therein).

For example, specific PCR primers are designed to a member(s) of a candidate nucleotide library (e.g., a polynucleotide member of a system for detecting gene expression). cDNA is prepared from subject sample RNA by reverse transcription from a poly-dT oligonucleotide primer, and subjected to PCR. Double stranded cDNA may be prepared using primers suitable for reverse transcription of the PCR product, followed by amplification of the cDNA using in vitro transcription. The product of in vitro transcription is a sense-RNA corresponding to the original member(s) of the candidate library. PCR product may be also be evaluated in a number of ways known in the art, including real-time assessment using detection of labeled primers, e.g. TaqMan or molecular beacon probes. Technology platforms suitable for analysis of PCR products include the ABI 7700, 5700, or 7000 Sequence Detection Systems (Applied Biosystems, Foster City, Calif.), the MJ Research Opticon (MJ Research, Waltham, Mass.), the Roche Light Cycler (Roche Diagnostics, Indianapolis, Ind.), the Stratagene MX4000 (Stratagene, La Jolla, Calif.), and the Bio-Rad iCycler (Bio-Rad Laboratories, Hercules, Calif.). Alternatively, molecular beacons are used to detect presence of a nucleic acid sequence in an unamplified RNA or CDNA sample, or following amplification of the sequence using any method, e.g., IVT (in vitro transcription) or NASBA (nucleic acid sequence based amplification). Molecular beacons are designed with sequences complementary to member(s) of a candidate nucleotide library, and are linked to fluorescent labels. Each probe has a different fluorescent label with non-overlapping emission wavelengths. For example, expression of ten genes may be assessed using ten different sequence-specific molecular beacons.

Alternatively, or in addition, molecular beacons are used to assess expression of multiple nucleotide sequences simultaneously. Molecular beacons with sequences complimentary to the members of a diagnostic nucleotide set are designed and linked to fluorescent labels. Each fluorescent label used must have a non-overlapping emission wavelength. For example, 10 nucleotide sequences can be assessed by hybridizing 10 sequence specific molecular beacons (each labeled with a different fluorescent molecule) to an amplified or non-amplified RNA or cDNA sample. Such an assay bypasses the need for sample labeling procedures.

Alternatively, or in addition, bead arrays can be used to assess expression of multiple sequences simultaneously (see, e.g., LabMAP 100, Luminex Corp, Austin, Tex.). Alternatively, or in addition, electric arrays can be used to assess expression of multiple sequences, as exemplified by the e-Sensor technology of Motorola (Chicago, Ill.) or Nanochip technology of Nanogen (San Diego, Calif.).

Of course, the particular method elected will be dependent on such factors as quantity of RNA recovered, practitioner preference, available reagents and equipment, detectors, and the like. Typically, however, the elected method(s) will be appropriate for processing the number of samples and probes of interest. Methods for high-throughput expression analysis are discussed below.

Alternatively, expression at the level of protein products of gene expression is performed. For example, protein expression in a sample can be evaluated by one or more method selected from among: western analysis, two-dimensional gel analysis, chromatographic separation, mass spectrometric detection, protein-fusion reporter constructs, calorimetric assays, binding to a protein array (e.g., antibody array), and characterization of polysomal mRNA. One particularly favorable approach involves binding of labeled protein expression products to an array of antibodies specific for members of the candidate library. Methods for producing and evaluating antibodies are well known in the art, see, e.g., Coligan, supra; and Harlow and Lane (1989) Antibodies: A Laboratory Manual, Cold Spring Harbor Press, NY (“Harlow and Lane”). Additional details regarding a variety of immunological and immunoassay procedures adaptable to the present invention by selection of antibody reagents specific for the products of candidate nucleotide sequences can be found in, e.g., Stites and Terr (eds.) (1991) Basic and Clinical Immunology, 7th ed. Another approach uses systems for performing desorption spectrometry. Commercially available systems, e.g., from Ciphergen Biosystems, Inc. (Fremont, Calif.) are particularly well suited to quantitative analysis of protein expression. Protein Chip.RTM. arrays (see, e.g., the website, ciphergen.com) used in desorption spectrometry approaches provide arrays for detection of protein expression. Alternatively, affinity reagents, (e.g., antibodies, small molecules, etc.) may be developed that recognize epitopes of one or more protein products. Affinity assays are used in protein array assays, e.g., to detect the presence or absence of particular proteins. Alternatively, affinity reagents are used to detect expression using the methods described above. In the case of a protein that is expressed on a cell surface, labeled affinity reagents are bound to a sample, and cells expressing the protein are identified and counted using fluorescent activated cell sorting (FACS).

High Throughput Expression Assays

A number of suitable high throughput formats exist for evaluating gene expression. Typically, the term high throughput refers to a format that performs at least about 100 assays, or at least about 500 assays, or at least about 1000 assays, or at least about 5000 assays, or at least about 10,000 assays, or more per day. When enumerating assays, either the number of samples or the number of candidate nucleotide sequences evaluated can be considered. For example, a northern analysis of, e.g., about 100 samples performed in a gridded array, e.g., a dot blot, using a single probe corresponding to a polynucleotide sequence as described herein can be considered a high throughput assay. More typically, however, such an assay is performed as a series of duplicate blots, each evaluated with a distinct probe corresponding to a different polynucleotide sequence of a system for detecting gene expression. Alternatively, methods that simultaneously evaluate expression of about 100 or more polynucleotide sequences in one or more samples, or in multiple samples, are considered high throughput.

Numerous technological platforms for performing high throughput expression analysis are known. Generally, such methods involve a logical or physical array of either the subject samples, or the candidate library, or both. Common array formats include both liquid and solid phase arrays. For example, assays employing liquid phase arrays, e.g., for hybridization of nucleic acids, binding of antibodies or other receptors to ligand, etc., can be performed in multiwell, or microtiter, plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., the ORCA.TM. system from Beckman-Coulter, Inc. (Fullerton, Calif.) and the Zymate systems from Zymark Corporation (Hopkinton, Mass.).

Alternatively, a variety of solid phase arrays can favorably be employed to determine expression patterns in the context of the invention. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Detailed discussion of methods for linking nucleic acids and proteins to a chip substrate, are found in, e.g., U.S. Pat. No. 5,143,854, “Large Scale Photolithographic Solid Phase Synthesis Of Polypeptides And Receptor Binding Screening Thereof,” to Pirrung et al., issued, Sep. 1, 1992; U.S. Pat. No. 5,837,832, “Arrays Of Nucleic Acid Probes On Biological Chips,” to Chee et al., issued Nov. 17, 1998; U.S. Pat. No. 6,087,112, “Arrays With Modified Oligonucleotide And Polynucleotide Compositions,” to Dale, issued Jul. 11, 2000; U.S. Pat. No. 5,215,882, “Method Of Immobilizing Nucleic Acid On A Solid Substrate For Use In Nucleic Acid Hybridization Assays,” to Bahl. et al., issued Jun. 1, 1993; U.S. Pat. No. 5,707,807, “Molecular Indexing For Expressed Gene Analysis,” to Kato, issued Jan. 13, 1998; U.S. Pat. No. 5,807,522, “Methods For Fabricating Microarrays Of Biological Samples,” to Brown et al., issued Sep. 15, 1998; U.S. Pat. No. 5,958,342, “Jet Droplet Device,” to Gamble et al., issued Sep. 28, 1999; U.S. Pat. No. 5,994,076, “Methods Of Assaying Differential Expression,” to Chenchik et al., issued Nov. 30, 1999; U.S. Pat. No. 6,004,755, “Quantitative Microarray Hybridization Assays,” to Wang, issued Dec. 21, 1999; U.S. Pat. No. 6,048,695, “Chemically Modified Nucleic Acids And Method For Coupling Nucleic Acids To Solid Support,” to Bradley et al., issued Apr. 11, 2000; U.S. Pat. No. 6,060,240, “Methods For Measuring Relative Amounts Of Nucleic Acids In A Complex Mixture And Retrieval Of Specific Sequences Therefrom,” to Kamb et al., issued May 9, 2000; U.S. Pat. No. 6,090,556, “Method For Quantitatively Determining The Expression Of A Gene,” to Kato, issued Jul. 18, 2000; and U.S. Pat. No. 6,040,138, “Expression Monitoring By Hybridization To High Density Oligonucleotide Arrays,” to Lockhart et al., issued Mar. 21, 2000.

For example, cDNA inserts corresponding to candidate nucleotide sequences, in a standard TA cloning vector, are amplified by a polymerase chain reaction for approximately 30-40 cycles. The amplified PCR products are then arrayed onto a glass support by any of a variety of well-known techniques, e.g., the VSLIPS.TM. technology described in U.S. Pat. No. 5,143,854. RNA, or cDNA corresponding to RNA, isolated from a subject sample, is labeled, e.g., with a fluorescent tag, and a solution containing the RNA (or cDNA) is incubated under conditions favorable for hybridization, with the “probe” chip. Following incubation, and washing to eliminate non-specific hybridization, the labeled nucleic acid bound to the chip is detected qualitatively or quantitatively, and the resulting expression profile for the corresponding candidate nucleotide sequences is recorded. Multiple cDNAs from a nucleotide sequence that are non-overlapping or partially overlapping may also be used.

In another approach, oligonucleotides corresponding to members of a candidate nucleotide library are synthesized and spotted onto an array. Alternatively, oligonucleotides are synthesized onto the array using methods known in the art, e.g. Hughes, et al. supra. The oligonucleotide is designed to be complementary to any portion of the candidate nucleotide sequence. In addition, in the context of expression analysis for, e.g. diagnostic use of diagnostic nucleotide sets, an oligonucleotide can be designed to exhibit particular hybridization characteristics, or to exhibit a particular specificity and/or sensitivity, as further described below.

Oligonucleotide probes may be designed on a contract basis by various companies (for example, Compugen, Mergen, Affymetrix, Telechem), or designed from the candidate sequences using a variety of parameters and algorithms as indicated at the website genome.wi.mit.edu/cgi-bin/prtm-er/primer3.cgi. Briefly, the length of the oligonucleotide to be synthesized is determined, preferably at least 16 nucleotides, generally 18-24 nucleotides, 24-70 nucleotides and, in some circumstances, more than 70 nucleotides. The sequence analysis algorithms and tools described above are applied to the sequences to mask repetitive elements, vector sequences and low complexity sequences. Oligonucleotides are selected that are specific to the candidate nucleotide sequence (based on a Blast n search of the oligonucleotide sequence in question against gene sequences databases, such as the Human Genome Sequence, UniGene, dbEST or the non-redundant database at NCBI), and have<50% G content and 25-70% G+C content. Desired oligonucleotides are synthesized using well-known methods and apparatus, or ordered from a commercial supplier.

A hybridization signal may be amplified using methods known in the art, and as described herein, for example use of the Clontech kit (Glass Fluorescent Labeling Kit), Stratagene kit (Fairplay Microarray Labeling Kit), the Micromax kit (New England Nuclear, Inc.), the Genisphere kit (3DNA Submicro), linear amplification, e.g., as described in U.S. Pat. No. 6,132,997 or described in Hughes, T R, et al. (2001) Nature Biotechnology 19:343-347 (2001) and/or Westin et al. (2000) Nat Biotech. 18:199-204. In some cases, amplification techniques do not increase signal intensity, but allow assays to be done with small amounts of RNA.

Alternatively, fluorescently labeled cDNA are hybridized directly to the microarray using methods known in the art. For example, labeled cDNA are generated by reverse transcription using Cy3-and Cy5-conjugated deoxynucleotides, and the reaction products purified using standard methods. It is appreciated that the methods for signal amplification of expression data useful for identifying diagnostic nucleotide sets are also useful for amplification of expression data for diagnostic purposes.

Microarray expression may be detected by scanning the microarray with a variety of laser or CCD-based scanners, and extracting features with numerous software packages, for example, Imagene (Biodiscovery), Feature Extraction Software (Agilent), Scanalyze (Eisen, M. 1999. SCANALYZE User Manual; Stanford Univ., Stanford, Calif. Ver 2.32.), GenePix (Axon Instruments).

In another approach, hybridization to microelectric arrays is performed, e.g., as described in Umek et al (2001) J Mol Diagn. 3:74-84. An affinity probe, e.g., DNA, is deposited on a metal surface. The metal surface underlying each probe is connected to a metal wire and electrical signal detection system. Unlabelled RNA or cDNA is hybridized to the array, or alternatively, RNA or cDNA sample is amplified before hybridization, e.g., by PCR. Specific hybridization of sample RNA or cDNA results in generation of an electrical signal, which is transmitted to a detector. See Westin (2000) Nat Biotech. 18:199-204 (describing anchored multiplex amplification of a microelectronic chip array); Edman (1997) NAR 25:4907-14; Vignali (2000) J Immunol Methods 243:243-55.

Evaluation of Expression Patterns

Expression patterns can be evaluated by qualitative and/or quantitative measures. Certain of the above described techniques for evaluating gene expression (e.g., as RNA or protein products) yield data that are predominantly qualitative in nature, i.e., the methods detect differences in expression that classify expression into distinct modes without providing significant information regarding quantitative aspects of expression. For example, a technique can be described as a qualitative technique if it detects the presence or absence of expression of a candidate nucleotide sequence, i.e., an on/off pattern of expression. Alternatively, a qualitative technique measures the presence (and/or absence) of different alleles, or variants, of a gene product.

In contrast, some methods provide data that characterize expression in a quantitative manner. That is, the methods relate expression on a numerical scale, e.g., a scale of 0-5, a scale of 1-10, a scale of +-+++, from grade 1 to grade 5, a grade from a to z, or the like. It will be understood that the numerical, and symbolic examples provided are arbitrary, and that any graduated scale (or any symbolic representation of a graduated scale) can be employed in the context of the present invention to describe quantitative differences in nucleotide sequence expression. Typically, such methods yield information corresponding to a relative increase or decrease in expression.

Any method that yields either quantitative or qualitative expression data is suitable for evaluating expression of candidate nucleotide sequences in a subject sample. In some cases, e.g., when multiple methods are employed to determine expression patterns for a plurality of candidate nucleotide sequences, the recovered data, e.g., the expression profile, for the nucleotide sequences is a combination of quantitative and qualitative data.

In some embodiments, qualitative and/or quantitative expression data from a sample is compared with a reference molecular signature that is indicative of, for example, presence or absence of a disease condition, symptom, or criterion, extent of progression of disease, effectiveness of treatment of disease, or prognosis for prophylaxis, therapy, or cure of disease. The reference molecular signature may be from a reference healthy individual (e.g., an individual who does not exhibit symptoms of the disease condition to be evaluated) or an individual with a disease condition for comparison with the sample (e.g., an individual with the same or different stage of disease for comparison with the individual being evaluated, or with a genotype or phenotype that indicates, for example, prognosis for successful treatment), or the reference molecular signature may be established from a compilation of data from multiple individuals

In some applications, expression of a plurality of candidate polynucleotide sequences is evaluated sequentially. This is typically the case for methods that can be characterized as low-to moderate throughput. In contrast, as the throughput of the elected assay increases, expression for the plurality of candidate polynucleotide sequences in a sample or multiple samples is typically assayed simultaneously. Again, the methods (and throughput) are largely determined by the individual practitioner, although, typically, it is preferable to employ methods that permit rapid, e.g. automated or partially automated, preparation and detection, on a scale that is time-efficient and cost-effective.

Genotyping

In addition to, or in conjunction with, the correlation of expression profiles and clinical data, it is often desirable to correlate expression patterns with a subject's genotype at one or more genetic loci or to correlate both expression profiles and genetic loci data with clinical data. The selected loci can be, for example, chromosomal loci corresponding to one or more member of the candidate library, polymorphic alleles for marker loci, or alternative disease related loci (not contributing to the candidate library) known to be, or putatively associated with, a disease (or disease criterion). Indeed, it will be appreciated that where a (polymorphic) allele at a locus is linked to a disease (or to a predisposition to a disease), the presence of the allele can itself be a disease criterion.

Numerous well known methods exist for evaluating the genotype of an individual, including southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Often, these methods can be performed on nucleic acid samples recovered via simple procedures from the same sample as yielded the material for expression profiling. Exemplary techniques are described in, e.g., Sambrook, and Ausubel, supra.

Samples

Samples which may be evaluated for differential expression of the polynucleotide sequences described herein include any blood vessel or portion thereof with atherosclerotic and/or inflammatory disease. Such blood vessels include, but are not limited to, the aorta, a coronary artery, the carotid artery, and peripheral blood vessels such as, for example, iliac or femoral arteries. In one embodiment, the sample is derived from an arterial biopsy. In another embodiment, the sample is derived from an atherectomy. Samples may also be derived from peripheral blood cells or serum.

Samples may be stabilized for storage by addition of reagents such as Trizol. Total RNA and/or protein may be isolated using standard techniques known in the art for expression profiling experiments.

Methods for RNA isolation include those described in standard molecular biology textbooks. Commercially available kits such as those provided by Qiagen (RNeasy Kits) may also be used for RNA isolation.

Methods for Diagnosing Atherosclerotic Disease

The invention provides methods for diagnosing an atherosclerotic disease condition in an individual. Diagnosis includes, for example, determining presence or absence of a disease condition or a symptom of a disease condition in an individual who has, who is suspected of having, or who may be suspected of being predisposed to an atherosclerotic disease. In accordance with methods of the invention for diagnosing atherosclerotic disease, gene expression products (e.g., RNA or proteins) from a sample from an individual are contacted with a system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a test sample are compared with levels of expression in a molecular signature that is indicative of presence or absence of an atherosclerotic disease condition for which diagnosis is desired. To obtain a diagnosis, the levels of gene expression in a sample may be compared to one or more than one molecular signature, each of which may be indicative of presence or absence one or more than one atherosclerotic disease condition.

In some embodiments, polynucleotides derived from a sample from an individual (e.g., mRNA or polynucleotides derived from mRNA, for example cDNA) are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of presence or absence of an atherosclerotic disease in the individual. In some embodiments, presence, absence, or amount of the polynucleotides derived from the sample is compared with presence, absence, or amount of polynucleotides in a molecular signature indicative of presence or absence of a disease condition, criterion, or symptom for which diagnosis is desired.

In some embodiments, polypeptides derived from a sample from an individual are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of presence or absence of an atherosclerotic disease in the individual. In some embodiments, presence, absence, or amount of the polypeptides derived from the sample is compared with presence, absence, or amount of polypeptides in a molecular signature indicative of presence or absence of a disease condition, criterion, or symptom for which diagnosis is desired.

Methods for Assessing Extent of Progression of Atherosclerotic Disease

The invention provides methods for assessing extent of progression of an atherosclerotic disease condition in an individual. For example, a stage to which a disease condition or particular symptom has progressed may be assessed. In accordance with methods of the invention for assessing extent of progression of atherosclerotic disease, gene expression products (e.g., RNA or proteins) from a sample from an individual are contacted with a system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a test sample are compared with levels of expression in a molecular signature that is indicative of extent of progression of an atherosclerotic disease condition for which assessment is desired. The levels of gene expression may be compared to one or more than one molecular signature, each of which may be indicative of extent of progression of one or more than one atherosclerotic disease condition.

In some embodiments, polynucleotides derived from a sample from an individual (e.g., mRNA or polynucleotides derived from mRNA, for example CDNA) are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of extent of progression of an atherosclerotic disease in the individual. In some embodiments, presence, absence, or amount of the polynucleotides derived from the sample is compared with presence, absence, or amount of polynucleotides in a molecular signature indicative of extent of progression of a disease condition for which diagnosis is desired.

In some embodiments, polypeptides derived from a sample from an individual are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of extent of progression of an atherosclerotic disease in the individual. In some embodiments, presence, absence, or amount of the polypeptides derived from the sample is compared with presence, absence, or amount of polypeptides in a molecular signature indicative of extent of progression of a disease condition for which diagnosis is desired.

Methods for Assessing Efficacy of Treatment of Atherosclerotic Disease

The invention provides methods for assessing extent of progression of an atherosclerotic disease condition in an individual. For example, a stage to which a disease condition or particular symptom has progressed may be assessed by the methods of the invention. In accordance with methods of the invention for assessing extent of progression of atherosclerotic disease, gene expression products (e.g., RNA or proteins) from a sample from an individual are contacted with the system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a test sample are compared with levels of expression in a molecular signature that is indicative of extent of progression of an atherosclerotic disease condition for which assessment is desired. The levels of gene expression may be compared to one or more than one molecular signature, each of which may be indicative of extent of progression of one or more than one atherosclerotic disease condition.

In some embodiments, polynucleotides derived from a sample from an individual (e.g, mRNA or polynucleotides derived from mRNA, for example cDNA) are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of extent of progression of an atherosclerotic disease in the individual. In some embodiments, presence, absence, or amount of the polynucleotides derived from the sample is compared with presence, absence, or amount of polynucleotides in a molecular signature indicative of extent of progression of a disease condition for which assessment is desired.

In some embodiments, polypeptides derived from a sample from an individual are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of extent of progression of an atherosclerotic disease in the individual. In some embodiments, presence, absence, or amount of the polypeptides derived from the sample is compared with presence, absence, or amount of polypeptides in a molecular signature indicative of extent of progression of a disease condition for which assessment is desired.

Methods for Assessing Efficacy of Treatment

The invention provides methods for assessing efficacy of treatment of an atherosclerotic disease symptom or condition in an individual. As used herein, “efficacy of treatment” refers to achievement of a desired therapeutic outcome (e.g., reduction or elimination of one or more symptoms of atherosclerotic disease). “Treatment” as used herein may refer to prophylaxis, therapy, or cure with respect to one or more symptoms of an atherosclerotic disease or condition. Treatment includes administration of one or more compounds or biological substances with potential therapeutic benefit and/or alterations in environmental factors, such as, for example, diet and/or exercise. In one embodiment, administration of the one or more compounds or biological substances comprises administration via a medical device such as, for example, a drug eluting stent. In other embodiments, treatment may include gene therapy or any other method that alters expression of the polynucleotide sequences described herein. In accordance with methods of the invention for assessing efficacy of treatment of atherosclerotic disease, gene expression products (e.g., RNA or proteins) from a sample from an individual are contacted with a system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a test sample are compared with levels of expression in a molecular signature that is indicative of efficacy of treatment of an atherosclerotic disease symptom or condition for which assessment is desired. The levels of gene expression may be compared to one or more than one molecular signature, each of which may be indicative of extent of effectiveness of treatment of one or more than one atherosclerotic disease symptom or condition.

In some embodiments, polynucleotides derived from a sample from an individual (e.g., mRNA or polynucleotides derived from mRNA, for example cDNA) are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of efficacy of treatment of an atherosclerotic disease symptom or condition in the individual. In some embodiments, presence, absence, or amount of the polynucleotides derived from the sample is compared with presence, absence, or amount of polynucleotides in a molecular signature indicative of efficacy of treatment of a disease symptom or condition for which assessment is desired.

In some embodiments, polypeptides derived from a sample from an individual are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of efficacy of treatment of an atherosclerotic disease condition in the individual. In some embodiments, presence, absence, or amount of the polypeptides derived from the sample is compared with presence, absence, or amount of polypeptides in a molecular signature indicative of efficacy of treatment of a disease condition for which assessment is desired.

Methods for Identifying Compounds Effective for Treatment of Atherosclerotic Disease

The invention provides methods for identifying compounds effective for treatment of an atherosclerotic disease symptom or condition in an individual. In accordance with methods of the invention for identifying compounds effective for treatment of atherosclerotic disease, at least one test compound (i.e., one or more than one test compound) is administered, for example as a pharmaceutical composition comprising the at least one test compound and a pharmaceutically acceptable excipient, to an individual with an atherosclerotic disease symptom or condition or suspected of having an atherosclerotic disease symptom or condition, or to an individual who is predisposed to or suspected of being predisposed to development of an atherosclerotic disease symptom or condition. Gene expression products (e.g., RNA or proteins) from a sample from the individual are contacted with a system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a test sample from the individual to whom the at least one test compound has been administered are compared with levels of expression in a molecular signature that is indicative of efficacy of treatment of the atherosclerotic disease symptom or condition for which assessment is desired. The levels of gene expression may be compared to one or more than one molecular signature, each of which may be indicative of extent of effectiveness of treatment of one or more than one atherosclerotic disease symptom or condition.

In some embodiments, polynucleotides derived from a sample from an individual (e.g., mRNA or polynucleotides derived from mRNA, for example cDNA) to whom at least one test compound has been administered are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of efficacy of treatment of an atherosclerotic disease symptom or condition in the individual. In some embodiments, presence, absence, or amount of the polynucleotides derived from the sample is compared with presence, absence, or amount of polynucleotides in a molecular signature indicative of efficacy of treatment of a disease symptom or condition for which assessment is desired.

In some embodiments, polypeptides derived from a sample from an individual to whom at least one test compound has been administered are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of efficacy of treatment of an atherosclerotic disease condition in the individual. In some embodiments, presence, absence, or amount of the polypeptides derived from the sample is compared with presence, absence, or amount of polypeptides in a molecular signature indicative of efficacy of treatment of a disease condition for which assessment is desired.

Methods for Determining prognosis of Atherosclerotic Disease

The invention provides methods for determining prognosis of atherosclerotic disease in an individual, comprising contacting polynucleotides derived from a sample from the individual with a system for detecting gene expression as described above. “Prognosis” as used herein refers to the probability that an individual will develop an atherosclerotic disease symptom or condition, or that atherosclerotic disease will progress in an individual who has an atherosclerotic disease. Prognosis is a determination or prediction of probable course and/or outcome of a disease condition, i.e., whether an individual will exhibit or develop symptoms of the disease, i.e., a clinical event. In cardiovascular medicine, a common measure of prognosis is (but is not limited to) MACE (major adverse cardiac event). MACE includes mortality as well as morbidity measures, such as myocardial infarction, angina, stroke, rate of revascularization, hospitalization, etc.

For determination of prognosis of atherosclerotic disease, gene expression products (e.g., RNA or proteins) from a sample from an individual are contacted with the system for detecting gene expression as described above. In one embodiment, the genes for which expression is detected are selected from the group of genes corresponding to SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 133, 745, 806, 824, 886, 882, 901, 905, 913, and 927. In another embodiment, the genes for which expression is detected are selected from the group of genes corresponding to SEQ ID NOs: 1-927.

In some embodiments, qualitative and/or quantitative levels of gene expression in a sample from the individual are compared with levels of expression in a molecular signature that is indicative of prognosis of the atherosclerotic disease symptom or condition for which assessment is desired. The levels of gene expression may be compared to one or more than one molecular signature, each of which may be indicative of prognosis for one or more than one atherosclerotic disease symptom or condition.

In some embodiments, polynucleotides derived from a sample from an individual (e.g., mRNA or polynucleotides derived from mRNA, for example cDNA) are contacted with isolated polynucleotide molecules in a system for detecting gene expression as described above, wherein each isolated polynucleotide molecule detects an expressed product of a gene that is differentially expressed in atherosclerotic disease in a mammal, and hybridization complexes formed, if any, are detected, wherein presence, absence, or amount of hybridization complexes formed from at least one of the isolated polynucleotides is indicative of prognosis for development or progression an atherosclerotic disease symptom or condition in the individual. In some embodiments, presence, absence, or amount of the polynucleotides derived from the sample is compared with presence, absence, or amount of polynucleotides in a molecular signature indicative of prognosis for development or progression of a disease symptom or condition for which assessment is desired.

In some embodiments, polypeptides derived from a sample from an individual are contacted with a system for detecting gene expression as described above which comprises molecules capable of detectably binding to polypeptides that are differentially expressed in atherosclerotic disease, for example, antibodies or antigen binding fragments thereof, that detect expressed polypeptide products of genes corresponding to polynucleotide sequences depicted in the Sequence Listing, wherein presence, absence, or amount of bound polypeptide is indicative of prognosis for development or progression of an atherosclerotic disease symptom or condition in the individual. In some embodiments, presence, absence, or amount of the polypeptides derived from the sample is compared with presence, absence, or amount of polypeptides in a molecular signature indicative of prognosis for development or progression of an atherosclerotic disease symptom or condition for which assessment is desired.

Novel Polynucleotide Sequences

The invention provides novel polynucleotide sequences that are differentially expressed in atherosclerotic disease. We have identified unnamed (not previously described as corresponding to a gene or an expressed gene, and/or for which no function has previously been assigned) polynucleotide sequences herein. The novel differentially expressed nucleotide sequences of the invention are useful in a system for detecting gene expression, such as a diagnostic oligonucleotide set, and are also useful as probes in a diagnostic oligonucleotide set immobilized on an array. The novel polynucleotide sequences may be useful as disease target polynucleotide sequences and/or as imaging reagents as described herein.

As used herein, “novel polynucleotide sequence” refers to (a) a polynucleotide sequence containing at least one of the polynucleotide sequences disclosed herein (as depicted in the Sequence Listing); (b) a polynucleotide sequence that encodes the amino acid sequence encoded by a polynucleotide sequence disclosed herein; (c) a polynucleotide sequence that hybridizes to the complement of a coding sequence disclosed herein under highly stringent conditions, e.g., hybridization to filter-bound DNA in 0.5 M NaHPO₄, 7% sodium dodecyl sulfate (SDS), 1 mM EDTA at 65° C., and washing in 0.×SSC/0.1% SDS at 68° C. (Ausubel, F.M. et al., eds. (1989) Current Protocols in Molecular Biology, Vol. 1, Green Publishing Associates, Inc., and John Wiley & Sons, Inc., New York, at p. 2.01.3); (d) a polynucleotide sequence that hybridizes to the complement of a coding sequence disclosed herein under less stringent conditions, such as moderately stringent conditions, e.g., washing in 0.2×SSC/0.1% SDS at 42° C. (Ausubel et al. (1989), supra), yet which still encodes a functionally equivalent gene product; and/or (e) a polynucleotide sequence that is at least 90% identical, at least 80% identical, or at least 70% identical to the coding sequences disclosed herein, wherein % identity is determined using standard algorithms known in the art.

The invention also includes polynucleotide molecules that hybridize to, and are therefore the complements of, novel polynucleotide molecules as described in (a) through (c) in the preceding paragraph. Such hybridization conditions may be highly stringent or less highly stringent, as described above. In instances wherein the polynucleotide molecules are deoxyoligonucleotides, highly stringent conditions may refer to, e.g., washing in 6×SSC/0.05% sodium pyrophosphate at 37° C. (for 14-base oligonucleotides), 48° C. (for 17-base oligonucleotides), 55° C. (for 20-base oligonucleotides, and 60° C. (for 23-base oligonucleotides). These polynucleotide molecules may act as target nucleotide sequence antisense molecules, useful, for example, in target nucleotide sequence regulation and/or as antisense primers in amplification reactions of target nucleic acid sequences. Further, such sequences may be used as part of ribozyme and/or triple helix sequences, also useful for target nucleotide sequence regulation. Such molecules may also be used as components of diagnostic methods whereby the presence of a disease-causing allele may be detected.

The invention also encompasses nucleic acid molecules contained in full-length gene sequences that are related to or derived from novel polynucleotide sequences as described above and as depicted in the Sequence Listing. One sequence may map to more than one full-length gene.

The invention also encompasses (a) polynucleotide vectors that contain any of the foregoing novel polynucleotide sequences and/or their complements; (b) polynucleotide expression vectors that contain any of the foregoing novel polynucleotide sequences and/or their complements; and (c) genetically engineered host cells that contain any of the foregoing novel polynucleotide sequences operatively associated with a regulatory element that directs expression of the polynucleotide in the host cell. As used herein, regulatory elements include, but are not limited to, inducible and non-inducible promoters, enhancers, operators, and other elements known to those skilled in the art that drive and regulate gene expression.

The invention includes fragments of the novel polynucleotide sequences described above. Fragments may be any of at least 5, 10, 15, 20, 25, 50, 100, 200, or 500 nucleotides, or larger.

Novel Polypeptide Products

The invention includes novel polypeptide products, encoded by genes corresponding to the novel polynucleotide sequences described above, or functionally equivalent polypeptide gene products thereof. “Functionally equivalent,” as used herein, refers to a protein capable of exhibiting a substantially similar in vivo function, e.g., activity, as a novel polypeptide gene product encoded by a novel polynucleotide of the invention.

Equivalent novel polypeptide products may include deletions, additions, and/or substitutions of amino acid residues within the amino acid sequence encoded by a gene corresponding to a novel polynucleotide sequence of the invention as described above, but which results in a “silent” change (i.e., a change which does not substantially change the functional properties of the polypeptide). Amino acid substitutions may be made on the basis of similarity in polarity, charge, solubility, hydrophobicity, hydrophilicity, and/or the amphipathic nature of the residues involved.

Novel polypeptide products of genes corresponding to novel polynucleotide sequences described herein may be produced by recombinant nucleic acid technology using techniques that are well known in the art. For example, methods that are well known to those skilled in the art may be used to construct expression vectors containing novel polynucleotide coding sequences and appropriate transcriptional/translational control signals. These methods include, for example, in vitro recombinant DNA techniques, synthetic techniques and in vivo recombination/genetic recombination. See, for example, the techniques described in Sambrook et al., 1989, supra, and Ausubel et al., 1989, supra. Alternatively, PNA capable of encoding novel nucleotide sequence protein sequences may be chemically synthesized using, for example, synthesizers. See, for example, the techniques described in “Oligonucleotide Synthesis” (1984) Gait, M. J. ed., IRL Press, Oxford. A variety of host-expression vector systems may be utilized to express the novel nucleotide sequence coding sequences of the invention. Ruther et al. (1983) EMBO J 2:1791; Inouye & Inouye (1985) Nucleic Acids Res. 13:3101-3109; Van Heeke & Schuster (1989) J Biol. Chem. 264:5503; Smith et al. (1983) J Virol. 46: 584; Smith, U.S. Pat. No. 4,215,051; Logan & Shenk (1984) Proc. Natl. Acad Sci. USA 81:3655-3659; Bittner et al. (1987) Methods in Enzymol. 153:516-544; Wigler, et al. (1977) Cell 11:223; Szybalska & Szybalski (1962) Proc. Natl. Acad. Sci. USA 48:2026; Lowy, et al. (1980) Cell 22:817; Wigler, et al. (1980) Proc. Natl. Acad. Sci. USA 77:3567; O'Hare, et al. (1981) Proc. Natl. Acad. Sci. USA 78:1527; Mulligan & Berg (1981) Proc. Natl. Acad. Sci. USA 78:2072; Colberre-Garapin, etal. (1981) J Mol. Biol. 150:1; Santerre, etal. (1984) Gene 30:147; Janknecht, etal. (1991) Proc. Natl. Acad. Sci. USA 88: 8972-8976. When recombinant DNA technology is used to produce the protein encoded by a gene corresponding to the novel polynucleotide sequence, it may be advantageous to engineer fusion proteins that can facilitate labeling, immobilization and/or detection.

Antibodies

The invention also provides antibodies or antigen binding fragments thereof that specifically bind to novel polypeptide products encoded by genes that correspond to novel polynucleotide sequences as described above. Antibodies capable of specifically recognizing one or more novel nucleotide sequence epitopes may be prepared by methods that are well known in the art. Such antibodies include, but are not limited to, polyclonal antibodies, monoclonal antibodies (mAbs), humanized or chimeric antibodies, single chain antibodies, Fab fragments, F(ab')₂ fragments, fragments produced by a Fab expression library, anti-idiotypic (anti-Id) antibodies, and epitope-binding fragments of any of the above. Such antibodies may be used, for example, in the detection of a novel polynucleotide sequence in a biological sample, or, alternatively, as a method for the inhibition of abnormal gene activity, for example, the inhibition of a disease target nucleotide sequence, as further described below. Thus, such antibodies may be utilized as part of a disease treatment method, and/or may be used as part of diagnostic techniques whereby patients may be tested for abnormal levels of novel nucleotide sequence encoded proteins, or for the presence of abnormal forms of the such proteins.

For the production of antibodies that bind to a polypeptide encoded by a novel nucleotide sequence, various host animals may be immunized by injection with a novel protein encoded by the novel nucleotide sequence, or a portion thereof. Such host animals may include, but are not limited to rabbits, mice, and rats. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as novel polypeptide gene product, or an antigenic functional derivative thereof. For the production of polyclonal antibodies, host animals such as those described above, may be immunized by injection with novel polypeptide gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975) Nature 256:495-497; and U.S. Pat. No. 4,376,110, the human B-cell hybridoma technique (Kosbor et al. (1983) Immunology Today 4:72; and Cole et al. (1983) Proc. Natl. Acad. Sci. USA 80:2026-2030), and the EBV-hybridoma technique (Cole et al. (1985) Monoclonal Antibodies And Cancer Therapy, Alan R. Liss, Inc., pp. 77-96). Such antibodies may be of any immunoglobulin class including IgG, IgM, IgE, IgA, IgD and any subclass thereof. A hybridoma producing a mAb may be cultivated in vitro or in vivo.

In addition, techniques developed for the production of “chimeric antibodies” by splicing the genes from a mouse antibody molecule of appropriate antigen specificity together with genes from a human antibody molecule of appropriate biological activity can be used. Morrison et al. (1984) Proc. Natl. Acad. Sci. 81:6851-6855; Neuberger et al. (1984) Nature 312:604-608; Takeda et al. (1985) Nature 314:452-454. A chimeric antibody is a molecule in which different portions are derived from different animal species, such as those having a variable region derived from a murine mAb and a human immunoglobulin constant region.

Alternatively, techniques described for the production of single chain antibodies can be adapted to produce novel nucleotide sequence-single chain antibodies. (U.S. Pat. No. 4,946,778; Bird (1988) Science 242:423-426; Huston et al. (1988) Proc. NatL. Acad. Sci. USA 85:5879-5883; and Ward et al. (1989) Nature 334:544-546) Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab')₂ fragments which can be produced by pepsin digestion of the antibody molecule and the Fab fragments which can be generated by reducing the disulfide bridges of the F(ab')₂ fragments. Alternatively, Fab expression libraries may be constructed (Huse et al. (1989) Science 246:1275-1281) to allow rapid and easy identification of monoclonal Fab fragments with a desired specificity.

Disease Specific Target Polynucleotide Sequences

The invention also provides disease specific target polynucleotide sequences, and sets of disease specific target polynucleotide sequences. The diagnostic oligonucleotide sets, individual members of the diagnostic oligonucleotide sets and subsets thereof, and novel polynucleotide sequences, as described above, may also serve as disease specific target polynucleotide sequences. In particular, individual polynucleotide sequences that are differentially regulated or have predictive value that is strongly correlated with an atherosclerotic disease or disease criterion are especially favorable as atherosclerotic disease specific target polynucleotide sequences. Sets of genes that are co-regulated may also be identified as disease specific target polynucleotide sets. Such polynucleotide sequences and/or their complements and/or the expression products of genes corresponding to such polynucleotide sequences (e.g., mRNA, proteins) are targets for modulation by a variety of agents and techniques. For example, disease specific target polynucleotide sequences (or the expression products of genes corresponding to such polynucleotide sequences, or sets of disease specific target polynucleotide sequences) can be inhibited or activated by, e.g., target specific monoclonal antibodies or small molecule inhibitors, or delivery of the polynucleotide sequence or an expression product of a gene corresponding to the polynucleotide sequence to patients. Also, sets of genes can be inhibited or activated by a variety of agents and techniques. The specific usefulness of the target polynucleotide sequence(s) depends on the subject groups from which they were discovered, and the disease or disease criterion with which they correlate.

Kits

The invention provides kits containing a system for detecting gene expression, a diagnostic nucleotide set, candidate nucleotide library, one or novel polynucleotide sequence, one or more polypeptide products of the novel polynucleotide sequences, and/or one or more antibodies that recognize polypeptide expression products of the differentially regulated polynucleotide sequences described herein. A kit may contain a diagnostic nucleotide probe set, or other subset of a candidate library (e.g., as a cDNA, oligonucleotide or antibody microarray or reagents for performing an assay on a diagnostic gene set using any expression profiling technology), packaged in a suitable container. The kit may further comprise one or more additional reagents, e.g., substrates, labels, primers, reagents for labeling expression products, tubes and/or other accessories, reagents for collecting tissue or blood samples, buffers, hybridization chambers, cover slips, etc., and may also contain a software package, e.g., for analyzing differential expression using statistical methods as described herein, and optionally a password and/or account number for accessing the compiled database. The kit optionally further comprises an instruction set or user manual detailing preferred methods of performing the methods of the invention, and/or a reference to a site on the Internet where such instructions may be obtained.

TABLE 1

Polynucleotide sequences which detect differentially expressed
genes in atherosclerotic disease

| | | | | | | | |
|---|---|---|---|---|---|---|---|
| SEQ | | | | | | | |
| ID | | GENE | GENE | CLONE | UG | CHR_LOCATION | 6O mer |
| NO: | CLONE ID | SYMBOL | NAME | NAME | CLUSTER | PENG [A] | SEQUENCE |

| | | | | | | | |
|---|---|---|---|---|---|---|---|
| 1. | C0267B04-3 | | C0267B04-5N | C0267B04 | | No chromosome | ATGAGCCTAGA |
| | | | NIA Mouse | | | location | ACTCACATGCA |
| | | | 7.5 dpc Whole | | | info available | TTTTCCTGACT |
| | | | Embryo cDNA | | | | TCTATCATTAG |
| | | | Library (Long) | | | | AATAAGTTCAT |
| | | | Mus musculus | | | | CAAGA |
| | | | cDNA clone | | | | |
| | | | NIA:C0267B04 | | | | |
| | | | IMAGE:30017 | | | | |
| | | | 007 5′, MRNA | | | | |
| | | | sequence | | | | |
| 2. | M29697.1 | I17r | interleukin 7 | M29697 | Mm.389 | Chromosome 15 | CCTATTGTTGA |
| | | | receptor | | | | GTGTCAAACAT |
| | | | | | | | CACCACTAAGT |
| | | | | | | | GGATGGTTATG |
| | | | | | | | TAGTCCATTAT |
| | | | | | | | CCAAA |
| 3. | L0304D03-3 | Wnt4 | wingless- | L0304D03 | Mm.103301 | Chromosome 4 | TACCTGAACCA |
| | | | related MMTV | | | | CTCTCTACTGT |
| | | | integration site | | | | TGTTGTCACAA |
| | | | 4 | | | | GGCAAAAGTG |
| | | | | | | | GCATTCCTTCC |
| | | | | | | | TCCAAG |
| 4. | L0237D12-3 | Cstd | cathepsin D | L0237D12 | Mm.231395 | Chromosome 7 | CCCTTTGCTGT |
| | | | | | | | GTGGGCAGTAC |
| | | | | | | | TCTGAAGCAGG |
| | | | | | | | CAAATGGGTCT |
| | | | | | | | TAGGATCCCTC |
| | | | | | | | CCAGA |
| 5. | C0266b08-3 | BM204200 | ESTs | C0266B08 | Mm.222000 | Chromosome 6 | TCCAAAGATAA |
| | | | BM204200 | | | | AATGAGCAAC |
| | | | | | | | CGCACTGGCTT |
| | | | | | | | AGCCATAGATG |
| | | | | | | | ACTGACAGTGA |
| | | | | | | | TTGGAA |
| 6. | J0537C05-3 | Pfdn2 | prefoldin 2 | J0537C05 | Mm.10756 | Chromosome 1 | TGCCTTGGAGG |
| | | | | | | | GCAACAAGGA |
| | | | | | | | GCAGATACAG |
| | | | | | | | AAGATCATTGA |
| | | | | | | | GACACTGTTCA |
| | | | | | | | CAGCAGC |
| 7. | L0216F02-3 | C430008C19Rik | RIKEN cDNA | L0216F02 | Mm.268474 | Chromosome 10 | CATGAATTCCA |
| | | | C430008C19 | | | | AACCAGTTATT |
| | | | gene | | | | ATTAACATGAA |
| | | | | | | | CCTGAACCTGA |
| | | | | | | | ACAATTATGAC |
| | | | | | | | TGTGC |
| 8. | NM_017372.1 | Lyzs | lysozyme | NM_017372 | Mm.45436 | Chromosome 10 | TTTCTGTCACT |
| | | | | | | | GCTCAGGCCAA |
| | | | | | | | GGTCTATGAAC |
| | | | | | | | GTTGTGAGTTT |
| | | | | | | | GCCAGAACTCT |
| | | | | | | | GAAAA |
| 9. | C0271B02-3 | 4732437J24Rik | RIKEN cDNA | C0271B02 | Mm.39102 | Chromosome 4 | TTCATACCAAG |
| | | | 4732437J24 | | | | GAACCTGACCT |
| | | | gene | | | | CTCTGACAATT |
| | | | | | | | GCATTTTGAAC |
| | | | | | | | ATTGTTGTCCC |
| | | | | | | | CAAAG |
| 10. | H3022C10-3 | AA408868 | expreexpressed | H3022C10 | Mm.247272 | Chromosome 16 | CATTGGAAACA |
| | | | sequence | | | | GACACGTTTGT |
| | | | AA408868 | | | | AGGCATTTGCG |
| | | | | | | | TATTCTTGAAG |
| | | | | | | | AGACTGTTTTA |
| | | | | | | | TGAAT |
| 11. | L0806E05-3 | Gtl2 | GTL2, | L0806E05 | Mm.200506 | Chromosome 12 | GTAATGGAGA |
| | | | imprinted | | | | ATGTATCTGAA |
| | | | maternally | | | | CCCATATCAAG |
| | | | expressed | | | | CCATCTCTCTT |
| | | | untranslated | | | | CCTTAACATGT |
| | | | mRNA | | | | TAAGCA |
| 12. | H3111E06-5 | Acas21 | acetyl- | H3111E06 | Mm.7044 | Chromosome 2 | ACACCTCTAAC |
| | | | Coenzyme A | | | | TCCCAAGAAG |
| | | | synthetase 2 | | | | ACGGAGTGAA |
| | | | (AMP | | | | TGTCCTCTCCT |
| | | | forming)-like | | | | ATCATTT |
| 13. | H3091H05-3 | Hras1 | Harvey rat | H3091H05 | Mm.6793 | Chromosome 7 | GTGAGATTCGG |
| | | | sarcoma virus | | | | CAGCATAAATT |
| | | | oncogene 1 | | | | GCGGAAACTG |
| | | | | | | | AACCCACCCGA |
| | | | | | | | TGAGAGTGGTC |
| | | | | | | | CTGGCT |
| 14. | K0324B10-3 | Timp1 | tissue inhibitor | K0324B10 | Mm.8245 | Chromosome X | TCATAAGGGCT |
| | | | of | | | | AAATTCATGGG |
| | | | metalloproteina | | | | TTCCCCAGAAA |
| | | | se 1 | | | | TCAACGAGACC |
| | | | | | | | ACCTTATACCA |
| | | | | | | | GCGTT |
| 15. | K0508B06-3 | | transcribed | K0508B06 | Mm.217234 | Chromosome 5 | AAAGACTGAG |
| | | | sequence with | | | | AGGAGTCATG |
| | | | moderate | | | | AACCAGGGTA |
| | | | similarity to | | | | AAACTTATTGG |
| | | | protein | | | | TGCTTTGAGAC |
| | | | ref:NP_077285.1 | | | | TTCCAGCA |
| | | | (H. spaiens) | | | | |
| | | | A20-binding | | | | |
| | | | inhibitor of NF- | | | | |
| | | | kappaB | | | | |
| | | | activation 2; | | | | |
| | | | LKB1- | | | | |
| | | | interacting | | | | |
| | | | protein [Homo | | | | |
| | | | sapiens] | | | | |
| 16. | C0176A01-3 | Syngr1 | synaptogyrin 1 | C0176A01 | Mm.230301 | Chromosome 15 | GCAGCATCGCT |
| | | | | | | | TCCTTGGTTTA |
| | | | | | | | TTCTTTGTGTTT |
| | | | | | | | GTTCCTTCAGT |
| | | | | | | | AAACATTTATT |
| | | | | | | | GAGC |
| 17. | J0748G02-3 | | AU018093 | J0748G02 | | Chromosome 2 | TTTTAACGGAG |
| | | | Mouse two-cell | | | | CCTGAATATAG |
| | | | stage embryo | | | | CAGGTTTAAAA |
| | | | cDNA Mus | | | | TTTAAACAGGT |
| | | | musculus | | | | ATAAAATGAA |
| | | | cDNA clone | | | | AAATAA |
| | | | J0748G02 3′, | | | | |
| | | | MRNA | | | | |
| | | | sequence | | | | |
| 18. | J0035G10-3 | C77672 | ESTs C77672 | J0035G10 | Mm.36571 | Chromosome 4 | TAGCATGAACC |
| | | | | | | | ACCATGTTTGG |
| | | | | | | | CAATACTGTAT |
| | | | | | | | TTTAGAAAGAA |
| | | | | | | | TTAATGGACTG |
| | | | | | | | GAGAG |
| 19. | C0630C02-3 | Cxcl16 | chemokine (C- | C0630C02 | Mm.46424 | Chromosome 11 | CCTGAGCTCAC |
| | | | X-C motif) | | | | TGTTTCTCATG |
| | | | ligand 16 | | | | CTGTCTTGAGA |
| | | | | | | | CAAAGTATCCA |
| | | | | | | | TATGGAACCTA |
| | | | | | | | GGTTA |
| 20. | K0313A10-3 | 5430435G22Rik | RIKEN cDNA | K0313A10 | Mm.44508 | Chromosome 1 | GCTGGTGTTTG |
| | | | 5340435G22 | | | | TGTCAAGAAA |
| | | | gene | | | | ATGGCTGAAGC |
| | | | | | | | TTGTTTCCAGG |
| | | | | | | | CTGTAGGAATG |
| | | | | | | | TTGAAC |
| 21. | L0070E11-3 | Cbfa2t1H | CBFA2T1 | L0070E11 | Mm.4909 | Chromosome 4 | ACTTAAGTTAT |
| | | | identified gene | | | | CTGCATAGAGG |
| | | | homolog | | | | CAATCCTCCTG |
| | | | (human) | | | | GGTTTGCTTTA |
| | | | | | | | TGTCTCGAAAA |
| | | | | | | | TCTAA |
| 22. | H3072E02-3 | BG069076 | ESTs | H3072E02 | Mm.26437 | Chromosome 12 | GGGCAAAGGT |
| | | | BG069076 | | | | ACTTTCTGACA |
| | | | | | | | AACTGAGTACC |
| | | | | | | | TGAGATCAACC |
| | | | | | | | CCCAAGAAGG |
| | | | | | | | GAAAAAA |
| 23. | H3079B06-3 | | Mus musculus | H3079B06 | Mm.295683 | Chromosome 5 | ACTATGCAATT |
| | | | unkknown | | | | GGACAGATGG |
| | | | mRNA | | | | ATTACCAAGGA |
| | | | | | | | GACTAAAAAT |
| | | | | | | | ATATTCTTTGA |
| | | | | | | | CTTTGGG |
| 24. | H3002D08-3 | 4833412N02Rik | RIKEN cDNA | H3002D08 | Mm.195099 | Chromosome 5 | TCACTGACCTC |
| | | | 4833412N02 | | | | AACCCCTCCTG |
| | | | gene | | | | CAGAGAAGCC |
| | | | | | | | TGAAGACCCCA |
| | | | | | | | AAAGCTGCCA |
| | | | | | | | GTCCAAA |
| 25. | H3159A08-3 | Gp49b | glycoprotein 49 | H3159A08 | Mm.196617 | Chromosome 10 | GATATAATGTG |
| | | | B | | | | ATAAAGTTCCA |
| | | | | | | | AAAGGATCTCT |
| | | | | | | | CTGGCTGAAGG |
| | | | | | | | AGATACTGGAT |
| | | | | | | | GGAAC |
| 26. | C0612F12-3 | BM207436 | ESTs | C0612F12 | Mm.260421 | No Chromosome | CTGAACCCCAA |
| | | | BM207436 | | | location | TTAATAGCAAA |
| | | | | | | info available | GGATATATCTC |
| | | | | | | | TCTTCAAAAAC |
| | | | | | | | GGATAGATTTC |
| | | | | | | | TGAAG |
| 27. | H3108A03-3 | Apobec1 | apolipoprotein | H3108A03 | Mm.3333 | Chromosome 6 | TTTTGTTCTCTC |
| | | | B editing | | | | CATCTGTTAGC |
| | | | | | | | CGTTCTGAGGA |
| | | | | | | | CTGAATGCAGA |
| | | | | | | | TTGTCAGCTCA |
| | | | | | | | AAAA |
| 28. | C0180G01-3 | BI076556 | ESTs BI076556 | C0180601 | Mm.37657 | Chromosome 16 | GCCAATCTCAG |
| | | | | | | | AACCCACATAG |
| | | | | | | | AAGGGTCTGCA |
| | | | | | | | GTATTATTCCT |
| | | | | | | | GTTTCATGTGT |
| | | | | | | | GCACA |
| 29. | C0938A03-3 | Sf3a1 | splicing factor | C0938A03 | Mm.156914 | Chromosome 11 | AGTGCAAAATT |
| | | | 3a, subunit 1 | | | | TGGTTTGTTGG |
| | | | | | | | TGTGCTTTTCT |
| | | | | | | | GGTTTAGGAGC |
| | | | | | | | CTGAAACAAG |
| | | | | | | | CACACT |
| 30. | J0703E02-3 | Ogdh | oxoglutarate | J0703E02 | Mm.30074 | Chromosome 11 | CATGAGTAAGT |
| | | | dehydrogenase | | | | TGTGAAGGCTG |
| | | | (lipoamide) | | | | GACCCACATCT |
| | | | | | | | TGATACTTGTT |
| | | | | | | | TTCTGCATCTT |
| | | | | | | | GGGCA |
| 31. | C0274D12-3 | | transcribed | C0274D12 | Mm.217705 | Chromosome 12 | TAGACGTTGTA |
| | | | sequence with | | | | AAAAGGAGCC |
| | | | moderate | | | | AAGTTTATCAT |
| | | | similarity to | | | | TTTGTTCCTTA |
| | | | protein | | | | AATCCGTCATA |
| | | | pir:S12207 | | | | TGTGGG |
| | | | (M. musculus) | | | | |
| | | | S12207 | | | | |
| | | | hypothetical | | | | |
| | | | protein (B2 | | | | |
| | | | element)- | | | | |
| | | | mouse | | | | |
| 32. | H3097H03-3 | Expi | extracellular | H3097H03 | Mm.1650 | Chromosome 11 | ACTGTGGTGAC |
| | | | proteinase | | | | AGCTTCCTAAC |
| | | | inhibitor | | | | GTGTTTGTGTC |
| | | | | | | | TAAAATAAACT |
| | | | | | | | ATCCTTAGCAT |
| | | | | | | | CCTTC |
| 33. | H3074D10-3 | | transcribed | H3074D10 | Mm.103987 | Chromosome 15 | TATAAATAGAA |
| | | | sequence with | | | | AGTGAACCTGT |
| | | | weak similarity | | | | AACCTACCACG |
| | | | to protein | | | | GTATCTATCAT |
| | | | ref:NP_081764.1 | | | | AACACTAGACT |
| | | | (M. musculus) | | | | TTCAG |
| | | | RIKEN cDNA | | | | |
| | | | 5730493B19 | | | | |
| | | | [Mus musculus] | | | | |
| 34. | M14222.1 | Ctsb | cathepsin B | M14222 | Mm.22753 | Chromosome 14 | CATCCTACAAA |
| | | | | | | | GAGGATAAGC |
| | | | | | | | ACTTTGGGTAC |
| | | | | | | | ACTTCCTACAG |
| | | | | | | | CGTGTCTAACA |
| | | | | | | | GTGTGA |
| 35. | C0176G01-3 | 2400006H24Rik | RIKEN cDNA | C0176G01 | Mm.143774 | Chromosome Multiple | CCTGAAAATCT |
| | | | 2400006H24 | | | Mappings | GTCATGTCCAC |
| | | | gene | | | | CTTGGAGCCTG |
| | | | | | | | AGTAACTTTGA |
| | | | | | | | ACAGCTGGTAA |
| | | | | | | | CTAGT |
| 36. | H3092F08-5 | | UNKNOWN: | H3092F08 | | Chromosome 17 | AGTCAAGGAG |
| | | | Similar to Mus | | | | CCTAAAGATTA |
| | | | musculus | | | | TTATGTCAGAG |
| | | | immediate- | | | | AGACCAGCTTT |
| | | | early antigen | | | | AGATACACCCC |
| | | | (E-beta) gene | | | | TGAGCA |
| | | | partial intron 2 | | | | |
| | | | sequence | | | | |
| 37. | H3054F02-3 | 1200003C15Rik | RIKEN cDNA | H3054F02 | Mm.19325 | Chromosome 10 | TTATGCTGCAG |
| | | | 1200003C15 | | | | TTTCACTTGGA |
| | | | gene | | | | AAAGGGACAA |
| | | | | | | | GGAGCCTTCTA |
| | | | | | | | TTGTCCCCTGT |
| | | | | | | | TTGTAG |
| 38. | C0012F07-3 | 3010021M21Rik | RIKEN cDNA | C0012F07 | Mm.100525 | Chromosome 9 | GTAACCAAGA |
| | | | 3010021M21 | | | | GCCCTGAATAA |
| | | | gene | | | | GGAATTCATTG |
| | | | | | | | TAGTAGTGAAA |
| | | | | | | | GGGAAACTAA |
| | | | | | | | TGCTCTT |
| 39. | L0955A10-3 | 9030409G11Rik | RIKEN cDNA | L0955A10 | Mm.32810 | Chromosome 4 | TCCCATGCCTT |
| | | | 9030409G11 | | | | CCCAGAGGGA |
| | | | gene | | | | ATTTTAACAAT |
| | | | | | | | GTAACAATAA |
| | | | | | | | ATGCTTGGCCT |
| | | | | | | | TGAAGCT |
| 40. | L0045B05-3 | | transcribed | L0045b05 | Mm.182645 | Chromosome 9 | AGGACATCTTC |
| | | | sequence with | | | | CCAGATCTCAA |
| | | | moderate | | | | AAGAAGAAGA |
| | | | similarity to | | | | GAGCCTGTAAC |
| | | | protein | | | | CACCTCCATGA |
| | | | ref:NP_081764.1 | | | | CCTAAA |
| | | | (M. musculus) | | | | |
| | | | RIKEN cDNA | | | | |
| | | | 5730493B19 | | | | |
| | | | [Mus musculus] | | | | |
| 41. | H3049A10-3 | BG066966 | ESTs | H3049A10 | Mm.262549 | Chromosome 6 | TCCTGTGGGAG |
| | | | BG066966 | | | | ATCCCATAAAT |
| | | | | | | | CCTGAACCTCA |
| | | | | | | | CGTAGTGTTAC |
| | | | | | | | TTTTCCAGGTC |
| | | | | | | | ATTCT |
| 42. | X70298.1 | Sox4 | SRY-box | X70298 | Mm.253853 | Chromosome 13 | GGACGACGAG |
| | | | containing gene | | | | TTCGAAGACGA |
| | | | 4 | | | | CCTGCTCGACC |
| | | | | | | | TGAACCCCAGC |
| | | | | | | | TCAAACTTTGA |
| | | | | | | | GAGCAT |
| 43. | L0001C09-3 | | transcribed | L0001C09 | Mm.171544 | Chromosome 12 | GAAGAGATGG |
| | | | sequence with | | | | AAGATGGTAGT |
| | | | weak similarity | | | | GCCTTGAACAC |
| | | | to protein | | | | AGCCACCCAA |
| | | | ref:NP_081764.1 | | | | GCAAAGTTGA |
| | | | (M. musculus) | | | | AGAACAGG |
| | | | RIKEN cDNA | | | | |
| | | | 570493B19 | | | | |
| | | | [Mus musculus] | | | | |
| 44. | H3010D12-5 | | UNKNOWN: | H3010D12 | Data not found | Chromosome 9 | GCCTGCAGGA |
| | | | Similar to Mus | | | | GTTTGTGTTGG |
| | | | musculus | | | | TAGCCTCCAAG |
| | | | RIKEN cDNA | | | | GAGCTGAAGAT |
| | | | 8430421I07 | | | | GTGCTGAAGAT |
| | | | gene | | | | CCAGGCT |
| | | | (8430421I07Ri | | | | |
| | | | k), mRNA | | | | |
| 45. | C0923E12-3 | Ptpns1 | protein tyrosine | C0923E12 | Mm.1682 | Chromosome 2 | CTGTCTTCTAA |
| | | | phosphatase, | | | | TTCCAAAGGGT |
| | | | non-receptor | | | | TGGTTGGTAAA |
| | | | type substrate 1 | | | | GCTCCACCCCC |
| | | | | | | | TTTTCCTTTGC |
| | | | | | | | CTAAA |
| 46. | C0941E09-3 | D330001F17Rik | RIKEN cDNA | C0941E09 | Mm.123240 | No Chromosome | TTCACAGGGTT |
| | | | D330001F17 | | | location | CCTGGTGTTGC |
| | | | gene | | | info available | ATGCAGAGCCT |
| | | | | | | | GAACAAAAGA |
| | | | | | | | CTCAGGTGGAC |
| | | | | | | | CTGGAA |
| 47. | K0534C04-3 | Tce1 | T-complex | K0534C04 | Mm.41932 | Chromosome 17 | TCTACAAGGAA |
| | | | expressed gene | | | | GCATTCAACCA |
| | | | 1 | | | | CCAAGAGGAG |
| | | | | | | | CTTGGACCACG |
| | | | | | | | TTCACTCTGTA |
| | | | | | | | TTCTTT |
| 48. | H3064E11-3 | BG068254 | ESTs | H3064E11 | Mm.173544 | Chromosome 4 | GGGCCTGAACT |
| | | | BG068354 | | | | ATGGCTTAATT |
| | | | | | | | TACATTAATTA |
| | | | | | | | GTTAACATTAA |
| | | | | | | | TCACACAGTAA |
| | | | | | | | GGAGC |
| 49. | L0957C02-3 | E130319B15Rik | RIKEN cDNA | L0957C02 | Mm.149539 | Chromosome 2 | TGTGTTGTGAT |
| | | | E130319B15 | | | | TTCAACTCCCA |
| | | | gene | | | | AGACGCCCTTT |
| | | | | | | | ATGTCCATTCT |
| | | | | | | | GGAAAAATAC |
| | | | | | | | AATAAA |
| 50. | L0240C12-3 | Clqa | conplement | L0240C12 | Mm.370 | Chromosome 4 | ACTGATGTTTC |
| | | | component 1, q | | | | TGCACACTGCC |
| | | | subcomponent, | | | | CAGTGGTTTCT |
| | | | alpha | | | | TTAAGCACTTT |
| | | | polypeptide | | | | CTGGAATAAAC |
| | | | | | | | GATCC |
| 51. | J0018H07-3 | Rnf149 | ring finger | J0018H07 | Mm.28614 | Chromosome 1 | TCACAGATGTA |
| | | | protein 149 | | | | TGTGGAGGGGT |
| | | | | | | | TGTTTTCTGAG |
| | | | | | | | TACTAGACTAC |
| | | | | | | | CCTCTGTGGTT |
| | | | | | | | ATAAA |
| 52. | K0508E12-3 | Rin3 | Ras and Rab | K0508E12 | Mm.24145 | Chromosome 12 | TCGGGGATGG |
| | | | interactor 3 | | | | AGCTGAGATGT |
| | | | | | | | TCCCACCACAAC |
| | | | | | | | CCAAGATCTAA |
| | | | | | | | GAGTATTGTTT |
| | | | | | | | TGAAGA |
| 53. | L0208A01-3 | 4933437L13Rik | RIKEN cDNA | L0208A01 | Mm.159218 | Chromosome 16 | GGAGACTGAA |
| | | | 4933437K13 | | | | GCTTTTATTGT |
| | | | gene | | | | TTAATGTTGAA |
| | | | | | | | GATATTGATCT |
| | | | | | | | ACAAGGTGGG |
| | | | | | | | AATGGTG |
| 54. | C0239G03-3 | BM202478 | EST | C0239G03 | Mm.217664 | Chromosome 2 | AACTGTGGGTA |
| | | | BM202478 | | | | TAATTGTAAGA |
| | | | | | | | GCCTGAAACTT |
| | | | | | | | CCAGAACTGG |
| | | | | | | | AGAAACTGTCA |
| | | | | | | | CTGGGA |
| 55. | L0518C11-3 | 1700016K05Rik | RIKEN cDNA | L0518C11 | Mm.221743 | Chromosome 17 | GTGTTGTGATT |
| | | | 1700016K05 | | | | GTCGTCCCTGC |
| | | | gene | | | | TTAATGAACCC |
| | | | | | | | ACCTGAGGGA |
| | | | | | | | CAGTTAGTGTC |
| | | | | | | | TTACCC |
| 56. | H3054C09-3 | Oas1c | 2′-5′ | H3054C09 | Mm.206775 | Chromosome 5 | CTATATGAACT |
| | | | oligoadenylate | | | | GAGAAACAAC |
| | | | synthetase 1C | | | | ACGTATGCTGA |
| | | | | | | | ACCCCAATTCT |
| | | | | | | | ACAACAAAGT |
| | | | | | | | CTACGCC |
| 57. | L0811E07-3 | 3110087O12Rik | RIKEN cDNA | L0811E07 | Mm.32373 | Chromosome 3 | GGAATATATTA |
| | | | 3110057O12 | | | | TGTAGACTATT |
| | | | gene | | | | CTGGCCTGAAC |
| | | | | | | | CTTGTGGTTGA |
| | | | | | | | CTGATGCTCTG |
| | | | | | | | CCTCC |
| 58. | JO948A06-3 | | Mus musculus | J0948A06 | Mm.261771 | Chromosome 14 | TTGGGTGATCC |
| | | | mRNA similar | | | | ATATTTTTCAA |
| | | | to RIKEN | | | | ACCCATACTCC |
| | | | cDNA | | | | CAAAAGGAGA |
| | | | 4930503E14 | | | | CCTACTTAAAT |
| | | | gene (cDNA | | | | TTCTCT |
| | | | clone | | | | |
| | | | MGC:58418 | | | | |
| | | | IMAGE:67081 | | | | |
| | | | 14,) complete | | | | |
| | | | cds | | | | |
| 59. | C0931B05-3 | | transcribed | C0931B05 | Mm.252843 | Chromosome 10 | GTTCCTGAAGC |
| | | | sequence with | | | | TCTTGATATTT |
| | | | weak similarity | | | | TAGGACAAAA |
| | | | to protein | | | | CCCACCACGAC |
| | | | ref:NP_081764.1 | | | | AAAATGAGAA |
| | | | (M. musculus) | | | | GGAATTT |
| | | | RIKEN cDNA | | | | |
| | | | 5730493B19 | | | | |
| | | | [Mus usculus] | | | | |
| 60. | H3022A09-3 | Esp812 | EPS8-like | H3022A09 | Mm.27451 | Chromosome 7 | TGACTTCAAAT |
| | | | | | | | GTCCCATCCCA |
| | | | | | | | CCCAAAGAGC |
| | | | | | | | CTGTGATAACA |
| | | | | | | | GATGTCTCTGG |
| | | | | | | | CTATAT |
| 61. | G0118B03-3 | Usf2 | upstream | G0118B03 | Mm.15781 | Chromosome 7 | TGGGTAGGTTC |
| | | | transcription | | | | CTAGGTCTCCC |
| | | | factor 2 | | | | TGATATCTAA |
| | | | | | | | CTACAGTTATA |
| | | | | | | | CTGTAGCTGTG |
| | | | | | | | TGACA |
| 62. | H3156C12-3 | Ms4a6d | membrane- | H3156C12 | Mm.170657 | Chromosome 19 | CCTGTCTCAGA |
| | | | spanning 4- | | | | ACTCAAGAAT |
| | | | domains, | | | | AAATCCAGTGT |
| | | | subfamily A, | | | | ATCTTCAGAGT |
| | | | member 6D | | | | CACTTTGTAAC |
| | | | | | | | CCTAC |
| 63. | H3074G06-3 | 9530020G05Rik | RIKEN cDNA | H3074G06 | Mm.15120 | Chromosome 6 | TACTCCCTGGA |
| | | | 9530020G05 | | | | GACTAGAACC |
| | | | gene | | | | GTGGCTATAGC |
| | | | | | | | GGAGCATGCTC |
| | | | | | | | CAGAGCACAG |
| | | | | | | | GACTGAT |
| 64. | NM_003254.1 | TIMP1 | tissue inhibitor | NM_003254 | Hs.5831 | No Chromosome | GGGACACCAG |
| | | | of | | | location | AAGTCAACCA |
| | | | metalloproteinase | | | info available | GACCACCTTAT |
| | | | 1 (erythroid | | | | ACCAGCGTTAT |
| | | | potentiating | | | | GAGATCAAGA |
| | | | activity, | | | | TGACCAAG |
| | | | collagenase | | | | |
| | | | inhibitor) | | | | |
| 65. | K0647H07-3 | I17r | interleukin 7 | K0647H07 | Mm.389 | Chromosome 15 | GAAAACCAAA |
| | | | receptor | | | | ACTCTTGGTCA |
| | | | | | | | GAGACAATAT |
| | | | | | | | GCAAAACAGA |
| | | | | | | | GATGTCAAGTA |
| | | | | | | | CTATGTCC |
| 66. | J0257F12-3 | Rnf25 | ring finger | J0257F12 | Mm.86910 | Chromosome 1 | TCAAGGAGACT |
| | | | protein 25 | | | | GTAGACTTAAA |
| | | | | | | | GGCAGAACCC |
| | | | | | | | CGTAACAAAG |
| | | | | | | | GGCTCACAGGT |
| | | | | | | | CATCCTC |
| 67. | H3083G02-3 | Lcn2 | lipocalin 2 | H3083G02 | Mm.9537 | Chromosome 2 | CACCACGGACT |
| | | | | | | | ACAACCAGTTC |
| | | | | | | | GCCATGGTATT |
| | | | | | | | TTTCCGAAAGA |
| | | | | | | | CTTCTGAAAAC |
| | | | | | | | AAGCA |
| 68. | M64086.1 | Serpina3n | serine (or | M64086 | Mm.22650 | Chromosome 12 | GTACCCTCTGA |
| | | | cysteine) | | | | CTGTATATTTC |
| | | | proteinase | | | | AATCGGCCTTT |
| | | | inhibitor, clade | | | | CCTGATAATGA |
| | | | A, member 3N | | | | TCTTTGACACA |
| | | | | | | | GAAAC |
| 69. | C0906B05-3 | Cenpc | centromere | C0906B05 | Mm.221600 | Chromosome 5 | AAGAACTACTG |
| | | | autoantigen C | | | | ATACAGAACC |
| | | | | | | | ACTTCAGTTGT |
| | | | | | | | TCAGTTAGAAT |
| | | | | | | | CTTTTTAAGAC |
| | | | | | | | TCTCTC |
| 70. | H3094B08-3 | BG071051 | ESTs | H3094B08 | Mm.173358 | Chromosome 2 | CTTGACCTTTA |
| | | | BG071051 | | | | GATGGAAATTG |
| | | | | | | | TACCTAGAGAC |
| | | | | | | | GAGAAGGAGC |
| | | | | | | | CAAACTAAGGT |
| | | | | | | | CTGTCA |
| 71. | K0110F02-3 | Pstpip1 | proline-serine- | K0110F02 | Mm.2534 | Chromosome 9 | GGAACGGACA |
| | | | threonine | | | | ACGTGGCTTTG |
| | | | phosphatase- | | | | TCCCTGGGTCG |
| | | | interacting | | | | TACTTGGAGAA |
| | | | protein 1 | | | | GCTCTGAGGAA |
| | | | | | | | AGGCTA |
| 72. | L0072G08-3 | Renbp | renin binding | L0072G08 | Mm.28280 | Chromosome X | TTCGAATGCAC |
| | | | protein | | | | ATCATTGACAA |
| | | | | | | | GTTTCTCTTAT |
| | | | | | | | TGCCTTTCCAC |
| | | | | | | | TCTGGATGGGA |
| | | | | | | | CCCTG |
| 73. | J0088G06-3 | 49304272G13Rik | RIKEN cDNA | J0088G06 | Mm.23172 | No Chromosome | GCCTGGAGACT |
| | | | 4930475G13 | | | loction | GAAGGCAGTTT |
| | | | gene | | | info available | TACAAAGGAA |
| | | | | | | | AACTTAGATTT |
| | | | | | | | CTATTCATTTG |
| | | | | | | | CTTTTG |
| 74. | K0121F05-3 | Fcgr2b | Fc receptor, | K0121F05 | Mm.10809 | Chromosome 1 | CTGGATGAAG |
| | | | IgG, low | | | | AAACAGAGCA |
| | | | affinity IIb | | | | TGATTACCAGA |
| | | | | | | | ACCACATTTAG |
| | | | | | | | TCTCCCTTGGC |
| | | | | | | | ATTGGGA |
| 75. | K0124E12-3 | Wbscr5 | Williams- | K0124E12 | Mm.23955 | Chromosome 5 | TTAATATTGTC |
| | | | Beuren | | | | AATGTCAGGG |
| | | | syndrome | | | | GGTTCCCTGTC |
| | | | chromosome | | | | TCAGAGCATTA |
| | | | region 5 | | | | TGTGTACTAAC |
| | | | homolog | | | | TGTAGC |
| | | | (human) | | | | |
| 76. | K0649H05-3 | F730038I15Rik | RIKEN cDNA | K0649H05 | Mm.268680 | No Chromosome | CCAGAGTTTTT |
| | | | F730038I15 | | | location | TCCATCATGTT |
| | | | gene | | | info available | TTGCCCCAAAG |
| | | | | | | | ACCTCGGTTTG |
| | | | | | | | TAGAAGCCCA |
| | | | | | | | AGGAAA |
| 77. | K0154C05-3 | D230024O04 | hypothetical | K0154C05 | Mm.90241 | Chromosome 6 | GACAGGGTCA |
| | | | protein | | | | ATGTTTATTAT |
| | | | D230024O04 | | | | ACATACTGCAC |
| | | | | | | | TGATGAGAAC |
| | | | | | | | AATATCATATG |
| | | | | | | | TGAAGAG |
| 78. | C0185E05-3 | Hmox1 | heme | C0182E05 | Mm.230635 | Chromosome 8 | ACTCTCAGCTT |
| | | | oxygenase | | | | CCTGTTGGCAA |
| | | | (decycling) 1 | | | | CAGTGGCAGTG |
| | | | | | | | GGAATTTATGC |
| | | | | | | | CATGTAAATGC |
| | | | | | | | AATAC |
| 79. | L0823E04-3 | | transcribed | L0823E04 | Mm.270136 | Chromosome 7 | GACAGGGACT |
| | | | sequence with | | | | CCATATGGAAG |
| | | | weak similarity | | | | TAAGGACGTTT |
| | | | to protein | | | | ACCTCATTACT |
| | | | pir:T26134 | | | | AAGTCTCGTCA |
| | | | (C. elegans) | | | | AAAGAA |
| | | | T26134 | | | | |
| | | | hypothectical | | | | |
| | | | protein | | | | |
| | | | W04A4.5- | | | | |
| | | | Caenorhabditis | | | | |
| | | | elegans | | | | |
| 80. | K0310E05-3 | 9830126M18 | hypothetical | K0130E05 | Mm.266485 | Chromosome 15 | CTCGGATCTTC |
| | | | protein | | | | ATGTTCTTCAG |
| | | | 9830126M18 | | | | TAAGAATCTCT |
| | | | | | | | CTGTGGATTTG |
| | | | | | | | GAACAATCGTA |
| | | | | | | | AATAA |
| 81. | C0908B11-3 | P2ry6 | pyrimidinergic | C0908B11 | Mm.3929 | Chromosome 7 | CTAAGACACCT |
| | | | receptor P2Y, | | | | GTGATTTGGCA |
| | | | G-protein | | | | ACTGGTCAATT |
| | | | coupled, 6 | | | | CATGCTTGTTA |
| | | | | | | | CATTCAGAACT |
| | | | | | | | CAGGA |
| 82. | K0438A08-3 | Ccl2 | chemokine (C- | K0438A08 | Mm.145 | Chromosome 11 | TCCCTCTCTGT |
| | | | C motif) ligand | | | | GAATCCAGATT |
| | | | 2 | | | | CAACACTTTCA |
| | | | | | | | ATGTATGAGAG |
| | | | | | | | ATGAATTTTGT |
| | | | | | | | AAAGA |
| 83. | H3082C12-3 | Spp1 | secreted | H3082C12 | Mm.288474 | Chromosome 5 | TTCTCAGTTCA |
| | | | phosphoprotein | | | | GTGGATATATG |
| | | | 1 | | | | TATGTAGAGAA |
| | | | | | | | AGAGAGGTAA |
| | | | | | | | TATTTTGGGCT |
| | | | | | | | CTTAGC |
| 84. | H3014A12-3 | Capg | capping protein | H3014A12 | Mm.18626 | Chromosome 6 | CTGACCAAGGT |
| | | | (actin filament), | | | | GGCTGACTCCA |
| | | | gelsolin-like | | | | GCCCTTTTGCC |
| | | | | | | | TCTGAACTGCT |
| | | | | | | | AATTCCAGATG |
| | | | | | | | ACTGC |
| 85. | H3089C11-3 | BG070621 | ESTs | H3089C11 | Mm.173282 | Chromosome 4 | GATACCTGGCT |
| | | | BG070621 | | | | TATCTTTTATC |
| | | | | | | | AACAGCAAATT |
| | | | | | | | ATGCAGTGGTG |
| | | | | | | | GAAATGTCATC |
| | | | | | | | ACAGA |
| 86. | X67783.1 | Vcam1 | vascular cell | X67783 | Mm.76649 | Chromosome 3 | GTTTGAGAAGA |
| | | | adhesion | | | | GACATTATTTA |
| | | | molecule 1 | | | | TAAAACCCAG |
| | | | | | | | ATCCTTAATAC |
| | | | | | | | TGTTTATTACA |
| | | | | | | | GCCCCG |
| 87. | J0509D03-3 | | AU018874 | J0509D03 | | Chromosome 13 | CTCTGATACTG |
| | | | Mouse eight- | | | | AATAAACCTGA |
| | | | cell stage | | | | TGTGATGTACT |
| | | | embryo cDNA | | | | TATAGTCCTTA |
| | | | Mus musculus | | | | AGTCTTGAGAG |
| | | | cDNA clone | | | | TTAGA |
| | | | J0509D03 3′, | | | | |
| | | | MRNA | | | | |
| | | | sequence | | | | |
| 88. | H3055A11-5 | | UNKNOWN: | H3055A11 | Data not found | Chromosome 3 | GGCAACTACG |
| | | | Similar to | | | | ACTTTGTAGAG |
| | | | Homo sapiens | | | | GCCATGATTGT |
| | | | KIAA1363 | | | | GAACAATCAC |
| | | | protein | | | | ACTTCACTTGA |
| | | | (KIAA1363), | | | | TGTAGAA |
| | | | mRNA | | | | |
| 89. | C0455A05-3 | AW413625 | expressed | C0455A05 | Mm.1643 | Chromosome 19 | ACTTCATAGGA |
| | | | sequence | | | | TTCACAATGGA |
| | | | AW413625 | | | | GAGGGCTAGG |
| | | | | | | | AAGATACTGG |
| | | | | | | | ACAATTTTCAG |
| | | | | | | | CAGTGTG |
| 90. | NM_019732.1 | Runx3 | runt related | NM_019732 | Mm.247493 | Chromosome 4 | CACCTCTTGTC |
| | | | transcription | | | | TCCAGCCATGC |
| | | | factor 3 | | | | CCAGGATCAAT |
| | | | | | | | TCTAGAATCAG |
| | | | | | | | AGGCTACCCCT |
| | | | | | | | GCCTG |
| 91. | L0008A03-3 | AW546412 | ESTs | L0008A03 | Mm.182599 | Chromosome 16 | CGTCAGTGACC |
| | | | AW546412 | | | | CACTCAATACT |
| | | | | | | | GTGGTGGGAA |
| | | | | | | | GTAAGATGATG |
| | | | | | | | CCAAATCTATA |
| | | | | | | | ACCTGT |
| 92. | K0329C10-3 | Thbs1 | thrombospondin | K0329C10 | Mm.4159 | Chromosome 12 | CGAATGAGAA |
| | | | 1 | | | | TGCATCTTCCA |
| | | | | | | | AGACCATGAA |
| | | | | | | | GAGTTCCTTGG |
| | | | | | | | GTTTGCTTTTG |
| | | | | | | | GGAAAGC |
| 93. | H3115H03-3 | BC019206 | cDNA sequence | H3115H03 | Mm.259061 | Chromosome 10 | CCGGCGGGCCC |
| | | | BC019206 | | | | TAGTTTCTATG |
| | | | | | | | TATTTAGAATG |
| | | | | | | | AACTCGTGTAC |
| | | | | | | | ATATGTAAAGA |
| | | | | | | | TCTTT |
| 94. | C0643F09-3 | Usp18 | ubiquitin | C0643F09 | Mm.27498 | Chromosome 6 | CAAGCTGGTTG |
| | | | specific | | | | GAGCCTCCAGC |
| | | | protease 18 | | | | CTTCAAAATTC |
| | | | | | | | TGAATCTAATA |
| | | | | | | | AACATTAATGC |
| | | | | | | | ACACT |
| 95. | X84046.1 | Hgf | hepatocyte | H84046 | Mm.267078 | Chromosome 5 | CAATCCTAGAA |
| | | | growth factor | | | | CAACTACTTGA |
| | | | | | | | GTGTTGTGAGT |
| | | | | | | | GTTCAGATACT |
| | | | | | | | CATTAATATAT |
| | | | | | | | ATGGG |
| 96. | L0236C05-3 | Aldh1b1 | aldehyde | L0236C05 | Mm.24457 | Chromosome 4 | TCCCACCTCTC |
| | | | dehydrogenase | | | | TGATGAGTTAT |
| | | | 1 family, | | | | AGCCAAGAAG |
| | | | member B1 | | | | CCTTAGGAGTC |
| | | | | | | | TCCATAAGGCA |
| | | | | | | | TATTCA |
| 97 | H3055E08-3 | Mcoln2 | mucolipin 2 | H3055E08 | Mm.116862 | Chromosome 3 | AAGAAATATTC |
| | | | | | | | CCACTTCAGAG |
| | | | | | | | TGTGTAAGCAA |
| | | | | | | | TATTTAAACCC |
| | | | | | | | AGATAAAGAT |
| | | | | | | | GCATGC |
| 98. | H3009F12-3 | BG06369 | ESTs | H3009F12 | Mm.196869 | Chromosome 5 | TTTGGGAGTGG |
| | | | BG063639 | | | | GCTTCATGAAT |
| | | | | | | | GCGCTCTTACC |
| | | | | | | | AAAGGAGCCA |
| | | | | | | | TGTTTCCATTG |
| | | | | | | | TATCAA |
| 99. | J0208G12-3 | Cxc11 | chemokine (C- | J0208G12 | Mm.21013 | No Chromosome | TTTCATTAAAC |
| | | | X-C motif) | | | location | TAATATTTATT |
| | | | ligand 1 | | | info available | GGGAGACCAC |
| | | | | | | | TAAGTGTCAAC |
| | | | | | | | CACTGTGCTAG |
| | | | | | | | TAGAAG |
| 100. | K0300C11-3 | 9130025P16Rik | RIKEN cDNA | K0300C11 | Mm.153315 | Chromosome 1 | AAGTGACTCCA |
| | | | 9130025P16 | | | | TTTTCATATGT |
| | | | gene | | | | ACTTAAACACA |
| | | | | | | | GAGTTCCTGTG |
| | | | | | | | GCCTCTGTAAG |
| | | | | | | | CTCAG |
| 101. | H3104F03-5 | Krt1-18 | keratin complex | H3104F03 | Mm.22479 | Chromosome 15 | CAAGGTGAAG |
| | | | 1, acidic, gene | | | | AGCCTGGAAA |
| | | | 18 | | | | CTGAGAACAG |
| | | | | | | | GAGACTGGAG |
| | | | | | | | AGCAAAATCC |
| | | | | | | | GGGAACATCT |
| 102. | L0858D08-3 | Trim2 | tripartite motif | L0858D08 | Mm.44876 | Chromosome 3 | GCATGTGATTG |
| | | | protein | | | | ATTCATGATTT |
| | | | | | | | CCCCTTAGAGA |
| | | | | | | | GCAAGTGTTAC |
| | | | | | | | CAAAGTTCTGT |
| | | | | | | | TGAGC |
| 103. | L0508H09-3 | BY564994 | EST BY564994 | L0508H09 | Mm.290934 | Chromosome 12 | TGCTCCAGATG |
| | | | | | | | TGAAACTTATA |
| | | | | | | | GACGTAGACTA |
| | | | | | | | CCCTGAAGTGA |
| | | | | | | | ATTTCTATACA |
| | | | | | | | GGAAG |
| 104. | L0701G07-3 | BM194833 | ESTs | L0701G07 | Mm.221788 | Chromosome 2 | TGTACAACTGA |
| | | | BM194833 | | | | ACTCACCTCTT |
| | | | | | | | GTGAAGAATTA |
| | | | | | | | TGATTGTCTTA |
| | | | | | | | CTTGTAAAGAA |
| | | | | | | | AGCAC |
| 105. | K0102A10-3 | E430015L02Rik | RIKEN cDNA | K0102A10 | Mm.33498 | Chromosome 16 | TTTTGCAGGGG |
| | | | E430025L02 | | | | TCGAGTGTGAT |
| | | | gene | | | | GCATTGAAGGT |
| | | | | | | | TAAAACTGAA |
| | | | | | | | ATTTGAAAGAG |
| | | | | | | | TTCCAT |
| 106. | C0190H11-3 | Spn | sialophorin | C0190H11 | Mm.87180 | Chromosome 7 | CAAACAGAAA |
| | | | | | | | ACAGGGAGAT |
| | | | | | | | GTAAAACAGTT |
| | | | | | | | TCAACTCCATC |
| | | | | | | | AGTTATGAAAC |
| | | | | | | | CATAGCT |
| 107. | L0514A11-3 | 2810457I06Rik | RIKEN cDNA | L0514A11 | Mm.133615 | Chromosome 9 | TCAGCAAATTG |
| | | | 2810457I06 | | | | GCGATTTCGGA |
| | | | gene | | | | ATCCTATGACA |
| | | | | | | | CCTACATCAAT |
| | | | | | | | AGGAGTTTCCA |
| | | | | | | | GGTGA |
| 108. | J0911E11-3 | Nefl | neurofilament, | J0911E11 | Mm.1956 | Chromosome 14 | CATGTGCAACC |
| | | | light | | | | TCATGGGAAA |
| | | | polypeptide | | | | AATAGTAACTT |
| | | | | | | | GAATCTTCAGT |
| | | | | | | | GGTTAGAAATT |
| | | | | | | | AAAGAC |
| 109. | K0647E02-3 | Def6 | differentially | K0647E02 | Mm.60230 | Chromosome 17 | GTCTCAAGGAT |
| | | | expressed in | | | | CTGGGACCAG |
| | | | FDCP 6 | | | | AACTGGGAAA |
| | | | | | | | GAAAAGGAAT |
| | | | | | | | GACCAAGACA |
| | | | | | | | AGATCATAC |
| 110. | H3091E09-3 | Eifla | eukaryotic | H3091E09 | Mm.143141 | Chromosome Un | TGAATCAGAG |
| | | | translation | | | | AAAAGAGAGT |
| | | | initiation factor | | | | TGGTGTTTAAA |
| | | | 1A | | | | GAATATGGGC |
| | | | | | | | AAGAGTATGCT |
| | | | | | | | CAGGTGAC |
| 111. | AF286725.1 | Pdgfc | platelet-derived | AF286725 | Mm.40268 | Chromosome 3 | AAAGGAAATC |
| | | | growth factor, | | | | ATATCAGGATA |
| | | | C polypeptide | | | | AGATTTGTATC |
| | | | | | | | TGATGAGTATT |
| | | | | | | | TTCCATCTGAA |
| | | | | | | | CCCGGA |
| 112. | D31942.1 | Osm | oncostatin M | D31942 | 18413 | Chromosome 11 | CAGTCCTCTTG |
| | | | | | | | AAAGGTCTCAG |
| | | | | | | | AAGCTGGTGA |
| | | | | | | | GCAATTACTTG |
| | | | | | | | GAGGGACATG |
| | | | | | | | ACTAATT |
| 113. | L0046b04-3 | Alcam | activated | L0046B04 | Mm.2877 | Chromosome 16 | AGAGGAGTCTC |
| | | | leukocyte cedl | | | | CTTATATTAAT |
| | | | adhesion | | | | GGCAGGCATTA |
| | | | molecule | | | | TAGTAAAATTA |
| | | | | | | | TCATTTCCCCT |
| | | | | | | | GAGGA |
| 114. | K0131D09-3 | LOC217304 | similar to | K0131D09 | Mm.297591 | Chromosome 11 | GCATGAGTGTA |
| | | | triggering | | | | TAGGTGAAGGT |
| | | | receptor | | | | TTCACTTTAAG |
| | | | expressed on | | | | ATGCTGTCTTC |
| | | | myeloid cells 5 | | | | AGTTCTCTTGC |
| | | | (LOC217304), | | | | CTATG |
| | | | mRNA | | | | |
| 115. | H3024C07-3 | Hexa | hexosaminidase | H3024C07 | Mm.2284 | Chromosome 9 | ATCGTCTCTGA |
| | | | A | | | | TTATGACAAGG |
| | | | | | | | GCTATGTGGTG |
| | | | | | | | TGGCAGGAGG |
| | | | | | | | TATTTGATAAT |
| | | | | | | | AAAGTG |
| 116. | L0251A07-3 | B4galt1 | UDP- | L0251A07 | Mm.15622 | Chromosome 4 | CTGTTCGTGTT |
| | | | Gal:betaGlcNA | | | | GGGTTTTGTTC |
| | | | c beta 1,4- | | | | ATGTCAGATAC |
| | | | galactosyl- | | | | GTGGTTCATTC |
| | | | transferase, | | | | TCAGGACCAA |
| | | | polypeptide 1 | | | | GGGAAA |
| 117. | C0612G04-3 | Grip 1 | glutamate | C0612G04 | Mm.196692 | Chromosome 10 | GTGCAATAGA |
| | | | receptor | | | | AATATATGATT |
| | | | interacting | | | | TCAAACACATT |
| | | | protein 1 | | | | TCTGAACTGCC |
| | | | | | | | AGGGCAAGAA |
| | | | | | | | AGTATAG |
| 118. | C0357B04-3 | | C0357B04-3 | C0357B04 | | No Chromosome | CTTGTCGTTTT |
| | | | NIA Mouse | | | loction | TGGGGGTTGTA |
| | | | Undifferentiated | | | info available | ATATCTAAGGG |
| | | | ES Cell | | | | TGAAAAAATTA |
| | | | cDNA Library | | | | ATTTCCAAAGC |
| | | | (Short) Mus | | | | CAAGA |
| | | | musculus | | | | |
| | | | cDNA clone | | | | |
| | | | C0357B04 3′, | | | | |
| | | | MRNA | | | | |
| | | | sequence | | | | |
| 119. | L0529E02-3 | Egfl3 | EGF-like- | L0529E02 | Mm.29268 | Chromosome 4 | CAACTGTTTAC |
| | | | domain, | | | | CTGGAAATGTA |
| | | | multiple 3 | | | | GTCCAGACCAT |
| | | | | | | | ATTTATATAAG |
| | | | | | | | GTATTTATGGG |
| | | | | | | | CATCT |
| 120. | L0218E05-3 | Dnase2a | deoxyribonuclease | L0218E05 | Mm.220988 | Chromosome 8 | CCTTCCAGAGC |
| | | | II alpha | | | | TTTGCCAAATT |
| | | | | | | | TGGAAAATTTG |
| | | | | | | | GAGATGACCTG |
| | | | | | | | TACTCCGGATG |
| | | | | | | | GTTGG |
| 121. | H3074C12-3 | Dutp | deoxyuridine | H3074C12 | Mm.173383 | Chromosome 2 | TAGGTGAGTTA |
| | | | triphosphatase | | | | GGAATCTGCCA |
| | | | | | | | TAAGGTCGTTT |
| | | | | | | | ATAGGATCTGT |
| | | | | | | | TTATATGAAGT |
| | | | | | | | AATGG |
| 122. | H3072F09-3 | Icsbp1 | interferon | H3072F09 | Mm.249937 | Chromosome 8 | ATGACTTTCTC |
| | | | consensus | | | | TGCTTGGTTGG |
| | | | sequence | | | | AGAAGAAGAA |
| | | | binding protein | | | | TCTTTACTATT |
| | | | 1 | | | | CAGCTTCTTTT |
| | | | | | | | CTTTTT |
| 123. | c0829f05-3 | 4632404H22Rik | RIKEN cDNA | C0829F05 | Mm.28559 | Chromosome X | CCGGGGTGGG |
| | | | 4632404H22 | | | | AAGTTGTTTTT |
| | | | gene | | | | TCCTGGGGGTT |
| | | | | | | | TTTTCCCCTTA |
| | | | | | | | TTTGTTTTGGG |
| | | | | | | | GCCCCT |
| 124. | L0063A12-3 | | similar to | L0063A12 | Mm.38094 | Chromosome X | GGAAGATGGG |
| | | | ubiquitin- | | | | TAAATAGTAGA |
| | | | conjugating | | | | CTGTGGTGTAT |
| | | | enzyme UBCi | | | | TTGGAACAAG |
| | | | (LOC245350), | | | | GTAGCTTTAAA |
| | | | mRNA | | | | GACACAA |
| 125. | C0143E09-3 | 6330548O06Rik | RIKEN cDNA | C0143E09 | Mm.41694 | Chromosome 5 | CCAGGTTCAGA |
| | | | 6330548O06 | | | | GCGGACTGCTA |
| | | | gene | | | | ATAATAATGTG |
| | | | | | | | TGTATTGATCG |
| | | | | | | | AGGAAAAAGT |
| | | | | | | | GCGGAG |
| 126. | K0127G03-3 | | transcribed | K0127G03 | Mm.32947 | Chromosome 14 | TGCATGGGAA |
| | | | sequence with | | | | ATTTCTACGTG |
| | | | weak similarity | | | | GCTCACTTCAC |
| | | | to protein | | | | CAAGGCTTATT |
| | | | ref:NP_000072.1 | | | | GCACTGGGAA |
| | | | (H. spaiens) | | | | AAGAAGA |
| | | | beige protein | | | | |
| | | | homolong; | | | | |
| | | | Lysosomal | | | | |
| | | | trafficking | | | | |
| | | | regulator | | | | |
| | | | [Homo sapiens] | | | | |
| 127. | H3109D03-3 | Lamp2 | lysosomal | H3109D03 | Mm.486 | Chromosome X | TTAACCTAAAG |
| | | | membrane | | | | GTGCAACCTTT |
| | | | glycoprotein 2 | | | | TAATGTGACAA |
| | | | | | | | AAGGACAGTA |
| | | | | | | | TTCTACAGCTC |
| | | | | | | | AAGACT |
| 128. | J0034B02-3 | Dhx16 | DEAH (Asp-) | J0034B02 | Mm.5624 | Chromosome 17 | TCCCCACTACT |
| | | | Glu-Ala-His) | | | | ATAAGGCCAA |
| | | | box polypeptide | | | | GGAGCTAGAA |
| | | | 16 | | | | GATCCCCATGC |
| | | | | | | | TAAGAAAATG |
| | | | | | | | CCCAAAAA |
| 129. | K0428C07-3 | Plcb3 | phospholipase | K0428C07 | Mm.6888 | Chromosome 19 | ATAGGTACTCC |
| | | | C, beta 3 | | | | CCGATTCCCAA |
| | | | | | | | GGAGCAGCTA |
| | | | | | | | GTGGAACCCTG |
| | | | | | | | GAGTTTTGGGT |
| | | | | | | | AGTAGA |
| 130. | K0119F10-3 | Ccl9 | chemokine (C- | K0119F10 | Mm.2271 | No Chromosome | AGTAGTATTTC |
| | | | C motif) ligand | | | location | CAGTATTCTTT |
| | | | 9 | | | info available | ATAAATTCCCC |
| | | | | | | | TTGACATGACC |
| | | | | | | | ATCTTGAGCTA |
| | | | | | | | CAGCC |
| 131. | J0046B07-3 | Tuba4 | tubulin, alpha 4 | J0046B07 | Mm.1155 | Chromosome 1 | ACCGCTACTTG |
| | | | | | | | GAGCCTGTTCA |
| | | | | | | | CTGTGTTTATT |
| | | | | | | | GCAAAATCCTT |
| | | | | | | | TCGAAATAAAC |
| | | | | | | | AGTCT |
| 132. | C0117E11-3 | Neu1 | neuraminidase | C0117E11 | Mm.8856 | Chromosome 17 | TGAACTCTGAC |
| | | | 1 | | | | CTTTTGCAACT |
| | | | | | | | TCTCATCAACA |
| | | | | | | | GGGAAGTCTCT |
| | | | | | | | TGGTTATGACT |
| | | | | | | | TAACA |
| 133. | C0101C01-3 | Sdc1 | sydecan 1 | C0101C01 | Mm.2580 | No Chromosome | GTCTGTTCTTG |
| | | | | | | location | GGAATGGTTTA |
| | | | | | | info available | AGTAATTGGGA |
| | | | | | | | CTCTAGCTCAT |
| | | | | | | | CTTGACCTAGG |
| | | | | | | | GTCAC |
| 134. | K0245A03-3 | 9130012B15Rik | RIKEN cDNA | K0245A03 | Mm.35104 | No Chromosome | CCAGCCTGACC |
| | | | 9130012B15 | | | location | AGATTTTAGTT |
| | | | gene | | | info available | ACCTTTTAAGG |
| | | | | | | | AAGAGAGATTT |
| | | | | | | | ATTCTAATGCC |
| | | | | | | | ATAAA |
| 135. | H3109A02-3 | Fcerlg | Fc receptor, | H3109A02 | Mm.22673 | Chromosome 1 | CACCTCTGTGC |
| | | | lgE, high | | | | TTTGAAGGTTG |
| | | | affinity I, | | | | GCTGACCTTAT |
| | | | gamma | | | | TCCCATAATGA |
| | | | polypeptide | | | | TGCTAGGTAGG |
| | | | | | | | CTTTA |
| 136. | L0819C05-3 | Mapk8ip | mitogen | L0819C05 | Mm.2720 | Chromosome 2 | CTGAGCTCAGG |
| | | | activated | | | | CTGAGCCCACG |
| | | | protein kinase 8 | | | | CACCTCCAAAG |
| | | | interacting | | | | GACTTTCCAGT |
| | | | protein | | | | AAGGAAATGG |
| | | | | | | | CAACGT |
| 137. | U77083.1 | Anpep | alanyl | U77083 | Mm.4487 | Chromosome 7 | AGAACAGCAG |
| | | | (membrane) | | | | TTAGTTCCTGG |
| | | | aminopeptidase | | | | TTCTGAGAACC |
| | | | | | | | ACTTGTCCCAG |
| | | | | | | | TATGACACCTC |
| | | | | | | | TTACTA |
| 138. | C0164B01-3 | Tnfaip2 | tumor necrosis | C0164B01 | Mm.4348 | Chromosome 12 | ATGTGTGTACT |
| | | | factor, alpha- | | | | CAGGACAGAA |
| | | | induced protein | | | | TCCAGAGATTT |
| | | | 2 | | | | CTTTTTTATAT |
| | | | | | | | AGCTTGATATA |
| | | | | | | | AAACAG |
| 139. | H3085G03-3 | Cyba | cytochrome b- | H3085G03 | Mm.448 | Chromosome 8 | ACGTTTCACAC |
| | | | 245, alpha | | | | AGTGGTATTTC |
| | | | polypeptide | | | | GGCGCCTACTC |
| | | | | | | | TATCGCTGCAG |
| | | | | | | | GTGTGCTCATC |
| | | | | | | | TGTCT |
| 140. | H3074F04-3 | Abcc3 | ATP-binding | H3074F04 | Mm.23942 | Chromosome 11 | TTTTTTAATTCT |
| | | | cassette, sub- | | | | GCAAATTGTCT |
| | | | family C | | | | CACAGTGGAAT |
| | | | (CFTR/MRP), | | | | GAGGAAATGA |
| | | | member 3 | | | | GTTAGAGATCA |
| | | | | | | | CAGCC |
| 141. | H3145E02-3 | Wbp1 | WW domain | H3145Eo2 | Mm.1109 | Chromosome 6 | GTGCTATCTTT |
| | | | binding protein | | | | ACTCACTCCCA |
| | | | 1 | | | | AGACATACAC |
| | | | | | | | AGGAGCCTTTA |
| | | | | | | | ATCTCATTAAA |
| | | | | | | | GAGACA |
| 142. | K0609F07-3 | Cd53 | CD53 antigen | K0609F07 | Mm.2692 | Chromosome 3 | GAGGTCCAAGT |
| | | | | | | | TTAAATGTTAG |
| | | | | | | | TCTCCTAACAA |
| | | | | | | | CTGTCAAATCA |
| | | | | | | | ATTTCTAGCCT |
| | | | | | | | CTAAA |
| 143. | K0205H04-3 | 9830148O20Rik | RIKEN cDNA | K0205H04 | Mm.21630 | Chromosome 9 | CTTCTAGATCC |
| | | | 9830148O20 | | | | TTCTGCAGAAA |
| | | | gene | | | | TCATCGTCCTA |
| | | | | | | | AAGGAGCCTCC |
| | | | | | | | AACTATTCGAC |
| | | | | | | | CGAAT |
| 144. | H3095H04-3 | 2410002I16Rik | RIKEN cDNA | H3095H04 | Mm.17537 | Chromosome 18 | ACTTATTCATC |
| | | | 2410002I16 | | | | CTTGCCTATAC |
| | | | gene | | | | CCACCCCCCAA |
| | | | | | | | AAACAGGTTTT |
| | | | | | | | ATTAATAAAAA |
| | | | | | | | ATGTG |
| 145. | C0623H08-3 | Tm7sfl | transmembrane | C0623H08 | Mm.1585 | Chromosome 13 | TACAGTAACAA |
| | | | 7 superfamily | | | | GCAAGCTATCA |
| | | | member 1 | | | | TCCATTTTTAC |
| | | | | | | | AATAAAGTTGT |
| | | | | | | | CAGCATTCATG |
| | | | | | | | TCAGC |
| 146. | L0242F05-3 | 2700088M22Rik | RIKEN cDNA | L0242F05 | Mm.103104 | Chromosome 15 | TTATTTACTTT |
| | | | 2700088M22 | | | | ATCTTAGTATG |
| | | | gene | | | | TAACCTTAGCT |
| | | | | | | | GACCTGAAACC |
| | | | | | | | CACTGGTAGAC |
| | | | | | | | TAGAC |
| 147. | C0177F02-3 | Sdc3 | sydecan 3 | C0177F02 | Mm.206536 | Chromosome 4 | CCTGTCCTGAG |
| | | | | | | | TTCATGGCCAA |
| | | | | | | | AACTTAAATAA |
| | | | | | | | GAGAAGGAGG |
| | | | | | | | AGAGGGTCAG |
| | | | | | | | ATGGATA |
| 148. | L0803B02-3 | Ppp1r9a | protein | L0803B02 | Mm.156600 | Chromosome 6 | AAAGGGGCCT |
| | | | phosphatase 1, | | | | GAGTATACGCT |
| | | | regulatory | | | | GTTGCAAGCTG |
| | | | (inhibitor) | | | | TATACTTCATT |
| | | | subunit 9A | | | | TCCTTCGGCTG |
| | | | | | | | GTTTAT |
| 149. | H3061D01-3 | BB172728 | ESTs | H3061D01 | Mm.254385 | Chromosome 3 | TATCCGGACAG |
| | | | BB172728 | | | | TCTATGTGAAA |
| | | | | | | | TAGGACCAAG |
| | | | | | | | GTCGAAAGCC |
| | | | | | | | GGAAAGACAT |
| | | | | | | | CAACAGAA |
| 150. | L0259D11-3 | Clqb | complement | L0259D11 | Mm.2570 | Chromosome 4 | CTGCTTTTCCC |
| | | | component 1, q | | | | TGACATGGATG |
| | | | subcomponent, | | | | CGTAATCACGG |
| | | | beta | | | | GGTCAAATTAC |
| | | | polypeptide | | | | ACCTATCCAAC |
| | | | | | | | ACCAT |
| 151. | H3011D10-3 | Lcpl | lymphocyte | H3011D10 | Mm.153911 | Chromosome 14 | AACAAAGAGG |
| | | | cytosolic | | | | ACAGTATGAAT |
| | | | protein 1 | | | | TTGAATAGCTC |
| | | | | | | | CCACTAGATAA |
| | | | | | | | GCAATTTCCAC |
| | | | | | | | GAGAAC |
| 152. | H3052B11-3 | Pctk3 | PCTAIRE- | H3052B11 | Mm.28130 | Chromosome 1 | CTGACTGTGAA |
| | | | motif protein | | | | TGTCGTGACTC |
| | | | kinase 3 | | | | AGAGCAAAGA |
| | | | | | | | CAGAGAATAT |
| | | | | | | | ATTTAATTCAT |
| | | | | | | | GTTGTAC |
| 153. | k0413h04-3 | Anxa8 | annexin A8 | K0413H04 | Mm.3267 | Chromosome 14 | GCCTGAAGAA |
| | | | | | | | CATGACAGAA |
| | | | | | | | CTCTTCTCAAT |
| | | | | | | | ATTCGTTGGGC |
| | | | | | | | TTTCAGAATCA |
| | | | | | | | TAAACAT |
| 154. | H3054F05-3 | Lyzs | lysozyme | H3054F05 | Mm.45436 | Chromosome 10 | CCTGTGTGAAT |
| | | | | | | | AAAAATACAA |
| | | | | | | | GAACTGCTTAT |
| | | | | | | | AGGAGACCAG |
| | | | | | | | TTGATCTTGGG |
| | | | | | | | AAACAGC |
| 155. | H3060F11-3 | Cybb | cytochrome b- | H3060F11 | Mm.200362 | Chromosome X | GTAAGAAATAT |
| | | | 245, beta | | | | TAGACTGATTG |
| | | | polypeptide | | | | GAGTTAAAGTA |
| | | | | | | | GCACTCTACAT |
| | | | | | | | TTACCATGGTG |
| | | | | | | | TTTGG |
| 156. | H3012F08-3 | 9430068N19Rik | RIKEN cDNA | H3012F08 | Mm.143819 | Chromosome 1 | TGTGAAAGATT |
| | | | 9430068N19 | | | | GTGCATCTGCA |
| | | | gene | | | | TTCAACTACCC |
| | | | | | | | TGAACCCTTAG |
| | | | | | | | GGAAGAAATG |
| | | | | | | | GATTCC |
| 157. | G0106B08-3 | Abr | active BCR- | G0106B08 | Mm.27923 | Chromosome 11 | AGCTGCCTACT |
| | | | related gene | | | | AGCAGTTTAAC |
| | | | | | | | AAGGAGCCTTG |
| | | | | | | | CTGTCTCAGAC |
| | | | | | | | AGGTGAAAGA |
| | | | | | | | AAATGT |
| 158. | L0287A12-3 | Tdrkh | tudor and KH | L0287A12 | Mm.40894 | Chromosome 3 | CCATGTTTGAA |
| | | | domain | | | | AGTATGTAATG |
| | | | containing | | | | AAGAGGAGCC |
| | | | protein | | | | TATTAACCATA |
| | | | | | | | TGAAAGACAG |
| | | | | | | | GAATACT |
| 159. | H3083D01-3 | AY007814 | hypothetical | H3083D01 | Mm.160389 | Chromosome 7 | GTGAATTGGAT |
| | | | protein, | | | | GCATAGCATGT |
| | | | 12H19.01.T7 | | | | TTTGTATGTAA |
| | | | | | | | ATGTTCCTTAA |
| | | | | | | | AAGTGTCACCA |
| | | | | | | | TGAAC |
| 160. | H313F02-3 | BGO74151 | ESTs | H3131F02 | Mm.142524 | Chromosome 8 | ACCCACTGACT |
| | | | BG074151 | | | | AGGATAACTG |
| | | | | | | | GAAAGGAGTC |
| | | | | | | | TGACCTGAATG |
| | | | | | | | ACGCATTAAAC |
| | | | | | | | TCCTGCA |
| 161. | C0172H02-3 | Lgals3 | lectin, galactose | C0172H02 | Mm.2970 | Chromsome 14 | CCCGCTTCAAT |
| | | | binding, soluble | | | | GAGAACAACA |
| | | | 3 | | | | GGAGAGTCATT |
| | | | | | | | GTGTGTAACAC |
| | | | | | | | GAAGCAGGAC |
| | | | | | | | AATAACT |
| 162. | K0542E07-3 | Cd44 | CD44 antigen | K0542E07 | Mm.24138 | Chromosome 2 | ATATTAACTCT |
| | | | | | | | ATAAAAATAAG |
| | | | | | | | GCTGTCTCTAA |
| | | | | | | | AATGGAACTTC |
| | | | | | | | CTTTCTAAGGG |
| | | | | | | | TCCCAC |
| 163. | C0450H11-3 | E430019N21Rik | RIKEN cDNA | C0450H11 | Mm.275894 | Chromosome 14 | TGTGGGTTTTT |
| | | | E430019N21 | | | | TGAAGAATTAA |
| | | | gene | | | | TGAGCATGTAC |
| | | | | | | | ATAGAAATAGT |
| | | | | | | | GACTGCTTGAA |
| | | | | | | | TCCTG |
| 164. | K0216A08-3 | Orc51 | origin | K0216A08 | Mm.566 | Chromosome 5 | CTACTCTTAAT |
| | | | recognition | | | | AGATGTTAT- |
| | | | complex, | | | | CTT |
| | | | subunit 5-like | | | | AACACTGAAAT |
| | | | (S. cerevisiaae) | | | | TGCCTGAAACC |
| | | | | | | | CATTTACTTAG |
| | | | | | | | GACTG |
| 165. | H3122D03-3 | Pdgfc | platelet-derived | H3122D03 | Mm.40268 | Chromosome 3 | TCAGACCA- |
| | | | growth factor, | | | | TTTC |
| | | | C polypeptide | | | | TAGGCACAGTG |
| | | | | | | | TTCTGGGCTAT |
| | | | | | | | GGCGCTGTATG |
| | | | | | | | GACATATCCTA |
| | | | | | | | TTTAT |
| 166. | C0037H07-3 | Il13ral | interleukin 13 | C0037H07 | Mm.24208 | Chromosome X | TCTGAATCTGG |
| | | | receptor, alpha | | | | GCACTGAAGG |
| | | | 1 | | | | GATGCATAAA |
| | | | | | | | ATAATGTTAAT |
| | | | | | | | GTTTTCAGTAA |
| | | | | | | | TGTCTTC |
| 167. | H30554F04-3 | 2610318I15Rik | RIKEN cDNA | H3054F04 | Mm.34490 | Chromosome 11 | GATCCTTAGGT |
| | | | 2610318I15 | | | | CTCCATAGGAT |
| | | | gene | | | | GATTTTTGAGG |
| | | | | | | | TAGTTAATCAG |
| | | | | | | | TGTAAACTCTT |
| | | | | | | | ACACA |
| 168. | L0908A12-3 | Blnk | B-cell linker | L0908A12 | Mm.9749 | Chromosome 19 | CTCAGCAGTAA |
| | | | | | | | CAGAGAAAAG |
| | | | | | | | ATGAATGAAG |
| | | | | | | | CCACTGAGGCT |
| | | | | | | | TCGTGAATGAA |
| | | | | | | | TGAATCT |
| 169. | G0111E06-3 | Car7 | carbonic | G0111E06 | Mm.154804 | Chromosome 8 | CTTTGTTCCTA |
| | | | anhydrase 7 | | | | CCCAGCCACCA |
| | | | | | | | AAGCCACCTAC |
| | | | | | | | ATAACAATCCA |
| | | | | | | | CTCATGTACTA |
| | | | | | | | GCAAA |
| 170. | L0284B06-3 | Ngfrap1 | nerve growth | L0284b06 | Mm.90787 | Chromosome X | AAATTGTCTAC |
| | | | factor receptor | | | | GCATCCTTATG |
| | | | (TNFRSF16) | | | | GGGGAGCTGTC |
| | | | associated | | | | TAACCACCACG |
| | | | protein 1 | | | | ATCACCATGAT |
| | | | | | | | GAATT |
| 171. | K0145G06-3 | Tcfec | transcription | K0145G06 | Mm.36217 | Chromosome 6 | ACATGATGTGA |
| | | | factor EC | | | | AAGAATCATTG |
| | | | | | | | AAGATCACAGT |
| | | | | | | | TGTCTACCGAG |
| | | | | | | | TTCAGATTTCC |
| | | | | | | | TTACA |
| 172. | H3001B08-3 | Lyn | Yamaguchi | H3001B08 | Mm.1834 | Chromosome 4 | CACCCCCCAGA |
| | | | sarcoma viral | | | | AAATGAGACT |
| | | | (v-yes-1) | | | | ATTGAACATTT |
| | | | oncogene | | | | TCCTTTGTGGT |
| | | | homolog | | | | AAGATCACTGG |
| | | | | | | | ACAGGA |
| 173. | G0117F12-3 | Prkcsh | protein kinase | G0117F12 | Mm.214593 | Chromosome 9 | AGTGATGGGG |
| | | | C substrate | | | | ACCATGACGA |
| | | | 80K-H | | | | GCTGTAGCCTG |
| | | | | | | | AACCTCAAGGC |
| | | | | | | | CTGAACCAGT |
| | | | | | | | CTACTGA |
| 174. | C0903A11-3 | 2510004l01Rik | RIKEN cDNA | C0903A11 | Mm.24045 | Chromosome 12 | AAAGGTCCCA |
| | | | 2510004L01 | | | | GGTTTCGATCT |
| | | | gene | | | | GTTTGGAGTTT |
| | | | | | | | GGAGTCTAATG |
| | | | | | | | GTTGCATAGAT |
| | | | | | | | AAACAG |
| 175. | L0062C10-3 | Rasa3 | RAS p21 | L0062C10 | Mm.18517 | Chromosome 8 | TCTATGTGCAT |
| | | | protein | | | | TAGGGGGTGA |
| | | | activator 3 | | | | CCCAGGGAAA |
| | | | | | | | TCCAAAGGGA |
| | | | | | | | ACAGTATTTGA |
| | | | | | | | TTTCTCAC |
| 176. | L0939G09-3 | Cd38 | CD38 antigen | L0939G09 | Mm.249873 | Chromosome 5 | CTACACATGTA |
| | | | | | | | CTTTAGGATTC |
| | | | | | | | TAGGTTTCTCC |
| | | | | | | | CTGAGCCCTGC |
| | | | | | | | TTTCGATGTAA |
| | | | | | | | CACTG |
| 177. | H3115B07-3 | S100a9 | S100calcium | H3115B07 | Mm.2128 | Chromosome 3 | AAGTCTAAAG |
| | | | binding protein | | | | GGAATGGCTTA |
| | | | A9 (calgranulin | | | | CTCAATGGCCT |
| | | | B) | | | | TTGTTCTGGGA |
| | | | | | | | AATGATAAGAT |
| | | | | | | | AAATAA |
| 178. | K0608H07-3 | Fyb | FYN binding | K0608H07 | Mm.254240 | Chromosome 15 | GGAAGAAAAA |
| | | | protein | | | | GACCTCAGGA |
| | | | | | | | AAAAATTTAAG |
| | | | | | | | TACGACGGTGA |
| | | | | | | | AATTCGAGTTC |
| | | | | | | | TATATTC |
| 179. | C0104E07-3 | Tcirg1 | T-cell, immune | C0104E07 | Mm.19185 | Chromosome 19 | GGATGAAGAA |
| | | | regulator 1 | | | | ACTGAGTTTGT |
| | | | | | | | CCCTTCTGAGA |
| | | | | | | | TCTTCATGCAC |
| | | | | | | | CAAGCAATCCA |
| | | | | | | | CACCAT |
| 180. | K0431D02-3 | Wisp1 | WNT1 | K0431D02 | Mm.10222 | Chromosome 15 | CTGTTCAGGCT |
| | | | inducible | | | | CAAACAATGG |
| | | | signaling | | | | GTTCCTCCTTG |
| | | | pathway protein | | | | GGGACATTCTA |
| | | | 1 | | | | CATCATTCCAA |
| | | | | | | | GGAAAA |
| 181. | L0837H10-3 | Igfbp2 | insulin-like | L0837H10 | Mm.141936 | Chromosome 1 | AGGAGTTCCCA |
| | | | growth factor | | | | GTTTTGACACA |
| | | | binding protein | | | | TGTATTTATAT |
| | | | 2 | | | | TTGGAAAGAG |
| | | | | | | | ACCAACACTGA |
| | | | | | | | GCTCAG |
| 182. | C0159A08-3 | Mta3 | metastasis | C0159A08 | Mm.18821 | Chromosome 17 | CTCAATAAAAG |
| | | | associated 3 | | | | CTCTAAGGAGA |
| | | | | | | | CATCACAACCC |
| | | | | | | | AGTCTTAAGGG |
| | | | | | | | TTCATGAGGTT |
| | | | | | | | TTAAT |
| 183. | K0649D06-3 | Ms4a6b | membrane- | K0649D06 | Mm.29487 | Chromosome 19 | ACTTAAAATGT |
| | | | spanning 4- | | | | AGACTGTTCAT |
| | | | domains, | | | | ACAGTGGGTAC |
| | | | subfamily A, | | | | CAGTATGAGTT |
| | | | member 6B | | | | GAATGTGTGTA |
| | | | | | | | TTACT |
| 184. | K0609D11-3 | Manla | mannosidase 1, | K0609D11 | Mm.117294 | Chromosome 10 | TTTCATAATAG |
| | | | alpha | | | | AACCGTCTACC |
| | | | | | | | AGTGACCTCTT |
| | | | | | | | GATTATGATTT |
| | | | | | | | GATTTGACTGC |
| | | | | | | | AAAAC |
| 185. | C0907B04-3 | Mcoln3 | mucolipin 3 | C0907B04 | Mm.114683 | Chromosome 3 | ATCCATGTGGC |
| | | | | | | | ATCAATTCAAT |
| | | | | | | | TATGTATAATA |
| | | | | | | | ATGACTTTACA |
| | | | | | | | AGGGCCCCTTA |
| | | | | | | | AAACC |
| 186. | H3020D08-3 | Edem 1 | ER degradation | H3020D08 | Mm.21596 | Chromosome 6 | CACAAAAGTC |
| | | | enhancer, | | | | AAATGTGGATA |
| | | | mannosidase | | | | TCGTACGCTGC |
| | | | alpha-like 1 | | | | ATCACGTCATA |
| | | | | | | | GACAAGTCTAA |
| | | | | | | | AGAAGA |
| 187. | J0039F05-3 | Gdf3 | growth | J0039F05 | Mm.4213 | Chromosome 6 | CTATCAGGATA |
| | | | differentiation | | | | GTGATAAGAA |
| | | | factor 3 | | | | CGTCATTCTCC |
| | | | | | | | GACATTATGAA |
| | | | | | | | GACATGGTAGT |
| | | | | | | | CGATGA |
| 188. | C0906C11-3 | BM218094 | ESTs | C0906C11 | Mm.212279 | Chromosome 6 | GGAGATCATCA |
| | | | BM218094 | | | | CTCTTGTATGA |
| | | | | | | | AATATACTAAC |
| | | | | | | | TCCAAACCTTT |
| | | | | | | | TTAGAGCAGAT |
| | | | | | | | TAGGC |
| 189. | L0266E10-3 | B930060C03 | hypothetical | L0266E10 | Mm.89568 | Chromosome 12 | ACTATTAAGCA |
| | | | protein | | | | CTCAGGAGAAT |
| | | | B930060C03 | | | | GTAGGAAAGA |
| | | | | | | | TTTCCTTTGCT |
| | | | | | | | ACAGTTTTTGT |
| | | | | | | | TCAGTA |
| 190. | H3060D11-3 | M115 | myeloid/lymph | H3060D11 | Mm.10878 | Chromosome 5 | AAAGAGAAAA |
| | | | oid or mixed- | | | | TATGTCAGATG |
| | | | lineage | | | | GTGATACCAGT |
| | | | leukemia 5 | | | | GCAACTGAAA |
| | | | | | | | GTGGTGATGAA |
| | | | | | | | GTTCCTG |
| 191. | L0062E01-3 | Tnc | tenascin C | L0062E01 | Mm.980 | Chromosome 4 | GAGAGAGGAA |
| | | | | | | | TGGGGCCCAG |
| | | | | | | | AGAAAAGAAA |
| | | | | | | | GGATTTTTACC |
| | | | | | | | AAAGCATCAA |
| | | | | | | | CACAACCAG |
| 192. | K0132G08-3 | A1662270 | expressed | K0132G08 | Mm.37773 | No Chromosome | GTTGTACTACT |
| | | | sequence | | | location | GGAAAGATTTT |
| | | | A1662270 | | | info available | GCTGGGACATA |
| | | | | | | | CAATATGTGTG |
| | | | | | | | AGAAAAATAG |
| | | | | | | | AGTTGT |
| 193. | H3114D08-3 | Arpc3 | actin related | H3114D08 | Mm.24498 | Chromosome 5 | AGACCAAAGA |
| | | | protein 2/3 | | | | CACGGACATTG |
| | | | complex, | | | | TGGATGAAGCC |
| | | | subunit 3 | | | | ATCTACTACTT |
| | | | | | | | CAAGGCCAAT |
| | | | | | | | GTCTTCT |
| 194. | C0649E02-3 | Unc93b | unc-93 | C0649E02 | Mm.28406 | Chromosome 19 | CAGAGCAGGG |
| | | | homolog B (C. | | | | GGCTTTTATTT |
| | | | elegans) | | | | TTATTTTTTAA |
| | | | | | | | TGGAAAATAAT |
| | | | | | | | CAATAAAGACT |
| | | | | | | | TTTGTA |
| 195. | L0293H10-3 | 2510048K03Rik | RIKEN cDNA | L0293H10 | Mm.39856 | Chromosome 7 | CTTGGCAGCTC |
| | | | 2510048K03 | | | | TCCTTACTTCT |
| | | | gene | | | | GGGACATTTGC |
| | | | | | | | CACTGTGGTAC |
| | | | | | | | TGCCAGGAAG |
| | | | | | | | GAATCT |
| 196. | H3024C03-3 | 1110008B24Rik | RIKEN cDNA | H3024C03 | Mm.275813 | Chromosome 12 | ACTTATAGAAA |
| | | | 1110008B24 | | | | AGGACAGGTT |
| | | | gene | | | | GAAGCCTAAG |
| | | | | | | | AAGAAAGAGA |
| | | | | | | | AGAAAGATCC |
| | | | | | | | GAGCGCGCT |
| 197. | H3055002-3 | Ctsc | cathepsin C | H3055G02 | Mm.684 | Chromosome 7 | TAGTTCAGTGA |
| | | | | | | | ACAAGTATCTG |
| | | | | | | | TCAATGAGTGA |
| | | | | | | | GCTGTGTCAAA |
| | | | | | | | ATCAAGTTATA |
| | | | | | | | TGTTC |
| 198. | K0518A04-3 | BM238476 | ESTs | K0518A04 | Mm.217227 | Chromosome 2 | CATGAATGTCA |
| | | | BM238476 | | | | AAACCTAATTA |
| | | | | | | | CAAAGCATCG |
| | | | | | | | GTCTCTTTGTT |
| | | | | | | | GTGAGGTATCA |
| | | | | | | | GAACCC |
| 199. | K0128H01-3 | Parvg | parvin, gamma | K0128H01 | Mm.202348 | Chromosome 15 | CCTGTCTCATG |
| | | | | | | | GGAGATTTGAA |
| | | | | | | | TCATAAGGAG |
| | | | | | | | AATCACTTTTT |
| | | | | | | | GTAACTTTATT |
| | | | | | | | GAGGAA |
| 200. | K0649F04-3 | Ccr2 | chemokine (C- | K0649F04 | Mm.6272 | Chromosome 9 | AAGTAAATATG |
| | | | C) receptor 2 | | | | CAAAGGAGAG |
| | | | | | | | AAGTTAGAGA |
| | | | | | | | AACTCCTCTCA |
| | | | | | | | TAAGAAAAAT |
| | | | | | | | GTCTTCCC |
| 201. | K0603E03-3 | Vav1 | vav 1 oncogene | K0603E03 | Mm.254859 | Chromosome 17 | TCGGAACTGTC |
| | | | | | | | CCTTAAGGAGG |
| | | | | | | | GTGATATCATC |
| | | | | | | | AAGATCCTCAA |
| | | | | | | | TAAGAAGGGA |
| | | | | | | | CAGCAA |
| 202. | K0649A02-3 | Stat1 | signal | K0649A02 | Mm.8249 | Chromosome 1 | TTAGTGGGCTG |
| | | | transducer and | | | | AACCTATCGGT |
| | | | activator of | | | | TTTAACTGGTT |
| | | | transcription 1 | | | | GTCTTAATTAA |
| | | | | | | | CCATAAACTTG |
| | | | | | | | GAGAA |
| 203. | H3013D11-3 | Mt2 | metallothionein | H3013D11 | Mm.147226 | Chromosome 8 | TTTTGTACAAC |
| | | | 2 | | | | CCTGACTCGTT |
| | | | | | | | CTCCACAACTT |
| | | | | | | | TTTCTATAAAG |
| | | | | | | | CATGTAACTGA |
| | | | | | | | CAATA |
| 204. | H3013B02-3 | Atp6vlb2 | ATPase, H + | H3013B02 | Mm.10727 | Chromosome 8 | AGACTTGGAA |
| | | | transporting, | | | | AAGGCTTGGGT |
| | | | V1 subunit B, | | | | ACAATTAAGA |
| | | | isoform 2 | | | | AAAACCCTACA |
| | | | | | | | TCCCACCCTCC |
| | | | | | | | TCTTGAC |
| 205. | L0541H09-3 | | transcribed | L0541H09 | Mm.221768 | Chromosome 6 | TAATAAAGAA |
| | | | sequence with | | | | ACTGTGGAAAT |
| | | | weak similarity | | | | ACTTGGATTTC |
| | | | to protein | | | | TACTGAAGACA |
| | | | pir:S12207 | | | | AAAGACTTCTA |
| | | | (M. musculus) | | | | GGCTGG |
| | | | S12207 | | | | |
| | | | hypothetical | | | | |
| | | | protein (B2 | | | | |
| | | | element) - | | | | |
| | | | mouse | | | | |
| 206. | K0516E03-3 | | Mus musculus | K0516E03 | Mm.214742 | Chromosome 10 | AGGTTAAACAT |
| | | | 12 days embryo | | | | ATATTCTTGGA |
| | | | embryonic | | | | AACATGAAATC |
| | | | body between | | | | ACAACTCTCAA |
| | | | diaphragm | | | | AAACCGTGAA |
| | | | region and neck | | | | CCACCA |
| | | | cDNA, RIKEN | | | | |
| | | | full-length | | | | |
| | | | enriched | | | | |
| | | | library, | | | | |
| | | | clone:9430012 | | | | |
| | | | B12 | | | | |
| | | | product:unknown | | | | |
| | | | EST, full | | | | |
| | | | insert sequence. | | | | |
| 207. | H3034A10-3 | Plaur | urokinase | H3034A10 | Mm.1359 | Chromosome 7 | CCTCGTGTTGT |
| | | | plasminogen | | | | CTTCTTTGGAC |
| | | | activator | | | | CTCAGTTTTTC |
| | | | receptor | | | | CATGAACCAG |
| | | | | | | | AAGAGAATTG |
| | | | | | | | GAACAAG |
| 208. | C0910G05-3 | BM218419 | ESTs | C0910G05 | Mm.217839 | Chromosome 10 | AATAGCAATGT |
| | | | BM218419 | | | | ATCAAACAATG |
| | | | | | | | GATGTGAAAA |
| | | | | | | | AGATGCGCTCT |
| | | | | | | | ATCATCATGAA |
| | | | | | | | AATGCC |
| 209. | C0262H12-3 | Msh2 | mutS homolog | C0262H12 | Mm.4619 | Chromosome 17 | TCTCTGGAGAA |
| | | | 2 (E. coli) | | | | ATCAGTAACTG |
| | | | | | | | CAAAAGGAAG |
| | | | | | | | AGAGGGTCTTT |
| | | | | | | | AAAGCACATGT |
| | | | | | | | AGTAAT |
| 210. | H3078C11-3 | BG069620 | ESTs | H3078C11 | Mm.173427 | Chromosome 2 | TGGAATGTTGA |
| | | | BG069620 | | | | AGAATGAAAT |
| | | | | | | | CTCGAGGGAAT |
| | | | | | | | TAGAGGTTGAG |
| | | | | | | | GTCATCTGGAT |
| | | | | | | | ATTCAG |
| 211. | L0926H09-3 | 6030440G05Rik | RIKEN cDNA | L0926H09 | Mm.27789 | Chromosome 6 | ATAGAACCAAT |
| | | | 6030440G05 | | | | GTAGGAAAAT |
| | | | gene | | | | CAGGCAAAAT |
| | | | | | | | AAAATGATGAT |
| | | | | | | | CAGTCCATGTC |
| | | | | | | | ATCATGG |
| 212. | J0076H03-3 | | C80125 Mouse | J0076H03 | | No Chromosome | AGATGGGAAA |
| | | | 3.5-dpc | | | location | AAGTACTGTAG |
| | | | blastocyst | | | info available | GTTCCTGAACT |
| | | | cDNA Mus | | | | CTGGATCTCAA |
| | | | musculus | | | | GCAGAAATGT |
| | | | cDNA clone | | | | ACTGTCT |
| | | | J0076H03 3′, | | | | |
| | | | MRNA | | | | |
| | | | sequence | | | | |
| 213. | L0817B08-3 | transcribed | L0817B08 | | Mm.221816 | Chromosome 18 not | AGGAAAACCC |
| | | | sequence with | | | placed | CGGTAGTTAGG |
| | | | strong | | | | ACATCTGAATT |
| | | | similarity to | | | | CTCAATTATTG |
| | | | protein | | | | GATTGCCAAAA |
| | | | sp:P00722 (E. | | | | GTGAAA |
| | | | coli) | | | | |
| | | | BGAL_ECOLI | | | | |
| | | | Beta | | | | |
| | | | galactosidase | | | | |
| | | | (Lactase) | | | | |
| 214. | H3065D11-3 | Crnkl1 | Cm, crooked | H3065D11 | Mm.273506 | Chromosome 2 | GTTTTTGGAAT |
| | | | neck-like 1 | | | | TTGGACCTGAA |
| | | | (Drosophila) | | | | AATTGTACCTC |
| | | | | | | | ATGGATTAAGT |
| | | | | | | | TTGCAGAATTA |
| | | | | | | | GAGAC |
| 215. | H3157E02-3 | 5630401J11Rik | RIKEN cDNA | H3157E02 | Mm.21104 | Chromosome 17 | TGGGACCTGTG |
| | | | 5630401J11 | | | | AAGCGACTGA |
| | | | gene | | | | AGAAAATGTTT |
| | | | | | | | GAAACAACAA |
| | | | | | | | GATTGCTTGCA |
| | | | | | | | ACAATTA |
| 216. | H3007C11-3 | BG063444 | ESTs | H3007C11 | Mm.182542 | No Chromosome | TCCATTATTAC |
| | | | BG063444 | | | location | ATACAACAATC |
| | | | | | | info available | AAGAAAAAGA |
| | | | | | | | CAGAAAACTA |
| | | | | | | | CCCTTAGAGAG |
| | | | | | | | ATCAGGG |
| 217. | K0517E07-3 | C53005OH1ORik | RIKEN cDNA | K0517E07 | Mm.260378 | Chromosome 4 | ATTCAACAGCA |
| | | | C530050H10 | | | | TTCTAGGAAAA |
| | | | gene | | | | TGGCAAGAAA |
| | | | | | | | GTAAATTATCA |
| | | | | | | | TCCATTTCAGG |
| | | | | | | | TCTGTG |
| 218. | H3150B11-5 | Ptpn2 | protein tyrosine | H3150B11 | Mm.260433 | Chromosome 18 | CCATATGATCA |
| | | | phosphatase, | CAGTCGTGTTA | | | |
| | | | non-receptor | AACTGCAAAGT | | | |
| | | | type 2 | ACTGAAAATG | | | |
| | | | | | | | ATTATATTAAT |
| | | | | | | | GCCAGC |
| 219. | C0199C01-3 | 9930104E21Rik | RIKEN cDNA | C0199C01 | Mm.29216 | Chromosome 18 | GGGCCATATTT |
| | | | 9930104E21 | | | | TAAAGATAAG |
| | | | gene | | | | GAGAGAGAAA |
| | | | | | | | CTAGCATACAG |
| | | | | | | | AATTTTCCTCA |
| | | | | | | | TATTGAG |
| 220. | H3063A09-3 | Rassf5 | Ras association | H3063A09 | Mm.248291 | Chromosome 1 | GAAAGGCGTTT |
| | | | (RaLGDS/AF-6) | | | | ATTCAGAAAAT |
| | | | domain family | | | | GATGGTAAGAT |
| | | | 5 | | | | TCAGACTTTAA |
| | | | | | | | AGCACAGTTAG |
| | | | | | | | ACCCA |
| 221. | K0445A07-3 | Hfe | hemochromatosis | K0445A07 | Mm.2681 | Chromosome 13 | TAAGGTGTTTT |
| | | | | | | | CTCCAGTTAAG |
| | | | | | | | TTCAGTTCCTG |
| | | | | | | | AATAGTAGTGA |
| | | | | | | | TTGCCCCAGTT |
| | | | | | | | GCAAC |
| 222. | H3123G07-3 | C630007C17Rik | RIKEN cDNA | H3123G07 | Mm.119383 | Chromosome 2 | CCACCATAAAG |
| | | | C630007C17 | | | | GAAAAAGGAC |
| | | | gene | | | | ATGTGTATGAG |
| | | | | | | | TAGGTGTTCAT |
| | | | | | | | CTATGTGCATA |
| | | | | | | | ATTGGC |
| 223. | H3094C03-3 | Bazla | bromodomain | H3094C03 | Mm.263733 | Chromosome 12 | GCACAAGATG |
| | | | adjacent to zinc | | | | GAGTCATTAAA |
| | | | finger domain | | | | ATTAAGGCATC |
| | | | 1A | | | | ATCATTTTCAG |
| | | | | | | | CATATAACATA |
| | | | | | | | GCAGAG |
| 224. | L0845H04-3 | BM117070 | ESTs | L0845H04 | Mm.221860 | Chromosome 1 | GATTAAAAAC |
| | | | BM117070 | | | | ATTAGGGATGA |
| | | | | | | | GAAATAATAA |
| | | | | | | | GGGCTTGCAAC |
| | | | | | | | TGTGTAGAAGC |
| | | | | | | | TAGAGCC |
| 225. | C0161F01-3 | BC010311 | cDNA sequence | C0161F01 | Mm.46455 | Chromosome 4 | TGAAGTACACT |
| | | | BC010311 | | | | CTCTAAATGAA |
| | | | | | | | AATGGGCTATA |
| | | | | | | | AATATGTTTGA |
| | | | | | | | GTAGGATAGG |
| | | | | | | | AGGAAG |
| 226. | H3034E07-3 | BG065726 | ESTs | H3034E07 | Mm.5522 | Chromosome 9 | GTGTAAGAAA |
| | | | BG065726 | | | | AGATGGGACT |
| | | | | | | | GACAATAAAA |
| | | | | | | | ATGAAGGTCA |
| | | | | | | | GGTAAGAAGT |
| | | | | | | | ACCAGACTCC |
| 227. | J0419G11-3 | Cldn8 | claudin 8 | J0419G11 | Mm.25836 | Chromosome 16 | GGGAAATATG |
| | | | | | | | CAGCGTTCTAT |
| | | | | | | | GTTTCCATAAG |
| | | | | | | | TGATTTTAGCA |
| | | | | | | | GAATGAGGTAT |
| | | | | | | | TATGTG |
| 228. | C0040C08-3 | Cxcr4 | chemokine (C- | C0040C08 | Mm.1401 | Chromosome 1 | GTAGGACTGTA |
| | | | X-C motif) | | | | GAACTGTAGA |
| | | | receptor 4 | | | | GGAAGAAACT |
| | | | | | | | GAACATTCCAG |
| | | | | | | | AATGTGTGGTA |
| | | | | | | | AATTGAA |
| 229. | K0612H02-3 | BM241159 | ESTs | K0612H02 | Mm.222325 | Chromosome 16 | TCATAGGTCTC |
| | | | BM241159 | | | | CATTTAGTTCA |
| | | | | | | | AGTGTTTTATG |
| | | | | | | | GACAATCAGC |
| | | | | | | | AAGTTTAGGCT |
| | | | | | | | CATAGG |
| 230. | J0460B09-3 | | AU024759 | J0460B09 | | No Chromosome | TTGGAATATAT |
| | | | Mouse | | | location | GAATGACAAA |
| | | | unfertilized egg | | | info available | GAAATGGGAA |
| | | | cDNA Mus | | | | AAACTGCTGAA |
| | | | musculus | | | | CCCGAGTCTCT |
| | | | cDNA clone | | | | GAATGTC |
| | | | J0460B09 3′, | | | | |
| | | | MRNA | | | | |
| | | | sequence | | | | |
| 231. | H3103F07-3 | | Mus musculus | H3103F07 | Mm.174026 | Chromosome 10 | CTATCTTGAAT |
| | | | transcribed | | | | TGCTAGATTAA |
| | | | sequence with | | | | AGAGAAAGAA |
| | | | weak similarity | | | | AATGTTAGAGC |
| | | | to protein | | | | AAAATAGGAA |
| | | | ref:NP_081764.1 | | | | CCTGGCC |
| | | | (M. musculus) | | | | |
| | | | RIKEN cDNA | | | | |
| | | | 5730493B19 | | | | |
| | | | [Mus musculus] | | | | |
| 232. | H3079H09-3 | BG069769 | ESTs | H3079H09 | Mm.173446 | Chromosome 9 | AATCCCTAGAG |
| | | | BG069769 | | | | AAAATGGGAA |
| | | | | | | | TAGAAATAAG |
| | | | | | | | CTGCATACAAA |
| | | | | | | | CTCAAAGACAC |
| | | | | | | | AGATACT |
| 233. | H3130D06-3 | BG074061 | ESTs | H3130D06 | Mm.182873 | Chromosome 1 | AGACTGAAGA |
| | | | BG074061 | | | | AAACCTTAAAA |
| | | | | | | | TACCCAAAATT |
| | | | | | | | CAGGGGAGAC |
| | | | | | | | ATAGCAACTGA |
| | | | | | | | GTCTCAT |
| 234. | H3071D08-3 | Lcp2 | lymphocyte | H3071D08 | Mm.1781 | Chromosome 11 | AGAGGACTTCC |
| | | | cytosolic | | | | TGTCTGTATCA |
| | | | protein 2 | | | | GATATTATTGA |
| | | | | | | | CTACTTCAGGA |
| | | | | | | | AAATGACGCTG |
| | | | | | | | TTGCT |
| 235. | K0218E07-3 | | Mus musculus | K0218E07 | Mm.216167 | Chromosome 10 | ATGGAGATGTG |
| | | | 10 days neonate | | | | TAAACAGTAG |
| | | | olfactory brain | | | | GACATTTCGAT |
| | | | cDNA, RIKEN | | | | AACTATGTCAG |
| | | | full-length | | | | GTCAGTTCTTA |
| | | | enriched | | | | GTTCAG |
| | | | library, | | | | |
| | | | clone:E530016 | | | | |
| | | | P10 | | | | |
| | | | product:weakly | | | | |
| | | | similar to | | | | |
| | | | ONCOGENE | | | | |
| | | | TLM [Mus | | | | |
| | | | musculus], full | | | | |
| | | | insert sequence. | | | | |
| 236. | C0907H07-3 | BM218221 | ESTs | C0907H07 | Mm.221604 | Chromosome 12 | GAGGCTATTAT |
| | | | BM218221 | | | | AAATAACCTGA |
| | | | | | | | AATGCATATGA |
| | | | | | | | GAACTGAACGT |
| | | | | | | | GTAATAATTCA |
| | | | | | | | GCTCC |
| 237. | K0605B09-3 | BM240642 | ESTs | K0605B09 | Mm.222320 | Chromosome X | AAGTCGGAAT |
| | | | BM240642 | | | | ATGTCTTAGTG |
| | | | | | | | TTCTTCTCACT |
| | | | | | | | TAGCTCAGTGT |
| | | | | | | | AAGATGGTAG |
| | | | | | | | CTCAAGT |
| 238. | C0322F05-3 | Eya3 | eyes absent 3 | C0322F05 | Mm.1430 | Chromosome 4 | CACTTTTCTAT |
| | | | homolog | | | | GAAGAAAGCC |
| | | | (Drosophila) | | | | GTGTGTAAAGT |
| | | | | | | | TTCCGTGACAG |
| | | | | | | | TAGTAATGGAA |
| | | | | | | | ATATCT |
| 239. | J0004A01-3 | C76123 | ESTs C76123 | J0004A01 | Mm.24905 | Chromosome 15 | TGTAAGAATAC |
| | | | | | | | AAGGTAAAAC |
| | | | | | | | AAAATAGAGA |
| | | | | | | | AATACAGGCAT |
| | | | | | | | CATATCTGCAA |
| | | | | | | | ATCGCCG |
| 240. | K0139H06-3 | BM223668 | ESTs | K0139H06 | Mm.221718 | Chromosome 3 | CAGAAACAGT |
| | | | BM223668 | | | | AGTATGGGGTT |
| | | | | | | | AAATCACAATG |
| | | | | | | | AGGGAAATTAT |
| | | | | | | | AGGGATATGC |
| | | | | | | | AGCCAAG |
| 241. | L0941F06-3 | BM120591 | ESTs | L0941F06 | Mm.217090 | Chromosome 9 | ACTGAAAGTTG |
| | | | BM120591 | | | | GGGAGATACA |
| | | | | | | | TGTAATTTAAT |
| | | | | | | | AGGATAGGGT |
| | | | | | | | ACTTAGGTCCA |
| | | | | | | | GACAACC |
| 242. | C0300G03-3 | 3021401C12Rik | RIKEN cDNA | C0300G03 | Mm.102470 | Chromosome 15 | AAGCTGTTGAA |
| | | | 3021401C12 | | | | TATGGACGTAA |
| | | | gene | | | | CTGTAAATCCC |
| | | | | | | | AGAGTGTTTTA |
| | | | | | | | TtTTGAGATGA |
| | | | | | | | GAGTT |
| 243. | C0925E03-3 | | transcribed | C0925E03 | Mm.217865 | Chromosome 6 | TTTATCAAACA |
| | | | sequence with | | | | TGGAAACATCT |
| | | | moderate | | | | AGAGACTATG |
| | | | similarity to | | | | GGAGAGAAAA |
| | | | protein | | | | TGGGTTTTTAG |
| | | | pir:S12207 | | | | ATATGGG |
| | | | (M. musculus) | | | | |
| | | | S12207 | | | | |
| | | | hypothetical | | | | |
| | | | protein (B2 | | | | |
| | | | element) - | | | | |
| | | | mouse | | | | |
| 244. | H3083B07-5 | BG082983 | ESTs | H3083B07 | Mm.203206 | No Chromosome | GGAAGTTAATA |
| | | | BG082983 | | | location | GAACTGTTCAA |
| | | | | | | info available | AATGTGAAAGT |
| | | | | | | | GGAAATAGCG |
| | | | | | | | TCAATAAGGA |
| | | | | | | | AAGCCCC |
| 245. | H3056F01-3 | Gdf9 | growth | H3056F01 | Mm.9714 | Chromosome 11 | AGTGTAGTTTT |
| | | | differentiation | | | | CAGTGGACAG |
| | | | factor 9 | | | | ATTTGTTAGCA |
| | | | | | | | TAAGTCTCGAG |
| | | | | | | | TAGAATGTAGC |
| | | | | | | | TGTGAA |
| 246. | J0259A06-3 | C88243 | EST C88243 | J0259A06 | Mm.249965 | No Chromosome | GAAAGTGGGG |
| | | | | | | location | AATGAAAAGT |
| | | | | | | info available | ATAACAAAGT |
| | | | | | | | AAAAAGAGAA |
| | | | | | | | TTTCTAGGCCC |
| | | | | | | | TTTAGGCCC |
| 247. | C0124B09-3 | BC0425 13 | cDNA sequence | C0124B09 | Mm.11186 | Chromosome 11 | GGTTTTCTCTT |
| | | | BC0425 13 | | | | GTTTTATCATG |
| | | | | | | | ATTCTTTTTAT |
| | | | | | | | GAAGCAATAA |
| | | | | | | | ATCCATTTCCC |
| | | | | | | | TGTTGG |
| 248. | L0933E02-3 | | L0933E02-3 | L0933E02 | | No Chromosome | CTTTTTGAGGT |
| | | | NIA Mouse | | | location | TTATTTTTCCA |
| | | | Newbom | | | info available | CAGTTTTCATT |
| | | | Kidney cDNA | | | | TGTTCATTAGG |
| | | | Library (Long) | | | | CATTTTCCCTT |
| | | | Mus musculus | | | | TTACT |
| | | | cDNA clone | | | | |
| | | | L0933E02 3′, | | | | |
| | | | MRNA | | | | |
| | | | sequence | | | | |
| 249. | H3072B12-3 | BG069052 | ESTs | H3072B12 | Mm.250102 | Chromosome 9 | AGTGTTTTTCT |
| | | | BG069052 | | | | TTAATTCTTGA |
| | | | | | | | GGTTGTTATTG |
| | | | | | | | TAATATTTACA |
| | | | | | | | TATAGTGCAAG |
| | | | | | | | AATGT |
| 250. | L0266C03-3 | D930020B18Rik | RIKEN cDNA | L0266C03 | Mm.138048 | Chromosome 10 | TAAAGTATCCA |
| | | | D930020B18 | | | | CTGAAGTCACT |
| | | | gene | | | | ATGGAAAACA |
| | | | | | | | GCCTTTTGATT |
| | | | | | | | TATGGACTATT |
| | | | | | | | TAGCTC |
| 251. | K0423B04-3 | Zfp91 | zinc finger | K0423B04 | Mm.212863 | Chromosome 19 | GCCTAGTTTTT |
| | | | protein 91 | | | | TCAGCATCAAT |
| | | | | | | | TTTGGAAAACC |
| | | | | | | | TTAGACCACAG |
| | | | | | | | GCATATTTCGT |
| | | | | | | | CAAGT |
| 252. | J0403C04-3 | | AUO21859 | J0403C04 | | No Chromosome | TCATTTTTCAA |
| | | | Mouse | | | location | GTCGTCAAGGG |
| | | | unfertilized egg | | | info available | GATGTTTCTCA |
| | | | cDNA Mus | | | | TTTTCCGTGAC |
| | | | musculus | | | | GACTTGAAAA |
| | | | cDNA clone | | | | ATGACG |
| | | | J0403C04 3′, | | | | |
| | | | MRNA | | | | |
| | | | sequence | | | | |
| 253. | J0248E12-3 | 1700011103Rik | RIKEN cDNA | J0248E12 | Mm.78729 | No Chromosome | CTGAAAATCAC |
| | | | 1700011103 | | | location | GGAAAATGAG |
| | | | gene | | | info available | AAATACACACT |
| | | | | | | | TTAGGACGTGA |
| | | | | | | | AATATGTCGAG |
| | | | | | | | GAAAAC |
| 254. | J0908H04-3 | Rpl24 | ribosomal | J0908H04 | Mm.107869 | No Chromosome | GCGAGAAAAC |
| | | | protein L24 | | | location | TGAAAATCACG |
| | | | | | | info available | GAAAATGAGA |
| | | | | | | | AATACACACTT |
| | | | | | | | TAGGACGTGA |
| | | | | | | | AATATGGC |
| 255. | K0205H10-3 | Madd | MAP-kinase | K0205H10 | Mm.36410 | Chromosome 2 | AGAAAGCTAT |
| | | | activating death | | | | GGACTGGATA |
| | | | domain | | | | GGAGGAGAAT |
| | | | | | | | GTAAATATTTC |
| | | | | | | | AGCTCCACATT |
| | | | | | | | ATTTATAG |
| 256. | C0507E09-3 | Gpr22 | G protein- | C0507E09 | Mm.68486 | Chromosome 12 | ACAAAAAGGT |
| | | | coupled | | | | TACCTATGAAG |
| | | | receptor 22 | | | | ACAGTGAAAT |
| | | | | | | | AAGAGAGAAA |
| | | | | | | | TGTTTAGTACC |
| | | | | | | | TCAGGTTG |
| 257. | J0005B1 1-3 | | Mus musculus | J0005B11 | Mm.249862 | Chromosome 7 | CTAAGGGAGG |
| | | | transcribed | | | | AAATGTTGGTA |
| | | | sequence with | | | | TAAAATGTTTA |
| | | | weak similarity | | | | AAAGAACTTG |
| | | | to protein | | | | GAGGCAAACTT |
| | | | ref:NP_083358.1 | | | | GGAGTGG |
| | | | (M. musculus) | | | | |
| | | | RIKEN cDNA | | | | |
| | | | 5830411J07 | | | | |
| | | | [Mus musculus] | | | | |
| 258. | L0201E08-3 | AW551705 | ESTs | L0201E08 | Mm.182670 | Chromosome 6 | CCACATCATTG |
| | | | AW551705 | | | | GAAAGAAATA |
| | | | | | | | CACTTATCTTA |
| | | | | | | | ATTGCCATGGA |
| | | | | | | | ATAGGAGCAT |
| | | | | | | | GAAAGTC |
| 259. | J0426H03-3 | AU023164 | ESTs | J0426H03 | Mm.221086 | Chromosome 4 | ATGAGAAATA |
| | | | AU023164 | | | | CACACTTTAGG |
| | | | | | | | ACGTGAAATAT |
| | | | | | | | GGCGAGGAAA |
| | | | | | | | ACTGAAAAAG |
| | | | | | | | GTCTATTC |
| 260. | C0649D06-3 | Cdkn2b | cyclin- | C0649D06 | Mm.269426 | Chromosome 4 | CCTGTGAACTG |
| | | | dependent | | | | AAAATGCAGA |
| | | | kinase inhibitor | | | | TGATCCACAGG |
| | | | 2B (p15, | | | | CTAAATGGGA |
| | | | inhibits CDK4) | | | | AACCTGGAGA |
| | | | | | | | GTAGATGA |
| 261. | J0421D03-3 | Rpl24 | ribosomal | J0421D03 | Mm.107869 | No Chromosome | GCGAGAAAAC |
| | | | protein L24 | | | location | TGAAAATCACG |
| | | | | | | info available | GAAAATGAGA |
| | | | | | | | AATACACACTT |
| | | | | | | | TAGGACCAGA |
| | | | | | | | AATATGGC |
| 262. | K0643F07-3 | | ESTs | K0643F07 | Mm.25571 | Chromosome X | TGGAGGAAATT |
| | | | BQ563001 | | | | GATTGAAAAA |
| | | | | | | | CGATTGGTCAA |
| | | | | | | | ATCGAAAATG |
| | | | | | | | GAGAAAACTC |
| | | | | | | | ATGTTCAC |
| 263. | H3103C12-3 | Slamfl | signaling | H3103C12 | Mm.103648 | Chromosome 1 | CTTCATCCTGG |
| | | | lymphocytic | | | | TTTTCACGGCA |
| | | | activation | | | | ATAATAATGAT |
| | | | molecule | | | | GAAAAGACAA |
| | | | family member | | | | GGTAAATCAA |
| | | | 1 | | | | ATCACTG |
| 264. | J0416H11-3 | Pscdbp | pleckstrin | J0416H11 | Mm.123225 | No Chromosome | ACTGAAAATCA |
| | | | homology, Sec7 | | | location | TGGAAAATGA |
| | | | and coiled-coil | | | info available | GAAACATCCAC |
| | | | domains, | | | | TTGACGACTTG |
| | | | binding protein | | | | AAAAATGACG |
| | | | | | | | AAATCAC |
| 265. | AF015770.1 | Rfng | radical fringe | AF015770 | Mm.871 | Chromosome 11 | CAAGCACTGTG |
| | | | gene homolog | | | | CTGCAAAATGT |
| | | | (Drosophila) | | | | CGGTGGAATAT |
| | | | | | | | GATAAGTTCCT |
| | | | | | | | AGAATCTGGAC |
| | | | | | | | GAAAA |
| 266. | C0933C05-3 | | ESTs | C0933C05 | Mm.217877 | Chromosome 1 | TTTGAGAAGAA |
| | | | BQ551952 | | | | AGGCATACACT |
| | | | | | | | TGAAATAAAG |
| | | | | | | | GCAAAAACATT |
| | | | | | | | ATACTGTCTAC |
| | | | | | | | CGAGAC |
| 267. | C0931A05-3 | E130304F04Rik | RIKEN cDNA | C0931A05 | Mm.38058 | Chromosome 13 | GAAGAAAACG |
| | | | E130304F04 | | | | AGGTGAAGAG |
| | | | gene | | | | CACTTTAGAAC |
| | | | | | | | ACTTGGGGATT |
| | | | | | | | ACAGACGAAC |
| | | | | | | | ATATCCGG |
| 268. | J0030C02-3 | C77383 | ESTs C77383 | J0030C02 | Mm.43952 | Chromosome 13 | ATCATAAAAAC |
| | | | | | | | TGTGGAAATCC |
| | | | | | | | ATATTGCCCTT |
| | | | | | | | TTAAAAGAAA |
| | | | | | | | ACTATGGGGAT |
| | | | | | | | GGAGAG |
| 269. | H3061A07-3 | Srpk2 | serine/arginine- | H3061A07 | Mm.8709 | Chromosome 5 | AAATGGCAGA |
| | | | rich protein | | | | AGAAAGGGTT |
| | | | specific kinase | | | | AATGGCTGGA |
| | | | 2 | | | | AAAATGGATC |
| | | | | | | | AGTAGTCTTGC |
| | | | | | | | AGAGGAACC |
| 270. | J0823B08-3 | | AUO41035 | J0823B08 | | Chromosome 10 | ATTUAGGGGG |
| | | | Mouse four- | | | | CTTTATTGUA |
| | | | cell-embryo | | | | CTTGACGTGGA |
| | | | cDNA Mus | | | | ATTTGAAAACT |
| | | | musculus | | | | AAAAAGATGA |
| | | | cDNA clone | | | | GTCTGG |
| | | | J0823B08 3′, | | | | |
| | | | MRNA | | | | |
| | | | sequence | | | | |
| 271. | L0942H08-3 | | Mus musculus | L0942H08 | Mm.276728 | Chromosome 11 | GTGGAAATCA |
| | | | transcribed | | | | GAGATCTAAGT |
| | | | sequence with | | | | ACGTTTATGCA |
| | | | moderate | | | | TAGGAGTAGG |
| | | | similarity to | | | | AATGAGGGGTT |
| | | | protein | | | | ATTAAAG |
| | | | ref:NP_081764.1 | | | | |
| | | | (M. musculus) | | | | |
| | | | RIKEN cDNA | | | | |
| | | | 5730493B19 | | | | |
| | | | [Mus musculus] | | | | |
| 272. | C0280H06-3 | Mrp150 | mitochondrial | C0280H06 | Mm.30052 | Chromosome 4 | AAACCCCCCAA |
| | | | ribosomal | | | | GTAGCCCAAA |
| | | | protein L50 | | | | GGCCCGCTTCC |
| | | | | | | | CACCAAAATGT |
| | | | | | | | TTTTTATGTTTT |
| | | | | | | | AAGGA |
| 273. | L0534E07-3 | 4632417D23 | hypothetical | L0534E07 | Mm.105080 | Chromosome 16 | ATTATGATGCC |
| | | | protein | | | | TGTAACACACA |
| | | | 4632417D23 | | | | GAAGTATCTGA |
| | | | | | | | CTGTGAACGAA |
| | | | | | | | TCAACCTCATG |
| | | | | | | | GATGA |
| 274. | U22339.1 | Il15ra | interleukin 15 | U22339 | 16169 | Chromosome 2 | AGAAGAGATA |
| | | | receptor, alpha | | | | CTGAGCCAATG |
| | | | chain | | | | AACCCTTTCGT |
| | | | | | | | GACAAAACCA |
| | | | | | | | AACTCAG |
| 275. | L0533C12-3 | | L0533C12-3 | L0533C12 | | No Chromosome | CTGCCTTCCCA |
| | | | NIA Mouse | | | location | TAAAAATAAA |
| | | | Newborn Heart | | | | AGGCATGCAA |
| | | | cDNA Library | | | | AACCAATTTTT |
| | | | Mus musculus | | | | GGCCAGGCCC |
| | | | cDNA clone | | | | AGTTAAGA |
| | | | L0533C12 3′, | | | | |
| | | | MRNA | | | | |
| | | | sequence | | | | |
| 276. | C0909E04-3 | Mvk | mevalonate | C0909E04 | Mm.28088 | Chromosome 5 | ACAAGCCCTGG |
| | | | kinase | | | | GCCTCTGAGAC |
| | | | | | | | CACCCGACACA |
| | | | | | | | CCATCCTACCA |
| | | | | | | | AGAAGCCTCTA |
| | | | | | | | AGTAT |
| 277. | J0093B09-3 | Bhmt2 | betaine- | J0093B09 | Mm.29981 | Chromosome 13 | CAAGTCAGCA |
| | | | homocysteine | | | | AGAAGCCAAC |
| | | | methyltransferase | | | | CTTGGTGAAAT |
| | | | 2 | | | | AATTCTGGTTG |
| | | | | | | | TTTGAAAGCTA |
| | | | | | | | GGTCTTG |
| 278. | H3066D09-3 | BG068517 | ESTs | H3066D09 | Mm.250067 | Chromosome 1 | GGTCAAGAGA |
| | | | BG068517 | | | | GTGCCAACTAG |
| | | | | | | | CTTTGTTTAAA |
| | | | | | | | AAATCCTAGTC |
| | | | | | | | CTGAATCCACA |
| | | | | | | | AGCCTG |
| 279. | C0346F01-3 | BM197260 | ESTs | C0346F01 | Mm.222100 | Chromosome 9 | AGTGGAAGCCT |
| | | | BM197260 | | | | TATAAGCATTG |
| | | | | | | | AACCCAGGAT |
| | | | | | | | GAGTCGCTCGT |
| | | | | | | | ATTTCCACCTT |
| | | | | | | | ACTCAT |
| 280. | K0125A06-3 | Hdac7a | histone | K0125A06 | Mm.259829 | Chromosome 15 | CTTCCCACAAC |
| | | | deacetylase 7A | | | | CCCACCGTACC |
| | | | | | | | TTGTCTATGTA |
| | | | | | | | TGCATGTTTTT |
| | | | | | | | GTAAAAAAGA |
| | | | | | | | AAAAAG |
| 281. | J0214H07-3 | | C85807 Mouse | J0214H07 | | No Chromosome | TGCCTGACTCC |
| | | | fertilized one- | | | location | AAGAAAAGAA |
| | | | cell-embryo | | | info available | GCCAGAACTCG |
| | | | cDNA Mus | | | | GAACCATAGTC |
| | | | musculus | | | | ATCTTTAAAGA |
| | | | cDNA clone | | | | TCTTCT |
| | | | J0214H07 3′, | | | | |
| | | | MRNA | | | | |
| | | | sequence | | | | |
| 282. | C0309H10-3 | 5930412E23Rik | RIKEN cDNA | C0309H10 | Mm.45194 | No Chromosome | GTTAATATTAT |
| | | | 5930412E23 | | | location | TAACTGAGCCT |
| | | | gene | | | info available | GCCCATACCCC |
| | | | | | | | CCGTGGTCATT |
| | | | | | | | GGTGTTGGGTG |
| | | | | | | | CAGTG |
| 283. | C0351C04-3 | 2610034E13Rik | RIKEN cDNA | C0351C04 | Mm.157778 | Chromosome 7 | GGAGGACGAC |
| | | | 2610034E13 | | | | ATCCTCATGGA |
| | | | gene | | | | CCTCATCTGAA |
| | | | | | | | CCCAACACCCA |
| | | | | | | | ATAAAGTTCCT |
| | | | | | | | TTTAAC |
| 284. | K0204G07-3 | Arf3 | ADP- | K0204G07 | Mm.295706 | Chromosome 15 not | TCTGAACCTCA |
| | | | ribosylation | | | placed | ACCCATCACCA |
| | | | factor 3 | | | | ACCCCGTGTCT |
| | | | | | | | TCAACATTACT |
| | | | | | | | TCCAAAAAAG |
| | | | | | | | TCTGG |
| 285. | L0928B09-3 | | transcribed | L0928B09 | Mm.217064 | Chromosome 10 | AGGAGCCTGTG |
| | | | sequence with | | | | TCCTTATAGAG |
| | | | strong | | | | TTGGAATTAAC |
| | | | similarity to | | | | TTCAGCCCTCT |
| | | | protein | | | | ATCTCACTTCC |
| | | | pir:S12207 | | | | TCTGT |
| | | | (M. musculus) | | | | |
| | | | S12207 | | | | |
| | | | hypothetical | | | | |
| | | | protein (B2 | | | | |
| | | | element) - | | | | |
| | | | mouse | | | | |
| 286. | H3059A09-3 | C430004E15Rik | RIKEN cDNA | H3059A09 | Mm.29587 | Chromosome 2 | GAAAAAAGAT |
| | | | C430004E 15 | | | | GAGATCTCCTC |
| | | | gene | | | | CATGACAAGA |
| | | | | | | | GCCTGCATACA |
| | | | | | | | ACATTTGAGTA |
| | | | | | | | CCCTTCT |
| 287. | C0949D03-3 | | UNKNOWN | C0949D03 | Data not found | No Chromosome | TTTGATTTTAG |
| | | | C0949D03 | | | location | CAGAAACCAC |
| | | | | | | info available | CACCAAAATTG |
| | | | | | | | TGCCTTAGCTG |
| | | | | | | | TATTTCTGTTT |
| | | | | | | | AGGGGA |
| 288. | K0118A04-3 | Rgs1 | regulator of G- | K0118A04 | Mm.103701 | Chromosome 1 | AGATACTATGG |
| | | | protein | | | | TACTGTCATGA |
| | | | signaling 1 | | | | AATGCAGTGG |
| | | | | | | | GACTCTATTCA |
| | | | | | | | AACAACCCTCC |
| | | | | | | | AAAATG |
| 289. | H3123F11-3 | | transcribed | H3123F11 | Mm.157781 | Chromosome 7 | AGAGAACCCA |
| | | | sequence with | | | | CACTCCTTTCA |
| | | | moderate | | | | TCAAGACTTGC |
| | | | similarity to | | | | AGAGCATCCCA |
| | | | protein | | | | CAACCAAGAT |
| | | | ref:NP_081764.1 | | | | GCTATTT |
| | | | (M. musculus) | | | | |
| | | | RIKEN cDNA | | | | |
| | | | 5730493B19 | | | | |
| | | | [Mus musculus] | | | | |
| 290. | H3154A06-3 | Gng13 | guanine | H3154A06 | Mm.218764 | Chromosome 17 | TATGAGCCTGA |
| | | | nucleotide | | | | CCCACACTCTC |
| | | | binding protein | | | | TGTAAGGTGTG |
| | | | 13, gamma | | | | ACTTTATAAAT |
| | | | | | | | AGACTTCTCCG |
| | | | | | | | GGTGT |
| 291. | L0534E01-3 | | L0534E01-3 | L0534E01 | | Chromosome 9 | ATACCCCACCA |
| | | | NIA Mouse | | | | CAACCTCTCAA |
| | | | Newbom Heart | | | | AAGAGGGCTCT |
| | | | cDNA Library | | | | TAACTTGGAAG |
| | | | Mus musculus | | | | GATAAAATAA |
| | | | cDNA clone | | | | ATCAGG |
| | | | L0534E01 3′, | | | | |
| | | | MRNA | | | | |
| | | | sequence | | | | |
| 292. | L0250B10-3 | Ap4m1 | adaptor-related | L0250B10 | Mm.1994 | No Chromosome | TATCCTCCCAC |
| | | | protein | | | location | AAAGATGAGA |
| | | | complex AP-4, | | | info available | GGAGCCCATCC |
| | | | mu 1 | | | | AGTGTTACTGT |
| | | | | | | | TAGAAGTCACA |
| | | | | | | | GTGAAA |
| 293. | L0518G04-3 | BM12304S | ESTs | L0518004 | Mm.221745 | Chromosome 3 | TATTGTCCAAT |
| | | | BM123045 | | | | GAAACCCACA |
| | | | | | | | AACTACCCTCT |
| | | | | | | | ATCTGGAGTTG |
| | | | | | | | GAACATTTATC |
| | | | | | | | TGCATT |
| 294. | J1020E03-3 | | transcribed | J1020E03 | Mm.250157 | Chromosome 9 | TAAGGAGACT |
| | | | sequence with | | | | GCCCTACAAAA |
| | | | moderate | | | | CTACGATACTA |
| | | | similarity to | | | | CTATCACTTTA |
| | | | protein | | | | AAAATTAGTGT |
| | | | pir:S12207 | | | | AAAGGG |
| | | | (M. musculus) | | | | |
| | | | S12207 | | | | |
| | | | hypothetical | | | | |
| | | | protein (B2 | | | | |
| | | | element). | | | | |
| | | | mouse | | | | |
| 295. | X12616.1 | Fes | feline sarcoma | X12616 | Mm.48757 | Chromosome 7 | TCAAGGCCAA |
| | | | oncogene | | | | GTTTCTGCAAG |
| | | | | | | | AAGCAAGGAT |
| | | | | | | | CCTGAAACAGT |
| | | | | | | | ACAACCACCCC |
| | | | | | | | AACATTG |
| 296. | J0026H02-3 | C77164 | expressed | J0026H02 | 97587 | Chromosome X | GATTGCCAGAG |
| | | | sequence | | | | ACTTACACTTA |
| | | | C77164 | | | | ATAGAGTCATA |
| | | | | | | | AAGCCCATAG |
| | | | | | | | AGCCTGAGTGA |
| | | | | | | | GAGCCA |
| 297. | H3154D11-5 | Taf71 | TAF7-like | H3154D11 | Mm.103259 | Chromosome X | TTATTCCTGAA |
| | | | RNA | | | | GCCCCCGCTAC |
| | | | polymerase II, | | | | AGATGTTTCCA |
| | | | TATA box | | | | CAACCGAAGA |
| | | | binding protein | | | | AGCGGTCTCCA |
| | | | (TBP)- | | | | AAGAGC |
| | | | associated | | | | |
| | | | factor | | | | |
| 298. | H3054H04-3 | Kcnn4 | potassium | H3054H04 | Mm.9911 | Chromosome 7 | AGCTCCACATG |
| | | | intermediate/sm | | | | AACTCACAGA |
| | | | all conductance | | | | AGAACCAGGC |
| | | | calcium- | | | | TAAGTACCCAA |
| | | | activated | | | | GGACCGAGCTC |
| | | | channel, | | | | AAGGACA |
| | | | subfamily N, | | | | |
| | | | member 4 | | | | |
| 299. | J0425B03-3 | R75183 | expressed | J0425B03 | Mm.276293 | Chromosome 15 | ACCATTATTCT |
| | | | sequence | | | | TTTAAAAAACC |
| | | | R75183 | | | | CAAAAACCAC |
| | | | | | | | CAGCAAGGGG |
| | | | | | | | GCCTTTGGTTG |
| | | | | | | | GCCTCAA |
| 300. | C0930C02-3 | 0610037D15Rik | RIKEN cDNA | C0930C02 | Mm.218714 | No Chromosome | CTTCATCTTAA |
| | | | 0610037D15 | | | location | AACTCCAGAAC |
| | | | gene | | | info available | AACTCCCTTCC |
| | | | | | | | TAACCTGGAAC |
| | | | | | | | CCAGCAGCTTT |
| | | | | | | | CAGTT |
| 301. | L0812A11-3 | | ESTs B1793430 | L0812A11 | Mm.261348 | No Chromosome | CTGCACGCCCC |
| | | | | | | location | AGGAGCCTGG |
| | | | | | | info available | GTGAAGCATCA |
| | | | | | | | CAGCACTAAGT |
| | | | | | | | CATGTTAAAAG |
| | | | | | | | GAGTCT |
| 302. | J0243F04-3 | 9530020D24Rik | RIKEN cDNA | J0243F04 | Mm.200585 | Chromosome 2 | CACTGGAGCAC |
| | | | 9530020D24 | | | | TGAACATGATG |
| | | | gene | | | | TACAAGTATCA |
| | | | | | | | CACAGAAAAG |
| | | | | | | | CAGCACTGGAC |
| | | | | | | | TGTACT |
| 303. | C0335A03-311 | 10035014Rik | RIKEN cDNA | C0335A03 | Mm.202727 | Chromosome 12 | ATAAGAACTTA |
| | | | 1130035014 | | | | TAGGAACCCCA |
| | | | gene | | | | ACTCCCCATGA |
| | | | | | | | AAAATATAAG |
| | | | | | | | ACCTCAAGGCC |
| | | | | | | | TGGGGA |
| 304 | H3003B10-3 | BG063111 | ESTs | H3003B10 | Mm.100527 | Chromosome 3 | GCCCACCAACT |
| | | | BG063111 | | | | CTAATTTGTGC |
| | | | | | | | TACTTATATAT |
| | | | | | | | ATTCCTGGGAG |
| | | | | | | | TAGGACTGTCC |
| | | | | | | | TCCTG |
| 305 | U97073.1 | Prtn3 | proteinase 3 | U97073 | Mm.2364 | Chromosome 10 | CAGTCAGGTCT |
| | | | | | | | TCCAGAACAAT |
| | | | | | | | TACAACCCCGA |
| | | | | | | | GGAGAACCTC |
| | | | | | | | AATGACGTGCT |
| | | | | | | | TCTCCT |
| 306. | K0300D08-3 | Afmid | arylformamidasc | K0300D08 | Mm.169672 | Chromosome 11 | CGTAGCTCGCT |
| | | | | | | | GGTAGAAAGC |
| | | | | | | | CTGACCACCAT |
| | | | | | | | GCATACGATCC |
| | | | | | | | TGGGTTTCAAC |
| | | | | | | | AAGGAA |
| 307. | H3029H06-3 | Sf3b2 | splicing factor | H3029H06 | Mm.196532 | Chromosome 19 | GAGCCTGAGAT |
| | | | 3b, subunit 2 | | | | CTACGAGCCCA |
| | | | | | | | ATTTCATCTTC |
| | | | | | | | TTCAAGAGGAT |
| | | | | | | | TTTTGAGGCTT |
| | | | | | | | TCAAG |
| 308. | H3074D09-3 | Drg2 | developmentally | H3074D09 | Mm.41803 | Chromosome 11 | GAGTCTGTGGG |
| | | | regulated | | | | TAUCGCCTGA |
| | | | GIP binding | | | | ACAAGCATAA |
| | | | protein 2 | | | | GCCCAACATCT |
| | | | | | | | ATTTCAAGCCC |
| | | | | | | | AAGAAA |
| 309. | K0647G12-3 | Plek | pleckstrin | K0647G12 | Mm.98232 | Chromosome 11 | AGCATCAAAC |
| | | | | | | | AAAGCACATA |
| | | | | | | | AACTCGTACAT |
| | | | | | | | AAGCAAGGGA |
| | | | | | | | TGTCCTTATTG |
| | | | | | | | GTCAAACA |
| 310. | H3137A08-3 | | Mus musculus | H3137A08 | Mm.197271 | Chromosome 2 | GGGAAAAAAT |
| | | | transcribed | | | | AGCAAAACCC |
| | | | sequence with | | | | CAAACTCCACA |
| | | | moderate | | | | ACCACAAAAA |
| | | | similarity to | | | | CCTGTTAATTA |
| | | | protein | | | | TGGTGGCA |
| | | | pir:S12207 | | | | |
| | | | (M. musculus) | | | | |
| | | | S12207 | | | | |
| | | | hypothetical | | | | |
| | | | protein (B2 | | | | |
| | | | element) - | | | | |
| | | | mouse | | | | |
| 311. | C0166D06-3 | Slc38a3 | solute carrier | C0166D06 | Mm.30058 | Chromosome 9 | ACACAGAGCC |
| | | | family 38, | | | | AGAAAACCCA |
| | | | member 3 | | | | GGCCTGAAGA |
| | | | | | | | CATCCCCTAGT |
| | | | | | | | CCTGCTGAGAG |
| | | | | | | | ACCACAGT |
| 312. | K0406B07-3 | Sirt7 | sirtuin 7 (silent | K0406B07 | Mm.259849 | Chromosome 11 | CGACCAATCTG |
| | | | mating type | | | | CCTGGGAAAC |
| | | | information | | | | AACACCCCACA |
| | | | regulation 2, | | | | GAACGGGGCTT |
| | | | homolog) 7 (S. | | | | CAGAAACACG |
| | | | cerevisiae) | | | | TGAGTGA |
| 313. | H3085D10-3 | Gda | guanine | H3085D10 | Mm.45054 | Chromosome 19 | GTTTAGGTGAG |
| | | | deaminase | | | | TTTTCCATTGTA |
| | | | | | | | TCTTATAACAG |
| | | | | | | | AGAAACCCATT |
| | | | | | | | AGGCAGTAGTT |
| | | | | | | | AGTTC |
| 314. | H3099C09-3 | Igf1 | insulin-like | H3099C09 | Mm.268521 | Chromosome 10 | TCGAAACACCT |
| | | | growth factor 1 | | | | ACCAAATACCA |
| | | | | | | | ATAATAAGTCC |
| | | | | | | | AATAACATTAC |
| | | | | | | | AAAGATGGGC |
| | | | | | | | ATTTCC |
| 315. | H3099B07-5 | 2610028H24Rik | RIKEN cDNA | H3099B07 | 76964 | No Chromosome | TGCTACCCTCC |
| | | | 2610028H24 | | | location | AGGACCAACG |
| | | | gene | | | info available | ATGGATGCACC |
| | | | | | | | ACGGAGTCCCA |
| | | | | | | | AGAGCTGAAA |
| | | | | | | | AGCAGAA |
| 316. | H3114H10-3 | Rec8L1 | REC8-like 1 | H3114H10 | Mm.23149 | Chromosome 14 | CGGAGCTCTTC |
| | | | (yeast) | | | | AGAACCCCAA |
| | | | | | | | CTCTCTCTGGC |
| | | | | | | | TGGCTACCCCC |
| | | | | | | | AGAACTCCTAG |
| | | | | | | | GTTTAT |
| 317. | L0703E03-3 | Lipc | lipase, hepatic | L0703E03 | Mm.362 | Chromosome 9 | ATAAAGAGAA |
| | | | | | | | TTCCCACCACC |
| | | | | | | | CTGOGCGAAG |
| | | | | | | | GAATTACCAGC |
| | | | | | | | AATAAAACCTA |
| | | | | | | | TTCCTTC |
| 318. | H3074H08-3 | BG069302 | ESTs | H3074H08 | Mm.11484 | Chromosome 7:not | ACTTTCAAGTC |
| | | | BG069302 | | | placed | TGAATCCTATG |
| | | | | | | | AGCCTGAAGTG |
| | | | | | | | AGATCTTATTT |
| | | | | | | | AGAAACAGAA |
| | | | | | | | CCCCAA |
| 319. | K0443D01-3 | Bazlb | bromodomain | K0443D01 | Mm.40331 | Chromosome 5 | GACAAGCCCTT |
| | | | adjacent to zinc | | | | AGGGAGCCAG |
| | | | finger domain, | | | | AAAAAGAGCA |
| | | | 1B | | | | GGAAGAAGTT |
| | | | | | | | AAAATGTTTAA |
| | | | | | | | TTTTTTAA |
| 320. | J0409E10-3 | AU022163 | ESTs | J0409E10 | Mm.188475 | Chromosome 16 | GCCCAAGAGCT |
| | | | AU022163 | | | | AGAAAACCTA |
| | | | | | | | CTCTATGTGTA |
| | | | | | | | GAGATACTTCC |
| | | | | | | | TATTAAAATAA |
| | | | | | | | TAGTAC |
| 321. | L0528E01-3 | BM123655 | EST | L0528E01 | Mm.216782 | Chromosome 9 | CTCCACTTTTA |
| | | | BM123655 | | | | AAGTCTGTAGG |
| | | | | | | | AATAGGAGCC |
| | | | | | | | GATTAGACAAC |
| | | | | | | | TCTCGGTCTCA |
| | | | | | | | TGCTCA |
| 322. | L0031B11-3 | Alcam | activated | L0031B11 | Mm.2877 | Chromosome 16 | TTTCTGGGATC |
| | | | leukocyte cell | | | | CCACTGCACCG |
| | | | adhesion | | | | CCATTTCTTCC |
| | | | molecule | | | | CAGATTTATGT |
| | | | | | | | GTATAACTTAA |
| | | | | | | | ACTGG |
| 323. | G0115A06-3 | Femla | feminization 1 | G0115A06 | Mm.27723 | Chromosome 17 | ATACAGTAGAT |
| | | | homolog a (C. | | | | GCTGAACACAC |
| | | | elegans) | | | | TTGAGTCCATC |
| | | | | | | | ATGAGGGGGT |
| | | | | | | | AATAAGTCTCA |
| | | | | | | | CCAGCA |
| 324. | L0947C07-3 | Mal | myelin and | L0947C07 | Mm.39040 | Chromosome 2 | TCTTATACTTT |
| | | | lymphocyte | | | | CAACAAAGCT |
| | | | protein, T-cell | | | | GAACCCTAACA |
| | | | differentiation | | | | TTACACTAACC |
| | | | protein | | | | AGCAGCTCAAC |
| | | | | | | | ACGAGT |
| 325. | H3101A05-3 | AU040576 | expressed | H3101A05 | Mm.26700 | Chromosome 7 | CTGAATGTATA |
| | | | sequence | | | | CACACCCACAG |
| | | | AU040576 | | | | GAGACTGTGGC |
| | | | | | | | TGAGCGTTCAT |
| | | | | | | | CCAAATAAATT |
| | | | | | | | TGAAT |
| 326. | H3064E10-3 | BG068353 | ESTs | H3064E10 | Mm.35046 | Chromosome 4 | GTTCCTGTTCA |
| | | | BG068353 | | | | GAGTGCCTGAA |
| | | | | | | | AACCCAAAGT |
| | | | | | | | GTCTGAGAGTC |
| | | | | | | | TGAAGGAATTC |
| | | | | | | | AACTGT |
| 327. | K0505H05-3 | Ian6 | immune | K0505H05 | Mm.24781 | Chromosome 6 | AAACACCCAC |
| | | | associated | | | | ACTTGAAACTT |
| | | | nucleotide 6 | | | | CCATGAACCCA |
| | | | | | | | CTCAAATTCAT |
| | | | | | | | TTCTATCCCCC |
| | | | | | | | TTTGGA |
| 328. | H3082E12-3 | Ptpre | protein tyrosine | H3082E12 | Mm.945 | Chromosome 7 | TCATGGAGATA |
| | | | phosphatase, | | | | TAACTATAGAG |
| | | | receptor type, E | | | | ATAAAGAGCG |
| | | | | | | | ACACCCTGTCT |
| | | | | | | | GAAGCAATCA |
| | | | | | | | GCGTCCG |
| 329. | H3088A06-3 | 2310047N01Rik | RIKEN cDNA | H3088A06 | Mm.31482 | Chromosome 4 | GGACACTGTGA |
| | | | 2310047N01 | | | | ACACTGTGTGG |
| | | | gene | | | | ACAGAGCCCA |
| | | | | | | | CAACTTCTCCA |
| | | | | | | | TTTGTGTCTGG |
| | | | | | | | CAGCAA |
| 330. | K0635B07-3 | Ccr5 | chemokine (C- | K0635B07 | Mm.14302 | Chromosome 9 | AGGAAAGAAA |
| | | | C motif) | | | | GGGGTTAGAAT |
| | | | receptor 5 | | | | CTCTCAGGAGA |
| | | | | | | | TTAAAGTTTTCT |
| | | | | | | | GCCTAACAAG |
| | | | | | | | AGGTGTT |
| 331. | C0153A12-3 | 1110025F24Rik | RIKEN cDNA | C0153A12 | Mm.28451 | Chromosome 16 | CTCAAGACTTT |
| | | | 1110025F24 | | | | GCCAACATGTT |
| | | | gene | | | | CCGTTTCTTAC |
| | | | | | | | ACCCTGAACCC |
| | | | | | | | TGATCGGAACA |
| | | | | | | | TTCAT |
| 332. | C0143E02-3 | BC022145 | cDNA sequence | C0143E02 | Mm.200891 | Chromosome 11 | TCTGTACATGG |
| | | | BC022145 | | | | CCGAAAATCA |
| | | | | | | | GAGTCCACCAT |
| | | | | | | | ATTCTTTTGAA |
| | | | | | | | TATCCAGGGTT |
| | | | | | | | CTCTGA |
| 333. | L0863F12-3 | Nr2c2 | nuclear receptor | L0863F12 | Mm.193835 | Chromosome 6 | TTCTGGCTCCT |
| | | | subfamily 2, | | | | TATTTCAGTTC |
| | | | group C, | | | | TCTTTAAAACC |
| | | | member 2 | | | | AGTTCAACACC |
| | | | | | | | AGTGTGTTAAA |
| | | | | | | | AAGAA |
| 334. | H3045F02-3 | LOC214424 | hypothetical | H3045F02 | Mm.31129 | Chromosome 9 | GCAGATTTAAC |
| | | | protein | | | | AACTAGCAACT |
| | | | LOC214424 | | | | CTGTCATCTTT |
| | | | | | | | TTCTAAAAATG |
| | | | | | | | ACCAACTGCTG |
| | | | | | | | ATTAC |
| 335. | H3035005-3 | BG065832 | ESTs | H3035G05 | Mm.154695 | Chromosome 17 | CTTAAAAAGG |
| | | | BG065832 | | | | GAGATACAGTT |
| | | | | | | | TTACTCTGATC |
| | | | | | | | CAGCAAATCTA |
| | | | | | | | GTTAAGACACT |
| | | | | | | | AGAATG |
| 336. | H3137D02-3 | Hnrpl | heterogeneous | H3137D02 | Mm.9043 | Chromosome 7 | CTTCCTGAACC |
| | | | nuclear | | | | ATTACCAGATG |
| | | | ribonucleoprote | | | | GAAAACCCAA |
| | | | in L | | | | ATGGCCCGTAC |
| | | | | | | | CCATATACTCT |
| | | | | | | | GAAGTT |
| 337 | H3097F07-3 | AU040829 | expressed | H3097F07 | Mm.134338 | Chromosome 11 | GTAACGGAGC |
| | | | sequence | | | | CTGGGGGTTGA |
| | | | AU040829 | | | | AGGTTATCTTT |
| | | | | | | | ACATATATGTA |
| | | | | | | | CAAACTGTTGT |
| | | | | | | | CAAGAG |
| 338. | J0029C02-3 | Frag 1-pending | FGF receptor | J0029C02 | Mm.259795 | Chromosome 7 | TCCCCACCACT |
| | | | activating | | | | CATGGGGATCT |
| | | | protein 1 | | | | TCAAGAAGCAT |
| | | | | | | | CACCATTCACT |
| | | | | | | | GAAAGGTCCTA |
| | | | | | | | AAAAA |
| 339. | BB416014.1 | | Mus musculus | BB416014 | Mm.24449 | Chromosome 10 | GCGCAGAGGC |
| | | | B6-derived | | | | AAACCAACGT |
| | | | CDII + ve | | | | GGAGCCAGAC |
| | | | dendritic cells | | | | ATTGGTGAACC |
| | | | cDNA, RIKEN | | | | CAACCTATCCA |
| | | | full-length | | | | CACCTTCA |
| | | | enriched | | | | |
| | | | library, | | | | |
| | | | clone:F730035 | | | | |
| | | | A01 | | | | |
| | | | product:similar | | | | |
| | | | to SWI/SNF | | | | |
| | | | COMPLEX | | | | |
| | | | 170 KDA | | | | |
| | | | SUBUNIT | | | | |
| | | | [Homo | | | | |
| | | | sapiens], full | | | | |
| | | | insert sequence. | | | | |
| 340. | H3087E01-3 | Anxa4 | annexin A4 | H3087E01 | Mm.259702 | Chromosome 6 | CTTATTTTAGA |
| | | | | | | | CAGATCCAAA |
| | | | | | | | GTTCTCACAAG |
| | | | | | | | CCCCCTTTCTT |
| | | | | | | | TGCTCTGCCTA |
| | | | | | | | TCATCG |
| 341. | H3088E08-3 | BG070548 | ESTs | H3088E08 | Mm.11161 | Chromosome 8 | AACCTCTGAAC |
| | | | BG070548 | | | | CTAATCACTGT |
| | | | | | | | GGATTCCCACC |
| | | | | | | | AACACCATATA |
| | | | | | | | TGAAAATGCA |
| | | | | | | | GGCCGA |
| 342. | AF179424.1 | | Mus musculus | AF179424 | Mm.1428 | Chromosome 14 | TGCGGAAGGA |
| | | | 13 days embryo | | | | GGGGATTCAA |
| | | | male testis | | | | ACCAGAAAAC |
| | | | eDNA, RIKEN | | | | GGAAGCCCAA |
| | | | full-length | | | | GAACCTGAATA |
| | | | enriched | | | | AATCTAAGA |
| | | | library, | | | | |
| | | | clone:6030408 | | | | |
| | | | M17 | | | | |
| | | | product:GATA | | | | |
| | | | binding protein | | | | |
| | | | 4, full insert | | | | |
| | | | sequence | | | | |
| 343. | J0258C01-3 | | Mus musculus | J0258C01 | Mm.275718 | Chromosome 2 | CCCTAGTCCGT |
| | | | mRNA for | | | | TTTCTGATCAG |
| | | | mKIAA1335 | | | | TCAGAACCCAC |
| | | | protein | | | | AATAACTACTA |
| | | | | | | | GTAGTCCTGTG |
| | | | | | | | GCTTT |
| 344. | K0507B09-3 | | ESTs | K0507B09 | Mm.218038 | Chromosome 9 | GTAGCCACCAA |
| | | | BM238095 | | | | GCCACAAGTA |
| | | | | | | | ACAAATGATCT |
| | | | | | | | CTGTGAATGCC |
| | | | | | | | ATATGGAAACT |
| | | | | | | | TTTATT |
| 345. | L0846F07-3 | BM117131 | ESTs | L0846F07 | Mm.216977 | Chromosome 9 | GGCTCCATTTC |
| | | | BM117131 | | | | TGAACTCTGTG |
| | | | | | | | TTAAGCTAATA |
| | | | | | | | AGATTTTAAAT |
| | | | | | | | AAACGCTGATG |
| | | | | | | | AAAGC |
| 346. | U48866.1 | CEBPE | CCAAT/enhancer | U48866 | Hs.158323 | No Chromosome | TGCTGGGGGCC |
| | | | binding | | | location | TAGAACCCTGA |
| | | | protein | | | info available | GACATAGACC |
| | | | (C/EBP), | | | | ATGGATAAATG |
| | | | epsilon | | | | GCAACCGGGG |
| | | | | | | | TGGCAAA |
| 347. | K0301B06-3 | Fech | ferrochelatase | K0301B06 | Mm.217130 | Chromosome 18 | AACGCAAAGA |
| | | | | | | | GCAAGAACCA |
| | | | | | | | AACAAAGACA |
| | | | | | | | GGAACAACTC |
| | | | | | | | GCAGAAGAAA |
| | | | | | | | TCCCGCCTGG |
| 348. | NM_009756.1 | Bmp10 | bone | NM_009756 | Mm.57171 | Chromosome 6 | TGTTTTCTGAT |
| | | | morphogenetic | | | | GACCAAAGCA |
| | | | protein 10 | | | | ATGACAAGGA |
| | | | | | | | GCAGAAAGAA |
| | | | | | | | GAACTGAACG |
| | | | | | | | AATTGATCA |
| 349. | NM_010100.1 | Edar | ectodysplasin-A | NM_010100 | Mm.174523 | Chromosome 10 | CCCACCACTGA |
| | | | receptor | | | | ATATAGACCAT |
| | | | | | | | ACTGTGAGAG |
| | | | | | | | GACCATAATTA |
| | | | | | | | GGTCCTGAATT |
| | | | | | | | TTTAAT |
| 350. | G0115E06-3 | C430014D17Rik | RIKEN cDNA | G0115E06 | Mm.103389 | Chromosome 3 | GTATGACTTCC |
| | | | C430014D17 | | | | AACCAGAAAA |
| | | | gene | | | | AGGCTCTAAAA |
| | | | | | | | GCTGAACACAC |
| | | | | | | | TAACCGGCTGA |
| | | | | | | | AAAACG |
| 351. | L0266D11-3 | Ppp3ca | protein | L0266D11 | Mm.80565 | Chromosome 3 | CTTCTGGCTCC |
| | | | phosphatase 3, | | | | CTTACATGAAG |
| | | | catalytic | | | | GACTGATTTAA |
| | | | subunit, alpha | | | | GAAACCAGAC |
| | | | isoform | | | | CATTCCTTTAC |
| | | | | | | | TTTGAA |
| 352. | L0526F10-3 | | Mus musculus | L0526F10 | Mm.215689 | Chromosome X | GCAGGGTGCTT |
| | | | 10 days neonate | | | | ACTTTCTCAGA |
| | | | cortex cDNA, | | | | GCCTGAAGTTA |
| | | | RIKEN full- | | | | CTTCCATTGTT |
| | | | length enriched | | | | TTGGCACTGAA |
| | | | library, | | | | TAACA |
| | | | clone:A830020 | | | | |
| | | | C2 I | | | | |
| | | | product:unknown | | | | |
| | | | EST, full | | | | |
| | | | insert sequence. | | | | |
| 353 | H3047C10-3 | Slc6a6 | solute carrier | H3047C10 | Mm.200518 | Chromosome 6 | TTAGCACAAGA |
| | | | family 6 | | | | GAAAAGCTGA |
| | | | (neurotransmitter | | | | GAACGTGGGTT |
| | | | transporter, | | | | TTGCCTCCTTC |
| | | | taurine), | | | | AGAAATATGTC |
| | | | member 6 | | | | TGGCTC |
| 354 | K0322G06-3 | BC042620 | cDNA sequence | K0322G06 | Mm.152289 | Chromosome 17 | ACACAGCACCC |
| | | | BC042620 | | | | ACAACTAATCT |
| | | | | | | | TGGGACACCCC |
| | | | | | | | TATCTGGTTGG |
| | | | | | | | AAGAGAGTAA |
| | | | | | | | ACTAAT |
| 355. | NM_009580.1 | Zp1 | zona pellucida | NM_009580 | Mm.24767 | Chromosome 19 | CAATGGCCTAT |
| | | | glycoprotein 1 | | | | TCTGTCAGATG |
| | | | | | | | GGTGTCCTTTC |
| | | | | | | | AAGGGTGACA |
| | | | | | | | ACTACAGAAC |
| | | | | | | | ACAAGTA |
| 356. | H3150E08-3 | Map4k5 | mitogen- | H3150E08 | Mm.260244 | Chromosome 12 | AAAGTAGGTTC |
| | | | activated | | | | ACACAGTAAA |
| | | | protein kinase | | | | GGGATAATACC |
| | | | kinase kinase | | | | ATCTGGAACAA |
| | | | kinase 5 | | | | TGATCAGTGTA |
| | | | | | | | GAGTTA |
| 357. | J0059G03-3 | C79059 | ESTs C79059 | J0059G03 | Mm.249888 | Chromosome 4 | CACCTGGGTCT |
| | | | | | | | ACAGCTACTCT |
| | | | | | | | GATTCTACAAA |
| | | | | | | | GACAGGGTCA |
| | | | | | | | AGCATCTCTAA |
| | | | | | | | CAAAGT |
| 358. | U93191.1 | Hdac2 | histone | U93191 | 15182 | Chromosome 10 | TATTAAACCCA |
| | | | deacetylase 2 | | | | GGAGATACAA |
| | | | | | | | GGAGTCTGCCA |
| | | | | | | | TTAACCTCTCT |
| | | | | | | | GTAACTCAAGA |
| | | | | | | | GTAGTT |
| 359. | H3033C04-5 | | H3033C04-5 | H3033C04 | | No Chromosome | TTCCTCCCAAA |
| | | | NIA Mouse | | | location | ATGGAGTTTCC |
| | | | 15K cDNA | | | info available | TCTTCAAACCA |
| | | | Clone Set Mus | | | | CAGCTCCCCCA |
| | | | musculus | | | | AGATCTATCCT |
| | | | cDNA clone | | | | GATAT |
| | | | H3033C04 5′, | | | | |
| | | | MRNA | | | | |
| | | | sequence | | | | |
| 360. | H3085C01-3 | 2700038N03Rik | RIKEN cDNA | H3085C01 | Mm.21836 | Chromosome 5 | TATGTCTTGAT |
| | | | 2700038N03 | | | | ACTGGACCCAC |
| | | | gene | | | | ACTACTGGGGC |
| | | | | | | | ACTCCAAAAA |
| | | | | | | | ACCGTTGTGAA |
| | | | | | | | CTACAA |
| 361. | J0412G02-3 | BB336629 | ESTs | J0412G02 | Mm.208743 | Chromosome 11 | AGTAAAGGGC |
| | | | BB336629 | | | | ACCGGAAATGT |
| | | | | | | | TAAATCCTTGT |
| | | | | | | | TTAGGATATGA |
| | | | | | | | AAGGAATTAG |
| | | | | | | | GGGATGG |
| 362. | K0527H09-3 | BM239048 | ESTs | K0527H09 | Mm.217288 | Chromosome 11 | GAATGTCTGAT |
| | | | BM239048 | | | | ACATGACCCAT |
| | | | | | | | CAGTTAGGAAC |
| | | | | | | | CACTGAACTAG |
| | | | | | | | AGGAGTAGCT |
| | | | | | | | AAACTC |
| 363. | H3009C10-3 | Serpinb9b | serine (or | H3009C10 | Mm.45371 | Chromosome 13 | GCTTCTACTGG |
| | | | cysteine) | | | | CTCTTGTATGC |
| | | | proteinase | | | | ATATGTGCACT |
| | | | inhibitor, dade | | | | TATCCAGACTG |
| | | | B, member 9b | | | | AGGATTTTACA |
| | | | | | | | AAGCA |
| 364. | H3142D11-3 | | Mus musculus | H3142D11 | Mm.113272 | Chromosome X | CTGTCTAAGCG |
| | | | mRNA similar | | | | CTGAACCACTT |
| | | | to hypothelical | | | | AGCAGAAATG |
| | | | protein | | | | ACACCCATATG |
| | | | FLJ2O811 | | | | AGAGCTTGTGC |
| | | | (cDNA clone | | | | CAAATA |
| | | | MGC:27863 | | | | |
| | | | IMAGE:34925 | | | | |
| | | | 16), complete | | | | |
| | | | cds | | | | |
| 365. | H3094B07-3 | | Mus musculus | H3094B07 | Mm.173357 | Chromosome 14 | AAAGGAGACT |
| | | | transcribed | | | | GCATCAGGTAT |
| | | | sequence with | | | | TCTGATAGAGA |
| | | | weak similarity | | | | GCTGAGGAAG |
| | | | to protein | | | | AGATTGAGGTA |
| | | | sp:P11369 | | | | TGGGATT |
| | | | (M. musculus) | | | | |
| | | | POL2_MOUSE | | | | |
| | | | Retrovirus | | | | |
| | | | related POL | | | | |
| | | | polyprotein | | | | |
| | | | [Contains: | | | | |
| | | | Reverse | | | | |
| | | | transcriptase; | | | | |
| | | | Endonuclease] | | | | |
| 366. | J0068F09-3 | C79588 | ESTs C79588 | J0068F09 | Mm.234023 | No Chromosome | TGACTGGAATC |
| | | | | | | location | ACCACCCTTGC |
| | | | | | | info available | CTGAGTTTGCG |
| | | | | | | | ATCTCACAGTT |
| | | | | | | | GGAACTGAGA |
| | | | | | | | GTTTCC |
| 367. | H3039B03-5 | EO30024M05Rik | RIKEN cDNA | H3039B03 | Mm.5675 | Chromosome 12 | GGATCAGATG |
| | | | E030024M05 | | | | ATGCACCATUG |
| | | | gene | | | | CTTTCCATTTGC |
| | | | | | | | TACATTTAAAA |
| | | | | | | | TCTTTTACTAG |
| | | | | | | | TCAACC |
| 368. | H3068B03-3 | BG068673 | ESTs | H3068B03 | Mm.11978 | Chromosome 1 | TTGAGACCTTA |
| | | | BG068673 | | | | AAGAAATAAC |
| | | | | | | | AAACTCAAGG |
| | | | | | | | AAGATTAGGGT |
| | | | | | | | CCAGTGTTTAA |
| | | | | | | | GTCATGG |
| 369. | C0250F05-3 | BM203195 | ESTs | C0250F05 | Mm.228379 | Chromosome 12 | GTCTCCTTTGT |
| | | | BM203195 | | | | GTTATTGCCTT |
| | | | | | | | CCCAACACTTC |
| | | | | | | | TAAGTCCCAGC |
| | | | | | | | TCAACAGCTAC |
| | | | | | | | TTCTA |
| 370. | H3110C11-3 | Mlph | melanophilin | H3110C11 | Mm.17675 | Chromosome 1 | CACAGCTGCTT |
| | | | | | | | GTAGTCATCAT |
| | | | | | | | TCCAGTGAGGA |
| | | | | | | | GTAAGAAGAA |
| | | | | | | | TTTTATGTGTG |
| | | | | | | | TCTCTA |
| 371. | H3121F01-3 | Wnt4 | wingless- | H3121F01 | Mm.20355 | Chromosome 4 | AACTTAAACAG |
| | | | related MMTV | | | | TCTCCCACCAC |
| | | | integration site | | | | CTACCCCAAAA |
| | | | 4 | | | | GATACTGGTTG |
| | | | | | | | TATTTTTTGTTT |
| | | | | | | | TGGT |
| 372. | J1012G09-3 | Brd3 | bromodomain | J1012G09 | Mm.28721 | Chromosome 2 | CAGCAGAAAA |
| | | | containing 3 | | | | GGCTCCCACCA |
| | | | | | | | AGAAGGCCAA |
| | | | | | | | CAGCACAACC |
| | | | | | | | ACAGCCAGCA |
| | | | | | | | GGATGTGTT |
| 373 | L0952B09-3 | Usp49 | ubiquitin | L0952B09 | Mm.25072 | Chromosome 17 | GGCTTCACATC |
| | | | specific | | | | TAAGTGGGGA |
| | | | protease 49 | | | | CTATTTTAACT |
| | | | | | | | TATTTACAGGT |
| | | | | | | | ATATGGTGTGG |
| | | | | | | | AAATAA |
| 374. | K0131B12-3 | I14ra | interleukin 4 | K0131B12 | Mm.233802 | Chromosome 7 | CGCTCAGTTGT |
| | | | receptor, alpha | | | | AGAAAGCAAC |
| | | | | | | | AAGGACACAA |
| | | | | | | | ACTTGATTGCC |
| | | | | | | | CAAAGTCACTG |
| | | | | | | | CCAGTTA |
| 375. | H3046E09-3 | Nfatc2ip | nuclear factor | H3046E09 | Mm.1389 | Chromosome 7 | GTCTGAACACA |
| | | | of activated T- | | | | CTATTATGTAT |
| | | | cells, | | | | CCATCCAATCT |
| | | | cytoplasmic 2 | | | | CAACTGAATAA |
| | | | interacting | | | | AGGGAGATGC |
| | | | protein | | | | CTTTTG |
| 376. | K0520805-3 | | transcribed | K0520B05 | Mm.221547 | Chromosome 14 | AAAGAATTTCA |
| | | | sequence with | | | | AGAACGAAGC |
| | | | weak similarity | | | | ATAGGTGGTTA |
| | | | to protein | | | | TGTAGTTTGAT |
| | | | pir:158401 | | | | TACAGAAAAG |
| | | | (M. musculus) | | | | AGATGCC |
| | | | 158401 protein | | | | |
| | | | tyrosine kinase | | | | |
| | | | (EC2.7.1.112) | | | | |
| | | | JAK3 - mouse | | | | |
| 377. | K0315G05-3 | Stat5a | signal | K0315G05 | Mm.4697 | Chromosome 11 | AAACCACCTTC |
| | | | transducer and | | | | AGTGTGAGGA |
| | | | activator of | | | | GCCCACGTCAG |
| | | | transcription | | | | TTGTAGTATCT |
| | | | 5A | | | | CTGTTCATACC |
| | | | | | | | AACAAT |
| 378. | H3086F07-3 | BC003332 | cDNA sequence | H3086F07 | Mm.100116 | Chromosome 6 | GCACTCCAGCC |
| | | | BC003332 | | | | TGATTCTTTGA |
| | | | | | | | GACTTTGGGGT |
| | | | | | | | ACACATATTGA |
| | | | | | | | AAGTACTTTGA |
| | | | | | | | ATTTG |
| 379. | H3156A10-5 | Ctsd | cathepsin D | H3156A10 | Mm.231395 | Chromosome 7 | ACTGTATCGGT |
| | | | | | | | TCCATGTAAGT |
| | | | | | | | CTGACCAGTCA |
| | | | | | | | AAGGCAAGAG |
| | | | | | | | GTATCAAGGTG |
| | | | | | | | GAGAAA |
| 380. | C0890D02-3 | | C0890D02-3 | C0890D02 | | Chromosome 18 | GTGTTTGAATT |
| | | | NIA Mouse | | | | AAAACCCCCAC |
| | | | Blastocyst | | | | CCTCGGAGGCC |
| | | | cDNA Library | | | | TTTAAAGAAAT |
| | | | (Long) Mus | | | | GGTTTTTGTCC |
| | | | musculus | | | | GTTGT |
| | | | cDNA clone | | | | |
| | | | C0890D02 3′, | | | | |
| | | | MRNA | | | | |
| | | | sequence | | | | |
| 381. | L0245G03-3 | 6430519N07Rik | RIKEN cDNA | L0245G03 | Mm.149642 | Chromosome 6 | CTCTCGACAAA |
| | | | 6430519N07 | | | | ATATAAATGGA |
| | | | gene | | | | CAGTACCAAAC |
| | | | | | | | TAAGAGGGAT |
| | | | | | | | ATAAGTGGGA |
| | | | | | | | GCAAAGG |
| 382. | J0447A10-3 | | Mus musculus | J0447A10 | Mm.202311 | Chromosome 11 | TATGGTACGAG |
| | | | cDNA clone | | | | TTTAGGGCTTA |
| | | | IMAGE:12820 | | | | GTCAGTTTACA |
| | | | 81, partial cds | | | | ATGGGGATTGA |
| | | | | | | | ATTTTGTGTCA |
| | | | | | | | AAACC |
| 383. | J1031A09-3 | | Mus musculus | J1031A09 | Mm.235234 | No Chromosome | CTGGCTCCTAC |
| | | | transcribed | | | location | TGGCAACAGG |
| | | | sequence with | | | info available | CATACTTGTGG |
| | | | weak similarity | | | | TTTAATACAGA |
| | | | to protein | | | | GAAACAAAAC |
| | | | pir:158401 | | | | ATTCATA |
| | | | (M. musculus) | | | | |
| | | | 158401 protein | | | | |
| | | | tyrosine kinase | | | | |
| | | | (EC2.7.1.112) | | | | |
| | | | JAK3 - mouse | | | | |
| 384. | L0072H04-3 | A630084M22Rik | RIKEN cDNA | L0072H04 | Mm.27968 | Chromosome 1 | TTTGACCTAAT |
| | | | A630084M22 | | | | GAAATACCCAT |
| | | | gene | | | | TTCATCTGTGA |
| | | | | | | | CAACACATAGC |
| | | | | | | | CCAGTAAACAT |
| | | | | | | | CACTG |
| 385. | J0050E03-3 | | transcribed | J0050E03 | Mm.37806 | Chromosome 14 | CCTGTTCCTAG |
| | | | sequence with | | | | TATCCTGOCGT |
| | | | weak similarity | | | | CCACATATACC |
| | | | to protein | | | | CAAAGTTAGGC |
| | | | ref:NP_081764.1 | | | | ATACTAACCAA |
| | | | (M. musculus) | | | | GAGAT |
| | | | RIKEN cDNA | | | | |
| | | | 5730493B19 | | | | |
| | | | [Mus musculus] | | | | |
| 386. | H3039C11-3 | Tyro3 | TYRO3 protein | H3039C11 | Mm.2901 | Chromosome 2 | CTGGAACTCAG |
| | | | tyrosine kinase | | | | CACTGCCCACC |
| | | | 3 | | | | ACACTTGGTCC |
| | | | | | | | GAAATGCCAG |
| | | | | | | | GTTTGCCCCTC |
| | | | | | | | TTAAGT |
| 387. | C0324F11-3 | 6720458F09Rik | RIKEN cDNA | C0324F11 | | Chromosome 12 | CCTGGAGGTCT |
| | | | 6720458F09 | | | | CCACCTGAAGT |
| | | | gene | | | | TCCCTGATGCA |
| | | | | | | | GGGTCAGTCCA |
| | | | | | | | GCCTTGGTAAG |
| | | | | | | | GGCCA |
| 388. | L0018F11-3 | AW547199 | ESTs | L0018F11 | Mm.182611 | Chromosome 12 | AAATGAGAAC |
| | | | AW547 199 | | | | CAGATTACCAA |
| | | | | | | | AATTACCACTA |
| | | | | | | | CCACCAAAATA |
| | | | | | | | ACCCCTCTGAT |
| | | | | | | | TCCTTG |
| 389. | X69902.1 | Itga6 | integrin alpha 6 | X69902 | Mm.225096 | Chromosome 2 | CAGATAGATG |
| | | | | | | | ACAGCAGGAA |
| | | | | | | | ATTTTCTTTATT |
| | | | | | | | TCCTGAAAGAA |
| | | | | | | | AATACCAGACT |
| | | | | | | | CTCAAC |
| 390. | H3105A09-3 | | transcribed | H3105A09 | Mm.174047 | No Chromosome | GGTGCCAAATG |
| | | | sequence with | | | location | CGGCCATGGTG |
| | | | weak similarity | | | info available | CTGAACAATTT |
| | | | to protein | | | | ATCGTCAGAGG |
| | | | ref:NP_416488.1 | | | | GGAAGAACAG |
| | | | (E. coli) | | | | TTGACC |
| | | | putative | | | | |
| | | | transport | | | | |
| | | | protein, | | | | |
| | | | shikimate | | | | |
| | | | [Escherichia | | | | |
| | | | coli K12]. | | | | |
| 391. | H3159F01-5 | | UNKNOWN | H3159F01 | Data not found | No Chromosome | CCAAAACAGA |
| | | | H3159F01 | | | location | GCCAACACCAC |
| | | | | | | info available | CGACAACAAC |
| | | | | | | | CCCACAGCAA |
| | | | | | | | ACCCGGAGAG |
| | | | | | | | AAACCCAAA |
| 392. | K0522B04-3 | F5 | coagulation | K0522B04 | Mm.12900 | Chromosome 1 | TTTCAACCCGC |
| | | | factor V | | | | CCATTATTTCC |
| | | | | | | | AGATTTATCCG |
| | | | | | | | CATCATTCCTA |
| | | | | | | | AAACATGGAA |
| | | | | | | | CCAGAG |
| 393 | C0123F08-3 | A1843918 | expressed | C0123F08 | Mm.143742 | Chromosome 5 | TGGAGACTGA |
| | | | sequence | | | | GTTCGACAATC |
| | | | A1843918 | | | | CCATCTACGAG |
| | | | | | | | ACTGGCGAAA |
| | | | | | | | CAAGAGAGTA |
| | | | | | | | TGAAGTTT |
| 394 | H3067G08-3 | BG068642 | ESTs | H3067008 | Mm.250079 | Chromosome 11 | GATACAACAG |
| | | | BG068642 | | | | CATCTGTTTTC |
| | | | | | | | CAAGGAGAAA |
| | | | | | | | TCATTTGAGGA |
| | | | | | | | ACAAAACCTAT |
| | | | | | | | CAAGAGA |
| 395. | K0349B03-3 | Stam2 | signal | K0349B03 | Mm.45048 | Chromosome 2 | AACTAGAAAA |
| | | | transducing | | | | CATAGATGCAC |
| | | | adaptor | | | | AGGACTCGGAT |
| | | | molecule (SH3 | | | | CCATGATATTT |
| | | | domain and | | | | ACACTGGGAA |
| | | | ITAM motif) 2 | | | | ATGTTCT |
| 396. | C0620D11-3 | Bid | BH3 interacting | C0620D11 | Mm.34384 | Chromosome 6 | ATCTCAAGATT |
| | | | domain death | | | | TCTATCCAAGT |
| | | | agonist | | | | GGAAACAAAC |
| | | | | | | | TGAATCATGCA |
| | | | | | | | CACGACTTATC |
| | | | | | | | TGTGTG |
| 397. | C0189H10-3 | 4930486L24Rik | RIKEN cDNA | C0189H10 | Mm.19839 | Chromosome 13 | AGAGGAGCCA |
| | | | 4930486L24 | | | | CACTTGATGTG |
| | | | gene | | | | AATTAAACTCA |
| | | | | | | | TAAACATTATG |
| | | | | | | | CCACTAACAGC |
| | | | | | | | TTTTAT |
| 398. | H3140A02-3 | Slc9a1 | solute carrier | H3140A02 | Mm.4312 | Chromosome 4 | CTGCCGCCTGT |
| | | | family 9 | | | | ACAAAGGAAA |
| | | | (sodium/hydrogen | | | | CTGAACCTTTT |
| | | | exchanger), | | | | TCATATTCTAA |
| | | | member 1 | | | | TAAATCAATGT |
| | | | | | | | GAGTTT |
| 399. | K0645B04-3 | Smc411 | SMC4 | K0645B04 | Mm.206841 | Chromosome 3 | AAGCTGAGATT |
| | | | structural | | | | AAACGGCTAC |
| | | | maintenance of | | | | ACAATACCATC |
| | | | chromosomes | | | | ATAGATATCAA |
| | | | 4-like 1 (yeast) | | | | CAACCGAAAA |
| | | | | | | | CTCAAGG |
| 400. | C0300008-3 | 6720460106Rik | RIKEN cDNA | C0300008 | Mm.28865 | Chromosome 4 | GACTTGGGAA |
| | | | 6720460106 | | | | AACAATGCAA |
| | | | gene | | | | CTCCCATAAAC |
| | | | | | | | CAAAACTCCAA |
| | | | | | | | TTCCATGCCTA |
| | | | | | | | ACTTGCT |
| 401. | M59378.1 | Tnfrsf1b | tumor necrosis | M59378 | Mm.2666 | Chromosome 4 | AGCAGGGAAC |
| | | | factor receptor | | | | AATTTGAGTGC |
| | | | superfamily, | | | | TGACCTATAAC |
| | | | member 1b | | | | ACATTCCTAAA |
| | | | | | | | GGATGGGCAG |
| | | | | | | | TCCAGAA |
| 402. | NM_009399.1 | Tnfrsfl 1a | tumor necrosis | NM_009399 | Mm.6251 | Chromosome 1 | AGCTCCAACTC |
| | | | factor receptor | | | | AACAGATGGCT |
| | | | superfamily, | | | | ACACAGGCAG |
| | | | member 11a | | | | TGGGAACACTC |
| | | | | | | | CTGGGGAGGA |
| | | | | | | | CCATGAA |
| 403. | C0168E12-3 | 2810442122Rik | RIKEN cDNA | C0168E12 | Mm.103450 | Chromosome 10 | ACTAGCTGCAT |
| | | | 2810442122 | | | | TGTAAAGAAA |
| | | | gene | | | | CAAATCGAAA |
| | | | | | | | CTGAGTCTTTT |
| | | | | | | | CACATATTGTG |
| | | | | | | | ACGGACA |
| 404. | L0228H10-3 | CLr | complement | L0228H10 | Mm.24276 | Chromosome 6 | GTAGGGTCATC |
| | | | component 1, r | | | | ATACACCCAGA |
| | | | subComponent | | | | CTACCGCCAAG |
| | | | | | | | ATGAACCTAAC |
| | | | | | | | AATTTTGAAGG |
| | | | | | | | AGACA |
| 405. | H3088B10-3 | BG070515 | ESTs | H3088B10 | Mm.11092 | Chromosome 11 | TCCCCACCACG |
| | | | BG070515 | | | | AATTATCGTGG |
| | | | | | | | CTAGTGGATGA |
| | | | | | | | AGGCCACTAAT |
| | | | | | | | ACAGGTTCAAA |
| | | | | | | | TTGTT |
| 406. | K0409D10-3 | Lrrc5 | leucine-rich | K0409D10 | Mm.23837 | Chromosome 5 | TATGTGCATAG |
| | | | repeat- | | | | GCTGGAGTTTT |
| | | | containing 5 | | | | GGTTATACATG |
| | | | | | | | GTACACTTTTG |
| | | | | | | | GGCCAATATAA |
| | | | | | | | TAGGA |
| 407. | H3056D02-3 | | transcribed | H3056D02 | Mm.9706 | Chromosome 12 | CCACACTCCCT |
| | | | sequence with | | | | GGAGACAATG |
| | | | moderate | | | | TCTGCCATTTT |
| | | | similarity to | | | | TGCATCACTTG |
| | | | protein | | | | TCAAACCACTA |
| | | | ref:NP_079108.1 | | | | ACTTCT |
| | | | (H. sapiens) | | | | |
| | | | hypothetical | | | | |
| | | | protein | | | | |
| | | | FLJ22439 | | | | |
| | | | [Homo sapiens] | | | | |
| 408. | J0430F08-3 | AU023357 | ESTs | J0430F08 | Mm.173615 | Chromosome 6 | TCGGTTGACCT |
| | | | AU023357 | | | | GATTCCACCAA |
| | | | | | | | GGAGAAGGAG |
| | | | | | | | ATCAAGGAAG |
| | | | | | | | AGTAAACTGTA |
| | | | | | | | AGAGCAT |
| 409. | H3158C06-3 | 2810457106Rik | RIKEN cDNA | H3158C06 | Mm.133615 | Chromosome 9 | GAGTGCTTTGA |
| | | | 2810457106 | | | | TGGTTGTTAGG |
| | | | gene | | | | GACCGTAAGA |
| | | | | | | | ATAGTCCTGTG |
| | | | | | | | TCAGACAGCA |
| | | | | | | | GATTCTA |
| 410. | M85078.1 | Csf2ra | colony | M85078 | Mm.255931 | Chromosome 19 | AACTGTCATAA |
| | | | stimulating | | | | AATCCAACGTG |
| | | | factor 2 | | | | CCTTCATGATC |
| | | | receptor, alpha, | | | | AAAGTTCGATA |
| | | | low-affinity | | | | GTCAGTAGTAC |
| | | | (granulocyte- | | | | TAGAA |
| | | | macrophage) | | | | |
| 411. | C0145E06-3 | Satb1 | special AT-rich | C0145E06 | Mm.289605 | Chromosome 5 | ACTCTCATCTG |
| | | | sequence | | | | TAAAGCCTTCC |
| | | | binding protein | | | | CATCTCATTAT |
| | | | 1 | | | | TCCTTGCACTA |
| | | | | | | | ACCACAGCCAC |
| | | | | | | | TAGGA |
| 412. | H3015B08-3 | BG064069 | ESTs | H3015B08 | Mm.197224 | Chromosome 11 | CAGACTGAAA |
| | | | BG064069 | | | | GGAAATTCCAA |
| | | | | | | | AGAAAACAAA |
| | | | | | | | AACCTTTCAAT |
| | | | | | | | CTATGAACTCA |
| | | | | | | | ATGGCTG |
| 413. | C0842H05-3 | Fbln1 | fibulin 1 | C0842H05 | Mm.219663 | Chromosome 15 | CTGAGAATAAC |
| | | | | | | | CTACTACCACC |
| | | | | | | | TCTCTTTTCCC |
| | | | | | | | ACCAACATCCA |
| | | | | | | | AGTGCCAGCG |
| | | | | | | | GTGGTT |
| 414. | G0117D07-3 | Otx2 | orthodenticle | G0117D07 | Mm.134516 | Chromosome 14 | AGCGACATGC |
| | | | homolog 2 | | | | AACCAAATACC |
| | | | (Drosophila) | | | | ACTCAAAACA |
| | | | | | | | AAAATCCAGC |
| | | | | | | | AAAACTGAGTT |
| | | | | | | | GTGAGGGA |
| 415. | L0806E03-3 | Stmn4 | stathmin-like | 4L0806E03 | Mm.35474 | Chromosome 14 | GTTTGTACATG |
| | | | | | | | TAAAAGATTGA |
| | | | | | | | CCAGTGAAGCC |
| | | | | | | | ATCCTATTTGT |
| | | | | | | | TTCTGGGGAAC |
| | | | | | | | AATGA |
| 416. | H3073B06-3 | BG069137 | ESTs | H3073B06 | Mm.173781 | Chromosome 3 | ACTTAGACCAC |
| | | | BG069137 | | | | AACAGCATCTA |
| | | | | | | | AGCATCATTAC |
| | | | | | | | CTTAAGTACTA |
| | | | | | | | AAGCAAAAAT |
| | | | | | | | CTAGTC |
| 417. | H3082G08-3 | Myo10 | myosin X | H3082G08 | Mm.60590 | Chromosome 15 | TAAACCACTCT |
| | | | | | | | TAAACTGCTGG |
| | | | | | | | CTCCAGTGTTT |
| | | | | | | | TTAGAATGATA |
| | | | | | | | TGAAGTCATTT |
| | | | | | | | TGGAG |
| 418. | C0141F07-3 | C3arl | complement | C0141F07 | Mm.2408 | Chromosome 6 | AGTAAGTGCCA |
| | | | component 3a | | | | TTATCCACCCA |
| | | | receptor 1 | | | | ACTACCAACCA |
| | | | | | | | ATGCCTAAGCA |
| | | | | | | | GATTCTATATC |
| | | | | | | | TTAGC |
| 419. | K0525G09-3 | 5830411120 | hypothetical | K0525G09 | Mm.31672 | Chromosome 5 | GCTTCTGGCAG |
| | | | protein | | | | AGATCTGTTTA |
| | | | 5830411120 | | | | GCATAGTGTGG |
| | | | | | | | TATTAATTATA |
| | | | | | | | GCAAATGTTAA |
| | | | | | | | GGTAG |
| 420. | H3064D01-3 | | transcribed | H3064D01 | Mm.250054 | Chromosome 15 | GTTGTCTGAAT |
| | | | sequence with | | | | AATAGCACCCA |
| | | | weak similarity | | | | AGAAAAAGTG |
| | | | to protein | | | | TGGAGATCAGT |
| | | | ref:NP_001362.1 | | | | AGGTATTCATT |
| | | | (H. sapiens) | | | | AAGCAT |
| | | | dynein, | | | | |
| | | | axonemal, | | | | |
| | | | heavy | | | | |
| | | | polypeptide 8 | | | | |
| | | | [Homo sapiens] | | | | |
| 421. | C0120F08-3 | 6330406L22Rik | RIKEN cDNA | C0120F08 | Mm.5202 | Chromosome 10 | TAAAGGAGCTT |
| | | | 6330406L22 | | | | TCCACATGAAC |
| | | | gene | | | | TCACAATTTTC |
| | | | | | | | TTGAAATAAAC |
| | | | | | | | TTCTTAACCAA |
| | | | | | | | CTGCC |
| 422. | H3105G04-3 | Map4k4 | mitogen- | H3105G04 | Mm.987 | Chromosome 1 | GTCACTTGGAT |
| | | | activated | | | | GGTGTATTTAT |
| | | | protein kinase | | | | GCACAAAAGG |
| | | | kinase kinase | | | | GCTCAGAGACT |
| | | | kinase 4 | | | | AAAGTTCCTGT |
| | | | | | | | GTGAAC |
| 423. | J0800D09-3 | 2310004L02Rik | RIKEN cDNA | J0800D09 | Mm.159956 | Chromosome 7 | GTCATGAACCC |
| | | | 2310004L02 | | | | AATACACTGTG |
| | | | gene | | | | GAAATGTGTGA |
| | | | | | | | TTCTTTATATT |
| | | | | | | | AAACGTCTGCT |
| | | | | | | | GTTCA |
| 424. | L0226H02-3 | 5830411120 | hypothetical | L0226H02 | Mm.31672 | Chromosome 5 | TGTCGATACCA |
| | | | protein | | | | TCTAAAGACCA |
| | | | 5830411120 | | | | CAACTTCTAGC |
| | | | | | | | CATAGGGTATT |
| | | | | | | | TCATATATGTC |
| | | | | | | | CATTT |
| 425. | L0529D10-3 | BM123730 | ESTs | L0529D10 | Mm.221754 | Chromosome 7 | ATGCAAACCTA |
| | | | BM123730 | | | | AAAAGCACCC |
| | | | | | | | AAAAAATTCAC |
| | | | | | | | ATTGGACTGAA |
| | | | | | | | GAAGAGTGAT |
| | | | | | | | CCAAGCA |
| 426. | H3088E05-3 | Gla | galactosidase, | H3088E05 | Mm.1114 | Chromosome X | TTTGAGACCCT |
| | | | alpha | | | | TTCATAAGCCC |
| | | | | | | | AATTATACAGA |
| | | | | | | | TATCCAATATT |
| | | | | | | | ACTGCAATCAT |
| | | | | | | | TGGAG |
| 427. | K0621H11-3 | | K0621H11-3 | K0621H11 | | Chromosome 13 | ACCTAAATTTC |
| | | | NIA Mouse | | | | CACAGGCAACT |
| | | | Hematopoictic | | | | TACTTTGTTAT |
| | | | Stem Cell (Lin- | | | | TAAATTTGGGG |
| | | | /c-Kit-/Sca-1+) | | | | ATCATATCCTG |
| | | | cDNA Library | | | | TGCCC |
| | | | (Long) Mus | | | | |
| | | | musculus | | | | |
| | | | cDNA clone | | | | |
| | | | NIA:K0621H11 | | | | |
| | | | IMAGE:30070 | | | | |
| | | | 846 3′, MRNA | | | | |
| | | | sequence | | | | |
| 428. | C0846H03-3 | D330025I23Rik | RIKEN cDNA | C0846H03 | Mm.260376 | Chromosome 9 | TTTTTTCAGAC |
| | | | D330025I23 | | | | TTAAGAACAGC |
| | | | gene | | | | TAAACAAAAC |
| | | | | | | | CTTCCTCTAGC |
| | | | | | | | TTTTTCATCAC |
| | | | | | | | ATCCAG |
| 429. | J0058E06-3 | C78984 | ESTs C78984 | J0058E06 | Mm.249886 | Chromosome 17 | ATAATGATGAT |
| | | | | | | | GATAACAACA |
| | | | | | | | AGAAAACAGA |
| | | | | | | | CTCGAACCTAA |
| | | | | | | | AGACGCTGGTC |
| | | | | | | | TCAGATA |
| 430. | K0325E09-3 | Ibsp | integrin binding | K0325E09 | Mm.4987 | Chromosome 5 | CGCAAACATAC |
| | | | sialoprotein | | | | CCTGTATAAGA |
| | | | | | | | AGGCTCCTAAC |
| | | | | | | | GAGAGATTTAT |
| | | | | | | | TAACAACACTA |
| | | | | | | | TATAT |
| 431. | K0336F07-3 | Pycs | pyrroline-5- | K0336F07 | Mm.233117 | Chromosome 19 | TTTGACTGGGA |
| | | | carboxylate | | | | CCAGCCCAGCC |
| | | | synthetase | | | | ATTCTCAGCCT |
| | | | (glutamate | | | | CTCGACATGTA |
| | | | gamma- | | | | ATTTCATTTCT |
| | | | semialdehyde | | | | TTTAC |
| | | | synthetase) | | | | |
| 432. | H3013B04-3 | B230106124Rik | RIKEN cDNA | H3013B04 | Mm.24576 | Chromosome 3 | AGGACTCATAG |
| | | | B230106124 | | | | ACTTACAGAAT |
| | | | gene | | | | GATGCCGAATG |
| | | | | | | | GAATGTTTTGT |
| | | | | | | | GCATGACCTTT |
| | | | | | | | TAACC |
| 433 | L0238A07-3 | Midn | midnolin | L0238A07 | Mm.143813 | No Chromosome | CCACCTCGCCC |
| | | | | | | location | AAGTCTCCTTT |
| | | | | | | info available | TACTGAAATAA |
| | | | | | | | AATTTGAGGGG |
| | | | | | | | AAGAGAAAAA |
| | | | | | | | ATTTAC |
| 434. | L0929C04-3 | Tnfrsfl lb | tumor necrosis | L0929C04 | Mm.15383 | Chromosome 15: not | GATGTTCTTCT |
| | | | factor receptor | | | placed | GTAAAAGTTAC |
| | | | superfamily, | | | | TAATATATCTG |
| | | | member 1 lb | | | | TAAGACTATTA |
| | | | (osteoprotegerin) | | | | CAGTATTGCTA |
| | | | | | | | TTTAT |
| 435. | L0020F05-3 | 6330583M11Rik | RIKEN cDNA | L0020F05 | Mm.23572 | Chromosome 2 | CTTAAGATTCA |
| | | | 6330583M11 | | | | GGAAAATGGTT |
| | | | gene | | | | CTTTCTGCCCT |
| | | | | | | | TCCTAGCGTTT |
| | | | | | | | ACAGAACAGA |
| | | | | | | | CTCCGA |
| 436. | H3012H07-3 | Cd44 | CD44 antigen | H3012H07 | Mm.24138 | Chromosome 2 | TATATTGACAT |
| | | | | | | | CCATAACACCA |
| | | | | | | | AAAACTGTCTT |
| | | | | | | | TTTAGCTAAAA |
| | | | | | | | TCGACCCAAGA |
| | | | | | | | CTGTC |
| 437. | K0240E11-3 | Myo5a | myosin Va | K0240E11 | Mm.3645 | Chromosome 9 | TCTTTAGTGCT |
| | | | | | | | GCATTTAAGTG |
| | | | | | | | GCATACAAAAT |
| | | | | | | | ACAATCCCATA |
| | | | | | | | TGTATGAACTG |
| | | | | | | | TTGTG |
| 438. | K0401C06-3 | Col8a1 | procollagen, | K0401C06 | Mm.86813 | Chromosome 16 | AATCTATGCCA |
| | | | type VIII, alpha | | | | GATACTGTATA |
| | | | 1 | | | | TTCTACCATGG |
| | | | | | | | TGCTAATATCA |
| | | | | | | | GAGCTAAATG |
| | | | | | | | ATACTC |
| 439. | C0917F02-3 | Frzb | frizzled-related | C0917F02 | Mm.136022 | Chromosome 2 | AATTTACACAT |
| | | | protein | | | | GTGGTAGTAGT |
| | | | | | | | AGGTCCAGATT |
| | | | | | | | CCTAAGTTACA |
| | | | | | | | GTGTGCTGAAA |
| | | | | | | | AATAA |
| 440. | H3104C03-3 | 1500015O10Rik | RIKEN cDNA | H3104C03 | Mm.11819 | Chromosome 1 | ATGAGGCTAA |
| | | | 1500015O10 | | | | ATTTGAAGATG |
| | | | gene | | | | ATGTCAACTAT |
| | | | | | | | TGGCTAAACAG |
| | | | | | | | AAATCGAAAC |
| | | | | | | | GGCCATG |
| 441. | K0438D09-3 | Col8al | procollagen, | K0438D09 | Mm.86813 | Chromosome 16 | TCTACTACTTT |
| | | | type VIII, alpha | | | | GCTTATCATGT |
| | | | 1 | | | | TCACTGCAAGG |
| | | | | | | | GAGGCAACGT |
| | | | | | | | ATGGGTTGCTC |
| | | | | | | | TCTTCA |
| 442. | H3152C04-3 | Usp16 | ubiquitin | H3152C04 | Mm.196253 | Chromosome 16 | GTACTGAACTC |
| | | | specific | | | | ACAAGCGTATC |
| | | | protease 16 | | | | TCCTATTTTAT |
| | | | | | | | GAGAGAATAC |
| | | | | | | | TGTGATAACAA |
| | | | | | | | AAAGTG |
| 443. | H3079D12-3 | Pld3 | phospholipase | H3079D12 | Mm.6483 | Chromosome 7 | TTGGCCCACCC |
| | | | D3 | | | | CCAAAGGGCC |
| | | | | | | | AAGATTATAAG |
| | | | | | | | TAAATAATTGT |
| | | | | | | | CTGTATAGCCT |
| | | | | | | | GTGCTT |
| 444. | L0020E08-3 | Clqg | complement | L0020E08 | Mm.3453 | Chromosome 4 | CTGGGAACCAC |
| | | | component 1, q | | | | CTAATGGTATT |
| | | | subcomponent, | | | | ATTCCTGTGGC |
| | | | gamma | | | | CATTTATCAAT |
| | | | polypeptide | | | | ACCTTATGAGA |
| | | | | | | | CTATT |
| 445. | J0025G01-3 | Yars | tyrosyl-tRNA | J0025G01 | Mm.22929 | Chromosome 4 | TCCTCTGGGGT |
| | | | synthetase | | | | AAATGAGCTTG |
| | | | | | | | ACCTTGTGCAA |
| | | | | | | | ATGGAGAGAC |
| | | | | | | | CAAAAGCCTCT |
| | | | | | | | GATTTT |
| 446. | L0832H09-3 | Mafb | v-maf | L0832H09 | Mm.233891 | Chromosome 2 | GCCGCAACGC |
| | | | musculoaponeu | | | | AACAGAAATT |
| | | | rotic | | | | GTTTTTAATTT |
| | | | fibrosarcoma | | | | CATGTAAAATA |
| | | | oncogene | | | | AGGGATCAATT |
| | | | family, protein | | | | TCAACCC |
| | | | B (avian) | | | | |
| 447. | C0451C02-3 | 2700094L05Rik | RIKEN cDNA | C0451C02 | Mm.25941 | No Chromosome | ACTTTTGGGTC |
| | | | 2700094L05 | | | location | TTTAGAACTGA |
| | | | gene | | | info available | GCCCACCTACT |
| | | | | | | | GAGTCTCAGTT |
| | | | | | | | TCTGTTGGTGT |
| | | | | | | | GACCT |
| 448. | H3063A08-3 | Lgmn | legumain | H3063A08 | Mm.17185 | Chromosome 12 | TGCTTACTAAG |
| | | | | | | | AAGCCAGTTTG |
| | | | | | | | GGTGGGTAAA |
| | | | | | | | GCTCTCTGGAA |
| | | | | | | | GAAGGAACTTT |
| | | | | | | | GCTTCT |
| 449. | K0629D05-3 | Evi2a | ecotropic viral | K0629D05 | Mm.3266 | Chromosome 11 | TCCCAATGTGT |
| | | | integration site | | | | AGAATTCAACT |
| | | | 2a | | | | ATGTAACGCAA |
| | | | | | | | TGGTACATTCT |
| | | | | | | | CACTGGATGAG |
| | | | | | | | ATAGA |
| 450. | G0111D11-3 | Cts1 | cathepsin L | G0111D11 | Mm.930 | Chromosome 13 | CTTATGGACAC |
| | | | | | | | TATGTCCAAAG |
| | | | | | | | GAATTCAGCTT |
| | | | | | | | AAAACTGACC |
| | | | | | | | AAACCCTTATT |
| | | | | | | | GAGTCA |
| 451. | H3077D05-3 | Npc2 | Niemann Pick | H3077D05 | Mm.29454 | Chromosome 12 | GCCATATGATG |
| | | | type C2 | | | | AACAGAATTTC |
| | | | | | | | AAGAATGCTGT |
| | | | | | | | TTTATGCCTTT |
| | | | | | | | TAACCTCCAAA |
| | | | | | | | GCAGT |
| 452. | G0104C04-3 | Dab2 | disabled | G0104C04 | Mm.288252 | Chromosome 15 | TCATTTTCCTG |
| | | | homolog 2 | | | | TCTAGGCTAAA |
| | | | (Drosophila) | | | | GCTAAACTTAA |
| | | | | | | | ACTATGGCTTT |
| | | | | | | | ACGTAAATTAA |
| | | | | | | | GCTCC |
| 453. | L0502D10-3 | Plala | phospholipase | L0502D10 | Mm.24223 | Chromosome 16 | CAACATCTAAC |
| | | | A1 member A | | | | GCTTTACATAA |
| | | | | | | | ATGCCCTTTTA |
| | | | | | | | GCTTCTCTATT |
| | | | | | | | TCGACACAACT |
| | | | | | | | GTGAT |
| 454. | H3126B08-3 | Pla2g7 | phospholipase | H3126B08 | Mm.9277 | Chromosome 17 | TTACCCAAATA |
| | | | A2, group VII | | | | AGCATTTTTTA |
| | | | (platelet- | | | | AATATACCCTG |
| | | | activating factor | | | | TACTGTAGGAT |
| | | | acetylhydrolase, | | | | AGTGATGAAC |
| | | | plasma) | | | | GCCTAG |
| 455. | J0034A07-3 | Creg | cellular | J0034A07 | Mm.459 | Chromosome 1 | ATAAGCCGTAT |
| | | | repressor of | | | | CTGGGTCTTGG |
| | | | EIA-stimulated | | | | ACTACTTTGGT |
| | | | genes | | | | GGACCTAAAGT |
| | | | | | | | AGTGACACCTG |
| | | | | | | | AAGAA |
| 456. | H3114B07-3 | Slcl2a4 | solute carrier | H3114B07 | Mm.4190 | Chromosome 8 | AAGTGGAATG |
| | | | family 12, | | | | GAGCCGGCCA |
| | | | member 4 | | | | AGCTGAGCCTG |
| | | | | | | | ACTTTTTTCAA |
| | | | | | | | TAAAACATTGT |
| | | | | | | | GTACTTC |
| 457. | K0339H12-3 | Thbs1 | thrombospondin | K0339H12 | Mm.4159 | Chromosome 2 | CTTAAAACTAC |
| | | | 1 | | | | TGTTGTGTCTA |
| | | | | | | | AAAAGTCGGT |
| | | | | | | | GTTGTACATAG |
| | | | | | | | CATAAAAATCC |
| | | | | | | | TTTGCC |
| 458. | H3028C09-3 | Adk | adenosine | H3028C09 | Mm.19352 | Chromosome 14 | CAGCTGCCTAA |
| | | | kinase | | | | CCCGCAACATT |
| | | | | | | | TGCATTATGTT |
| | | | | | | | CAGACTGTAAC |
| | | | | | | | CTGCTTACTGA |
| | | | | | | | TGGTA |
| 459. | L0277B06-3 | Psap | prosaposin | L0277B06 | Mm.233010 | Chromosome 10 | CTGTGGTACCA |
| | | | | | | | AGGAGTTATTT |
| | | | | | | | TGGATGATTAG |
| | | | | | | | AAGCACAGAA |
| | | | | | | | TGATCAGGCCT |
| | | | | | | | TTAGAG |
| 460. | H3013F05-3 | Sdc1 | syndecan 1 | H3013F05 | Mm.2580 | Chromosome Multiple | TTGTTTTTGTTT |
| | | | | | | Mappings | TTAACCTAGAA |
| | | | | | | | GAACCAAATCT |
| | | | | | | | GGACGCCAAA |
| | | | | | | | ACGTAGGCTTA |
| | | | | | | | GTTTG |
| 461. | H3084A06-3 | Spin | spindlin | H3084A06 | Mm.42193 | Chromosome 13 | TGCCTGAAAAC |
| | | | | | | | ACTTAACACTG |
| | | | | | | | ATTGTCTAAGA |
| | | | | | | | GATGAAAGTCC |
| | | | | | | | TCCAAAGATGA |
| | | | | | | | CACAG |
| 462. | H3077F04-3 | Osbpl8 | oxysterol | H3077F04 | Mm.134712 | Chromosome 10 | ACTTCAGTTAA |
| | | | binding protein- | | | | TGGGTTTATAA |
| | | | like 8 | | | | AGTCAAGCACT |
| | | | | | | | GGCATTGGTCA |
| | | | | | | | GTTTTGTATGA |
| | | | | | | | TAGGA |
| 463. | K0324A06-3 | Itgal 1 | integrin, alpha | K0324A06 | Mm.34883 | Chromosome 9 | TCCCCTATGCG |
| | | | 11 | | | | GTACGACCTTT |
| | | | | | | | ACTGTCAGAAA |
| | | | | | | | TATATTTAAGA |
| | | | | | | | AAATGTTCTAA |
| | | | | | | | ACGGT |
| 464. | C0115E05-3 | 2010110K16Rik | RIKEN cDNA | C0115E05 | Mm.9953 | Chromosome 9 | GATCCAGCCTT |
| | | | 2010110K16 | | | | CTATGAAGAAT |
| | | | gene | | | | GCAAACTGGA |
| | | | | | | | GTATCTCAAGG |
| | | | | | | | AAAGGGAAGA |
| | | | | | | | ATTCAGA |
| 465. | C0668G11-3 | Fabp5 | fatty acid | C0668G11 | Mm.741 | Chromosome Multiple | CATGACTGTTG |
| | | | binding protein | | | Mappings | AGTTCTCTTTA |
| | | | 5, epidermal | | | | TCACAAACACT |
| | | | | | | | TTACATGGACC |
| | | | | | | | TTCATGTCAAA |
| | | | | | | | CTTGG |
| 466. | L0030A03-3 | Alox5ap | arachidonate 5- | L0030A03 | Mm.19844 | Chromosome 5 | CTTGTAATCAG |
| | | | lipoxygenase | | | | ACACGTGTTTT |
| | | | activating | | | | CCTAAAATAAA |
| | | | protein | | | | GGGTATAGAC |
| | | | | | | | AAAATTTAAGC |
| | | | | | | | CCATGG |
| 467. | H3009E1 1-3 | Socs3 | suppressor of | H3009E11 | Mm.3468 | Chromosome 11 | TGTCTGAAGAT |
| | | | cytokine | | | | GCTTGAAAAAC |
| | | | signaling 3 | | | | TCAACCAAATC |
| | | | | | | | CCAGTTCAACT |
| | | | | | | | CAGACTTTGCA |
| | | | | | | | CATAT |
| 468. | L0010B01-3 | Abcal | ATP-binding | L0010B01 | Mm.369 | Chromosome 4 | TACTCCCATTA |
| | | | cassette, sub- | | | | CTATTTGCTGG |
| | | | family A | | | | TAATAGTGTAA |
| | | | (ABC1), | | | | CGCCACAGTAA |
| | | | member 1 | | | | TACTGTTCTGA |
| | | | | | | | TTCAA |
| 469. | G0116C07-3 | Ctsb | cathepsin B | G0116C07 | Mm.22753 | Chromosome 14 | CAGCCGATGCT |
| | | | | | | | TTTTCAATAGG |
| | | | | | | | ATTTTTATGCT |
| | | | | | | | TTGTGTACCTC |
| | | | | | | | AACCAAGTATG |
| | | | | | | | AAGAG |
| 470. | K0426E09-3 | Eps8 | epidermal | K0426E09 | Mm.2012 | Chromosome 6 | GGGACACTTAA |
| | | | growth factor | | | | TTTACATGTAC |
| | | | receptor | | | | TTTAACCCCAT |
| | | | pathway | | | | GAAAGAGTCT |
| | | | substrate 8 | | | | AGATAGAGAG |
| | | | | | | | AAGACAC |
| 471. | H3102F08-3 | AsahI | N- | H3102F08 | Mm.22547 | Chromosome 8 | GCCTGCCAGTA |
| | | | acylsphingosine | | | | ACCCCAGGAA |
| | | | amidohydrolase | | | | GAGTCTAGCTT |
| | | | 1 | | | | CAAAAACCCA |
| | | | | | | | CAAACTCATTA |
| | | | | | | | TTTTTAA |
| 472. | L0825G08-3 | Dcamk11 | double cortin | L0825G08 | Mm.39298 | Chromosome 3 | AATCTAGATGT |
| | | | and | | | | TAGAAATCAAT |
| | | | calcium/calmod | | | | GTGTATGATGT |
| | | | ulin-dependent | | | | ATTGTATTTAG |
| | | | protein kinase- | | | | ACCATACCCGT |
| | | | like 1 | | | | GACCG |
| 473. | K0306B10-3 | Fgf7 | fibroblast | K0306B10 | Mm.57177 | Chromosome 2 | ACGATGAGCA |
| | | | growth factor 7 | | | | GTGTTTGAAAG |
| | | | | | | | CTTTCCAGTGA |
| | | | | | | | GAACTATAATC |
| | | | | | | | CGGAAAAATG |
| | | | | | | | AATGTTT |
| 474. | H3127F04-3 | Chst11 | carbohydrate | H3127F04 | Mm.41333 | Chromosome 10 | GATGCGTGAA |
| | | | sulfotransferase | | | | ATGTTCCTCCA |
| | | | 11 | | | | GGAAAAGCCA |
| | | | | | | | TTCAAGCCTGA |
| | | | | | | | TTATTTTTCTA |
| | | | | | | | AGTAACT |
| 475. | L0208A08-3 | 1200013B22Rik | RIKEN cDNA | L0208A08 | Mm.100666 | Chromosome 1 | CATCTTAGATC |
| | | | 1200013B22 | | | | TCAGAGACTTG |
| | | | gene | | | | AACCTTGAAGC |
| | | | | | | | TGTTCCTAGTA |
| | | | | | | | CCCAGATGTGG |
| | | | | | | | ATGGA |
| 476. | H3026G09-3 | Col2a1 | procollagen, | H3026G09 | Mm.2423 | Chromosome 15 | CGTGTCCTACA |
| | | | type 11, alpha 1 | | | | CAATGGTGCTA |
| | | | | | | | TTCTGTGTCAA |
| | | | | | | | ACACCTCTGTA |
| | | | | | | | TTTTTTAAAAC |
| | | | | | | | ATCAA |
| 477 | C0218D02-3 | Madh1 | MAD homolog | C0218D02 | Mm.15185 | Chromosome 8 | AAGGAGCCAC |
| | | | 1 (Drosophila) | | | | GATAATACTTG |
| | | | | | | | ACCTCTGTGAC |
| | | | | | | | CAACTATTGGA |
| | | | | | | | TTGAGAAACTG |
| | | | | | | | ACAAGC |
| 478. | J1031F04-3 | Dfna5h | deafness, | J1031F04 | Mm.20458 | No Chromosome | GTTTATAGGTA |
| | | | autosomal | | | location | GACCTAAGAG |
| | | | dominant 5 | | | info available | ATAAAACTGCA |
| | | | homolog | | | | GGGTATCACAT |
| | | | (human) | | | | TAACGTTGGTT |
| | | | | | | | AAAAGA |
| 479. | L0276A08-3 | Rail4 | retinoic acid | L0276A08 | Mm.26786 | Chromosome 15 | AAACTTGAGAC |
| | | | induced 14 | | | | ATTTTGTAGGA |
| | | | | | | | CGCCTGACAAA |
| | | | | | | | GCGTAGCCTTT |
| | | | | | | | TTCTTGTGTCA |
| | | | | | | | GGATG |
| 480. | C0508H08-3 | Sptlc2 | serine | C0508H08 | Mm.565 | Chromosome 12 | CTCATACCAAA |
| | | | palmitoyltransf | | | | GAAATACTTGA |
| | | | erase, long | | | | CACTGCTTTGA |
| | | | chain base | | | | AGGAGATAGA |
| | | | subunit 2 | | | | TGAAGTTGGGG |
| | | | | | | | ATCTGC |
| 481. | J0042D09-3 | C78076 | ESTs C78076 | J0042D09 | Mm.290404 | Chromosome 12 | AAATCCAGCCT |
| | | | | | | | TTAAAAGCTCA |
| | | | | | | | GTTTCTTCCTC |
| | | | | | | | TAAGTGAATGT |
| | | | | | | | CATTACTCTGG |
| | | | | | | | TATAC |
| 482. | J0013B06-3 | Akrlb8 | aldo-keto | J0013B06 | Mm.5378 | Chromosome 6 | ACCAGGAACTC |
| | | | reductase | | | | TGGTAACATTT |
| | | | family 1, | | | | GAGGGCATGC |
| | | | member B8 | | | | AGATAAAATA |
| | | | | | | | ATAAAGAATG |
| | | | | | | | AGAACATT |
| 483. | H3158D11-3 | Mmp2 | matrix | H3158D11 | Mm.29564 | Chromosome 8 | TCAACATCTAT |
| | | | metalloproteinase | | | | GACCTTTTTAT |
| | | | 2 | | | | GGTTTCAGCAC |
| | | | | | | | TCTCAGAGTTA |
| | | | | | | | ATAGAGACTG |
| | | | | | | | GCTTAG |
| 484. | H3001D04-3 | Hist2h3c2 | histone 2, H3c2 | H3001D04 | Mm.261624 | Chromosome 13 | GACCGAGAGC |
| | | | | | | | CACCACAAGG |
| | | | | | | | CCAAGGGAAA |
| | | | | | | | ATAAGACCAG |
| | | | | | | | CCGTTCACTCA |
| | | | | | | | CCCGAAAAG |
| 485. | C0664G04-3 | Ppicap | peptidylprolyl | C0664G04 | Mm.3152 | Chromosome 11 | TTCTACCTCAC |
| | | | isomerase C- | | | | TAACTCCACTG |
| | | | associated | | | | ACATGGTGTAA |
| | | | protein | | | | ATGGTACATCT |
| | | | | | | | CAGTGGTGGTG |
| | | | | | | | ATGCA |
| 486. | H3091E10-3 | Nupr1 | nuclear protein | H3091E10 | Mm.18742 | Chromosome 7 | TTGGAGAAATT |
| | | | 1 | | | | AGGAGTTGTAA |
| | | | | | | | GCAGGACCTA |
| | | | | | | | GGCCTGCTTGA |
| | | | | | | | TTCTTTCCCAC |
| | | | | | | | CTAAGT |
| 487. | X98792.1 | Ptgs2 | prostaglandin- | X98792 | Mm.3137 | Chromosome 1 | TTATTGAAAAG |
| | | | endoperoxide | | | | TTTGAAGTTAG |
| | | | synthase 2 | | | | AACTTAGGCTG |
| | | | | | | | TTGGAATTTAC |
| | | | | | | | GCATAAAGCA |
| | | | | | | | GACTGC |
| 488. | L0908B12-3 | Ptpn1 | protein tyrosine | L0908B12 | Mm.227260 | Chromosome 2 | CACCATTTCCA |
| | | | phosphatase, | | | | ACTTGCTGTCT |
| | | | non-receptor | | | | CACTAATGGGT |
| | | | type 1 | | | | CTGCATTAGTT |
| | | | | | | | GCAACAATAA |
| | | | | | | | ATGTTT |
| 489. | H3081D02-3 | Bok | Bcl-2-related | H3081D02 | Mm.3295 | Chromosome 1 | AACAAGAGAT |
| | | | ovarian killer | | | | CCTGTGGATGA |
| | | | protein | | | | GGGGGTCTGTA |
| | | | | | | | TAAGTTATACT |
| | | | | | | | CCAATAAAGCT |
| | | | | | | | TTACCT |
| 490. | C0127E12-3 | Cln5 | ceroid- | C0127E12 | Mm.38783 | No Chromosome | TTTTGACCAGT |
| | | | lipofuscinosis, | | | location | TGAACCCATTT |
| | | | neuronal 5 | | | info available | TGTTTTCCTAG |
| | | | | | | | CGAACACTAGC |
| | | | | | | | ATAATATTGGA |
| | | | | | | | AAAGC |
| 491. | K0310G10-3 | Col5a2 | procollagen, | K0310G10 | Mm.257899 | Chromosome 1 | GTGAGGATTGG |
| | | | type V, alpha 2 | | | | AATTAGAACAT |
| | | | | | | | TCATAAGAAA |
| | | | | | | | ATATGACCCAA |
| | | | | | | | CATTTCTTAGC |
| | | | | | | | ATGACC |
| 492. | H3023H09-3 | Ftl 1 | ferritin light | H3023H09 | Mm.7500 | Chromosome 7 | CGCCCTGGAGC |
| | | | chain 1 | | | | CTCTGTCAAGT |
| | | | | | | | CTTGGACCAAG |
| | | | | | | | TAAAAATAAA |
| | | | | | | | GCTTTTTGAGA |
| | | | | | | | CAGCAA |
| 493. | G0104B11-3 | Slc7a7 | solute carrier | G0104B11 | Mm.142455 | Chromosome 14 | AAGATGGAGA |
| | | | family 7 | | | | GTTGTCCAAAC |
| | | | (cationic amino | | | | AAGATCCCAA |
| | | | acid transporter, | | | | GTCTAAATAGA |
| | | | y+ system), | | | | GCAAGGGATTC |
| | | | member 7 | | | | TGAGGTG |
| 494. | C0123F05-3 | B4galt5 | UDP- | C0123F05 | Mm.200886 | Chromosome 2 | GTTTTAAAAGG |
| | | | Gal:betaGlcNAc | | | | TGCCAGGGGTA |
| | | | beta 1,4- | | | | CATTTTTGCAC |
| | | | galactosyltrans- | | | | TGAAACCTAAA |
| | | | ferase, | | | | GATGTTTTAAA |
| | | | polypeptide 5 | | | | AACAC |
| 495. | H3082D01-3 | 1801105C04Rik | RIKEN cDNA | H3082D01 | Mm.25311 | Chromosome 15 | TCTGAGGTATT |
| | | | 1810015C04 | | | | AAAATATCTAG |
| | | | gene | | | | ACTGAATTTTG |
| | | | | | | | CCAAATGTAAG |
| | | | | | | | AGGGAGAAAG |
| | | | | | | | TTCCTG |
| 496. | C0121E07-3 | AW539579 | EST | C0121E07 | Mm.282049 | No Chromosome | AAGTATTGCTA |
| | | | AW539579 | | | location | GACTGAAACC |
| | | | | | | info available | ACTTGAACTTC |
| | | | | | | | TCAGAGAGGTT |
| | | | | | | | AGACTGACAG |
| | | | | | | | AAGGTGT |
| 497 | H3153H08-3 | Hs6st2 | heparan sulfate | H3153H08 | Mm.41264 | Chromosome X | ACATTTTTGTC |
| | | | 6-O- | | | | ATCATCATGTA |
| | | | sulfotransferase | | | | AATCCCACGAT |
| | | | 2 | | | | TTCAAACTGTA |
| | | | | | | | AACATCTGTTC |
| | | | | | | | AGTGG |
| 498. | J0238C08-3 | 4930579A11Rik | RIKEN cDNA | J0238C08 | Mm.24584 | Chromosome 11 | CTGGGGAAATT |
| | | | 4930579A11 | | | | GATCTTTAAAT |
| | | | gene | | | | TTTGAAACAGT |
| | | | | | | | ATAAGGAAAA |
| | | | | | | | TCTGGTTGGTG |
| | | | | | | | TCTCAC |
| 499 | L0942B10-3 | Msr2 | macrophage | L0942B10 | Mm.45173 | Chromosome 3 | AGGACTCAAA |
| | | | scavenger | | | | ACTATATTAAT |
| | | | receptor 2 | | | | CTGCTCTGAGA |
| | | | | | | | TAATGTTCCAA |
| | | | | | | | AAGCTCCAAA |
| | | | | | | | GAAAGCC |
| 500. | J0915B05-3 | Cdcal | cell division | J0915B05 | Mm.151315 | Chromosome 1 | GCTCCAACATG |
| | | | cycle associated | | | | CCATGTATTGT |
| | | | 1 | | | | ATAGACTTTTA |
| | | | | | | | CTACAATTCAA |
| | | | | | | | ATAACGTGTAC |
| | | | | | | | AGCTT |
| 501. | H3058B09-3 | Lypla3 | lysophospholipase | H3058B09 | Mm.25492 | Chromosome 8 | CAGCTGAATGG |
| | | | 3 | | | | GTTTTGGTTTG |
| | | | | | | | CAGGAAAACA |
| | | | | | | | GTCCAGAGCTT |
| | | | | | | | TGAAAAGGCTC |
| | | | | | | | CTAAGA |
| 502. | C0197E01-3 | D630023B12 | hypothetical | C0197E01 | Mm.227732 | Chromosome 3 | TGTTTTTATTG |
| | | | protein | | | | TGTTTGGTGGA |
| | | | D630023B12 | | | | GAAGAATAAT |
| | | | | | | | ACACTTCTTGC |
| | | | | | | | CTAAATCCAGA |
| | | | | | | | AGCCCC |
| 503. | J0802G04-3 | 0610011104Rik | RIKEN cDNA | J0802004 | Mm.27061 | Chromosome 6 | TCCAGTTCCCG |
| | | | 0610011104 | | | | AAGAAGCTGA |
| | | | gene | | | | TAGGAATTGCC |
| | | | | | | | CTTGTGCATAT |
| | | | | | | | ACTACACAAGC |
| | | | | | | | ATGCTA |
| 504. | H3039E08-3 | Sh3d3 | SH3 domain | H3039E08 | Mm.4165 | Chromosome 19 | CATAAAGACAT |
| | | | protein 3 | | | | AGTGGAGGTTC |
| | | | | | | | TGTTTACTCAG |
| | | | | | | | CCGAATGTGGA |
| | | | | | | | GCTGAACCAGC |
| | | | | | | | AGAAT |
| 505. | L0210A08-3 | B130023014Rik | RIKEN cDNA | L0210A08 | Mm.27098 | Chromosome 5 | GGATTCGGCTC |
| | | | B130023014 | | | | GATGAATGAA |
| | | | gene | | | | GCACTTTATGG |
| | | | | | | | ACTGCGGGGAT |
| | | | | | | | CAGTTACTGCC |
| | | | | | | | ACACCC |
| 506. | H3114C10-3 | Ppgb | protective | H3114C10 | Mm.7046 | Chromosome 2 | TGCTTTTACCA |
| | | | protein for beta- | | | | TGTTCTCGAGG |
| | | | galactosidase | | | | TTCCTGAACAA |
| | | | | | | | AGAGCCTTACT |
| | | | | | | | GATAGTTCCGC |
| | | | | | | | TGCAA |
| 507. | C0322A01-3 | 2810441C07Rik | RIKEN cDNA | C0322A01 | Mm.29329 | Chromosome 4 | TGAAGCAAAA |
| | | | 2810441C07 | | | | AACATAAAAC |
| | | | gene | | | | CTCACCACTGC |
| | | | | | | | CTGCTGAACCT |
| | | | | | | | AGAACCTTTTG |
| | | | | | | | TTGGGGC |
| 508. | L0256F11-3 | Adfp | adipose | L0256F11 | Mm.381 | Chromosome 4 | GAATCCTTAGA |
| | | | differentiation | | | | TGAAGTTATGG |
| | | | related protein | | | | ATTACTTTGTT |
| | | | | | | | AACAACACGC |
| | | | | | | | CTCTCAACTGG |
| | | | | | | | CTGGTA |
| 509. | L0939H06-3 | Mgat5 | mannoside | L0939H06 | Mm.38399 | Chromosome 1 | GATATTAGTAG |
| | | | acetylglucosami | | | | TATATCATAAA |
| | | | nyltransferase 5 | | | | ACTTGAGAAAT |
| | | | | | | | AAAGATGCGCT |
| | | | | | | | CACCCCCTATC |
| | | | | | | | TGTTG |
| 510. | C0503B05-3 | Dcanikl1 | double cortin | C0503B05 | Mm.39298 | Chromosome 3 | TGTGATAAAGT |
| | | | and | | | | TGTGACATACG |
| | | | calcium/calmod | | | | TATTAGTTGGC |
| | | | ulin-dependent | | | | ACATATTTAAG |
| | | | protein kinase- | | | | CTCCAAATCAG |
| | | | like 1 | | | | TTTGC |
| 511. | H3136H11-3 | Map4k5 | mitogen- | H3136H11 | Mm.260244 | Chromosome 12 | TAAAAGTTAAA |
| | | | activated | | | | GTAAGCGAAG |
| | | | protein kinase | | | | AAAGGAAGCT |
| | | | kinase kinase | | | | GTATCTACACT |
| | | | kinase 5 | | | | GCTTTCCAGTT |
| | | | | | | | TAATCAG |
| 512. | K0349A04-3 | Fnl | fibronectin 1 | K0349A04 | Mm.193099 | Chromosome 1 | GGAGATTTTTC |
| | | | | | | | TCTTCAGGGTG |
| | | | | | | | TCTACATACCT |
| | | | | | | | TACACACACTT |
| | | | | | | | GTGTCTTAATA |
| | | | | | | | AGCAA |
| 513. | C0177C04-3 | Ctsz | cathepsin Z | C0177C04 | Mm.156919 | Chromosome 2 | AATCCATGGGA |
| | | | | | | | GGGGGGAACA |
| | | | | | | | AGTCCAGACTG |
| | | | | | | | CTTAAGAAATG |
| | | | | | | | AGTAAAATATC |
| | | | | | | | TGGCTT |
| 514. | C0668D08-3 | Grn | granulin | C0668D08 | Mm.1568 | Chromosome 11 | AATGTGGAGTG |
| | | | | | | | TGGAGAAGGG |
| | | | | | | | CATTTCTGCCA |
| | | | | | | | TGATAACCAGA |
| | | | | | | | CCTGTTGTAAA |
| | | | | | | | GACAGT |
| 515. | C0106D12-3 | Anxal | annexin A1 | C0106D12 | Mm.14860 | Chromosome 19 | TGACATGAATG |
| | | | | | | | ATTTTACCAGA |
| | | | | | | | AGAAGTATGG |
| | | | | | | | AATCTCTCTTT |
| | | | | | | | GCCAAGC |
| 516. | H3078E09-3 | Hexb | hexosaminidase | H3078E09 | Mm.27816 | Chromosome 13 | ACTGGATACTG |
| | | | B | | | | TAACTATGAGA |
| | | | | | | | ATAAAATATAG |
| | | | | | | | AAGTGACAGA |
| | | | | | | | CGTCTACAGCA |
| | | | | | | | TTCCAG |
| 517. | L0033F05-3 | 2810442122Rik | RIKEN cDNA | L0033F05 | Mm.275696 | Chromosome 10 | ATACAAGCAA |
| | | | 2810442122 | | | | GCTGTTAAAGA |
| | | | gene | | | | TCTTGGATCCC |
| | | | | | | | ATTCTATAGTG |
| | | | | | | | TGTATACCTAA |
| | | | | | | | ATCAAC |
| 518. | K0144G04-3 | Ifi203 | interferon | K0144G04 | Mm.245007 | Chromosome 1 not | AGCATCAACTG |
| | | | activated gene | | | placed | TCCTGTCAAGC |
| | | | 203 | | | | ACAAAAAATG |
| | | | | | | | AAGAAGAAAA |
| | | | | | | | TAATTACCCAA |
| | | | | | | | AAGATGG |
| 519. | H3144E05-3 | 4933426M11Rik | RIKEN cDNA | H3144E05 | Mm.27112 | Chromosome 12 | CCTCTGTTCTG |
| | | | 4933426M11 | | | | AGGAACATTCT |
| | | | gene | | | | AGCATAGAAA |
| | | | | | | | ATGGAATATGC |
| | | | | | | | TGCAAACATTT |
| | | | | | | | CTAGAT |
| 520. | K0336D02-3 | Ifi16 | interferon, | K0336D02 | Mm.212870 | Chromosome 1 | GTGTAGAAGCC |
| | | | gamma- | | | | TATTGAAATAT |
| | | | inducible | | | | CAGTCCTATAA |
| | | | protein 16 | | | | AGACCATCTCT |
| | | | | | | | TAATTCTAGGA |
| | | | | | | | AATGG |
| 521. | H3004B12-3 | Hpn | hepsin | H3004B12 | Mm.19182 | Chromosome 7 | CTGATCCCGCC |
| | | | | | | | TCATCTCGCTG |
| | | | | | | | CTCCGTGCTGC |
| | | | | | | | CCTAGCATCCA |
| | | | | | | | AAGTCAAAGTT |
| | | | | | | | GGTTT |
| 522. | K0617G07-3 | Atp6vlb2 | ATPase, H+ | K0617G07 | Mm.10727 | Chromosome 8 | TGTAGAAAATG |
| | | | transporting, | | | | TGGCCTCTCGT |
| | | | V1 subunit B, | | | | TATAAATGAAA |
| | | | isofonn 2 | | | | ATAAATGTTTA |
| | | | | | | | ATTTAATGGGA |
| | | | | | | | GTTTC |
| 523. | L0849B10-3 | Pltp | phospholipid | L0849B10 | Mm.6105 | Chromosome 2 | GGTGCCACAG |
| | | | transfer protein | | | | AGAAGAGCCC |
| | | | | | | | AGTTGGAAGCT |
| | | | | | | | ATACCCGATTT |
| | | | | | | | AATTCCAGAAT |
| | | | | | | | TAGTCAA |
| 524. | L0019H03-3 | Fnl | fibronectin 1 | L0019H03 | Mm.193099 | Chromosome 1 | CAGTGTTGTTT |
| | | | | | | | AAGAGAATCA |
| | | | | | | | AAAGTTCTTAT |
| | | | | | | | GGTTTGGTCTG |
| | | | | | | | GGATCAATAG |
| | | | | | | | GGAAACA |
| 525. | J0099E12-3 | Slc6a6 | solute carrier | J0099E12 | Mm.200518 | Chromosome 6 | ATAACTATATA |
| | | | family 6 | | | | TACTTAGAGTC |
| | | | (neurotransmitter | | | | TGTCATACACT |
| | | | transporter, | | | | TTGCCACTTGA |
| | | | taurine), | | | | ATTGGTCTTGC |
| | | | member 6 | | | | CAGCA |
| 526. | J0023G04-3 | BC004044 | cDNA sequence | J0023G04 | Mm.6419 | Chromosome 5 | CCTTGGGACAT |
| | | | BC004044 | | | | TTTTGTGGAGT |
| | | | | | | | AGTTTGCAGTG |
| | | | | | | | AGATAACAGT |
| | | | | | | | GCAATAAAGA |
| | | | | | | | TACAGCA |
| 527. | C0913D04-3 | 4933433D23Rik | RIKEN cDNA | C0913D04 | Mm.46067 | Chromosome 14 | TCTATACCTGG |
| | | | 4933433D23 | | | | ATAAAAAGAA |
| | | | gene | | | | ACCTACACTTC |
| | | | | | | | ACTGTAAAACT |
| | | | | | | | TCATGTTTCAA |
| | | | | | | | GGCAAG |
| 528. | H3020C02-3 | Mt1 | metallothionein | H3020C02 | Mm.192991 | Chromosome 8 | CCTGTTTACTA |
| | | | 1 | | | | AACCCCCGTTT |
| | | | | | | | TCTACCGAGTA |
| | | | | | | | CGTGAATAATA |
| | | | | | | | AAAGCCTGTTT |
| | | | | | | | GAGTC |
| 529. | C0217B11-3 | Sema4d | sema domain, | C0217B11 | Mm.33903 | Chromosome 13 | ACCGTGTAGAC |
| | | | immunoglobulin | | | | ACTCATATTTT |
| | | | domain (Ig), | | | | GCATGACATGA |
| | | | transmembrane | | | | TCTACCATTCG |
| | | | domain (TM) | | | | GTGTAAACATT |
| | | | and short | | | | TGTGT |
| | | | cytoplasmic | | | | |
| | | | domain, | | | | |
| | | | (semaphorin) | | | | |
| | | | 4D | | | | |
| 530. | C0917E01-3 | Bhlhb2 | basic helix- | C0917E01 | Mm.2436 | Chromosome 6 | GCCAAAGGAA |
| | | | loop-helix | | | | AATGTTTCAGA |
| | | | domain | | | | TGTCTATTGT |
| | | | containing, | | | | ATAATTACTTG |
| | | | class B2 | | | | ATCTACCCAGT |
| | | | | | | | GAGGAA |
| 531. | H3132B12-5 | Deafi | deformed | H3132B12 | Mm.28392 | Chromosome 7 | TCCAGAAGCTG |
| | | | epidermal | | | | CATTGCCAACA |
| | | | autoregulatoiy | | | | TCACACCCCAA |
| | | | factor 1 | | | | AATTGTCCTGA |
| | | | (Drosophila) | | | | CATCGCTGCCC |
| | | | | | | | GCATT |
| 532. | L0270C04-3 | Mppl | membrane | L0270C04 | Mm.2814 | Chromosome X | AAGGACTCTGA |
| | | | protein, | | | | GGCCATCCGTA |
| | | | palmitoylated | | | | GTCAGTATGCT |
| | | | | | | | CATTACTTTGA |
| | | | | | | | CCTCTCTTTGG |
| | | | | | | | TGAAT |
| 533. | J0709H10-3 | | transcribed | J0709H10 | Mm.296913 | Chromosome 13 | ATCTCCCAAGG |
| | | | sequence with | | | | CAAAGAACTG |
| | | | moderate | | | | AAACTCAGAG |
| | | | similarity to | | | | CTGTCTGGATT |
| | | | protein | | | | GAAGAAATGT |
| | | | pir:A38712 | | | | GTGTTGTT |
| | | | (H. sapiens) | | | | |
| | | | A38712 | | | | |
| | | | fibrillarin | | | | |
| | | | [validated]- | | | | |
| | | | human | | | | |
| 534. | C0166A10-3 | Car2 | carbonic | C0166A10 | Mm.1186 | Chromosome 3 | ATGAAGGTAG |
| | | | anhydrase 2 | | | | GATAATTAATT |
| | | | | | | | ACAAGTCCACA |
| | | | | | | | TCATGAGACAA |
| | | | | | | | ACTGAAGTAAC |
| | | | | | | | TTAGGC |
| 535. | L0511A03-3 | BM122519 | ESTs | L0511A03 | Mm.296074 | Chromosome 1 | GGTGTAGCCAT |
| | | | BM122519 | | | | ACAATACACA |
| | | | | | | | AATACAATAG |
| | | | | | | | ATATTCTCTCT |
| | | | | | | | ACAATCTTTAT |
| | | | | | | | GGTGTGG |
| 536. | H3029F09-3 | Atp6v1e1 | ATPase, H+ | H3029F09 | Mm.29045 | Chromosome 6 | GGAGAAGCAG |
| | | | transporting, | | | | ATTATCTGTGT |
| | | | VI subunit E | | | | GGCTTCCTCTT |
| | | | isoform 1 | | | | TCTGTTCTAAT |
| | | | | | | | ACTGGTAATCA |
| | | | | | | | GTGGAC |
| 537. | J0716H11-3 | Kdtl | kidney cell line | J0716H11 | Mm.1314 | Chromosome 6 | GTGAACACCA |
| | | | derived | | | | GAATTTAATTT |
| | | | transcript 1 | | | | CCATACTTGTA |
| | | | | | | | CAGGTAGGACT |
| | | | | | | | ATTCTTCAGCT |
| | | | | | | | CTCTAC |
| 538. | C0102C01-3 | Acp5 | acid | C0102C01 | Mm.46354 | Chromosome 9 | GGCTTCACACA |
| | | | phosphatase 5, | | | | TGTGGAGATAA |
| | | | tartrate resistant | | | | GCCCCAAAGA |
| | | | | | | | AATGACCATCA |
| | | | | | | | TATATGTGGAA |
| | | | | | | | GCCTCT |
| 539. | C0641C07-3 | Pdgfb | platelet derived | C0641C07 | Mm.144089 | Chromosome 15 | GTTTGTAAAGT |
| | | | growth factor, | | | | TGGTGATTATA |
| | | | B polypeptide | | | | TTTTTTGGGGG |
| | | | | | | | CTTTCTTT- |
| | | | | | | | TTAT |
| | | | | | | | TTTTTAAATGT |
| | | | | | | | AAAG |
| 540. | C0147C09-3 | Tct7 | tetratricopeptide | C0147C09 | Mm.77396 | Chromosome 17 | ATGGAATTCTG |
| | | | repeat domain | | | | TTAGAGTAAAA |
| | | | 7 | | | | AAGAGAAAAG |
| | | | | | | | CAGATACTATT |
| | | | | | | | GGCTGGCCTTG |
| | | | | | | | GAGGTC |
| 541. | K0301G02-3 | 94300025M21Rik | RIKEN cDNA | K0301G02 | Mm.87452 | Chromosome 1 | AATAGTGCTGA |
| | | | 9430025M21 | | | | ATTTGTCTAAA |
| | | | gene | | | | CAGAATTGAG |
| | | | | | | | AGGTCATAGA |
| | | | | | | | AATCCTTAACA |
| | | | | | | | GGGTAAC |
| 542. | H3002D05-3 | Tpbpb | trophoblast | H3022E05 | Mm.297991 | Chromosome 13 | TATGAAGATTT |
| | | | specific protein | | | | GGGAAAGAAC |
| | | | beta | | | | AGCTATCTGAC |
| | | | | | | | ACCTGGAAGG |
| | | | | | | | CTCAGCCAGAG |
| | | | | | | | TAACAGT |
| 543. | H3007C09 | Sh3bgr13 | SH3 domain | H3007C09 | Mm.22240 | Chromosome 4 | GAGGCAACATT |
| | | | binding | | | | CCTTATTCACC |
| | | | glutamic acid- | | | | AACTAGTCTCA |
| | | | rich protein-like | | | | AAAGATTGTCT |
| | | | 3 | | | | TAAGCCCTGAC |
| | | | | | | | GATGG |
| 544. | L0820G02-3 | Igsf4 | immunoglobulin | L0820G02 | Mm.248549 | Chromosome 9 | TAATGAAGGAT |
| | | | superfamily, | | | | GTATAATTGAT |
| | | | member 4 | | | | GCCAAATAAG |
| | | | | | | | CTTGTTCTTTA |
| | | | | | | | GTCACGATGAC |
| | | | | | | | GTCTTG |
| 545. | C0120H11-3 | 4933433D23Rik | RIKEN cDNA | C0120H11 | Mm.46067 | Chromosome 14 | CAGTTTGCGAA |
| | | | 4933433D23 | | | | GTAGAATTTTG |
| | | | gene | | | | TTTCTAAAAGT |
| | | | | | | | AAAAGCTAAG |
| | | | | | | | TTGAAGTCCTC |
| | | | | | | | ACGAG |
| 546. | J1016E08-3 | 1810046J19Rik | RIKEN cDNA | J1016E08 | Mm.259614 | Chromosome 11 | TAGAAAAGAT |
| | | | 1810046J19 | | | | CACCAACAGCC |
| | | | gene | | | | GGCCTCCCTGT |
| | | | | | | | GTCATCCTGTG |
| | | | | | | | ACTAAGAAAT |
| | | | | | | | GATTCTT |
| 547. | L0822D10-3 | Prkcb | protein kinase | L0822D10 | Mm.4182 | Chromosome 7 | TATCTAAGAGC |
| | | | C, beta | | | | CAAGTCTATGG |
| | | | | | | | CATTAGCTGTG |
| | | | | | | | AGAAGTAGTTA |
| | | | | | | | CCACTGTAATT |
| | | | | | | | CACCT |
| 548. | H3050H09-3 | Ppp2r5c | protein | H3050H09 | Mm.36389 | Chromosome 12 | AAATTATCACT |
| | | | phosphatase 2, | | | | TGATACGGA |
| | | | regulatory | | | | GGAACATGACT |
| | | | subunit B | | | | AGGCACATTTT |
| | | | (B56), gamma | | | | ATGAATACTCC |
| | | | isoform | | | | AAATCC |
| 549. | J0442H09-3 | | Mus musculus | J0442H09 | Mm.11982 | Chromosome 10 | AACTATGGTG |
| | | | hypothetical | | | | GTATATTTTTG |
| | | | LOC237436 | | | | AACACAGGTTA |
| | | | (LOC237436), | | | | ACTGTGGAGGT |
| | | | mRNA | | | | TATCTGCTAAT |
| | | | | | | | AGCAA |
| 550. | H3141E06-3 | Sra1 | steroid receptor | H3151E06 | Mm.29058 | Chromosome 18 | ACCTCTGGAAC |
| | | | RNA activator | | | | AGGCATTGGA |
| | | | 1 | | | | GGACTGCCATG |
| | | | | | | | GTCACACAAA |
| | | | | | | | GAAACAGAAC |
| | | | | | | | TTTTACAT |
| 551. | C0171H06-3 | Adss2 | adenylosuccinate | C0170H06 | Mm.132946 | Chromosome 1 | CCAGTATACCT |
| | | | synthetase 2, | | | | ACAAAATGAC |
| | | | non muscle | | | | CCACAAGTAAC |
| | | | | | | | CCGCATGAGTC |
| | | | | | | | CAAGTTGTCAG |
| | | | | | | | CCATAT |
| 552. | K0344C08-3 | Emp1 | epithelial | K0344C08 | Mm.30024 | Chromosome 6 | GTAAAGGGAC |
| | | | membrane | | | | CATTACTAAGT |
| | | | protein 1 | | | | GTATTTCTCTA |
| | | | | | | | GCATATTATGT |
| | | | | | | | TTAAGGGACTG |
| | | | | | | | TTCAAG |
| 553. | J0907F03-3 | Npl | N- | J0907F03 | Mm.24887 | Chromosome 1 | CTCTAAGTCAT |
| | | | acetylneuraminate | | | | TCATTTTGTAA |
| | | | pyruvatelyase | | | | AATTATTATAG |
| | | | | | | | AGAAATCTCTA |
| | | | | | | | CTTATACAGAT |
| | | | | | | | GCAAT |
| 554. | J1008C10-3 | Ptpn1 | protein tyrosine | J1008C10 | Mm.2668 | Chromosome 2 | TCTAATCTCAG |
| | | | phosphatase, | | | | GGCCTTAACCT |
| | | | non-receptor | | | | GTTCAGGAGA |
| | | | type 1 | | | | AGTAGAGGAA |
| | | | | | | | ATGCCAAATAC |
| | | | | | | | TCTTCTT |
| 555. | K0103F09-3 | 2500002K03Rik | K0103F09 | Mm.29181 | Chromosome 6 | ATTCAGATCAG | |
| | | | 2500002K03 | | | | GAAAGGTTGA |
| | | | gene | | | | AATGGTCTTCG |
| | | | | | | | TTACCAGGAGG |
| | | | | | | | TCTACATTTAT |
| | | | | | | | TAATTT |
| 556. | C0837H01-3 | Adam9 | a disinegrin | C0837H01 | Mm.28908 | Chromosome 8 | CAGTTATGGGC |
| | | | and | | | | TTCCATTTTCA |
| | | | metalloproteinase | | | | AATATCTTTTC |
| | | | domain 9 | | | | AACTGTAATGA |
| | | | (meltrin | | | | CTATGACAGGA |
| | | | gamma) | | | | ACTGA |
| 557. | J0207H07-3 | Runx2 | runt related | J0207H07 | Mm.4509 | Chromosome 17 | GCTTTCTATGC |
| | | | transcription | | | | ACGTATTGTAC |
| | | | factor 2 | | | | AAATTGTGCTT |
| | | | | | | | TGTGCCACAGG |
| | | | | | | | TCATGATCGTG |
| | | | | | | | GATGA |
| 558. | J0246C10-3 | Tpd52 | tumor protein | J0246C10 | Mm.2777 | Chromosome Multiple | TGGCTAGATTT |
| | | | D52 | | | Mappings | AATTGAGGATA |
| | | | | | | | AGGTTTCTGCA |
| | | | | | | | AACCAGAATTG |
| | | | | | | | AAAAGCCACA |
| | | | | | | | GTGTCG |
| 559. | H3158E12-3 | BC003324 | cDNA sequence | H3158E12 | Mm.29656 | Chromosome 5 | AGAGGACCATT |
| | | | BC003324 | | | | ATGAAGAAGC |
| | | | | | | | TGTTCTCTTTC |
| | | | | | | | CGGTCAGGGA |
| | | | | | | | AGCATACCTAG |
| | | | | | | | ACTGAAA |
| 560. | H3094A04-3 | Dnajc3 | DnaJ (Hsp40) | H3094A04 | Mm.12616 | Chromosome 14 | AGAAAAGAAA |
| | | | homolog, | | | | AAGCAGAGA |
| | | | subfamily C, | | | | AAAAGTTCATT |
| | | | member 3 | | | | GACATAGCAG |
| | | | | | | | CTGCTAAAGAA |
| | | | | | | | GTCCTCTC |
| 561. | L0231F01-3 | Evl | Ena-vasodilator | L0231F01 | Mm.2144 | Chromosome 12 | ATATTTGCTTA |
| | | | stimulated | | | | TTTAAGCGTAC |
| | | | phosphoprotein | | | | GTTCCTTTGGT |
| | | | | | | | TTATAGAGAAC |
| | | | | | | | ACCCCCAAATC |
| | | | | | | | ACCTG |
| 562. | K0512E10-3 | Myo5a | myosin Va | K0512E10 | Mm.222258 | Chromosome 9 | GACTCTCCCAAC |
| | | | | | | | TTACAGACTTT |
| | | | | | | | TATCAGATATG |
| | | | | | | | GAGAAGATAA |
| | | | | | | | TGTTAAGAGAC |
| | | | | | | | TTCACA |
| 563. | K0608H09-3 | Ptprc | protein tyrosine | K0608H09 | Mm.143846 | Chromosome 1 | TAAAATCCCAT |
| | | | phosphatase, | | | | TGAAAGTGGA |
| | | | receptor type, C | | | | CTCAGTTGTAA |
| | | | | | | | GAATAACAAT |
| | | | | | | | GTGTACCATTC |
| | | | | | | | TGGAATG |
| 564. | L0842E04-3 | Prkcb | protein kinase | L0842E04 | Mm.4182 | Chromosome 7 | CCAATGAACCG |
| | | | C, beta | | | | ACAGTGTCAAA |
| | | | | | | | ACTTAACTGTG |
| | | | | | | | TCCAATACCAA |
| | | | | | | | AATGCTTCAGT |
| | | | | | | | ATTTG |
| 565. | H3121G01-3 | BG073361 | ESTs | H3121G01 | Mm.182649 | Chromosome 11 | TCAAATCAGTT |
| | | | BG073361 | | | | TCAACTTTCAT |
| | | | | | | | AAAATGGATTC |
| | | | | | | | TTTAATGGATG |
| | | | | | | | GAGACTTACTC |
| | | | | | | | GTCGG |
| 566. | C0947F04-3 | 5830411K21Rik | RIKEN cDNA | C0947F04 | Mm.160141 | Chromosome 2 | CTATACACAAG |
| | | | 5830411K21 | | | | ATATGCTAGGA |
| | | | gene | | | | GATGTGAAAG |
| | | | | | | | ATAATGGAGA |
| | | | | | | | CTTTCCAGTAA |
| | | | | | | | GCACTTT |
| 567. | H3009D03-5 | Plac8 | placenta- | H3009D03 | Mm.34609 | Chromosome 5 | CTGAGATTTTT |
| | | | specific 8 | | | | CAAATCTTTGG |
| | | | | | | | CAACTGAGATG |
| | | | | | | | GGATGGATCCA |
| | | | | | | | TTTAATTAGAG |
| | | | | | | | AACGG |
| 568. | H3132E07-3 | Lxn | latexin | H3132E07 | Mm.2632 | Chromosome 3 | AAATGTCTTTC |
| | | | | | | | CAACAGTAATG |
| | | | | | | | GTACTATGTCT |
| | | | | | | | ATCCCCTAATA |
| | | | | | | | AAACTTCACTT |
| | | | | | | | CAGCC |
| 569. | H3054C01-3 | Nr2e3 | nuclear receptor | H3054C01 | Mm.9652 | Chromosome X | TGAACATTCAC |
| | | | subfamily 2, | | | | AGGATTTCTAA |
| | | | group E, | | | | CTATACTGATA |
| | | | member 3 | | | | TAAACCCAGTG |
| | | | | | | | TTTTCTGGACT |
| | | | | | | | CAGGG |
| 570. | H3013h03-3 | Manla | mannosidase 1, | H3013H03 | Mm.117294 | Chromosome 10 | CAACAAAGTTG |
| | | | alpha | | | | ATTTACATGTA |
| | | | | | | | TAATCCACACC |
| | | | | | | | CTTAAAGATGA |
| | | | | | | | ACAGTTAGAGT |
| | | | | | | | AGCAC |
| 571. | J0058F02-3 | ank | progressive | J0058F02 | Mm.142714 | Chromsome 15 | TGGACACAGTT |
| | | | ankylosis | | | | CACTAAATTCC |
| | | | | | | | TGATTTAGTCA |
| | | | | | | | AAGTAACTAG |
| | | | | | | | ACTGAAAGAA |
| | | | | | | | CCTAAAC |
| 572. | L0829D10-3 | Snca | synuclein, alpha | L0829D10 | Mm.17484 | Chromosome 6 | TTGTTGTGGCT |
| | | | | | | | TCACACTTAAA |
| | | | | | | | TTGTTAGAAGA |
| | | | | | | | AACTTAAAACA |
| | | | | | | | CCTAAGTGACT |
| | | | | | | | ACCAC |
| 573. | H3037H02-3 | 1110018O12Rik | RIKEN cDNA | H3037H02 | Mm.28252 | Chromosome 18 | TGAACACATCA |
| | | | 1110018O12 | | | | AGTATTCTGGA |
| | | | gene | | | | GCTTCAGCGGC |
| | | | | | | | AGTTAAATGCC |
| | | | | | | | AGTGACGAAC |
| | | | | | | | ATGGAA |
| 574. | K0105H12-3 | Cdk6 | cyclin- | K0105H12 | Mm.88747 | Chromsome 5 | AAGGTCCAAA |
| | | | dependent | | | | ATACAGACATT |
| | | | kinase 6 | | | | TTTGCTAGGGC |
| | | | | | | | CTAGAAATCGA |
| | | | | | | | CCATAAAACAC |
| | | | | | | | ACTGCA |
| 575. | C0105D10-3 | | C0105D10-3 | C0105D10 | | No Chromosome | GACTGAAATG |
| | | | NIA Mouse | | | location | AAAGTTCCACT |
| | | | E7.5 | | | info available | AACGGTATTTG |
| | | | Extraembryonic | | | | CTCTAGTGATA |
| | | | Portion cDNA | | | | TGTGGACATTG |
| | | | Library Mus | | | | TGATAT |
| | | | musculus | | | | |
| | | | cDNA clone | | | | |
| | | | C0105D10 3′, | | | | |
| | | | MRNA | | | | |
| | | | sequence | | | | |
| 576. | L0229E05-3 | Prkx | putative | L0229E05 | Mm.106185 | Chromosome X | TCAAATAAAA |
| | | | serine/threonine | | | | AACCCTTAATC |
| | | | kinase | | | | AGGCTGTAAAT |
| | | | | | | | CAAATGACACT |
| | | | | | | | ATGCGATGTCA |
| | | | | | | | CTACAG |
| 577. | L0931H07-3 | | ESTs | L0931H07 | Mm.221935 | Chromosome 1 | GCACTATAAAT |
| | | | BQ557106 | | | | TTCATCTTTTG |
| | | | | | | | AAGGTTGTTGA |
| | | | | | | | CTACAAGGGTA |
| | | | | | | | CAAAAATGAT |
| | | | | | | | ACAGGC |
| 578. | K0138B11-3 | Trim25 | tripartite motif | K0138B11 | Mm.4973 | Chromosome 11 | CTTGCATGAGT |
| | | | protein 25 | | | | GCGTGTTTAAG |
| | | | | | | | TTCTCGGAATT |
| | | | | | | | TCCTGAGAGGA |
| | | | | | | | TGGAGTGCCAT |
| | | | | | | | TGTTA |
| 579. | H3019H03-3 | Lass6 | longevity | H3019H03 | Mm.265620 | Chromosome 2 | AGTGTTAGCTG |
| | | | assurance | | | | CAAAGCTACA |
| | | | homolog 6 (S. | | | | AAGCTCTGGA |
| | | | cerevisiae) | | | | TGGTTACATTA |
| | | | | | | | TGATTCTGGAA |
| | | | | | | | CGTTCG |
| 580. | J0051F04-3 | Ifi30 | interferon | J0051F04 | Mm.30241 | Chromosome 8 | TCCAGACTTCT |
| | | | gamma | | | | CAGAGACAAG |
| | | | inducible | | | | GATCTTGCCTT |
| | | | protein 30 | | | | ATTTTCAAATG |
| | | | | | | | GTGCTAAATTT |
| | | | | | | | AAATTC |
| 581. | H3106G04-3 | Cacnald | calcium | H3106G04 | Mm.9772 | Chromosome 14 | AGTGACTTCCA |
| | | | channel, | | | | CCTTTTAATGT |
| | | | voltage- | | | | CATTAAAAGCA |
| | | | dependent, L | | | | GGAGCTTAAAC |
| | | | type, alpha 1D | | | | TAAAAGCAGC |
| | | | subunit | | | | ATTCCA |
| 582. | L0701D10-3 | Arhgdib | Rho, GDP | L0701D10 | Mm.2241 | Chromosome 6 | ACATACATTTC |
| | | | dissociation | | | | ATCACCAATAT |
| | | | inhibitor (GDI) | | | | GTTTTATCTTA |
| | | | beta | | | | CCCCATCTCTC |
| | | | | | | | AGAGTGTTCCC |
| | | | | | | | TGCAA |
| 583. | H3137A02-3 | | Mus Musculus | H3137A02 | Mm.21657 | Chromosome 4 | TTTTTTGTATT |
| | | | 10 days neonate | | | | ATTGTGTTTTG |
| | | | cerebellum | | | | TGCTACTGTAG |
| | | | cDNA RIKEN | | | | TTTTGGTGTGG |
| | | | full-length | | | | CACTATTATAA |
| | | | enriched | | | | TTAAA |
| | | | library | | | | |
| | | | clone:B930053 | | | | |
| | | | B19 | | | | |
| | | | product:unknown | | | | |
| | | | EST, full | | | | |
| | | | insert sequence. | | | | |
| 584. | L0043D10-3 | A5310090O1Rik | RIKEN cDNA | L0043D10 | Mm.40298 | Chromosome 15 | CTTAGGGAGAC |
| | | | A530090O15 | | | | TACTAACATGG |
| | | | gene | | | | AGAGAATGCC |
| | | | | | | | GTGTATACCTC |
| | | | | | | | ACGTACTGTGT |
| | | | | | | | GCTTTA |
| 585. | H3087D06-3 | Etfl | eukaryotic | H3087D06 | Mm.3845 | Chromosome 18 | CATACATAGAA |
| | | | translation | | | | GCAAAATACTT |
| | | | termination | | | | TAACTGCTGTA |
| | | | factor 1 | | | | AACCTTCAAAA |
| | | | | | | | GTTAGTAGACG |
| | | | | | | | TGAGG |
| 586. | C0827E01-3 | | Mus musculus | C0827E01 | Mm.45759 | Chromosome 10 | ACTTCCTGCAA |
| | | | 15 days embryo | | | | TACATCCCAGT |
| | | | head cDNA | | | | AGGTACACCTA |
| | | | RIKEN full- | | | | GTTTACAATTT |
| | | | length enriched | | | | AAACTAGTTTG |
| | | | library, | | | | TGAAA |
| | | | clone:D930031 | | | | |
| | | | H08 | | | | |
| | | | product:unknown | | | | |
| | | | EST, full | | | | |
| | | | insert sequence. | | | | |
| 587. | H3053E01-3 | B130024B19Rik | RIKEN cDNA | H3053E01 | Mm.34557 | Chromosome 10 | GGAGGCACAT |
| | | | B130024B19 | | | | AATTCCAAGCA |
| | | | gene | | | | ATACAGGCTGT |
| | | | | | | | TAAAATATAAA |
| | | | | | | | TAATGGGAACT |
| | | | | | | | GTGATT |
| 588. | K0117C08-3 | BM222243 | ESTs | K0117C08 | Mm.221706 | Chromosome 1 | AAGCGTTAGG |
| | | | BM222243 | | | | AAGGAAATTTC |
| | | | | | | | CTGGAAGGAT |
| | | | | | | | AGGTTGTCTTC |
| | | | | | | | CTAGCAGCCTC |
| | | | | | | | GTCAATA |
| 589. | H3056D11-3 | Ptgfm | prostaglandin | H3056D11 | Mm.24807 | Chromosome 3 | TTTTTTAACTT |
| | | | F2 receptor | | | | CACTCATGACA |
| | | | negative | | | | ACAGAGGAAG |
| | | | regulator | | | | AAAGGAATTG |
| | | | | | | | AGGTTTAGGTA |
| | | | | | | | AGTTCTC |
| 590. | C0228C02-3 | 2510004L01Rik | RIKEN cDNA | C0228C02 | Mm.24045 | Chromosome 12 | AGGCATATCTC |
| | | | 2510004L01 | | | | ATAGAGCCTTA |
| | | | gene | | | | AGTTAGAATCT |
| | | | | | | | TACTCTTATGG |
| | | | | | | | AAGGAGTTATT |
| | | | | | | | TCCTA |
| 591. | H3144F09-3 | Rab711 | RAB7, member | H3144F09 | Mm.34027 | Chromosome 1 | GATCACCTCAT |
| | | | RAS oncogene | | | | TCCTCGACTGT |
| | | | family-like 1 | | | | GAGATGAGTTT |
| | | | | | | | ATGAAAAGAA |
| | | | | | | | TTAAAAGTGAG |
| | | | | | | | CACTTG |
| 592. | H3052B06-3 | Abcb1b | ATP-binding | H3052B06 | Mm.6404 | Chromosome 5 | TAAAGGTAACT |
| | | | cassette, sub- | | | | CCATCAAGATG |
| | | | family B | | | | AGAAGCCTTCC |
| | | | (MDR/TAP), | | | | GAGACTTTGTA |
| | | | member 1B | | | | ATTAAATGAAC |
| | | | | | | | CAAAA |
| 593. | L0273B08-3 | Tgif | TG interacting | L0273B08 | Mm.8155 | Chromosome 17 | GGCCAGGTATA |
| | | | factor | | | | TGTGTACCAGT |
| | | | | | | | GCTCTTCAAAG |
| | | | | | | | GGAGAACCATT |
| | | | | | | | AAAACCAACA |
| | | | | | | | TGGAAT |
| 594. | K0406A08-3 | Siat4c | sialytransferase | K0406A08 | Mm.2793 | Chromosome 9 | CCAAGAGATTA |
| | | | 4C (beta- | | | | TTTAACATTTT |
| | | | galactoside | | | | ATTTAATTAAG |
| | | | alpha-2,3- | | | | GGGTAGGAAA |
| | | | sialytransferase) | | | | ATGAATGGGCT |
| | | | | | | | GGTCCC |
| 595. | AF075136.1 | Sap30 | sin3 associated | AF075136 | Mm.118 | Chromosome 8 | AGTGAACGAA |
| | | | polypeptide | | | | AAAGACACCTT |
| | | | | | | | AACATGTTTCA |
| | | | | | | | TCTACTCAGTG |
| | | | | | | | AGGAACGACA |
| | | | | | | | AGAACAA |
| 596. | K0644H12-3 | Prkch | protein kinase | K0644H12 | Mm.8040 | Chromosome 12 | GATATTTATTG |
| | | | C, eta | | | | AGTGTCAAATA |
| | | | | | | | AAAAGGTGCC |
| | | | | | | | ATAATCTTCAG |
| | | | | | | | TAGCGTACACA |
| | | | | | | | GTAGAG |
| 597. | H3108A04-3 | Clu | clusterin | H3108A04 | Mm.200608 | Chromosome 14 | GTGTTACCAGA |
| | | | | | | | AGAAGTCTCTA |
| | | | | | | | AGGATAACCCT |
| | | | | | | | AAGTTTATGGA |
| | | | | | | | CACAGTGGCG |
| | | | | | | | GAGAAG |
| 598. | H3020F06-3 | Snx10 | sorting nexin 10 | H3020G06 | Mm.29101 | Chromosome 6 | TGTCTTTATTTT |
| | | | | | | | AATGCCAAAA |
| | | | | | | | GGAAGTGATTA |
| | | | | | | | TGCAGCTGTGT |
| | | | | | | | GTAGAGTTTCA |
| | | | | | | | GAGCA |
| 599. | L0066C05-3 | Uxs1 | UDP- | L0066C05 | Mm.201248 | Chromosome 1 | AGAACAAACT |
| | | | glucuronate | | | | GGAATTTTATT |
| | | | decarboxylase 1 | | | | CTGAAGCTTGC |
| | | | | | | | TTTAAAGACAC |
| | | | | | | | TGATGTGCCTA |
| | | | | | | | AACGCT |
| 600. | L0025F08-3 | Rgs19 | regulator of G- | L0025F08 | Mm.20156 | Chromosome 2 | TATGGTCTTTC |
| | | | protein | | | | AGTCACAGTGT |
| | | | signaling 19 | | | | AGTCACAGTGT |
| | | | | | | | CATCTTAATCT |
| | | | | | | | TACTGATCCAA |
| | | | | | | | TAAAAC |
| 601. | H3076F06-3 | Siat4a | sialytransferase | H3076F06 | Mm.248334 | Chromosome 15 | ATCCTCCTGAT |
| | | | 4A (beta- | | | | TGGTCTGAATG |
| | | | galactoside | | | | CATTTCCAATG |
| | | | alpha-2, 3- | | | | ATGTCAGGGA |
| | | | sialytransferase) | | | | TCAGCC |
| 602. | C0354G01-3 | | Mus musculus, | C0354G01 | Mm.259704 | Chromosome 13 | TAAGCCCTGTC |
| | | | Similar to IQ | | | | TTCTGGGAAAT |
| | | | motif | | | | ATCAGTTTTAA |
| | | | containing | | | | AGAGAACTTTT |
| | | | GTPase | | | | GTGCAATTCCA |
| | | | activating | | | | AATGA |
| | | | protein 2, clone | | | | |
| | | | IMAGE:35965 | | | | |
| | | | 08, mRNA, | | | | |
| | | | partial cds | | | | |
| 603. | C0191H09-3 | Atp6vla1 | ATPase, H+ | C0191H09 | Mm.29771 | No Chromosome | GGAAGATTAAT |
| | | | transporting | | | location | TTTCCAGGGAT |
| | | | V1 subunit A, | | | info available | TGTATCAATCA |
| | | | isoform 1 | | | | GGACCATTTTT |
| | | | | | | | GTGGGGCACTT |
| | | | | | | | GGGAC |
| 604. | H3050G04-3 | Dpp7 | dipeptidyl- | H3050G04 | Mm.21440 | Chromosome 2 | ATGTGATCTAC |
| | | | peptidase 7 | | | | AGTGGTGTGAC |
| | | | | | | | AACTTGCCTTG |
| | | | | | | | TATCTGATGGA |
| | | | | | | | CTGTCCAGATT |
| | | | | | | | TATGG |
| 605. | L0219A09-3 | Gatm | glycine | L0219A09 | Mm.29975 | Chromsome 2 | AAACGAAGTG |
| | | | amidinotransferase | | | | ACTTTCCATGA |
| | | | (L-arginine: | | | | ATGCCTTTAAC |
| | | | glycine amidino- | | | | ATTCTTGTGTC |
| | | | transferase) | | | | AACATTTGGTA |
| | | | | | | | CTAAAC |
| 606. | J0821E02-3 | AU040950 | expressed | J0821E02 | Mm.17580 | Chromosome 13 | AATACTCATTA |
| | | | sequence | | | | TGCTGTGTGGG |
| | | | AU040950 | | | | AATTTCCTGAT |
| | | | | | | | TACTAGAAGCT |
| | | | | | | | GACCTCTGCTA |
| | | | | | | | TCCTG |
| 607. | H3080a02-3 | Cbfb | core binding | H3080A02 | Mm.2018 | Chromsome 8 | GAATTATTATA |
| | | | factor beta | | | | AACAATAATGT |
| | | | | | | | GTTACAGAAGC |
| | | | | | | | TGATGCTGACC |
| | | | | | | | TTGTGTTACTG |
| | | | | | | | AGCAC |
| 608. | C0276B08-3 | Plscr1 | phospholipid | C0276B08 | Mm.14627 | Chromosome 9 | TTCTTGAGGTT |
| | | | scramblase 1 | | | | TAAGGACGAC |
| | | | | | | | AACTTTATGGA |
| | | | | | | | CCCTGAATGGA |
| | | | | | | | AACTGAGGAA |
| | | | | | | | TCACAAG |
| 609. | C0279E04-3 | Srd5a21 | steroid 5 alpha- | C0279E04 | Mm.86611 | Chromosome 5 | GTCACATGCCA |
| | | | reductase 2-like | | | | ATAAAAACAG |
| | | | | | | | GAAACTCTGAA |
| | | | | | | | AATAATATGAA |
| | | | | | | | TGTACAGTATC |
| | | | | | | | AGACCG |
| 610. | K043D04-3 | Pgd | phosphogluconate | K0434D04 | Mm.252080 | No Chromosome | CCCTATTGCAA |
| | | | dehydrogenase | | | location | ATTGATTTGTT |
| | | | | | | info available | TTCCCTTAACC |
| | | | | | | | CTGTTCCCTTT |
| | | | | | | | TAACCCCGGCT |
| | | | | | | | TTTTT |
| 611. | C0174H01-3 | Ddx21 | DEAD (Asp- | C0174H01 | Mm.25264 | Chromosome 10 | CATTGCATCGT |
| | | | Glu-Ala-Asp) | | | | TTTCCAACATA |
| | | | box polypeptide | | | | CTTTTAGATTT |
| | | | 21 | | | | ACAAAGTAAA |
| | | | | | | | ACCAACCATGG |
| | | | | | | | ATCTGC |
| 612. | H3085A07-3 | BG070224 | ESTs | H3085A07 | Mm.173217 | Chromosome 17 | TTGAGAAATTA |
| | | | BG070224 | | | | AAAACAAATA |
| | | | | | | | TCCAAAATCGA |
| | | | | | | | CTTTTCCTCAA |
| | | | | | | | GGCTATGTGCT |
| | | | | | | | TCGTCC |
| 613. | K0208E10-3 | Mmab | methylmalonic | K0208E10 | Mm.105182 | Chromosome 5 | ACGACTCTTGT |
| | | | aciduria | | | | TAATGTGCGTT |
| | | | (cobalamin | | | | TCTCATGGAGT |
| | | | deficiency) type | | | | AATTTTCAGAG |
| | | | B homolog | | | | CCTGAACTTGT |
| | | | (human) | | | | AGCAC |
| 614. | H3006F10-3 | Cops2 | COP9 | H3006F10 | Mm.3596 | Chromosome 2 | GTTGGTGTGTC |
| | | | (constitutive | | | | CTGAAAGGGA |
| | | | photomorphogenic) | | | | TGGAGTTATGG |
| | | | homolog, | | | | CAGAAGTGCTT |
| | | | subunit 2 | | | | TTGTGATCAAC |
| | | | (Arabidopisi- | | | | TGGTTT |
| | | | thaliana) | | | | |
| 615. | C0108A10-3 | Nek6 | NIMA (never in | C0108A10 | Mm.143818 | Chromosome 2 | CAGAAAACTC |
| | | | mitosis gene a)- | | | | AAGTCATGGAC |
| | | | related | | | | TATGCGAGTCA |
| | | | expressed | | | | AGAATTAAAAT |
| | | | kinase 6 | | | | ACAACTGTATT |
| | | | | | | | ATGTGC |
| 616. | H3028H10-3 | Ppic | peptidylprolyl- | H3028H10 | Mm.4587 | Chromosome Multiple | AAATTTCTCAT |
| | | | isomerase C | | | Mappings | TTAATTTTCCA |
| | | | | | | | GTCTCGATTGC |
| | | | | | | | AGTAACAAAG |
| | | | | | | | TCAACCACACA |
| | | | | | | | GTCAGA |
| 617. | H3121E08-3 | Ralgds | ral guanine | H3121E08 | Mm.5236 | Chromosome 2 | GGAGGAAGAC |
| | | | nucleotied | | | | AACTGAACATT |
| | | | dissociation | | | | TGTATAAAACG |
| | | | stimulator | | | | TAAAAGTTTA |
| | | | | | | | CTGATTGGGGT |
| | | | | | | | GGGACA |
| 618. | L0266H12-3 | Opal | optic atrophy 1 | L0266H12 | Mm.31402 | Chromosome 16 | CAGCAGCTTAC |
| | | | homolog | | | | AAACACTGAA |
| | | | (human) | | | | GTTAGGCGACT |
| | | | | | | | AGAGAAAAAC |
| | | | | | | | GTTAAAGAGGT |
| | | | | | | | ATTAGAA |
| 619. | K0635G02-3 | 2310046K10Rik | RIKEN cDNA | K0635G02 | Mm.68134 | Chromosome 14 | GAGAAATGTTA |
| | | | 2310046K10 | | | | GTAAAATGGTA |
| | | | gene | | | | AAAGGGAATC |
| | | | | | | | ACGTGACATTC |
| | | | | | | | AGGGTAGGAA |
| | | | | | | | GAGCTTG |
| 620. | L0704C05-3 | 2613018G18Rik | RIKEN cDNA | L0704C05 | Mm.180776 | Chromosome 3 | TCAGGAAAAA |
| | | | 2610318G18 | | | | TGTCATAAGCC |
| | | | gene | | | | ATCTGGTAAGT |
| | | | | | | | TTTCTTAAAGG |
| | | | | | | | ATGTTGTTAAG |
| | | | | | | | AAGTCC |
| 621. | C0303D10-3 | | UNKNOWN | C0303D10 | Data not found | No Chromosome | CAAAACAAAT |
| | | | C0303D10 | | | location | ACATATTATAA |
| | | | | | | info available | AATAAAAGAA |
| | | | | | | | AAGGCGTGAT |
| | | | | | | | AAATGGATGTG |
| | | | | | | | ACAAAATT |
| 622. | K0605C04-3 | BM240648 | ESTs | K0605C04 | Mm.265969 | Chromosome 15 | GTAGGGAAAA |
| | | | MN240648 | | | | TATGTCCATAG |
| | | | | | | | GTTTTAGGAAA |
| | | | | | | | CACTTAGCCTT |
| | | | | | | | TAATATACTGG |
| | | | | | | | TTGTAG |
| 623. | H3071G06-3 | BG069012 | ESTs | H3071G06 | Mm.26430 | Chromsome 4 | GTATACAGATG |
| | | | BG069012 | | | | GTAGTTAGAAA |
| | | | | | | | TACTGGATGAA |
| | | | | | | | CTGATCAGTTA |
| | | | | | | | TTGTGTGTAGA |
| | | | | | | | AAGTG |
| 624. | C0600A01-3 | Coro2a | coronin, actin | C0600A01 | Mm.171547 | Chromosome 4 | TTGTATCCCAA |
| | | | binding protein | | | | AGGGAAACGG |
| | | | 2A | | | | GAATCAAGAT |
| | | | | | | | ACGGACCTATG |
| | | | | | | | CTTTTCATATG |
| | | | | | | | AAACCGT |
| 625. | NM_007679.1 | Cebpd | CCAAT/enhancer | NM_007679 | Mm.4639 | Chromosome 16 | TGCAGCTAAGG |
| | | | binding | | | | TACATTTGTAG |
| | | | protein | | | | AAAAGACATTT |
| | | | (C/EBP), data | | | | CCGACAGACTT |
| | | | | | | | TTGTAGATAAG |
| | | | | | | | AGGAA |
| 626. | H3048A01-3 | Kras2 | Kirsten rat | H3048A01 | Mm.31530 | Chromosome 6 | GGCAATGGAA |
| | | | sarcoma | | | | AATGTTGAAAT |
| | | | oncogene 2, | | | | CCATTCGTTT |
| | | | expressed | | | | CCATGTTAGCT |
| | | | | | | | AAATTACTGTA |
| | | | | | | | AGATCC |
| 627. | C0267D12-3 | Tpp2 | tripeptidyl- | C0267D12 | Mm.28867 | Chromosome 1 | CCCCAAAGAA |
| | | | peptidase II | | | | AACTGGAAAA |
| | | | | | | | ATTGTTTTCCA |
| | | | | | | | CTCCTGAAATT |
| | | | | | | | TCTTGGATGGG |
| | | | | | | | CCCCCTG |
| 628. | J1012C06-3 | AU041997 | ESTs | J1012C06 | Mm.181004 | Chromosome 5 | CCAGACAGTGT |
| | | | AU041997 | | | | ATTCTTCGGAC |
| | | | | | | | AAATGGTGTGA |
| | | | | | | | AAGTGAAATA |
| | | | | | | | AGAATTCATAA |
| | | | | | | | TGTAAC |
| 629. | L0072f04-3 | Vav2 | Vav2 oncongene | L0072F04 | Mm.179011 | Chromosome 2 | AGCAAAAGTA |
| | | | | | | | TGTATATTTTA |
| | | | | | | | GCTTGTCATGA |
| | | | | | | | AATGTCAACGA |
| | | | | | | | AGGACACTGA |
| | | | | | | | GAAAGAG |
| 630. | L0836H04-3 | C030038J10Rik | RIKEN cDNA | L0836H04 | Mm.212874 | Chromosome 6 | TAGAATGGGA |
| | | | C030038J10 | | | | ATTTTCTGTCT |
| | | | gene | | | | CATAGTGACAT |
| | | | | | | | ATTGCTATGTT |
| | | | | | | | TAACAGTGAAC |
| | | | | | | | ACTCAC |
| 631. | K0614A10-3 | Sh3kbp1 | SH3-domain | K0614A10 | Mm.254904 | Chromosome X | TGACGGTATAT |
| | | | kinase binding | | | | TTGCAAAAAG |
| | | | protein 1 | | | | AGAAAGAAAA |
| | | | | | | | ATCTGGTATTT |
| | | | | | | | GCAATGATCTG |
| | | | | | | | TGCCTTC |
| 632. | H3156B08-3 | 6620401D04Rik | RIKEN cDNA | H3156B08 | Mm.86150 | Chromosome 16 | GAAATATCATT |
| | | | 6620401D04 | | | | TGTAGCTTTAA |
| | | | gene | | | | GGCTAGAAAA |
| | | | | | | | TGAAAAAGAA |
| | | | | | | | TCCAAGCCAGT |
| | | | | | | | AGAAGGC |
| 633. | C0334C11-3 | B230339H12Rik | RIKEN cDNA | C0334C11 | Mm.275985 | Chromosome 8 | ATACCAGGAA |
| | | | B230339H12 | | | | AATAAAAGTA |
| | | | gene | | | | CCAGTAAGGA |
| | | | | | | | AGCATCAAATC |
| | | | | | | | AAGATGTCATA |
| | | | | | | | GTCAGTGG |
| 634. | H3103G05-3 | BG071839 | ESTs | H3103G05 | Mm.17827 | Chromosome 3 | CAGTGTAAATA |
| | | | BG071839 | | | | TAGCATATGGT |
| | | | | | | | TAGGTGGTGAG |
| | | | | | | | AAAATGATCTT |
| | | | | | | | GAGACTGATA |
| | | | | | | | AGAATC |
| 635. | C0205H05-3 | 1600010D10Rik | RIKEN cDNA | C0205H05 | Mm.86385 | Chromosome 3 | ATCCTTTAGAT |
| | | | 1600010D10 | | | | GTTAGTACAGT |
| | | | gene | | | | GTTTATGAGAA |
| | | | | | | | AACTGTTACTA |
| | | | | | | | GAAGCTGAAG |
| | | | | | | | AACAGC |
| 636. | L0513G12-3 | Qk | quaking | L0513G12 | Mm.2655 | Chromsome 17 | AGTGTTCTATA |
| | | | | | | | TGTGTAAATTA |
| | | | | | | | GTATTTTCAAC |
| | | | | | | | TGGAAAATGTT |
| | | | | | | | GGCTGGTGCAA |
| | | | | | | | AAGGC |
| 637. | C0100E08-3 | Pdap1 | PDGFA | C0100E08 | Mm.188851 | Chromosome Multiple | GTCTGGGCTAG |
| | | | associated | | | Mappings | TGCCCGTTTTT |
| | | | protein 1 | | | | AACCCTACCCA |
| | | | | | | | TTGATCATTTC |
| | | | | | | | AAGAAACCTCT |
| | | | | | | | GGTTA |
| 638. | J0055B04-3 | | transcribed | J0055B04 | Mm.228682 | Chromsome 16 | TGTAAGACCAT |
| | | | sequence with | | | | TTCTAAATTGC |
| | | | strong | | | | TGGTAATAGAA |
| | | | similarity to | | | | ACTCATGGCAG |
| | | | protein | | | | TAAAAATGTAA |
| | | | pir:S12207 | | | | CCTCG |
| | | | (M. musculus) | | | | |
| | | | S12207 | | | | |
| | | | hypothetical | | | | |
| | | | protein (B2 | | | | |
| | | | element)- | | | | |
| | | | mouse | | | | |
| 639. | J0008D10-3 | Mbp | myelin basic | J0008D10 | Mm.2992 | Chromosome 18 | ACTGGAATAG |
| | | | protein | | | | GAATGTGATGG |
| | | | | | | | GCGTCGCACCC |
| | | | | | | | TCTGTAAATGT |
| | | | | | | | GGGAATGTTTG |
| | | | | | | | TAACTT |
| 640. | K0319D09-3 | Mtm1 | X-linked | K0319D09 | Mm.28580 | Chromosome X | TCTACTAGAAG |
| | | | myotubular | | | | GGTTAAAAGCC |
| | | | myopathy gene | | | | ATATGAATGCA |
| | | | 1 | | | | AGAAATCATTT |
| | | | | | | | GAGGCTTAAA |
| | | | | | | | ATGCTG |
| 641. | C0243H05-3 | Galnt7 | UDP-N-acetyl- | C0243H05 | Mm.62886 | Chromosome 8 | GGACACCATTT |
| | | | alpha-D- | | | | TTCATGTTAAA |
| | | | galactosamine: | | | | TAGATTTTAAC |
| | | | polypeptide N- | | | | CTCGTATCTAT |
| | | | acetylgalactosa | | | | GCATAGGCTAA |
| | | | minyltransferase | | | | GGTGG |
| | | | 7 | | | | |
| 642. | L0841H10-3 | BM116846 | ESTs | L0841h10 | Mm.65363 | Chromosome 2 | TAGATAAAGCC |
| | | | MN116846 | | | | CGTATGAGAA |
| | | | | | | | GAGAAAACCA |
| | | | | | | | AATTAATCCAC |
| | | | | | | | TTCAGCAAAAA |
| | | | | | | | GAAAGCC |
| 643. | K0334D05-3 | Ccn1 | cyclin D1 | K0334D05 | Mm.22288 | Chromosome 7 | CAATGTCAGAC |
| | | | | | | | TGCCATGTTCA |
| | | | | | | | AGTTTTAATTT |
| | | | | | | | CCTCATAGAGT |
| | | | | | | | GTATTTACAGA |
| | | | | | | | TGCCC |
| 644. | L0209B01-3 | | L0209B01-3 | L0209B01 | | No Chromosome | CTTTGGGGGGG |
| | | | NIA Mouse | | | location | GTTTTGGAAAA |
| | | | Newborn Ovary | | | info available | CCGGTTTTTTC |
| | | | cDNA Library | | | | GGGGGGGTTTC |
| | | | Mus musculus | | | | CTTTTGGGGGG |
| | | | cDNA clone | | | | TTTTT |
| | | | L0209B01 3′, | | | | |
| | | | MRNA | | | | |
| | | | sequence | | | | |
| 645. | K0151H10-3 | BB129550 | EST BB129550 | K0151H10 | Mm.283461 | No Chromosome | GCCATACAGCT |
| | | | | | | location | TATATTTGTAC |
| | | | | | | info available | TGGTATGTCCA |
| | | | | | | | GAAATCATGG |
| | | | | | | | AGGAAAGAAA |
| | | | | | | | AGTAAAA |
| 646. | L0505B11-3 | Ammecr1 | Alport | L0505B11 | Mm.143724 | Chromosome X | TGGTGTTTTGA |
| | | | syndrome, | | | | TTACAGTGAGA |
| | | | mental | | | | CATCACAGGTT |
| | | | retardation, | | | | ATCTAAAAGCC |
| | | | midface | | | | CTTCGTTATAA |
| | | | hypoplasia and | | | | CCAGC |
| | | | eliptocytosis | | | | |
| | | | chromosomal | | | | |
| | | | regoion gene 1 | | | | |
| | | | homolog | | | | |
| | | | (human) | | | | |
| 647. | L0944C06-3 | BM120800 | ESTs | L0944C06 | Mm.217092 | Chromosome 3: not placed | TATTTGGTGGT |
| | | | BM120800 | | | | AAAGAATATG |
| | | | | | | | GTTGAAAATTG |
| | | | | | | | TCATCCACATG |
| | | | | | | | CATGCATCAAG |
| | | | | | | | TAACAC |
| 648. | J0027C07-3 | Mrps25 | mitochondrial | J0027C07 | Mm.87062 | Chromosome 6 | CGAGGAGTTAT |
| | | | ribosomal | | | | TAGGGAGAAT |
| | | | protein S25 | | | | CATGGAGCCAC |
| | | | | | | | ATAAGAAAAT |
| | | | | | | | CTTGGGCAAGA |
| | | | | | | | AAAGAGG |
| 649. | L0855B04-3 | Wdr26 | WD repeat | L0855B04 | Mm.21126 | Chromosome 1 | TGGTGACAGG |
| | | | domain 26 | | | | ATTACGTGAAA |
| | | | | | | | ATCTCTGACAT |
| | | | | | | | TGTGATAAACT |
| | | | | | | | GGATAAAGGCT |
| | | | | | | | TAAGAG |
| 650. | H3060H05-3 | | Mus musculus | H3060H05 | Mm.11778 | Chromosome 1 | ACCCTTTGCTT |
| | | | cDNA clone | | | | AAATAGTGGG |
| | | | MGC:28609 | | | | AAAACGTGAA |
| | | | IMAGE:42185 | | | | TGTTTAGCATA |
| | | | 51, complete | | | | ATATAAAAAC |
| | | | cds | | | | ATGCAGGC |
| 651. | K0330609-3 | 5830461H18Rik | RIKEN cDNA | K0330G09 | Mm.261448 | Chromosome 14 | GTTGGACTCTA |
| | | | 5830461H18 | | | | ATACAACTGAC |
| | | | gene | | | | CATTGAAAAAT |
| | | | | | | | GAACAACGGC |
| | | | | | | | TTATTGTTTTG |
| | | | | | | | TAAACAG |
| 652. | L0803E07-3 | Dpys14 | dihydropyrmid- | L0803E07 | Mm.250414 | Chromosome 7 | TTCTACAAAG |
| | | | inase-like 4 | | | | TGTGTTTCTAT |
| | | | | | | | AGGATTACTAG |
| | | | | | | | AGTAGCGGTTT |
| | | | | | | | TGTACTGTGAG |
| | | | | | | | GAAAC |
| 653. | L0283B01-3 | Ivns1abp | influenza virus | L0283B01 | Mm.33764 | No Chromosome | TAGATAACAGT |
| | | | NS1A binding | | | location | GACTATTGACG |
| | | | protein | | | info available | ATTTTAGTAAA |
| | | | | | | | AGAAAGTTGA |
| | | | | | | | CATGCGTACCG |
| | | | | | | | CTACCT |
| 654. | L0065G02-3 | 6530401D17Rik | RIKEN cDNA | L0065G02 | Mm.27579 | Chromosome X | GGGGGGACAG |
| | | | 6530401D17 | | | | TTAATATCGTT |
| | | | gene | | | | TGTTAGATACC |
| | | | | | | | ATAAGTGGTGG |
| | | | | | | | AAATAAAGTG |
| | | | | | | | ACTAAAG |
| 655. | C0949A06-3 | | Mus musculus | C0949A06 | Mm.71633 | Chromosome 13 | AAAGAGGAAA |
| | | | 0 day neonate | | | | CTGTCCTATTT |
| | | | skin cDNA, | | | | CTCAACTGATA |
| | | | RIKEN full- | | | | AGTACTCCTGG |
| | | | length enriched | | | | TAAGATGTAAT |
| | | | library | | | | ATTTGC |
| | | | clone:4632424 | | | | |
| | | | N07 | | | | |
| | | | product:unknown | | | | |
| | | | EST, full | | | | |
| | | | insert sequence. | | | | |
| 656. | H3100C11-3 | BG071548 | ESTs | H3100C11 | Mm.173983 | Chromosome Un: not | CAAATGTACTG |
| | | | BG071548 | | | placed | AGAAACAAAA |
| | | | | | | | TCATGAACGAC |
| | | | | | | | CTTGAAATCAC |
| | | | | | | | CTTCTTATTTC |
| | | | | | | | AGCTCC |
| 657. | C0142H08-3 | 3110020O18Rik | RIKEN cDNA | C0142H08 | Mm.117055 | Chromosome 5 | AACATAAATCA |
| | | | 3110050O18 | | | | AAATATACTTA |
| | | | gene | | | | GGAATATTTAC |
| | | | | | | | AATTAAACATG |
| | | | | | | | ATGTTTTAAAC |
| | | | | | | | TTAGT |
| 658. | L0945G09-3 | Bcl2111 | BCL2-like 11 | L0945G09 | Mm.141083 | Chromosome 2 | GACTATTTATT |
| | | | (apoptosis | | | | AGATTAGAAA |
| | | | facilitator) | | | | GTCATGTTTCA |
| | | | | | | | CTCGTCAACTG |
| | | | | | | | AGCCAAATGTC |
| | | | | | | | TCTGTG |
| 659. | L0848H06-3 | E130318E12Rik | RIKEN cDNA | L0848H06 | Mm.198119 | Chromosome 1 | ACAAACACAT |
| | | | E130318E12 | | | | GAAAAAATCA |
| | | | gene | | | | AGTAGGAACT |
| | | | | | | | GGAGAAACGT |
| | | | | | | | CTCACAGTTAA |
| | | | | | | | GAATGTTTG |
| 660. | K0617B02-3 | Bmp2k | BMP2 | K0617B02 | Mm.6156 | Chromosome 5 | AATTCACAGAT |
| | | | inducible | | | | GGCTTACATTT |
| | | | kinase | | | | ATGTAAAGAAT |
| | | | | | | | TCCTGTAAGGC |
| | | | | | | | ACTCATGTTTG |
| | | | | | | | ACATC |
| 661. | C0203D07-3 | Pftk1 | PFTAIRE | C0203D07 | Mm.6456 | Chromosome 5 | TATACCAAACT |
| | | | protein kinase 1 | | | | GAAAACGTTTA |
| | | | | | | | AATCTCAAATG |
| | | | | | | | AAGTAAGCAA |
| | | | | | | | GGTTTTGTTCT |
| | | | | | | | CCCTGC |
| 662. | L0267A02-3 | 2210409B22Rik | RIKEN cDNA | L0267A02 | Mm.30015 | Chromosome 4 | TAGCCATTTAG |
| | | | 2210409B22 | | | | GAGATGTCCCT |
| | | | gene | | | | TCAAAGTGACG |
| | | | | | | | TGATGATGGAC |
| | | | | | | | TTGCACTTGGG |
| | | | | | | | AATCA |
| 663. | J0086F05-3 | | transcribed | J0086F05 | Mm.31079 | No Chromosome | GCTCAGCTTAG |
| | | | sequence with | | | location | GCTAGACTTTG |
| | | | moderate | | | info available | ACCAGGTAAG |
| | | | similarity to | | | | CAGAAGAAAT |
| | | | protein | | | | GAGAAACAAA |
| | | | sp:P00722 (E. | | | | ACTCAGCA |
| | | | coli) | | | | |
| | | | BGAL_ECOLI | | | | |
| | | | Beta- | | | | |
| | | | galactosidase | | | | |
| | | | (Lactase) | | | | |
| 664. | C06606A03-3 | Rps23 | ribosomal | C0606A03 | Mm.295618 | Chromosome X | TATCACTGGAA |
| | | | protein S23 | | | | TATTGAAAGGT |
| | | | | | | | TGTATGTAGTA |
| | | | | | | | TGGGAGATCA |
| | | | | | | | ACTTTCTTCCC |
| | | | | | | | TAAGGT |
| 665. | L0902D02-3 | Ncoaoip | nuclear receptor | L0902D02 | Mm.171323 | Chromosome 4 | ACTGCTGAGAA |
| | | | coactivator 6 | | | | AAACAAAATTC |
| | | | interacting | | | | ACTACATACCT |
| | | | protein | | | | CAATAGTTATT |
| | | | | | | | TACCATGAGAT |
| | | | | | | | TGGCG |
| 666. | H3060C12-3 | BG067974 | ESTs | H3060C12 | Mm.173106 | Chromosome 1 | GAAGGAAATG |
| | | | BG067974 | | | | CAAACACCTTT |
| | | | | | | | GAACTTCAATT |
| | | | | | | | CTTTCAGTAGG |
| | | | | | | | AAAACAAGAA |
| | | | | | | | TTGTCCC |
| 667. | C0611E01 | Tor3a | torsin family 3, | C0611E01 | Mm.206737 | Chromosome 1 | AGAAAAACAC |
| | | | member A | | | | TAAACTCCAAA |
| | | | | | | | TTAGTATAATA |
| | | | | | | | ACGAGCACTAC |
| | | | | | | | AGTGGTGAAA |
| | | | | | | | AAGCTCC |
| 668. | U54984.1 | Mmp14 | matrix | U54984 | Mm.19945 | Chromosome 14 | AAAGGAATCTT |
| | | | metalloproteinase | | | | AAGAGTGTAC |
| | | | 14 | | | | ATTTGGAGGTG |
| | | | (membrane- | | | | GAAAGATTGTT |
| | | | inserted) | | | | CAGTTTACCCT |
| | | | | | | | AAAGAC |
| 669. | H3089F08-3 | 0610013E23Rik | RIKEN cDNA | H3089F08 | Mm.182061 | Chromosome 11 | GAAATGGATTT |
| | | | 0610013E23 | | | | TGAGGCTTTGA |
| | | | gene | | | | AAATGAAAAT |
| | | | | | | | GGCTAGTQTCT |
| | | | | | | | CAAAGATGTCA |
| | | | | | | | GTATCC |
| 670. | K0633C04-3 | Ebi2 | Epstein-Barr | K0633C04 | Mm.265618 | Chromosome 14 | ACTATTTCTTG |
| | | | virus induced | | | | TCAATAGTTTG |
| | | | gene 2 | | | | GCAAAAGACG |
| | | | | | | | ACTAATTGCAC |
| | | | | | | | TGTATATTGCC |
| | | | | | | | AGTGTA |
| 671. | J0943E09-3 | Nup62 | nucleoporin 62 | J0943E09 | Mm.22687 | Chromosome 7 | TCCTCTAAAGA |
| | | | | | | | TGTGTCTTATA |
| | | | | | | | TACATGATTGT |
| | | | | | | | CATTGGTGGGC |
| | | | | | | | TCAAACAATAA |
| | | | | | | | GGGTG |
| 672. | L0267D03-3 | Dcn | decorin | :0267D03 | Mm.56769 | Chromosome 10 | TTGGAAACTAC |
| | | | | | | | AAGTAACCCTC |
| | | | | | | | AGACGGCCTA |
| | | | | | | | ATTCTTATAAT |
| | | | | | | | CCGGAAAAAC |
| | | | | | | | ACCCCAA |
| 673. | L0250B09-3 | 111031E24Rik | RIKEN cDNA | L0250B09 | Mm.34356 | Chromosome 8 | GTGTGATAATC |
| | | | 1110031E24 | | | | TTTTCATGTTTT |
| | | | gene | | | | CTAGAGCAAA |
| | | | | | | | GACAAAGCAG |
| | | | | | | | TTACTCTTCTA |
| | | | | | | | TCGCAA |
| 674. | L0915B12-3 | Etv3 | ets variant gene | L0915B12 | Mm.34510 | Chromosome 3 | GGCTTTAGAGA |
| | | | 3 | | | | AAACTTCGGTC |
| | | | | | | | TTCAAAGAACT |
| | | | | | | | CTTCTAATTAG |
| | | | | | | | TTCCTTCTTGG |
| | | | | | | | AAAAA |
| 675. | NM_009403.1 | Tnfsf8 | tumor necrosis | NM_009403 | Mm.4664 | Chromosome 4 | AAAGTAGGAG |
| | | | factor (ligand) | | | | ATGAGATTTAC |
| | | | superfamily, | | | | ATTTCCCCAAT |
| | | | member 8 | | | | ATTTTCTTCAA |
| | | | | | | | CTCAGAAGAC |
| | | | | | | | GAGACTG |
| 676. | C0308F04-3 | 2700064H14Rik | RIKEN cDNA | C0308F04 | Mm.24730 | Chromosome 2 | AGTCCTCTGCA |
| | | | 2700064H14 | | | | TGTTTCCAAAA |
| | | | gene | | | | TTTCCTTTACA |
| | | | | | | | TGAAGGCTATA |
| | | | | | | | TTGGATCAGAG |
| | | | | | | | CTTAC |
| 677. | C0288G12-3 | 6030400A10Rik | RIKEN cDNA | C0288G12 | Mm.159840 | Chromosome 5 | AAGAATAAAT |
| | | | 6030400A10 | | | | CACTTGAAATC |
| | | | gene | | | | ATACTGTTTTT |
| | | | | | | | GGAAATCCAA |
| | | | | | | | ACTGTTTAAAG |
| | | | | | | | AAAACTT |
| 678. | H3005A11-3 | Fancd2 | Fanconi | H3005A11 | Mm.291487 | Chromosome 6 | GTTAGATGCCA |
| | | | anemia, | | | | TTGAAGGGGA |
| | | | complementation | | | | AATAACTTTGG |
| | | | group D2 | | | | CTAATAGCTTG |
| | | | | | | | GAAAACTCAGT |
| | | | | | | | ACTAAG |
| 679. | H3121H07-3 | 2810405I11Rik | RIKEN cDNA | H3121H07 | Mm.73777 | Chromosome 18 | AGCAGATATGT |
| | | | 2810405I11 | | | | GACTTCTCATA |
| | | | gene | | | | TACACAGTTAC |
| | | | | | | | GCTAACTCAGG |
| | | | | | | | TGTATGATGAA |
| | | | | | | | TACAG |
| 680. | K0124A06-3 | BM222608 | ESTs | K0124A06 | Mm.221709 | Chromosome 19 | TGTCTATGGGA |
| | | | BM222608 | | | | GAAGTAATAG |
| | | | | | | | CCTGAAATAAG |
| | | | | | | | ATAAGGCTCAA |
| | | | | | | | ACAAACACTAC |
| | | | | | | | TTACTT |
| 681. | NM_010835.1 | Msx1 | homeo box, | NM_010835 | Mm.259122 | Chromosome 5 | GGGAAGAAAA |
| | | | msh-like 1 | | | | AGAATTGGTCG |
| | | | | | | | GAAGATGTTCA |
| | | | | | | | GGTTTTTCGAG |
| | | | | | | | TTTTTTCTAGA |
| | | | | | | | TTTACA |
| 682. | K0134C07-3 | Falz | fetal Alzheimer | K0134C07 | Mm.218530 | Chromosome 11 | CTTGAAGAAA |
| | | | antigen | | | | AGTATATCACG |
| | | | | | | | TAGGCATAGAT |
| | | | | | | | GAGAAAGCCG |
| | | | | | | | TTTGATCAAGT |
| | | | | | | | CTGGTTA |
| 683. | K0424H02-3 | Pfkp | phosphofructok- | K042H02 | Mm.108076 | Chromosome 13 | TCCTTCAGTCA |
| | | | inase, platelet | | | | GATATCTGTCC |
| | | | | | | | CAGAGAAAGG |
| | | | | | | | AAAATAAGGA |
| | | | | | | | GCATGGTAAG |
| | | | | | | | AAATGAGT |
| 684. | H3153G06-3 | 8030446C20Rik | RIKEN cDNA | H3153G06 | Mm.204920 | Chromosome 13 | TATGGAATGGA |
| | | | 8030446C20 | | | | GAAATAAATA |
| | | | gene | | | | CATCTGTGTTG |
| | | | | | | | AAGAACCTTTT |
| | | | | | | | GATGGAACTA |
| | | | | | | | ATACCGC |
| 685. | H3071C09-3 | BG068971 | ESTs | H3071C09 | Mm.162073 | Chromosome 6 | AGGTCAATGTT |
| | | | BG068971 | | | | AAGTTTTCTGA |
| | | | | | | | GTTTAATATAT |
| | | | | | | | AGTTAGGGTGA |
| | | | | | | | AAGACTTAGCA |
| | | | | | | | CACGG |
| 686. | L0243B07-3 | | Possibly | L0243B07 | Data not found | No Chromosome | AATGCTTAACT |
| | | | intronic in | | | location | TTGAGTCACAC |
| | | | U008124- | | | info available | TGTTTACCCTT |
| | | | L0243B07 | | | | CCTATGAGGTT |
| | | | | | | | GCATTTTGACA |
| | | | | | | | ACAAC |
| 687. | C0143D11-3 | Ii | Ia-associated | C0143D11 | Mm.248267 | Chromosome 18 | TAAAGGGAAC |
| | | | invariant chain | | | | CCCCATTTCTG |
| | | | | | | | ACCCATTAGTA |
| | | | | | | | GTCTTGAATGT |
| | | | | | | | GGGGCTCTGAG |
| | | | | | | | ATAAAG |
| 688. | L0512A02-3 | Snx5 | sorting nexin 5 | L0512A02 | Mm.20847 | No Chromosome | CCCCTTTTGT |
| | | | | | | location | AACTGGGATAT |
| | | | | | | info available | AAATCCTTGAA |
| | | | | | | | AGAAAGGAGA |
| | | | | | | | ATTTAGAGTTT |
| | | | | | | | TGCCCC |
| 689. | K0112C06-3 | Atp8a1 | ATPase, | K0112C06 | Mm.200366 | Chromosome 5 | GTCAGTGAGTT |
| | | | aminophospholipid | | | | GGTTTCCTTTC |
| | | | transporter | | | | CATCAGGAAA |
| | | | (APLT), class I, | | | | AATGGATTCTG |
| | | | type 8A, | | | | TAAAGAGTCA |
| | | | member 1 | | | | GGGCGTT |
| 690. | H3053A01-3 | Tnfsf13b | tumor necrosis | H3053A01 | Mm.28835 | Chromosome 8 | GAAAGCCGTC |
| | | | factor (ligand) | | | | AGCGAAAGTTT |
| | | | superfamily, | | | | TCTCGTGACCC |
| | | | member 13b | | | | GTTGAATCTGA |
| | | | | | | | TCCAAACCAGG |
| | | | | | | | AAATAT |
| 691. | C0668F08-3 | Atp6ap2 | ATPase, H+ | C0668F08 | Mm.25148 | Chromosome X | GAAATATGTTA |
| | | | transporting, | | | | ACTAAGAGCA |
| | | | lysosomal | | | | GCCCAAAAAT |
| | | | accessory | | | | ACTGGATATGC |
| | | | protein 2 | | | | TTATCCAATCG |
| | | | | | | | CTTAGTT |
| 692. | K0417E05-3 | Osmr | oncostatin M | K0417E05 | Mm.10760 | Chromosome 15 | GTATACAATGC |
| | | | receptor | | | | TATTTTTAGGT |
| | | | | | | | TAAGGCCTAAA |
| | | | | | | | CTTCTGAAGAT |
| | | | | | | | CTTGGTAACAG |
| | | | | | | | CAGAG |
| 693. | NM_010872.1 | Birclb | baculoviral IAP | NM_010872 | Mm.89961 | Chromosome 13 | GGATGAAGTG |
| | | | repeat- | | | | GAAGATTACTG |
| | | | containing 1b | | | | GCAGGTCCAA |
| | | | | | | | AAACCTGATTT |
| | | | | | | | TCTAGTACATT |
| | | | | | | | TCACTCT |
| 694. | L0262G06-3 | Cfh | complement | L0262G06 | Mm.8655 | Chromosome 1 | TTCAATCAAGA |
| | | | component | | | | AAGTAGATGTA |
| | | | factor h | | | | AGTTCTTCAAC |
| | | | | | | | ATCTGTTTCTA |
| | | | | | | | TTCAGAACTTT |
| | | | | | | | CTCAG |
| 695. | J0249F06-3 | 2210023K21Rik | RIKEN cDNA | J0249F06 | Mm.28890 | No Chromosome | AAATTTTCTTA |
| | | | 2210023K21 | | | location | AAGCTATGAAC |
| | | | | | | info available | TCTGACTTTTG |
| | | | | | | | ATTTTGTGTTT |
| | | | | | | | CCATTTAGTAG |
| | | | | | | | AAACT |
| 696. | C0170A02-3 | Serpinb9 | serine (or) | C0170A02 | Mm.3368 | Chromosome 13 | AGAATCTCACT |
| | | | cysteine) | | | | ACTAAAGTCAA |
| | | | proteinase | | | | GTATAGAAATA |
| | | | inhibitor, clade | | | | ACTGTTCTTAT |
| | | | B, member 9 | | | | GTTTTCCTCCA |
| | | | | | | | AGGCC |
| 697. | H3076C12-3 | Fac14 | fatty acid- | H3076C12 | Mm.143689 | Chromosome X | ATCTTTGGCTA |
| | | | Coenzyme A | | | | TATTTTCCTGG |
| | | | ligase, long | | | | TAGCATATGAC |
| | | | chain 4 | | | | AAATGTTTCTA |
| | | | | | | | CAGTGAGAAG |
| | | | | | | | CTGAGA |
| 698. | H3155C07-3 | 1810036L03Rik | RIKEN cDNA | H3155C07 | Mm.27385 | Chromosome 15 | GGGTTATAATG |
| | | | 1810036L03 | | | | CACTGAGATCC |
| | | | gene | | | | AGAAGTTGGG |
| | | | | | | | AAAACTCAATA |
| | | | | | | | AATGTACAAA |
| | | | | | | | GGAAAGC |
| 699. | K0331C04-3 | Sdccag8 | serologically | K0331C04 | Mm.171399 | Chromosome 1 | TACTTGTGTGA |
| | | | defined colon | | | | CAAGCTAGAG |
| | | | cancer antigen | | | | AAGTTACAGA |
| | | | 8 | | | | AGAGAAATGA |
| | | | | | | | CGAACTAGAA |
| | | | | | | | GAGCAATGC |
| 700. | J0538B04-3 | Laptm5 | lysosomal- | J0538B04 | Mm.4554 | Chromosome 4 | TAAATAATCCC |
| | | | associated | | | | TTCCCATGAGC |
| | | | protein | | | | CCACTGCTCTG |
| | | | transmembrane | | | | AATGGACAAG |
| | | | 5 | | | | CTGTCCTTATC |
| | | | | | | | TTCAAT |
| 701. | H3014E07-3 | 1810029G24Rik | RIKEN cDNA | H3014E07 | Mm.27800 | Chromosome 18 | AAATAGTTGTT |
| | | | 1810029G24 | | | | TTTAAGGTTGA |
| | | | gene | | | | AGGAAGAGAC |
| | | | | | | | ATTCCGATAGT |
| | | | | | | | TCACAGAGTAA |
| | | | | | | | TCAAGG |
| 702. | K0515H12-3 | 2900064A13Rik | RIKEN cDNA | K0515H12 | Mm.268027 | Chromosome 2 | TGAATCTACAG |
| | | | 2900064A13 | | | | GCAACTCTTCA |
| | | | gene | | | | TCTCTGTAATG |
| | | | | | | | CTACCTGACTT |
| | | | | | | | CTCTTGTGAGG |
| | | | | | | | AGCTG |
| 703. | H3159D10-3 | BG076403 | ESTs | H3159D10 | Mm.103300 | Chromosome 14 | TGGCAAAGAG |
| | | | BG076403 | | | | TAGATGAGAA |
| | | | | | | | AATGTTGGATT |
| | | | | | | | TAAATCAGCAG |
| | | | | | | | ACTCATTTCAT |
| | | | | | | | ACTTTGC |
| 704. | K0127F01-3 | Prg | proteoglyan, | K0127F02 | Mm.22194 | Chromosome 10 | ACCACGTTTAA |
| | | | secretory | | | | ATGACCAGTCT |
| | | | granule | | | | CAGGATAAAG |
| | | | | | | | AGTTTTACAGA |
| | | | | | | | AAATTTAAAAT |
| | | | | | | | GCCTGG |
| 705. | L0919B08-3 | Bnip31 | BCL2/adenovirus | L0919B08 | Mm.29820 | Chromosome 14 | GACATCGTTTT |
| | | | E1B 19kDa- | | | | CTCTCTAAATT |
| | | | interacting | | | | CAGTAGCAGTT |
| | | | protein 3-like | | | | TCATCGACAGT |
| | | | | | | | GCCATTGAACT |
| | | | | | | | ATGGG |
| 706. | J0904A09-3 | 1110060F11Rik | RIKEN cDNA | J0904A09 | Mm.4859 | Chromosome 4 | TCTGTGGGGTT |
| | | | 1110060F11 | | | | CTCATGCCAGT |
| | | | gene | | | | GTCTGAAATCT |
| | | | | | | | CACCTCACTAG |
| | | | | | | | AGATGTTTCTC |
| | | | | | | | GAATT |
| 707. | L0270B06-3 | D11Ertd759e | DNA segment, | L0270B06 | Mm.30111 | Chromosome 11 | TTCCAGTTCTC |
| | | | Chr 11, | | | | ATGTCTTGAGA |
| | | | ERATO Doi | | | | TTTCAAGTAAA |
| | | | 759, expressed | | | | GATGTGTTAGT |
| | | | | | | | GTAAGCTCAGA |
| | | | | | | | TCCGA |
| 708. | K0230D06-3 | Eafl | ELL associated | K0230D06 | Mm.37770 | Chromosome 14 | AACCATTGGGA |
| | | | factor 1 | | | | AAATGCAATAC |
| | | | | | | | AGATAAACTA |
| | | | | | | | GAGATTCGTAT |
| | | | | | | | AATGCCACGTG |
| | | | | | | | TTAGCT |
| 709. | K0611A03-3 | AI447904 | expressed | K0611A03 | Mm.447 | Chromosome 1 | GTGAATGGAGT |
| | | | sequence | | | | GTTTACTGTAT |
| | | | AI447904 | | | | GTAAGAAAGA |
| | | | | | | | AGAAAAGTGG |
| | | | | | | | AACTACATTTG |
| | | | | | | | CTATGAG |
| 710. | H3155A07-3 | BG076050 | ESTs | H3155A07 | Mm.182857 | Chromosome 5 | TTCACAATTTA |
| | | | BG076050 | | | | GACACAAGATT |
| | | | | | | | TGGAAGATTGA |
| | | | | | | | AACTGACATGA |
| | | | | | | | AAGTCTTCTTC |
| | | | | | | | CTGAG |
| 711. | H3028H11-3 | Ctsh | cathepsin H | H3028H11 | Mm.2277 | Chromosome Multiple | GAAGATTTTTT |
| | | | | | | Mappings | GATGTATAAAA |
| | | | | | | | GTGGCGTCTAC |
| | | | | | | | TCCAGTAAATC |
| | | | | | | | CTGTCATAAAA |
| | | | | | | | CTCCA |
| 712. | L0001D12-3 | 4833422F06Rik | RIKEN cDNA | L0001D12 | Mm.27436 | Chromosome 15 | AGAATGAACC |
| | | | 4833422F06 | | | | AGAATGGAGA |
| | | | gene | | | | AAACGTAAAA |
| | | | | | | | TTTGAAGAATC |
| | | | | | | | TCGTTGAAGAG |
| | | | | | | | CTATTTGC |
| 713. | L0951G01-3 | BG061831 | ESTs | L0951G01 | Mm.133824 | Chromosome 10 | TCGACAAGAG |
| | | | BG061831 | | | | GTAATCCGAGA |
| | | | | | | | AATGGAGCAG |
| | | | | | | | AAAACCTCCTT |
| | | | | | | | GCACTTCAGTG |
| | | | | | | | ATATACA |
| 714. | H3035G02-3 | A1314180 | expressed | H3035G02 | Mm.27829 | Chromosome 4 | TATATGCAACT |
| | | | sequence | | | | TCATAGATCCT |
| | | | A1314180 | | | | CTGCAATATGT |
| | | | | | | | ACTTAGCTACC |
| | | | | | | | TAAGCATGAA |
| | | | | | | | ATAGAC |
| 715. | C0925G02-3 | Fer113 | fer-1-like 3, | C0925G02 | Mm.34674 | Chromosome 19 | CGTCATATATC |
| | | | myoferlin (C. | | | | CTATTTGTAAT |
| | | | elegans) | | | | CAAGAGGAAA |
| | | | | | | | GACTACATTAA |
| | | | | | | | GAAGATAGGG |
| | | | | | | | TGCATAG |
| 716. | C0103H10-3 | Il17r | interleukin 17 | C0103H10 | Mm.4481 | Chromosome 6 | CTCAGATCAGT |
| | | | receptor | | | | TCTTTAGAAAG |
| | | | | | | | AGCTGGTATAG |
| | | | | | | | AAATGGGTGAT |
| | | | | | | | GTAAAACTTGA |
| | | | | | | | GAAGC |
| 717. | H3129F05-3 | Mrpl16 | mitochondrial | H3129F05 | Mm.203928 | Chromosome 19 | AATGAAAATCT |
| | | | ribosomal | | | | GCGTCTAACTT |
| | | | protein L16 | | | | TTGAAAGTAAG |
| | | | | | | | TGTTAACTTAC |
| | | | | | | | TTGAATGCTGG |
| | | | | | | | TTCCC |
| 718. | L0942B12-3 | | Mus musculus | L0942B12 | Mm.214553 | Chromosome 15 | AATCTTCGACC |
| | | | 12 days embryo | | | | AGACATTGGAT |
| | | | spinal ganglion | | | | ATTTGAACTAT |
| | | | cDNA, RIKEN | | | | CCTGAAACATT |
| | | | full-length | | | | TTAGAAATATC |
| | | | enriched | | | | CAGGC |
| | | | library, | | | | |
| | | | clone:D130046 | | | | |
| | | | C24 | | | | |
| | | | product:unknown | | | | |
| | | | EST, full | | | | |
| | | | insert sequence | | | | |
| 719. | L0009B09-3 | Plcg2 | phospholipase | L0009B09 | Mm.22370 | Chromosome 8 | TACCCCATTAA |
| | | | C, gamma 2 | | | | AGGCATCAAAT |
| | | | | | | | CCGGGTTTAGA |
| | | | | | | | TCAGTCCCTCT |
| | | | | | | | GAAGAATGGG |
| | | | | | | | TACAGT |
| 720. | C0665B08-3 | Sh3bp1 | SH3-domain | C0665B08 | Mm.4462 | Chromosome 15 | TTTTTTCTCTTG |
| | | | binding protein | | | | CCAATGTATTT |
| | | | 1 | | | | TTGTAAGGCTC |
| | | | | | | | GTAAATAAATT |
| | | | | | | | ATTTTGAACAA |
| | | | | | | | AACA |
| 721. | H3102F04-3 | Rgs10 | regulator of G- | H3102F04 | Mm.18635 | Chromosome 7 | CACACCCTCTG |
| | | | protein | | | | ATGTTCCAAAA |
| | | | signalling 10 | | | | GCTCCAGGACC |
| | | | | | | | AGATCTTCAAT |
| | | | | | | | CTCATGAAGTA |
| | | | | | | | TGACA |
| 722. | K0547F06-3 | | transcribed | K0547F06 | Mm.162929 | Chromosome 19 | CCCAGGTATTT |
| | | | sequence with | | | | CTAAGCATGCT |
| | | | moderate | | | | AGGTTTGAGGT |
| | | | similarity to | | | | CATTTACCATG |
| | | | protein | | | | TTCAAATAAAA |
| | | | sp:P00722 (E. | | | | GACGG |
| | | | coli) | | | | |
| | | | BGAL_ECOLI | | | | |
| | | | Beta- | | | | |
| | | | galactosidase | | | | |
| | | | (Lactase) | | | | |
| 723. | H3087C07-3 | Glb1 | galactosidase, | H3087C07 | Mm.255070 | Chromosome 9 | GGAGCAAAAC |
| | | | beta 1 | | | | TTGAATAATGT |
| | | | | | | | CCTTTATCCTG |
| | | | | | | | ATTTGAAATAA |
| | | | | | | | TCACGTCATCT |
| | | | | | | | TTCTGC |
| 724. | J0437D05-3 | AU023716 | ESTs | J0437D05 | Mm.173654 | Chromosome X | TGGAATAAGA |
| | | | AU023716 | | | | AAGAATCTGTG |
| | | | | | | | GTAGAAATAAT |
| | | | | | | | AGACTTGCTAC |
| | | | | | | | ATAGGGTTAGC |
| | | | | | | | TAAGGC |
| 725. | H3156A09-3 | Pex12 | peroxisomal | H3156A09 | Mm.30664 | Chromosome 11 | ACCACAGTTTA |
| | | | biogenesis | | | | TCAGCATTTGA |
| | | | factor 12 | | | | AGATTTCCTTG |
| | | | | | | | ATGATCCATAC |
| | | | | | | | TTGTCTTGGGA |
| | | | | | | | TAGGG |
| 726. | G0108H12-3 | Ly6e | lymphocyte | G0108H12 | Mm.788 | Chromosome 15 | AGGGTCAGCG |
| | | | antigen 6 | | | | CCGAATCTTGT |
| | | | complex, locus | | | | GGACACACTG |
| | | | E | | | | ACAAGGATGTC |
| | | | | | | | TAATCCAAATA |
| | | | | | | | GATGTAT |
| 727. | H3098D12-5 | Map2k1 | mitogen | H3098D12 | Mm.248907 | Chromosome 9 | AGTGGAGTATT |
| | | | activated | | | | CAGTCTGGAGT |
| | | | protein kinase | | | | TTCAGGATTTT |
| | | | kinase 1 | | | | GTGAATAAATG |
| | | | | | | | CTTAATAAAGA |
| | | | | | | | ACCCT |
| 728. | C0637C02-3 | Zmpste24 | zinc | C0637C02 | Mm.34399 | Chromosome 4 | TTTGGGCCCTT |
| | | | metalloproteinase, | | | | AAAAACATATT |
| | | | STE24 | | | | TCAGTTTTGCC |
| | | | homolog (S. | | | | CAAGTGAGGC |
| | | | cerevisiae) | | | | CTTAAAAATTG |
| | | | | | | | CCCATG |
| 729. | H3119B06-3 | Atplb3 | ATPase, | H3119B06 | Mm.424 | Chromosome Multiple | AAAGGAAAAT |
| | | | Na+/K+ | | | Mappings | AAAGTGGATCT |
| | | | transporting, | | | | GAAAGTAGAC |
| | | | beta 3 | | | | TCTGCTTCTGC |
| | | | polypeptide | | | | GCATGTGTGAG |
| | | | | | | | TGGTGCC |
| 730. | C0176B06-3 | Ubl1 | ubiquitin-like 1 | C0176B06 | Mm.259278 | Chromosome Multiple | TTCACTCCTGG |
| | | | | | | Mappings | ACTGTGATTTT |
| | | | | | | | CAGTGGGAGA |
| | | | | | | | TGGAAATTTTT |
| | | | | | | | CAGAGAACTG |
| | | | | | | | AACTGTG |
| 731. | C0626D04-3 | 9130404D14Rik | RIKEN cDNA | C0626D04 | Mm.219676 | Chromosome 2 | CACCATCCTTC |
| | | | 9130404D14 | | | | CAGAATATGGT |
| | | | gene | | | | ATGAAAAATCT |
| | | | | | | | ATGCAAACTGT |
| | | | | | | | GTAAGCTTTTG |
| | | | | | | | CTCAT |
| 732. | H3155E07-3 | Dock4 | dedicator of | H3155E07 | Mm.145306 | Chromosome 12 | TTGTGGAGTGT |
| | | | cytokinesis 4 | | | | GAAATAAAGG |
| | | | | | | | ATAATTGCCTA |
| | | | | | | | CCTCTAGCAAG |
| | | | | | | | TGGATCTTATT |
| | | | | | | | ATGTTG |
| 733. | C0106A05-3 | H2-Eb1 | histocompatibility | C0106A05 | Mm.22564 | Chromosome 17 | ACCAGAAAGG |
| | | | 2, class II | | | | ACAGTCTGGAC |
| | | | antigen E beta | | | | TTCAGCCAACA |
| | | | | | | | GGACTCCTGAG |
| | | | | | | | CTGAGATGAA |
| | | | | | | | GTAACAA |
| 734. | H3037B09-3 | | Mus musculus | H3037B09 | Mm.274876 | Chromosome 7 | GATACTGCCGG |
| | | | 12 days embryo | | | | CTTTGAAAATG |
| | | | spinal cord | | | | AAGAACAGAA |
| | | | cDNA, RIKEN | | | | GCTAAAATTCC |
| | | | full-length | | | | TGAAGCTTATG |
| | | | enriched | | | | GGTGGC |
| | | | library, | | | | |
| | | | clone:C530028 | | | | |
| | | | D16 | | | | |
| | | | product:231000 | | | | |
| | | | 8H09RIK | | | | |
| | | | PROTEIN | | | | |
| | | | homolog [Mus | | | | |
| | | | musculus], full | | | | |
| | | | insert sequence. | | | | |
| 735. | H3003b09-3 | F730017H24Rik | RIKEN cDNA | H3003B09 | Mm.205421 | Chromosome 14 | CCATTTGAGCC |
| | | | F730017H24 | | | | TCACTGCAATG |
| | | | gene | | | | TTAGTGCAGAG |
| | | | | | | | GAGAAAACAA |
| | | | | | | | TTTTTAATGTA |
| | | | | | | | ATCTTG |
| 736. | C0909E10-3 | Pign | phosphatidylino- | C0909E10 | Mm.268911 | Chromosome 1 | GGCAACTTGTA |
| | | | sitol glycan, | | | | AAGTGTGTTCA |
| | | | class N | | | | TTCTAACTGTT |
| | | | | | | | AAACTGAGAA |
| | | | | | | | AACTTGAGAAC |
| | | | | | | | ATACTG |
| 737. | H3045G01-3 | BG066588 | ESTs | H3045G01 | Mm.26804 | Chromosome 14 | CAGAAGAGAT |
| | | | BG066588 | | | | TCTGAAAATGT |
| | | | | | | | TAGTTGTGGTG |
| | | | | | | | ACTCTAATGTA |
| | | | | | | | GATCCATAATCT |
| | | | | | | | GAAAAG |
| 738. | H3006E10-3 | | transcribed | H3006E10 | Mm.218665 | Chromosome 15 | TATCGTAAGTT |
| | | | sequence with | | | | GCACCTATTGT |
| | | | weak similarity | | | | TAAGTGGAAA |
| | | | to protein | | | | ATGCTCTGATT |
| | | | sp:Q9H321 | | | | ACACTCAGGA |
| | | | (H. sapiens) | | | | AGCTGGG |
| | | | VCXC_HUMA | | | | |
| | | | N VCX-C | | | | |
| | | | protein | | | | |
| | | | (Variably | | | | |
| | | | charged protein | | | | |
| | | | X-C) | | | | |
| 739. | H3098H09-3 | 2310016E02Rik | RIKEN cDNA | H3098H09 | Mm.21450 | Chromosome 5 | TGTTTTGTCCC |
| | | | 2310016E02 | | | | TAAATCACCAC |
| | | | gene | | | | CACTCACTATT |
| | | | | | | | TCTCCCAGGGT |
| | | | | | | | CTGATAATGCC |
| | | | | | | | TTTAC |
| 740. | J0540D09-3 | Adam9 | a disintegrin | J0540D09 | Mm.28908 | Chromosome 8 | AGCCACTTTAA |
| | | | and | | | | CTCTAAACTCG |
| | | | metalloproteinase | | | | AATTTCAAAGC |
| | | | domain 9 | | | | CTTGAGTGAAG |
| | | | (meltrin | | | | TCCTCTAGAAT |
| | | | gamma) | | | | GTTTA |
| 741. | L0208C06-3 | Pknox1 | Pbx/knotted 1 | L0208C06 | Mm.259295 | Chromosome 17 | GCTTTGTTTAA |
| | | | homebox | | | | ATGGTCAGACT |
| | | | | | | | CCCAAACATTG |
| | | | | | | | GAGCCTTTTGA |
| | | | | | | | ATGTGTTCTGA |
| | | | | | | | GACCT |
| 742. | H3154G05-3 | Napg | N- | H3154G05 | Mm.154623 | Chromosome 18 | CCTTAGAAAGA |
| | | | ethylmaleimide | | | | TGGTAATTCAC |
| | | | sensitive fusion | | | | TTTAGGTAAAA |
| | | | protein | | | | GTACTATTTCA |
| | | | attachment | | | | CGCCATTATGA |
| | | | protein gamma | | | | AACCC |
| 743. | L0854E11-3 | 1500032M01Rik | RIKEN cDNA | L0854E11 | Mm.29628 | Chromosome 19 | TAAAATGAGG |
| | | | 1500032M01 | | | | CTTTTGGAAAG |
| | | | gene | | | | AAAGATGAAA |
| | | | | | | | ACGTAGAATGT |
| | | | | | | | AGTGCTAAGA |
| | | | | | | | ACGTTTCC |
| 744. | H3014C06-3 | B2m | beta-2 | H3014C06 | Mm.163 | Chromosome 2 | GCAGTTACTCA |
| | | | microglobulin | | | | TCTTTGGTCTA |
| | | | | | | | TCACAACATAA |
| | | | | | | | GTGACATACTT |
| | | | | | | | TCCTTTTGGTA |
| | | | | | | | AAGCA |
| 745. | K0538G12-3 | Ccr2 | chemokine (C- | K0538G12 | Mm.6272 | Chromosome 9 | TGCTTAGAACT |
| | | | C) receptor 2 | | | | ACATAGAATCA |
| | | | | | | | GAAGCAAAAT |
| | | | | | | | GGATGCCTTAG |
| | | | | | | | CACTGAGGAA |
| | | | | | | | AGGTTTC |
| 746. | J0819C09-3 | C030002B11Rik | RIKEN cDNA | J0819C09 | Mm.70065 | Chromosome 10 | GGTTTTCGAAC |
| | | | C030002B11 | | | | CACGTACCTTT |
| | | | gene | | | | ATGCCTCGTGA |
| | | | | | | | TTGTGAAACAT |
| | | | | | | | TGACTTTTGTA |
| | | | | | | | AACCC |
| 747. | C0175B11-3 | Histlh2bc | histone 1, h2bc | C0175B11 | Mm.21579 | Chromosome 13 | GTTCACTGTAG |
| | | | | | | | AAATTTGTGAT |
| | | | | | | | AAGAAAGACA |
| | | | | | | | CACAGACGTA |
| | | | | | | | GAAAATGAGA |
| | | | | | | | ATACTTGC |
| 748. | H3009B11-3 | Nufip1 | nuclear fragile | H3009B11 | Mm.21138 | Chromosome 14 | AAGACTTTTT |
| | | | X mental | | | | TGGACTTAATA |
| | | | retardation | | | | CTGATTCTGTG |
| | | | protein | | | | AAAACTGAAG |
| | | | interacting | | | | AAGTGTAGATG |
| | | | protein | | | | TCTCCC |
| 749. | H3135D02-3 | Lamp2 | lysosomal | H3135D02 | Mm.486 | Chromosome X | CTGGTGTGGGA |
| | | | membrane | | | | TATTTTCCACA |
| | | | glycoprotein 2 | | | | CTTTAGAATTT |
| | | | | | | | GTATAAGAAA |
| | | | | | | | CTGGTCCATGT |
| | | | | | | | AAGTAC |
| 750. | K0540G08-3 | 1200013B08Rik | RIKEN cDNA | k0540g08 | Mm.247440 | Chromosome X | TAAAGGTTTTA |
| | | | 1200013B08 | | | | GTGTCCTAACT |
| | | | gene | | | | CCCCAGGATCA |
| | | | | | | | GGAGATTATCC |
| | | | | | | | CAACTATTTCT |
| | | | | | | | GGGGT |
| 751. | H3089H05-3 | Lnx2 | ligand of numb- | H3089H05 | Mm.34462 | Chromosome 5 | CTGAATTTTGA |
| | | | protein X 2 | | | | TCACTTGTGGT |
| | | | | | | | TTCTCATGGTG |
| | | | | | | | ACCTCCATTTG |
| | | | | | | | CAACAAAAAG |
| | | | | | | | ATGTCT |
| 752. | J0203A08-3 | C85149 | ESTs C85149 | J0203A08 | Mm.154684 | Chromosome 2 | TGTGCTTTACC |
| | | | | | | | AAAATGGGAA |
| | | | | | | | ATAATTCTGCT |
| | | | | | | | TTAGAGGATAC |
| | | | | | | | TATCAAGACAA |
| | | | | | | | CCTTAC |
| 753. | H3119F01-3 | Mcfd2 | multiple | H3119F01 | Mm.30251 | Chromosome 17 | TCTGTGAGATG |
| | | | coagulation | | | | TTGTAGACATT |
| | | | factor | | | | CCGTAAGAGA |
| | | | deficiency 2 | | | | ATCCAGAATGA |
| | | | | | | | TAGCAGGATCA |
| | | | | | | | GGAAAG |
| 754. | H3134C05-3 | Mglap | matrix gamma- | H3134C05 | Mm.243085 | Chromosome 6 | CTTACATGATC |
| | | | carboxyglutamate | | | | TCCTAAAAGGA |
| | | | (gla) protein | | | | TGGGCCCCTCC |
| | | | | | | | TTCCTTTTGCG |
| | | | | | | | GGTTGAAAGTA |
| | | | | | | | ATGAA |
| 755. | C0147D11-3 | B230215M10Rik | RIKEN cDNA | C0147D11 | Mm.41525 | Chromosome 10 | CTGTTTAAAAA |
| | | | B230215M10 | | | | ATGAAATCAG |
| | | | gene | | | | GAAGCTTGAA |
| | | | | | | | GAAGACGATC |
| | | | | | | | AGACGAAAGA |
| | | | | | | | CATTTGAGC |
| 756. | C0949H10-3 | Sulf1 | sulfatase 1 | C0949H10 | Mm.45563 | Chromosome 1 | TGAATATAGTA |
| | | | | | | | GGGCCATGAGT |
| | | | | | | | ATATAAAATCT |
| | | | | | | | ATCCAGTCAAA |
| | | | | | | | ATGGCTAGAAT |
| | | | | | | | TGTGC |
| 757. | K0114E04-3 | BM222075 | ESTs | K0114E04 | Mm.221705 | Chromosome 19 | GGGGGAAATT |
| | | | BM222075 | | | | CTATATGAGCT |
| | | | | | | | TCGTTTTCTAA |
| | | | | | | | TGACTTACATG |
| | | | | | | | GATAGTATGGA |
| | | | | | | | AACTTC |
| 758. | H3012C03-3 | Cappa1 | capping protein | H3012C03 | Mm.19142 | Chromosome Multiple | AAACTTGAAA |
| | | | alpha 1 | | | Mappings | ACACAGACATT |
| | | | | | | | GAAGGAATCA |
| | | | | | | | TAGGTATTTTT |
| | | | | | | | GCTTTATGCTC |
| | | | | | | | TCTGGCA |
| 759. | C0507E11-3 | BE824970 | ESTs | C0507E11 | Mm.139860 | Chromosome 16 | AATAAGCAGG |
| | | | BE824970 | | | | AAGAATTTGAC |
| | | | | | | | TTGGAAAACTA |
| | | | | | | | ATACACGCATG |
| | | | | | | | TTAGGCATTCT |
| | | | | | | | CAAGGC |
| 760. | H3158D06-3 | Lnk | linker of T-cell | H3158D06 | Mm.200936 | Chromosome 5 | TCCCACTGTTT |
| | | | receptor | | | | ACAGATGTAGT |
| | | | pathways | | | | TCTTGTGCACA |
| | | | | | | | GGTGCCACTAG |
| | | | | | | | CTGGTACCCTA |
| | | | | | | | GGCCT |
| 761. | C0174C02-3 | Pold3 | polymerase | C0174C02 | Mm.37562 | Chromosome 7 | TATTTTTGTCA |
| | | | (DNA- | | | | TTGCCTCTAGT |
| | | | directed), delta | | | | GATTTTTGTAA |
| | | | 3, accessory | | | | ATGGGAATGG |
| | | | subunit | | | | AAAAGTACAA |
| | | | | | | | GGCAACC |
| 762. | C0130G10-3 | Cklfst7 | chemokine-like | C0130G10 | Mm.35600 | Chromosome 9 | TTAACTGGCCT |
| | | | factor super | | | | GTCAAACTGGT |
| | | | family 7 | | | | CTTGAAGCGTC |
| | | | | | | | TCTAAGTGAAG |
| | | | | | | | AGCCAGAAGA |
| | | | | | | | AACCCT |
| 763. | C0137F07-3 | Rik3cb | phosphatidylino- | C0137F07 | Mm.213128 | Chromosome 9 | CAATGTGATTT |
| | | | sitol 3-kinase, | | | | TTCAATGGTAT |
| | | | catalytic, beta | | | | TAGTTCAAATT |
| | | | polypeptide | | | | GACGTGGATTC |
| | | | | | | | ATGCCACATGG |
| | | | | | | | AAATC |
| 764. | H3115F01-3 | 2610027O18Rik | RIKEN cDNA | H3115F01 | Mm.46501 | Chromosome 12 | AACTGAATAA |
| | | | 2610027O18 | | | | AGTTGACCAGA |
| | | | gene | | | | AAGTGAAAGT |
| | | | | | | | CTTTAACATGG |
| | | | | | | | ATGGAAAAGA |
| | | | | | | | CTTCATCC |
| 765. | H3097F03-3 | | Mus musculus, | H3097F03 | Mm.227202 | Chromosome 3 | GGATATAAAGT |
| | | | clone | | | | GTATTTCTTTC |
| | | | IMAGE:53723 | | | | AGTGATTTCTC |
| | | | 38, mRNA | | | | AGTGCATAAG |
| | | | | | | | AAGTGCATAA |
| | | | | | | | GTCTCAG |
| 766. | H3059A05-3 | Mad211 | MAD2 (mitotic | H3059A05 | Mm.43444 | Chromosome 6 | TAGCTTTTTAA |
| | | | arrest deficient, | | | | AAGAAGTTTTT |
| | | | honolog)-like 1 | | | | CTACCTACAGT |
| | | | (yeast) | | | | GACCATTGTTA |
| | | | | | | | AAGGAATCCAT |
| | | | | | | | CCCAC |
| 767. | L0935E02-3 | Syk | spleen tyrosine | L0935E02 | Mm.248456 | Chromosome 13 | ATTTGCAAGGT |
| | | | kinase | | | | CAGAAACTAG |
| | | | | | | | CCAAGGTCCTT |
| | | | | | | | CTCAGGCATCT |
| | | | | | | | ATCCTTAACTT |
| | | | | | | | GGTCTC |
| 768. | C0946F08-3 | 1110014L17Rik | RIKEN cDNA | C0946F08 | Mm.30103 | Chromosome 11 | TTGGAATTTGA |
| | | | 1110014L17 | | | | GGAGGAGAAA |
| | | | gene | | | | TGAAAAAACA |
| | | | | | | | GTGTGTCCCTG |
| | | | | | | | GTGTCACCCTG |
| | | | | | | | GCATCAT |
| 769. | H3079F02-5 | | Possibly | H3079F02 | Data not found | Chromosome 10 | TCTTATGATTT |
| | | | intronic in | | | | AAGTGATTGGT |
| | | | U011488- | | | | GGATAAATGTA |
| | | | H3079F02 | | | | TAGGAATTTTA |
| | | | | | | | CACTCCAGCAG |
| | | | | | | | CATGG |
| 770. | H3137E07-3 | III0ra | interleukin 10 | H3137E07 | Mm.26658 | Chromosome 9 | GCCTCAAATGG |
| | | | receptor, alpha | | | | AACCACAAGT |
| | | | | | | | GGTGTGTGTTT |
| | | | | | | | TCATCCTAATA |
| | | | | | | | AAAAGTCAGG |
| | | | | | | | TGTTTTG |
| 771. | C0143H12-3 | Galns | galactosamine | C0143H12 | Mm.34702 | Chromosome 8 | CCGTACACAAA |
| | | | (N-acetyl)-6- | | | | AGTGAAGATTT |
| | | | sulfate sulfatase | | | | CAGCGAAATG |
| | | | | | | | CCAAGGAAGT |
| | | | | | | | GCCATCTATCT |
| | | | | | | | GGCTTCT |
| 772. | H3114D03-3 | Man2a1 | mannosidase 2, | H3114D03 | Mm.2433 | Chromosome 17 | AAGAAATGC |
| | | | alpha 1 | | | | TGTATGATGTT |
| | | | | | | | AGAAGACATT |
| | | | | | | | GTAATTATCAT |
| | | | | | | | CCCGTGTCTTT |
| | | | | | | | GCTGTAC |
| 773. | H3041H09-3 | BG066348 | ESTs | H3041H09 | Mm.270044 | Chromosome 8 | GGCATTTCAGT |
| | | | BG066348 | | | | TTATCTTGGGT |
| | | | | | | | TTGTAATTAGT |
| | | | | | | | TAAAACAAAA |
| | | | | | | | ACCAACCTAGG |
| | | | | | | | TCTGTG |
| 774. | C0628H04-3 | Slc2a12 | solute carrier | C0628H04 | Mm.268014 | Chromosome 10 | ATTAGCCAAGG |
| | | | family 2, | | | | AGTCCGGACAT |
| | | | memeber 12 | | | | AATATTTATCC |
| | | | | | | | AGATCTCTAAG |
| | | | | | | | CAGTTAGCTTT |
| | | | | | | | AAATT |
| 775. | K0125E07-3 | Ifngr | interferon | K0125E07 | Mm.549 | Chromosome 10 | TACATTAGCTA |
| | | | gamma receptor | | | | ATACTAACCAC |
| | | | | | | | ATAGAATATCA |
| | | | | | | | GACTTAGATAC |
| | | | | | | | GTGAATAGGG |
| | | | | | | | ATCCTG |
| 776. | G0115E02-3 | Sdcbp | syndecan | G0115E02 | Mm.276062 | Chromosome 4 | AAGATTTTCTA |
| | | | binding protein | | | | GTCACTGCATA |
| | | | | | | | AAGGAAACGC |
| | | | | | | | CTAAGAGTTGC |
| | | | | | | | CGTATTGCTTT |
| | | | | | | | CTGAGA |
| 777. | C0032B05-3 | Rap2b | RAP2B, | C0032B05 | Mm.26939 | Chromosome 3 | ACAAGAATTCA |
| | | | member of | | | | TTCTTAACATT |
| | | | RAS oncogene | | | | TGAACGAGTGT |
| | | | family | | | | ATTTGCTTAGG |
| | | | | | | | TCGATGAAAGT |
| | | | | | | | GTTGC |
| 778. | H3141C08-3 | Ofd1 | oral-facial- | H3141C08 | Mm.2474889 | Chromosome X | AGGATTTTCTC |
| | | | digital | | | | ATGAAGAACC |
| | | | syndrome 1 | | | | AGATGACATGT |
| | | | gene homolog | | | | GGTAATAACAT |
| | | | (human) | | | | TAGCTGTCTAG |
| | | | | | | | TTTCTC |
| 779. | H3157C05-3 | BG076236 | ESTs | H3157C05 | Mm.182877 | Chromosome 1 | TAGAGTCTGA |
| | | | BG076236 | | | | AGAACAGAAA |
| | | | | | | | TTCAAGGTCAT |
| | | | | | | | TTTCAATTACA |
| | | | | | | | GAGTGAGGTTA |
| | | | | | | | GAGCCA |
| 780. | H3076A01-3 | 5031439G07Rik | RIKEN cDNA | H3076A01 | Mm.121973 | Chromosome 15 | TCTAAAACATG |
| | | | 5031439G07 | | | | CCAAATGACTT |
| | | | gene | | | | ATGTCACAAAG |
| | | | | | | | AATAGGTCCTA |
| | | | | | | | ATATACTGTAT |
| | | | | | | | ACCCC |
| 781. | H3080D06-3 | BC01807 | cDNA sequence | H3080D06 | Mm.139738 | Chromosome 13 | GTGTTTCTTCC |
| | | | BC018507 | | | | CATTTGTAAAT |
| | | | | | | | GTCCTGAACCA |
| | | | | | | | TAAATTACTAT |
| | | | | | | | CAGGATTAACT |
| | | | | | | | GACAG |
| 782. | L0518D04-3 | Uap1 | UDP-N- | L0518D04 | Mm.27969 | Chromosome 1 | GAAGCTGGAA |
| | | | acetylglucosamine | | | | GCATTTGTTTT |
| | | | pyrophosphorylase | | | | TGAAGTTGTAC |
| | | | 1 | | | | ATATTGATAAG |
| | | | | | | | TCAGCGTATGT |
| | | | | | | | GTCAGA |
| 783. | K0541B11-3 | BM239901 | ESTs | K0542B11 | Mm.222307 | Chromosome 2 | TTACATGGCAA |
| | | | BM239901 | | | | ATCTGAAAGG |
| | | | | | | | AAGACTTAAGC |
| | | | | | | | AGGGTAAAGTT |
| | | | | | | | AATTGAAAGG |
| | | | | | | | AGGAGCT |
| 784. | L0959D03-3 | Tnfrsfla | tumor necrosis | L0959D03 | Mm.1258 | Chromosome 6 | AGCAATCTTTG |
| | | | factor receptor | | | | TATCAATTATA |
| | | | superfamily, | | | | TCACACTAATG |
| | | | member 1a | | | | GATGAACTGTG |
| | | | | | | | TAAGGTAAGG |
| | | | | | | | ACAAGC |
| 785. | H3035C07-3 | BG065787 | ESTs | H3035C07 | Mm.24933 | Chromosome 1 | GGTGTAGGAA |
| | | | BG065787 | | | | ATAAAGTTTAG |
| | | | | | | | TCAATGTTGAA |
| | | | | | | | AATCTCTCCTG |
| | | | | | | | GTTGAATGACT |
| | | | | | | | TGCTC |
| 786 | M29855.1 | Csf2rb2 | colony | M29855 | Mm.1940 | Chromosome 15 | CTTTCAGTCTC |
| | | | stimulating | | | | CTTCTGTGTCT |
| | | | factor 2 | | | | CGAACCTTGAA |
| | | | receptor, beta 2 | | | | CAGGATGTGAT |
| | | | low-affinity | | | | AACTTTTCTAG |
| | | | (granulocyte- | | | | ACCAC |
| | | | macrophage) | | | | |
| 787. | C0352C11-3 | BM197981 | ESTs | C0352C11 | Mm.215584 | Chromosome 2 | GACTGTTTCTG |
| | | | BM197981 | | | | GGAAAATAAG |
| | | | | | | | TATGTGAAGTG |
| | | | | | | | ATGCAGAAAA |
| | | | | | | | TCCATCTAGAC |
| | | | | | | | AGTTGAG |
| 788. | L0846B10-3 | BM117093 | ESTs | L0846B10 | Mm.216113 | No Chromosome | TGGTGGCTTGA |
| | | | MN117093 | | | location | TTGATTTGATC |
| | | | | | | info available | TGAGAGCAGTT |
| | | | | | | | TATAACATAAT |
| | | | | | | | GGAGAACTGTT |
| | | | | | | | TGCAG |
| 789. | L0227C06-3 | Serpinb6a | serine (or | L0227C06 | Mm.2623 | Chromosome 13 | AGAAGTCTACC |
| | | | cysteine) | | | | TTTAAGATGAC |
| | | | proteinase | | | | CTATATTGGAG |
| | | | inhibitor, clade | | | | AGATATTCACT |
| | | | B, member 6a | | | | AAGATTCTGTT |
| | | | | | | | GCTTC |
| 790. | J0214H09-3 | Serpina3g | serine (or | J0214H09 | Mm.264709 | No Chromosome | ACTCTCTGGTC |
| | | | cysteine) | | | location | ATGATGGTTTT |
| | | | proteinase | | | info available | CCGAAATCAG |
| | | | inhibitor, clade | | | | GTTCCTGACCT |
| | | | A, member 3G | | | | GAAAATTTGGG |
| | | | | | | | TTAATC |
| 791. | H3077F12-3 | Arhh | ras homolog | H3077F12 | Mm.20323 | Chromosome 5 | GTTTTTCAT- |
| | | | gene family, | | | | GCT |
| | | | member H | | | | TTGGAAGTCTT |
| | | | | | | | TTCTTTGAAAA |
| | | | | | | | GGCAAACTGCT |
| | | | | | | | GTATGAGGAG |
| | | | | | | | AAAATA |
| 792. | C0341D05-3 | BM196992 | ESTs | C0341D05 | Mm.222093 | Chromosome 1 | GTGTGTAGGAA |
| | | | BM196992 | | | | AATGTAATTAA |
| | | | | | | | GTACAAGGCTT |
| | | | | | | | GTTTATGGGTG |
| | | | | | | | GCTATGGAATG |
| | | | | | | | CAGTC |
| 793. | H3043H11-3 | BG066522 | ESTs | H3043H11 | Mm.25035 | Chromosome 6 | GTTTCCTCATC |
| | | | BG066522 | | | | AGGTGTAATGG |
| | | | | | | | CGTGTCCTAAT |
| | | | | | | | GAAGCTATTC |
| | | | | | | | TTATGTATAAC |
| | | | | | | | AGAGA |
| 794. | K0507D06-3 | | Mus musculus, | K0507d06 | Mm.103545 | Chromosome 11 | TGAAAAAATG |
| | | | clone | | | | AAAAGAATCA |
| | | | IMAGE:12632 | | | | GAGATGAAAT |
| | | | 53, mRNA | | | | AGGAGCGCTC |
| | | | | | | | AGAAGTTTTTA |
| | | | | | | | TGTTCTCCC |
| 795. | J0535D11-3 | AU020606 | ESTs | J0535D11 | Mm.26229 | Chromosome 11 | AAAGAAATGA |
| | | | AU020606 | | | | AAACCGTCATT |
| | | | | | | | TGCGATTTTCA |
| | | | | | | | GGGTACGTTTC |
| | | | | | | | TAATGTATCCA |
| | | | | | | | GAAGTC |
| 796. | H3152F04-3 | Sepp1 | selenoprotein P, | H3152F04 | Mm.22699 | Chromosome 15 | TTTCCAGTGTT |
| | | | plasma, 1 | | | | CTAGTTACATT |
| | | | | | | | AATGAGAACA |
| | | | | | | | GAAACATAAA |
| | | | | | | | CTATGACCTAG |
| | | | | | | | GGGTTTC |
| 797. | L0701F07-3 | H2-Ab1 | histocompatilility | L0701F07 | Mm.275510 | Chromosome 17 | TTTTGACTCAG |
| | | | 2, class II | | | | TTGACTGTCTC |
| | | | antigen A, beta | | | | AGACTGTAAG |
| | | | 1 | | | | ACCTGAATGTC |
| | | | | | | | TCTGCTCCGAA |
| | | | | | | | TTCCTG |
| 798 | L0227H07-3 | Clca1 | chloride | L0227H07 | Mm.275745 | Chromosome 3 | CCCGAGTTACT |
| | | | channel calcium | | | | AACAACATTCT |
| | | | activated 1 | | | | TTTGCTATATG |
| | | | | | | | TAGATCAAGAT |
| | | | | | | | TAACAGTTCCT |
| | | | | | | | CATTC |
| 799. | J1014C11-3 | 2900036G02Rik | RIKEN cDNA | J1014C11 | Mm.80676 | No Chromosome | GTTTTGGTGCA |
| | | | 2900036G02 | | | location | AAAGTCGTCCT |
| | | | gene | | | info available | GTGTCTCTTGT |
| | | | | | | | TCCCTTCATTA |
| | | | | | | | GAAAACATGCT |
| | | | | | | | AGAGG |
| 800. | H3134H09-3 | BG074421 | ESTs | H3134H09 | Mm.197381 | Chromosome 12 | AGGAAGGAAA |
| | | | BG074421 | | | | ATAGGCTTTGT |
| | | | | | | | TGTATGTACAT |
| | | | | | | | AAGTGGAATTA |
| | | | | | | | ACAAGAGTCTT |
| | | | | | | | TAGTCC |
| 801. | G0116A07-3 | Atp6vblc1 | ATPase, H+ | G0116A07 | Mm.276618 | Chromosome 15 | TACAGGGAAT |
| | | | transporting | | | | GGTCTAAGCAT |
| | | | V1 subunit C, | | | | ACCATTTCATT |
| | | | isoform 1 | | | | CACTGTATTAG |
| | | | | | | | TAGACATAACT |
| | | | | | | | GTTGAG |
| 802. | L0942F05-3 | Ostm1 | osteopetrosis | L0942F05 | Mm.46636 | Chromosome 10 | GAAACGGGCTT |
| | | | associated | | | | TGTTGTAAAGG |
| | | | transmembrane | | | | TAATGAATAGG |
| | | | protein 1 | | | | AAACTCCTCAG |
| | | | | | | | ATTCAATGGTT |
| | | | | | | | AAGAA |
| 803. | C0912H10-3 | 0610041E09Rik | RIKEN cDNA | C0912H10 | Mm.132926 | Chromosome 13 | AAGTTAAGGA |
| | | | 0610041E09 | | | | AATACTGAGA |
| | | | gene | | | | ATCGGTCAGTT |
| | | | | | | | AACACTCTGAA |
| | | | | | | | AAGCTATTCAA |
| | | | | | | | AGCATAG |
| 804. | C0304E12-3 | Pde1b | phosphodiesterase | C0304E12 | Mm.62 | Chromosome 15 | AAATACATGCA |
| | | | 1B, Ca2+- | | | | TTTGTACAGTG |
| | | | calmoduin | | | | GGCCCTGTTCT |
| | | | dependent | | | | TGTGAAGTCCA |
| | | | | | | | TCTCCATGGTC |
| | | | | | | | ATTAG |
| 805. | L0605C12-3 | 4930579K19Rik | RIKEN cDNA | L0605C12 | Mm.117473 | Chromosome 9 | CCGTTTTATTG |
| | | | 4930579K19 | | | | ATTGGAAATGT |
| | | | gene | | | | AAGACTCAAA |
| | | | | | | | GAACTCAGGTT |
| | | | | | | | TACTGGCCAAG |
| | | | | | | | ATGGCA |
| 806. | K0539A07-3 | Cd53 | CD53 antigen | K0529A07 | Mm.2692 | Chromosome 3 | GGAAAGAGAG |
| | | | | | | | ATCAAACTAGG |
| | | | | | | | AACCTACAAG |
| | | | | | | | ATAGTTCACTA |
| | | | | | | | GCCTAAGATCT |
| | | | | | | | TTACTTG |
| 807. | L0228H12-3 | 6430628I05Rik | RIKEN cDNA | L0228H12 | Mm.196533 | Chromosome 9 | TTGATTGGTGT |
| | | | 6430658I05 | | | | TTCTGAGCATT |
| | | | gene | | | | CAGACTCCGCA |
| | | | | | | | CCCTCATTTCT |
| | | | | | | | AATAAATGCA |
| | | | | | | | ACATTG |
| 808. | L0855B10-3 | BM117713 | ESTs | L0855B10 | Mm.216997 | Chromosome 10 | CTAGTGAAATT |
| | | | BM117713 | | | | TATGTCAGAAT |
| | | | | | | | GACATATCTGA |
| | | | | | | | ACTCTGAATTC |
| | | | | | | | ATCTCTAGTTT |
| | | | | | | | CCACG |
| 809. | H3075B10-3 | 2810404F18Rik | RIKEN cDNA | H3075B10 | Mm.29476 | Chromosome 11 | TAGTTAATACT |
| | | | 2810404F18 | | | | TCTCTGAAATA |
| | | | gene | | | | CATGGTAACAA |
| | | | | | | | CTAGTAAGCAA |
| | | | | | | | GAGATACCGC |
| | | | | | | | AGATTG |
| 810. | L0022G07-3 | | L0033G07-3 | L0022G07 | | No Chromosome | TGGATTATTCC |
| | | | NIA Mouse | | | location | CGCCAAAGCA |
| | | | E12.5 Female | | | info available | CCCAAGTCGGC |
| | | | Mesonephros | | | | CTGTTTAATTG |
| | | | and Gonads | | | | GAGAAAGATG |
| | | | cDNA Library | | | | GAATTAA |
| | | | Mus musculus | | | | |
| | | | cDNA clone | | | | |
| | | | L0022G07 3′, | | | | |
| | | | MRNA | | | | |
| | | | sequence | | | | |
| 811. | H3107C11-3 | Efemp2 | epidermal | H3107C11 | Mm.471781 | Chromosome 19 | GATCCAGGCA |
| | | | growth factor- | | | | ACCTCTGTTTA |
| | | | containing | | | | CCCTGGGGCCT |
| | | | fibulin-like | | | | ACAATGCCTTT |
| | | | extracellular | | | | CAGATCCGTTC |
| | | | matrix protein 2 | | | | TGGAAA |
| 812. | H3025H12-3 | 1200003O06Rik | RIKEN cDNA | H3025H12 | Mm.142104 | Chromosome 3 | GTTCCATCTGA |
| | | | 1200003O06 | | | | CTTAAACAAAA |
| | | | gene | | | | ACCGTAGTTTC |
| | | | | | | | CAGCTCAGAAT |
| | | | | | | | CATCCTAACAT |
| | | | | | | | AGAAA |
| 813. | J0040E05-3 | Stx3 | syntaxin 3 | J0040E05 | Mm.203928 | Chromosome 19 | GTAGGGGAAT |
| | | | | | | | AACTAACCAA |
| | | | | | | | AGTAGAGGGA |
| | | | | | | | ATTCTAAGTTT |
| | | | | | | | AGTAGTAAATG |
| | | | | | | | TGGCTTGG |
| 814. | H3075F03-3 | Cls | complement | H3075F03 | Mm.24128 | Chromosome 6 | GGTGTGGGACT |
| | | | component 1, s | | | | TATGGGGTCTA |
| | | | subcomponent | | | | CACAAAGGTA |
| | | | | | | | AAGAATTACGT |
| | | | | | | | GGACTGGATCC |
| | | | | | | | TGAAAA |
| 815. | L0600G09-3 | BM125147 | ESTs | L0600G09 | Mm.221784 | Chromosome 1 | AGGTATGACAT |
| | | | BM125147 | | | | TTTACATCCTT |
| | | | | | | | GAATCTTACTT |
| | | | | | | | ACTATGTGCTA |
| | | | | | | | AACAATTGGCA |
| | | | | | | | GAAGG |
| 816. | K0115H01-3 | KLHL6 | kelch-like 6 | K0115H01 | Mm.86699 | Chromosome 16 | TGCTTGTGTGA |
| | | | | | | | ACTACCTCAGG |
| | | | | | | | ATGAAGGGTA |
| | | | | | | | ATGTTTAACAT |
| | | | | | | | TCCATACATGC |
| | | | | | | | CTACTG |
| 817. | H3015B10-2 | Gus | beta- | H3015B10 | Mm.3317 | Chromosome 5 | CGATGGACCCA |
| | | | glucuronidase | | | | AGATACCGAC |
| | | | | | | | ATGAGAGTAGT |
| | | | | | | | GTTGAGGATCA |
| | | | | | | | ACAGTGCCCAT |
| | | | | | | | TATTAT |
| 818. | H3108A12-3 | 0910001A06Rik | RIKEN cDNA | H3108A12 | Mm.22383 | Chromosome 15 | GCAGCCAAAA |
| | | | 0910001A06 | | | | TGGAAATGTTT |
| | | | gene | | | | AAATTAACTGT |
| | | | | | | | GTTGTACAAT |
| | | | | | | | GACCCAACAC |
| | | | | | | | AAAACC |
| 819. | H3108H90-5 | | UNKNOWN: | H3108H09 | Data not found | Chromosome 13 | TTGACATGATA |
| | | | Similar to | | | | CATTACGCCTT |
| | | | Homo sapiens | | | | TGCAGTGAGCT |
| | | | KIAA1577 | | | | AATAAGCTAAC |
| | | | protein | | | | ATTTGTGCACA |
| | | | (KIAA1577), | | | | GATAA |
| | | | mRNA | | | | |
| 820. | K0645H01-3 | Fyb | FYN binding | K0645H01 | Mm.257567 | Chromosome 15 | TCTCAACTCAT |
| | | | protein | | | | CTCAGATTAGG |
| | | | | | | | AAGTATTTGGC |
| | | | | | | | AGTATTAGCA |
| | | | | | | | TCATGTGTCCC |
| | | | | | | | TGTGA |
| 821. | H3029A02-3 | Shyc | selective | H3029A02 | Mm.12912 | Chromosome 7 | ATTTTCATGCC |
| | | | hybridizing | | | | GAATATTCCAG |
| | | | clone | | | | CAGCTATTATA |
| | | | | | | | AAATGCTAAAT |
| | | | | | | | TCACTCATCCT |
| | | | | | | | GTACG |
| 822. | K0410D10-3 | Cxcl12 | chemokine (C- | K0410D10 | Mm.465 | Chromosome 6 | GAGAATTAATC |
| | | | X-C motif) | | | | ATAAACGGAA |
| | | | ligand 12 | | | | GTTTAAATGAG |
| | | | | | | | GATTTGGACTT |
| | | | | | | | TGGTAATTGTC |
| | | | | | | | CCTGAG |
| 823. | H3118H11-3 | Snrpg | small nuclear | H3118H11 | Mm.21764 | Chromosome 18 | CATGAGCAAA |
| | | | ribonucleoprotein | | | | GCCCACCCTCC |
| | | | polypeptide G | | | | CGAGCTGAAG |
| | | | | | | | AAGTTTATGGA |
| | | | | | | | CAAGAAGTTAT |
| | | | | | | | CATTGAA |
| 824. | K0517D08 | BM238427 | ESTs | K0517D08 | Mm.222266 | Chromosome 19 | CTCTGTAAAGT |
| | | | BM238427 | | | | CAAGTTGCATT |
| | | | | | | | GCATTTACAGT |
| | | | | | | | TAATTATGGAA |
| | | | | | | | AAGTCCTAAAT |
| | | | | | | | CTGGC |
| 825. | L0227G11-3 | Sh3d1B | SH3 domain | L0227G11 | Mm.40285 | Chromosome 12 | TTTTCAGGGCT |
| | | | protein 1B | | | | ATAAAAGTATT |
| | | | | | | | ATGTGGAAATG |
| | | | | | | | AGGCATCAGA |
| | | | | | | | CCACCGGACGT |
| | | | | | | | TACCAC |
| 826. | H3134B10-3 | 6530409L22Rik | RIKEN cDNA | H3134b10 | Mm.41940 | Chromosome Multiple | AAGAAGCTGA |
| | | | 6530409L22 | | | Mappings | GGAAAAACAG |
| | | | gene | | | | GAGAGTGAGA |
| | | | | | | | AACCGCTTTTG |
| | | | | | | | GAACTATGAGT |
| | | | | | | | TCTGCTCT |
| 827. | H3115A08-3 | Ly6a | lymphocyte | H3115A08 | Mm.263124 | Chromosome 15 | CCTGATGGAGT |
| | | | antigen 6 | | | | CTGTGTTACTC |
| | | | complex, locus | | | | AGGAGGCAGC |
| | | | A | | | | AGTTATTGTGG |
| | | | | | | | ATTCTCAAACA |
| | | | | | | | AGGAAA |
| 828. | C0120G03-3 | Csk | c-src tyrosin | C0120G03 | Mm.21974 | Chromosome 9 | AGCAAATGGG |
| | | | kinase | | | | CATTTTACAAG |
| | | | | | | | AAGTACGAATC |
| | | | | | | | TTATTTTTCCT |
| | | | | | | | GTCCTGCCCCT |
| | | | | | | | GGGGGT |
| 829. | H3094G08-3 | Tigd2 | tigger | H3094G08 | Mm.25843 | Chromosome 6 | CTGCACTTGAA |
| | | | transposable | | | | TGGACTGAAA |
| | | | element derived | | | | ACTTGCTGGAT |
| | | | 2 | | | | TATCTAGAACA |
| | | | | | | | ACAAGATGAC |
| | | | | | | | ATGCTAC |
| 830. | NM_008362.1 | IIlr1 | interleukin 1 | NM_008362 | Mm.896 | Chromosome 1 | AGATTTCACCG |
| | | | receptor, type 1 | | | | TACTTTCTGAT |
| | | | | | | | GGTGTTTTTAA |
| | | | | | | | AAGGCCAAGT |
| | | | | | | | GTTGCAAAAGT |
| | | | | | | | TTGCAC |
| 831. | C0300E10-3 | Trps1 | trichorhinophal | C0300E10 | Mm.30466 | Chromosome 15 | ATAAAACCAC |
| | | | angeal | | | | AAACTAGTATC |
| | | | syndrome I | | | | ATGCTTATAAG |
| | | | (human) | | | | TGCACAGTAGA |
| | | | | | | | AGTATAGAACT |
| | | | | | | | GATGGG |
| 832. | L0274A03-3 | Ptpn2 | protein tyrosine | L0274A03 | Mm.260433 | Chromosome 18 | ACCTAAATGTT |
| | | | phosphate, | | | | CATGACTTGAG |
| | | | non-receptor | | | | ACATTCTGCA |
| | | | type 2 | | | | GCTATAAAATT |
| | | | | | | | TGAACCTTTGA |
| | | | | | | | TGTGC |
| 833. | H3005H07-3 | 1810031K02Rik | RIKEN cDNA | H3005H07 | Mm.145384 | Chromosome 4 | TTTATAGTTCT |
| | | | 1810031K02 | | | | AGGTTTACACC |
| | | | gene | | | | AGAGAGGAGT |
| | | | | | | | TAATTTATCAA |
| | | | | | | | CAGCCTAAAAC |
| | | | | | | | TGTTGC |
| 834. | H3109H12-3 | 1810009M01Rik | RIKEN cDNA | H3109H12 | Mm.28385 | Chromosome Multiple | TTCTTCCACGA |
| | | | 1810009M01 | | | Mappings | ACAGATATTAT |
| | | | gene | | | | GTCATTTTATC |
| | | | | | | | CAATGCCCGATA |
| | | | | | | | AAGGAGAAAC |
| | | | | | | | AACTTG |
| 835. | J0008D01-3 | Enpp1 | ectonucleotide | J0008D01 | Mm.27254 | Chromosome 10 | TACGTGGTCTG |
| | | | pyrophosphatase/ | | | | GGGACCTGATG |
| | | | phosphodiesterase | | | | TTGGAATCCTA |
| | | | 1 | | | | TTGTTGTTAAT |
| | | | | | | | AAAACTGAGT |
| | | | | | | | AAAGGA |
| 836. | H3119HO5-3 | Mafb | v-maf | H3119H05 | Mm.233891 | Chromosome 10 | ACCAACTTCTG |
| | | | musculoaponiurotic | | | | TCAAAGAACA |
| | | | fibrosarcoma | | | | GTAAAGAACTT |
| | | | oncogene | | | | GAGATACATCC |
| | | | family, protein | | | | ATCTTTGTCAA |
| | | | B (avian) | | | | ATAGTC |
| 837. | H3048G11-3 | Blvrb | biliverdin | H3048G11 | Mm.24021 | Chromosome 7 | TGACACAAATA |
| | | | reductase B | | | | GAGGGGTCAA |
| | | | (flavin | | | | TAAATTTTTAG |
| | | | reductase | | | | CCAAAAGCTTC |
| | | | (NADPH)) | | | | AAATTCTTTCA |
| | | | | | | | GGAAGC |
| 838. | H3107D05-3 | 1110004C05Rik | RIKEN cDNA | H3107D05 | Mm.14102 | Chromosome 7 | ATCACCATTGT |
| | | | 1110004C05 | | | | TAGTGTCATCA |
| | | | gene | | | | TCATTGTTCTT |
| | | | | | | | AACGCTCAAA |
| | | | | | | | ACCTTCACACT |
| | | | | | | | TAATAG |
| 839. | H3006B01-3 | Cklfsf3 | chemokine-like | H3006B01 | Mm.292081 | Chromosome 8 | GCCGCTTTTTT |
| | | | factor super | | | | GTAACCTAAAA |
| | | | family 3 | | | | GGCCCCATGAA |
| | | | | | | | TAAGGGCCCAT |
| | | | | | | | GTTTTGGGCAT |
| | | | | | | | TTGTA |
| 840. | L0853H04-3 | | transcribed | L0853H04 | Mm.275315 | Chromosome 12 | CCAAGAACAA |
| | | | sequence with | | | | GTATAAACTTA |
| | | | weak similarity | | | | AGCTCTGTAGA |
| | | | to protein | | | | ACTGAAATTCT |
| | | | pir:A43932 | | | | TTCAAGTCCTT |
| | | | (H. sapiens) | | | | TCGATC |
| | | | A43932 mucin | | | | |
| | | | 2 precursor, | | | | |
| | | | intestinal- | | | | |
| | | | human | | | | |
| | | | (fragments) | | | | |
| 841. | C0949G05-3 | BM221093 | ESTs | C0949G05 | Mm.221696 | Chromosome 6 | AGGACATCTTG |
| | | | BM221093 | | | | CAACTTCTATG |
| | | | | | | | CASATAATAAG |
| | | | | | | | GATTTCCATCT |
| | | | | | | | GACAAATAAG |
| | | | | | | | ACAAGTG |
| 842. | K0648D10-3 | tlr1 | toll-like | K0648D10 | Mm.33922 | Chromosome 5 | GGGGAGTTCTA |
| | | | receptor 1 | | | | ATAATAGTACC |
| | | | | | | | ATTCATATCAG |
| | | | | | | | CAAGAACCTA |
| | | | | | | | AAAATGGTTCT |
| | | | | | | | GACTTT |
| 843. | H3014E09-3 | BC016443 | cDNA sequence | H3014E09 | Mm.27182 | Chromsome 11 | TGCCACTAGTT |
| | | | BC017643 | | | | CTGACTTGGGG |
| | | | | | | | AATATGGTCCC |
| | | | | | | | TTAAACATGCC |
| | | | | | | | AAAGTGAGCTT |
| | | | | | | | TTTAA |
| 844. | H3022D06-3 | Il2rg | interleukin 2 | H3022D06 | Mm.2923 | Chromosome X | CATCAATCCTT |
| | | | receptor, | | | | TGATGGAACCT |
| | | | gamma chain | | | | CAAAGTCCTAT |
| | | | | | | | AGTCCTAAGTG |
| | | | | | | | ACGCTAACCTC |
| | | | | | | | CCCTA |
| 845. | L0201A03-3 | 2410004H05Rik | RIKEN cDNA | L0201A03 | Mm.8766 | Chromosome 14 | CAGTTGGAAA |
| | | | 241114H05 | | | | AATGGATGAA |
| | | | gene | | | | GCTCAATGTAG |
| | | | | | | | AAGAGGGATT |
| | | | | | | | ATACAAGCAGA |
| | | | | | | | ACTCTGGCA |
| 846. | H3026E03-5 | | Mus musculus | H3026E03 | Mm.249306 | Chromosome Un: not | TCAGTCAAATG |
| | | | 2 days neonate | | | placed | TGCATAACTGT |
| | | | thymus thymic | | | | AAATCAACACT |
| | | | cells cDNA, | | | | AAGAGCTCTGG |
| | | | RIKEN full- | | | | AAGGTTAAAA |
| | | | length enriched | | | | AGGTCA |
| | | | library, | | | | |
| | | | clone:E430039 | | | | |
| | | | C10 | | | | |
| | | | product:unknown | | | | |
| | | | EST, full | | | | |
| | | | insert sequence. | | | | |
| 847. | H3091E12-3 | Abhd2 | abhydrolase | H3091E12 | Mm.87337 | Chromosome 7 | AGCAGGTGTTT |
| | | | domain | | | | CGGACTTGCAA |
| | | | containing 2 | | | | TGAGCAATGCA |
| | | | | | | | ATTTTTTCTAA |
| | | | | | | | ATATGAGGATA |
| | | | | | | | TTTAC |
| 848. | H3003E01-3 | Cutl1 | cut-like 1 | H3003E01 | Mm.258225 | Chromosome 5 | CTTGCTTCTTT |
| | | | (Drosophila) | | | | AGCAAAATATT |
| | | | | | | | CTGGTTTCTAG |
| | | | | | | | AAGAGGAAGT |
| | | | | | | | CTGTCCAACAA |
| | | | | | | | GGCCCC |
| 849. | H3016H08-5 | Crsp9 | cofactor | H3016H08 | Mm.24159 | Chromosome 11 | TCTCAATTTTC |
| | | | required for | | | | AAGGTGTATTT |
| | | | Sp1 | | | | CCTATCAGGAA |
| | | | transcriptional | | | | ACTTGAAGATA |
| | | | activation | | | | ATATGGTCTGA |
| | | | subunit 9, | | | | ACCCA |
| | | | 33kDa | | | | |
| 850. | C0118E09-3 | Oas1a | 2′-5′ | C0118E09 | Mm.14301 | Chromosome 5 | ACTGGACAAA |
| | | | oligoadenylate | | | | GTATTATGACT |
| | | | synthetase 1A | | | | TTCAACACCAG |
| | | | | | | | GAGGTCTCCAA |
| | | | | | | | ATACCTGCACA |
| | | | | | | | GACAGC |
| 851. | L0535B02-3 | Coll5a1 | procollagen, | L0535B02 | Mm.233547 | Chromosome 4 | GGCTGTTGAGT |
| | | | type XV | | | | GTAAAATGTGC |
| | | | | | | | TTTGTGTTTGC |
| | | | | | | | TTACAACATCA |
| | | | | | | | GCTTTTAGACA |
| | | | | | | | CACAG |
| 852. | L0500E02-3 | Sgcg | sarcoglycan, | L0500E02 | Mm.72173 | Chromosome 14 | TGAGTGCAATG |
| | | | gamma | | | | TGTCAGATTTC |
| | | | (dystrophin- | | | | ACCAAGAGAT |
| | | | associated | | | | CTCCAAGGTT |
| | | | glycoprotein) | | | | GTAGGTAATTT |
| | | | | | | | GTGGTT |
| 853. | H3077B08-3 | 5330431K02Rik | RIKEN cDNA | H3077B08 | Mm.101992 | Chromosome Multiple | GTCATTGTCCA |
| | | | 5330431K02 | | | Mappings | AGGTGACAGG |
| | | | gene | | | | AGGAACTCAGT |
| | | | | | | | CGTTAAAATGA |
| | | | | | | | CGAGCCTTATT |
| | | | | | | | TCATGA |
| 854. | J0209G02-3 | Gnb4 | guanine | J0209G02 | Mm.9336 | Chromosome 3 | TCTTAGAATT |
| | | | nucleotide | | | | GGAATTGAGTG |
| | | | binding protein, | | | | CCATATTTTCT |
| | | | beta 4 | | | | GTTCTCCAATG |
| | | | | | | | ATACCTGGAGA |
| | | | | | | | AATCC |
| 855. | C0661E01-3 | Lcn7 | lipocalin 7 | C0661E01 | Mm.15801 | Chromosome 4 | TGCTTTCTTAT |
| | | | | | | | TCTTTAAAGAT |
| | | | | | | | ATTTATTTTTCT |
| | | | | | | | TCTCATTAAAA |
| | | | | | | | TAAAACCAAA |
| | | | | | | | GTATT |
| 856. | K0221E09-3 | Scml2 | sex, comb on | K0221E09 | Mm.159173 | Chromosome X | CTGCATGTTAT |
| | | | midleg-like 2 | | | | AACTTTATATG |
| | | | (Drosophila) | | | | ATGGTGTAGTG |
| | | | | | | | CATATAAGCTA |
| | | | | | | | TGAGAATCATT |
| | | | | | | | TATAC |
| 857. | C0184F12-3 | D8Ertd594e | DNA segment, | C0184F12 | Mm.235074 | Chromosome 8 | CGTGCTGGAGG |
| | | | Chr 8, ERATO | | | | ACGAGAGATTC |
| | | | Doi 594, | | | | CAGAAGCTTCT |
| | | | expressed | | | | GAAGCAAGCA |
| | | | | | | | GAGAAGCAGG |
| | | | | | | | CTGAACA |
| 858. | L0602B03 | Myoz2 | myozenin 2 | L0602B03 | Mm.141157 | Chromosome 3 | TGGAGGCTTTG |
| | | | | | | | TACCCAAAACT |
| | | | | | | | TTTCAAGCCTG |
| | | | | | | | AAGGAAAAGC |
| | | | | | | | AGAACTGCGG |
| | | | | | | | GATTACA |
| 859. | C0944F04-3 | 1110055E19Rik | RIKEN cDNA | C0944F04 | Mm.39046 | Chromosome 6 | TGGAGGATCTG |
| | | | 1110055E19 | | | | TGTGAAAAAG |
| | | | gene | | | | AAGTCACCCTC |
| | | | | | | | ACAAACCGCC |
| | | | | | | | GTGCCTAAGGA |
| | | | | | | | CTCTGTC |
| 860. | L0004A03-3 | Gli2 | GL1-kruppel | L0004A03 | Mm.12090 | Chromosome 1 | CTATTTTGTGT |
| | | | family member | | | | AGACATCGTCT |
| | | | GL12 | | | | TGCCTGAATAG |
| | | | | | | | ACTGTGGGTGA |
| | | | | | | | ATCCAAATTTG |
| | | | | | | | GTCCA |
| 861. | L0860B03-3 | | ESTs | L0860B03 | Mm.221891 | Chromosome 5: not | TAATTATCTAC |
| | | | AV321020 | | | placed | ATTGGGGTAAT |
| | | | | | | | TGAAGTAGAA |
| | | | | | | | AGATCCATCTT |
| | | | | | | | AACTACGGTAA |
| | | | | | | | TCTCCG |
| 862. | L0841F10-3 | 2310045A20Rik | RIKEN cDNA | L0841F10 | Mm.235050 | Chromosome 5 | TTGGGTATCGT |
| | | | 2310045A20 | | | | TTATGTTTCCCA |
| | | | gene | | | | TCATAACACAT |
| | | | | | | | TCATAACACAT |
| | | | | | | | GCAATAACATC |
| | | | | | | | TAGGAAATCTT |
| 863. | L0008H10-3 | Agrn | agrin | L0008H10 | Mm.269006 | Chromosome 4 | TCTGATGTGGA |
| | | | | | | | AGTGCGGTCAT |
| | | | | | | | TCCTGGTTTAA |
| | | | | | | | CTCACAGCAAC |
| | | | | | | | TTTTAATTGGT |
| | | | | | | | CTAAG |
| 864. | C0128B02-3 | Casq1 | calsequestrin 1 | C0128B02 | Mm.12829 | Chromosome 1 | ATCTCCTGTTA |
| | | | | | | | ATGTATTTGGG |
| | | | | | | | TCAAATGCAAG |
| | | | | | | | GCCTTAATAAA |
| | | | | | | | GAAATCTGGG |
| | | | | | | | GCAGAA |
| 865. | C0645C09-3 | BM209340 | ESTs | C0645C09 | Mm.222131 | No Chromosome | GCAGCAAGAG |
| | | | BM209340 | | | location | AAAAGAGCAA |
| | | | | | | info available | GAGAGCCAAA |
| | | | | | | | GGCAAGAAAT |
| | | | | | | | CTCTCTGTCAC |
| | | | | | | | TCCCTTTTA |
| 866. | H3082B03-3 | Mylk | myosin, light | H3082B03 | Mm.288200 | Chromosome 16 | TGAGGAAAAG |
| | | | polypeptide | | | | CCCCATGTGAA |
| | | | kinase | | | | ACCTTATTTCT |
| | | | | | | | CTAAGACCATC |
| | | | | | | | CGTGATCTGGA |
| | | | | | | | AGTCGT |
| 867 | C0309D09-3 | | transcribed | C0309D09 | Mm.213420 | Chromosome 11 | ACCGGCTGTAC |
| | | | sequence with | | | | CCAAATAGAA |
| | | | moderate | | | | CGTCATTTTGA |
| | | | similarity to | | | | TATGAAGGATT |
| | | | protein | | | | TCAGCCCCTGA |
| | | | sp:P00722 (E. | | | | AGATTT |
| | | | coli) | | | | |
| | | | BGAL_ECOLI | | | | |
| | | | Beta- | | | | |
| | | | galatosidase | | | | |
| | | | (Lactase) | | | | |
| 868. | H3157H09-3 | BG076287 | ESTs | H3157H09 | Mm.131026 | Chromosome 2 | ATGGTTTCTTC |
| | | | BG076287 | | | | CAGCAATTTAG |
| | | | | | | | CATTGCCTGAG |
| | | | | | | | GGGTCTAAAA |
| | | | | | | | GAATAAGTTGG |
| | | | | | | | TTCTTG |
| 869. | H3061D03-3 | Pcsk5 | proprotein | H3061D03 | Mm.3401 | Chromosome 19 | ACAATCTCTGT |
| | | | convertase | | | | CAGCGAAAAG |
| | | | subtilisin/kexin | | | | TTCTACAACAG |
| | | | type 5 | | | | CTGTGCTGCAA |
| | | | | | | | AACATGTACAT |
| | | | | | | | TCCAAG |
| 870. | L0843D01-3 | 3732412D22Rik | RIKEN cDNA | L0843D01 | Mm.18830 | No Chromosome | AACTGTTACTG |
| | | | 3732412D22 | | | location | GATTGAAATTC |
| | | | gene | | | info available | CCATCCCCTTT |
| | | | | | | | CCCTAAAAATT |
| | | | | | | | GTGCCTTAGAA |
| | | | | | | | AACCC |
| 871. | L0702H07-3 | 5830415L20Rik | RIKEN cDNA | L0702H07 | Mm.46184 | Chromosome 5 | CGACTGAGGTT |
| | | | 5830415L20 | | | | ATGACATCCTT |
| | | | gene | | | | AGACTTTGTTG |
| | | | | | | | TATGCTGCTTC |
| | | | | | | | GAATGAACCA |
| | | | | | | | GAGATA |
| 872. | L0548G08-3 | Xin | cardiac | L0548G08 | Mm.10117 | Chromosome 9 | TGCCTCTTCAT |
| | | | morphogenesis | | | | CGCCAGTGGTC |
| | | | | | | | CAAAGGGCGC |
| | | | | | | | AGAGAGCGCA |
| | | | | | | | CTAGCAGTCAA |
| | | | | | | | TAGTGTT |
| 873. | L0803E02-3 | Nkdl | naked cuticle 1 | L0803E02 | Mm.30219 | Chromosome 8 | CCACTAATATT |
| | | | homolog | | | | TAGCCAGCCTT |
| | | | (Drosophila) | | | | CATGTAGAAG |
| | | | | | | | ACACATGGAA |
| | | | | | | | ACACAGAAGT |
| | | | | | | | AAACTTTT |
| 874. | C0925G12-3 | Fbxo30 | F-box protein | C0925G12 | Mm.276229 | Chromosome 10 | AGAAATGAAC |
| | | | 30 | | | | ATACATTGTCA |
| | | | | | | | GCATTTAGAAG |
| | | | | | | | TAAGTTGTGAA |
| | | | | | | | GACAGGGACA |
| | | | | | | | TTAAGTG |
| 875 | L0911A11-3 | 2010313D22Rik | RIKEN cDNA | L0911A11 | Mm.260594 | Chromosome 5 | CAAACGGGAT |
| | | | 2010313D22 | | | | CCTGTCTTCTT |
| | | | gene | | | | CTTTTCTAATA |
| | | | | | | | GAATTTTGTAA |
| | | | | | | | AGGAAATGAA |
| | | | | | | | TGTAGCC |
| 876. | AF084466.1 | rrad | Ras-related | Af084466 | Mm.29467 | Chromosome 8 | ACCGTTCTATC |
| | | | associated with | | | | ACTGTGGATGG |
| | | | diabetes | | | | AGAAGAAGCG |
| | | | | | | | TCACTATTGGT |
| | | | | | | | CTATGACATTT |
| | | | | | | | GGGAAG |
| 877. | H3073G09-3 | 1600029N02Rik | RIKEN cDNA | H0373G09 | Mm.154121 | Chromosome 7 | CTATTTTTGGG |
| | | | 1600029N02 | | | | AGATGTCTATT |
| | | | gene | | | | GCGGAGTACA |
| | | | | | | | GTAATATATAC |
| | | | | | | | CCAGAGTATGT |
| | | | | | | | CTATAG |
| 878. | L0815B08-3 | 1100001D19Rik | RIKEN cDNA | L0815B08 | Mm.260515 | Chromosome X | ACCCAACTCCA |
| | | | 110001D19 | | | | GTGCTCTCTGT |
| | | | gene | | | | CTTTTAGTACA |
| | | | | | | | GGATTTTCACC |
| | | | | | | | CATGTGCATGA |
| | | | | | | | AAAAT |
| 879. | J1037H05-3 | D230016N13Rik | RIKEN cDNA | J1037H05 | Mm.21685 | Chromosome 13 | TTACCATTTTT |
| | | | D230016N13 | | | | GGTTAAATGGC |
| | | | gene | | | | CAAATTCAGAA |
| | | | | | | | AATAACTCCAT |
| | | | | | | | TTGAATCTCCA |
| | | | | | | | GCAGG |
| 880. | K0421F09-3 | | transcribed | K0421F09 | Mm.222196 | Chromosome 6 | TCACCATACTT |
| | | | sequence with | | | | TGAAAGTGTAA |
| | | | weak similarity | | | | ACTACCACATA |
| | | | to protein | | | | TTAACATGTGT |
| | | | ref:NP_081764.1 | | | | GATTTAAGACC |
| | | | (M. musculus) | | | | CTCAG |
| | | | RIKEN cDNA | | | | |
| | | | 5730493B19 | | | | |
| | | | [Mus musculus] | | | | |
| 881. | H3082E06-3 | 1110003B01Rik | RIKEN cDNA | H3082E06 | Mm.275648 | Chromosome 13 | TGTTGCCCTCA |
| | | | 1110003B01 | | | | GATATGTCAGA |
| | | | gene | | | | TCAACTTGGAA |
| | | | | | | | GGAAAGACCTT |
| | | | | | | | CTACTCCAAGA |
| | | | | | | | AGGAC |
| 882. | C0935B04-3 | Hhip | Hedgehog- | C0935B04 | Mm.254493 | Chromosome 8 | TCTAACAAGTG |
| | | | interacting | | | | TATTTGTGTTA |
| | | | protein | | | | TCTTTAAAATA |
| | | | | | | | GAACAATTGTA |
| | | | | | | | TCTTGAAATGG |
| | | | | | | | TAAAT |
| 883. | H3116B02-3 | 1110007C05Rik | RIKEN cDNA | H3116B02 | Mm.27571 | Chromosome 7 | CGACACTGGGT |
| | | | 1110007C05 | | | | GGCCCTGCGAC |
| | | | gene | | | | AGGTAGATGG |
| | | | | | | | CATCTACTATA |
| | | | | | | | ATCTGGACTCA |
| | | | | | | | AAGCTG |
| 884. | C0945G10-3 | Tp53il1 | tumor protein | C0945G10 | Mm.41033 | Chromosome 2 | TCTCAGAGGTG |
| | | | p53 inducible | | | | TTGAAGATTTA |
| | | | protein 11 | | | | TCATCTTGAAT |
| | | | | | | | CCTCCACAAAT |
| | | | | | | | ACAGATACAGT |
| | | | | | | | CCCAA |
| 885. | K0440609-3 | Tgfb3 | transforming | K0440G09 | Mm.3992 | Chromosome 12 | TCTTTTCACCT |
| | | | growth factor | | | | CGATCAGCATC |
| | | | beta 3 | | | | ATGAGTCATCA |
| | | | | | | | CAGATCATGTA |
| | | | | | | | ATTAGTTTCTG |
| | | | | | | | GGCCA |
| 886. | L0916G12-3 | BM118833 | ESTs | L0916G12 | Mm.221415 | Chromosome 6 | TGGGAATTGCA |
| | | | BM118833 | | | | TTTAGGATAGA |
| | | | | | | | ATTGTATCTGA |
| | | | | | | | TTTGCAAAATC |
| | | | | | | | CATAAGCTCTC |
| | | | | | | | ATGCC |
| 887. | L0505A04-3 | Dnajb5 | DnaJ (Hsp40) | L0505A04 | Mm.20437 | Chromosome 4 | TACTCCCACAG |
| | | | homolog | | | | TTGTATAGAAG |
| | | | subfamily B, | | | | TCGAATAGTGA |
| | | | member 5 | | | | AGGAGCTGGG |
| | | | | | | | AGAAAACTGCT |
| | | | | | | | TCAGCT |
| 888. | L0542E08-3 | Usmg4 | unpregulated | L0542E08 | Mm.27881 | Chromosome 3 | CCGCACTTAGC |
| | | | during skeletal | | | | CTAGACCTTT |
| | | | muscle growth 4 | | | | CTTACATGATC |
| | | | | | | | TCAAGTTGAAC |
| | | | | | | | CGACTTCCTTA |
| | | | | | | | ACTCT |
| 889. | L0223E12-3 | Sparcll | SPARC-like 1 | L0223E12 | Mm.29027 | Chromosome 5 | GCTTTGGAATT |
| | | | (mast9, hevin) | | | | AAAGAGGAGG |
| | | | | | | | ATATAGATGAA |
| | | | | | | | AACCCCCTCTT |
| | | | | | | | TTGAATTAAGA |
| | | | | | | | TTTGAG |
| 890. | K0349C07-3 | 4631423F02Rik | RIKEN cDNA | K0349C07 | Mm.68617 | Chromosome 1 | AAATCAGATAT |
| | | | 4631423F02 | | | | GCAGGTCATCT |
| | | | gene | | | | GATAAATGAGT |
| | | | | | | | TAATGTTTGAT |
| | | | | | | | ATTCGGGGTAT |
| | | | | | | | CTCAC |
| 891. | C0302A11-3 | | EST B1988881 | C0302A11 | Mm.260261 | No Chromosome | GAACCATATGC |
| | | | | | | location | TGGAATGAAA |
| | | | | | | info available | CATAAGAGTTT |
| | | | | | | | TCAACAGTTAT |
| | | | | | | | CCTCTCACCTC |
| | | | | | | | TGTATG |
| 892. | C0930C11-3 | Fgfl3 | fibroblast | C0930C11 | Mm.7995 | Chromosome X | GTATCGTCAAT |
| | | | growth factor | | | | CCCAGTCAGTA |
| | | | 13 | | | | AGATAAGTTGA |
| | | | | | | | AACAAGATTAT |
| | | | | | | | CCTCAAGTGTA |
| | | | | | | | GATTT |
| 893. | H3022A11-3 | Cald1 | caldesmon 1 | H3022A11 | Mm.130433 | Chromosome 6 | GTCAAAAACG |
| | | | | | | | CCTTCAGGAAG |
| | | | | | | | CCTTAGAGCGT |
| | | | | | | | CAGAAGGAGT |
| | | | | | | | TTGATCCGACC |
| | | | | | | | ATAACAG |
| 894. | C0660B06-3 | Csrp1 | cysteine and | C0660B06 | Mm.196484 | Chromosome 1 | AATAGAATCTT |
| | | | glycine-rich | | | | TTCACTTAGGA |
| | | | protein1 | | | | ATGGAGAACA |
| | | | | | | | AGCCAGTTCAG |
| | | | | | | | AGGACCCCAA |
| | | | | | | | AGTCTAG |
| 895. | L0949F12-3 | Heyl | hairy/enhancer- | L0949F12 | Mm.103615 | Chromosome 4 | CGTGGAGGAT |
| | | | of-split related | | | | GGGCTAGCCTG |
| | | | with YRPW | | | | AGCTCTGGGAC |
| | | | motif-like | | | | TAATCTTTATT |
| | | | | | | | ACATACTTGTT |
| | | | | | | | AATGAG |
| 896. | K0225B06-3 | Unc5c | unc-5 homolog | K0225B06 | Mm.24430 | Chromosome 3 | CTTATAGGGAG |
| | | | C (C. elgans) | | | | AATGTTCTATT |
| | | | | | | | CCTCAATCCAT |
| | | | | | | | ACTCATTCCTA |
| | | | | | | | CAGTATGCGCT |
| | | | | | | | CTGGA |
| 897. | K0541E04-3 | Herc3 | hect domain | K0541E04 | Mm.33788 | Chromosome 6 | AGCAGGGGGA |
| | | | and RLD 3 | | | | TTATGTTAAGT |
| | | | | | | | CAAATGCGTGT |
| | | | | | | | GTCTCAAAAGT |
| | | | | | | | GACATGTTTAA |
| | | | | | | | CTGCTC |
| 898. | C0151A03-3 | BC026744 | cDNA sequence | C0151A03 | Mm.4079 | Chromosome 5 | ACTCTGTACCC |
| | | | BC026744 | | | | TACTGGAACCA |
| | | | | | | | CTCTGTAAAGA |
| | | | | | | | GACAAAGCTGT |
| | | | | | | | ATGTGCCACTT |
| | | | | | | | CAGTA |
| 899. | L0045C07-3 | 6-Sep | septin 6 | L0045C07 | Mm.258618 | Chromosome X | TTACAGGTCAC |
| | | | | | | | TGTTTGTCACT |
| | | | | | | | TTTGTGTACCA |
| | | | | | | | GCTTCCCCATT |
| | | | | | | | AGAATTCAACC |
| | | | | | | | GATAC |
| 900. | L0509E03-3 | Ryr2 | ryanodine | L0509E03 | Mm.195900 | Chromosome 13 | ATGGAAGCGA |
| | | | receptor 2, | | | | GGTCATTCTGC |
| | | | cardiac | | | | GAACATTGGA |
| | | | | | | | GATCTTTTATT |
| | | | | | | | ACAAGTCTGCT |
| | | | | | | | TGTTAAT |
| 901. | H3049B08-3 | Tes | tetis derived | H3049B08 | Mm.271829 | Chromosome 6 | TAAAATTAGTG |
| | | | transcript | | | | TCCTGGGAGAG |
| | | | | | | | ATGACCATTTT |
| | | | | | | | AACTTCTATGC |
| | | | | | | | TTATTTCACAT |
| | | | | | | | GGGAA |
| 902. | L0533C09-3 | BM123974 | ESTs | L0533C09 | Mm.213265 | Chromosome 14 | TCGACGTCAA |
| | | | BM123974 | | | | CTTACCTCTCT |
| | | | | | | | AGGCAACATGT |
| | | | | | | | TATCCCCGGAT |
| | | | | | | | GATCAGAAATT |
| | | | | | | | CCCAA |
| 903. | H3108C01-3 | 4930444A02Rik | RIKEN cDNA | H3108C01 | Mm.17631 | Chromosome 8 | ACCTGTGTTTT |
| | | | 4930444A02 | | | | GTTTTTGTTTT |
| | | | gene | | | | AAGAAACCAA |
| | | | | | | | AGTGCACCAA |
| | | | | | | | GATAGCATGCT |
| | | | | | | | CTTGAGA |
| 904. | C0110C06-3 | Epb4.111 | erythrocyte | C0110C06 | Mm.20852 | Chromosome 2 | CTGCAGGTAAC |
| | | | protein band | | | | TCTCATTGGAA |
| | | | 4.1-like 1 | | | | GAAAAAGAAA |
| | | | | | | | CTACAAGAGC |
| | | | | | | | AAACAGAAGC |
| | | | | | | | CATGGGAA |
| 905. | C032H08-3 | Enah | enabled | C0324H08 | Mm.87759 | Chromosome Multiple | AAAGATTTCAT |
| | | | homolog | | | Mappings | CCACGTCTGGC |
| | | | (Drosophila) | | | | GTAGTGGAAA |
| | | | | | | | ACCCGAAGGG |
| | | | | | | | AATATGTAATG |
| | | | | | | | ATCTTTC |
| 906. | C0917A09-3 | | ESTs | C0917A09 | Mm.242207 | No Chromosome | GTGTTGTACCC |
| | | | BB231855 | | | location | TAATTTGAATT |
| | | | | | | info available | TAAAGTAGGC |
| | | | | | | | AGTAGGTAGG |
| | | | | | | | GTTAATTGGTA |
| | | | | | | | GACTATC |
| 907. | L0854B10-3 | Anks1 | ankyrin repeat | L0854B10 | Mm.32556 | Chromosome 17 | CTTGGGTTTGA |
| | | | and SAM | | | | GCACTCAGAAC |
| | | | domain | | | | ACATGGCTGCA |
| | | | containing 1 | | | | ATCATCAAGAC |
| | | | | | | | AGTTCACAGTT |
| | | | | | | | AGCTT |
| 908. | K0326D08-3 | Ly75 | lymphocyte | K0326D08 | Mm.2074 | Chromosome 2 | CCCTAAGACAA |
| | | | antigen 75 | | | | TGAAACTCAGA |
| | | | | | | | ACTCTGTGATT |
| | | | | | | | CCTGTGGAAAT |
| | | | | | | | ATTTAAAACTG |
| | | | | | | | AAATG |
| 909. | H3074H01-3 | C430017H16 | hypothetical | H3074H01 | Mm.268854 | Chromosome 3 | ATTTATAGAGG |
| | | | protein | | | | TATCCTTAACA |
| | | | C430017H16 | | | | TGCTGACTTCA |
| | | | | | | | GTAACTGCCCT |
| | | | | | | | TGTTTCTAAGG |
| | | | | | | | AAGTC |
| 910. | H3131D02-3 | Tnk2 | tyrosine kinase, | H3131D02 | Mm.1483 | Chromosome 16 | ACCTGTAGCTT |
| | | | non-receptor | | | | CACTGTGAACT |
| | | | | | | | TGTGGGCTTGG |
| | | | | | | | CTGGTCTTAGG |
| | | | | | | | AACTTGTACCT |
| | | | | | | | ATAAA |
| 911. | C0112B03-3 | Heyl | hairy/enhancer- | C0112B03 | Mm.103615 | Chromosome 4 | TAATCCCTGGC |
| | | | of-split related | | | | AAAGTCAAGA |
| | | | with YRPW | | | | CTGTGGGAAAC |
| | | | motif-like | | | | TAGAACTGGTT |
| | | | | | | | ACTCACTACTG |
| | | | | | | | CTGGTA |
| 912. | L0514A09-3 | 6430511F03 | hypothetical | L0514A09 | Mm.19738 | Chromosome X | TTAGTCCCATG |
| | | | protein | | | | ACCCCAAGGTT |
| | | | 6430511F03 | | | | AAGGTTCTGCC |
| | | | | | | | AACAAGCATTC |
| | | | | | | | TGCCTGACATC |
| | | | | | | | TACTT |
| 913. | C0234D07-3 | Fbxo30 | F-box protein | C0234D07 | Mm.276229 | Chromosome 10 | AATAAAGGCC |
| | | | 30 | | | | CCTTAGAAGCT |
| | | | | | | | ACTGTAAGCT |
| | | | | | | | CTTCAAAGTTT |
| | | | | | | | TCATGTAATCA |
| | | | | | | | TAGGCA |
| 914. | H3152A02-3 | St6ga11 | beta galactoside | h3152A02 | Mm.149029 | Chromosome 16 | AGAGATGGAG |
| | | | alpha 2, 6 | | | | ACTACACTGGG |
| | | | sialyltransferase | | | | TAGATTCTAGT |
| | | | 1 | | | | TTTTAGTTCTT |
| | | | | | | | ATTAATGTGGG |
| | | | | | | | GGAGTA |
| 915. | H3075C04-3 | Ches1 | checkpoint | H3075C04 | Mm.268534 | Chromosome 12 | TATGGCCATTT |
| | | | suppressor 1 | | | | GGTTTCAGCAT |
| | | | | | | | GTCAGGAGATT |
| | | | | | | | TCTAATGATTT |
| | | | | | | | GATGGCAATATC |
| | | | | | | | AGCAA |
| 916. | L0600E02-3 | BM125123 | ESTs | L0600E02 | Mm.221782 | Chromosome 19 | TGTGTCAAGAT |
| | | | BM125123 | | | | AATCCTGAGTC |
| | | | | | | | AACCTGGACAC |
| | | | | | | | TTAATCCCTTT |
| | | | | | | | GGACCTCTATC |
| | | | | | | | TGGAG |
| 917. | K0501F10-3 | BM237456 | ESTs | K0501F10 | Mm.34527 | Chromosome X | CCACCCATTAA |
| | | | BM237456 | | | | AATGACAGTAC |
| | | | | | | | AAGTAGACCA |
| | | | | | | | CAGTTTAAAT |
| | | | | | | | AGTTAGTCTAA |
| | | | | | | | TTCTAC |
| 918. | K0301H08-3 | Oxct | 3-oxoacid CoA | K0301H08 | Mm.13445 | Chromosome 15 | CATAGTGGAA |
| | | | transferase | | | | ATATGCTCATC |
| | | | | | | | TTTTATGCTAT |
| | | | | | | | ATGTATTAAAC |
| | | | | | | | CTCGACTTAGC |
| | | | | | | | CCTGAA |
| 919. | L0229E07-3 | Lu | Lutheran blood | L0229E07 | Mm.29236 | Chromosome 7 | GTTGAGGCTGA |
| | | | group | | | | CGACCTCCCAG |
| | | | (Auberger b | | | | AGGCAATCTCT |
| | | | antigen | | | | GGATCTGGAAC |
| | | | included) | | | | TTTGGGCATCA |
| | | | | | | | TCGGA |
| 920. | H3077C06-3 | 4931430I01Rik | RIKEN cDNA | H3077C06 | Mm.12454 | Chromosome 1 | ACCAACCAGG |
| | | | 4931430I01 | | | | GACTAGTTTGA |
| | | | gene | | | | TGCTATCTTTG |
| | | | | | | | CCTGTCTCTTG |
| | | | | | | | GCTCTTAACAA |
| | | | | | | | TGCCTA |
| 921. | J0807D02-3 | | Mus musculus | J0807D02 | Mm.125975 | Chromosome 7 | CCAGGGAAGG |
| | | | 10 days neonate | | | | AACGATCCATT |
| | | | cerebellum | | | | CAGTGGTTTTA |
| | | | cDNA, RIKEN | | | | AAATATCTCTT |
| | | | full-length | | | | CCTCAACAGAA |
| | | | enriched | | | | AAAGAT |
| | | | library, | | | | |
| | | | clone:B930022I | | | | |
| | | | 23 | | | | |
| | | | product:unclass | | | | |
| | | | ifiable, full | | | | |
| | | | insert sequence. | | | | |
| 922. | H3118G11-3 | C130068N17 | hypothetical | H3118G11 | Mm.138073 | Chromosome 2 | GGTGCAAGCTA |
| | | | protein | | | | GTACTCACACT |
| | | | C130068N17 | | | | GTCACACCTTT |
| | | | | | | | ACGCATGCGA |
| | | | | | | | AAGGTAATGTG |
| | | | | | | | CTAAAT |
| 923. | L0818F01-3 | Smarcd3 | SWI/SNF | L0818F01 | Mm.140672 | Chromosome | AGATCAGTGCT |
| | | | related, matrix | | | | CTGGACAGTAA |
| | | | associated, | | | | GATCCATGAGA |
| | | | actin dependent | | | | CGATTGAGTCC |
| | | | regulator of | | | | ATAAACCAGCT |
| | | | chromatin, | | | | CAAGA |
| | | | subfamily d, | | | | |
| | | | member 3 | | | | |
| 924. | C0359A10-3 | BM198389 | ESTs | C0359A10 | Mm.218312 | Chromosome 1 | ATACCCTGCT |
| | | | BM198389 | | | | AACTTAACAGC |
| | | | | | | | AGTTAGTTTCC |
| | | | | | | | TTGTTATGAAT |
| | | | | | | | AAAAATGACA |
| | | | | | | | GTCTGG |
| 925. | G0108E12-3 | 1190009E20Rik | RIKEN cDNA | G0108E12 | Mm.260102 | AAAGCAAATG | |
| | | | 1190009E20 | | | | TTAGTAAAAAG |
| | | | gene | | | | CTGGTGTGCAT |
| | | | | | | | AGTCTTGTTAC |
| | | | | | | | ATTGATGCAGT |
| | | | | | | | TTTTCC |
| 926 | C0941C09-3 | Gja7 | gap, junction | C0941C09 | Mm.3096 | Chromosome 11 | CAACTTGCTGA |
| | | | membrane | | | | ATAATGACTTC |
| | | | channel protein | | | | CATTGAGTAAA |
| | | | alpha 7 | | | | CATTTGGCTCT |
| | | | | | | | GGTTATCTTCA |
| | | | | | | | GGGAT |
| 927. | H3111BO305 | | UNKNOWN | H3111B03 | Data not found | No Chromosome | AGGAATTAGTA |
| | | | H3111B03 | | | location | ACGTTTCATCC |
| | | | | | | info available | AAGTAACCTTG |
| | | | | | | | TTACAGTGAAC |
| | | | | | | | AAGTGTCAAGT |
| | | | | | | | GCTCA |

The following Examples are intended to illustrate, but not limit, the invention.

### EXAMPLES

### Example 1

### Signature Patterns of Gene Expression in Mouse Atherosclerosis and their Correlation to Human Coronary Disease

Mouse genetic models of atherosclerosis allow systematic analysis of gene expression, and provide a good representation of the human disease process (Breslow (1996) Science 272: 685-688). ApoE-deficient mice predictably develop spontaneous atherosclerotic plaques with numerous features similar to human lesions (Nakashima et al. (1994) Arterioscler Thromb 14: 133-140; Napoli et al. (2000) Nutr Metab Cardiovasc Dis 10: 209-215; Reddick et al. (1994) Arterioscler Thromb 14: 141-147. On a high-fat diet, the rate and extent of progression of lesions are accelerated. In addition to environmental influences such as diet, the genetic background of mice has also been found to have an important role in disease development and progression. Whereas C57B1/6 (C57) mice are susceptible to developing atherosclerosis, the C3H/HeJ (C3H) strain of mice is resistant (Grimsditch et al. (2000) Atherosclerosis 151:389-397. Previously, genetic-based diet and age induced transcriptional differences have been demonstrated between these two strains (Tabibiazar et L. (2005) Arterioscler Thromb Vasc Biol 25:302-308.

To more fully characterize the vascular wall gene expression patterns that are associated with atherosclerosis, a systematic large scale transcriptional profiling study was undertaken to take advantage of a longitudinal experimental design, and mouse genetic model and diet combinations that provide varying susceptibility to atherosclerosis. In this experiment, atherosclerosis-associated genes were studied independent of other variables. Primarily, these studies investigated differential gene expression over time in apoE-deficient mice on an atherogenic diet, with comparison to apoe-deficient mice (C57BL/6J-Apoe^{tmlUnc}) on normal diet as well as C57B1/6 and C3H/HeJ mice on both normal chow and atherogenic diet. Identification of atherosclerosis-associated genes was facilitated by development of permutation-based statistical tools for microarray analysis which takes advantage of the statistical power of time-course experimental design and multiple biological and technical replicates. Using these tools, hundreds of known and novel genes that are involved in all stages of atherosclerotic plaque, from fatty streak to end stage lesions, were identified. To further examine the expression of individual genes in the context of particular biological or molecular pathways, a pathway enrichment methodology with gene ontology (GO) terms for functional annotation was utilized. Using classification algorithms, a signature pattern of expression for a core group of mouse atherosclerosis genes was identified, and the significance of these classifier genes was validated with additional mouse and human atherosclerosis samples. These studies identified atherosclerosis related genes and molecular pathways.

Methods

Atherosclerotic Lesion Analysis

For select time points for various experimental groups, 5 to 7 female mice were used for histological lesion analysis. Atherosclerosis lesion area was determined as described previously (Tabibiazar et al. (2005), supra). Briefly, the arterial tree was perfused with PBS (pH 7.3) and then perfusion-fixed with phosphate-buffered paraformaldehyde (3%, pH 7.3). The heart and full length of the aorta to iliac bifurcation was exposed and dissected carefully from any surrounding tissues. Aortas were then opened along the ventral midline and dissected free of the animal and pinned out flat, intimal side up, onto black wax. Aortic images were captured with a Polaroid digital camera (DMC1) mounted on a Leica MZ6 stereo microscope, and analyzed using Fovea Pro (Reindeer Graphics, Inc. P. O. Box 2281, Asheville, N.C. 28802). Percent lesion area was calculated as total lesion area/total surface area.

Experimental Design, RNA Preparation and Hybridization to Microarrays

All experiments were performed following Stanford University animal care guidelines (Saadeddin et al. (2002) Med Sci Monit 8:RA5-12). Three week old female apoE knock-out mice (C57BL/6J-Apoe^{tmlUnc}), C57Bl/6J, and C3H/HeJ mice were purchased from Jackson Labs (Bar Harbor, Me.). At four weeks of age the mice were either continued on normal chow or were fed high fat diet which included 21% anhydrous milkfat and 0.15% cholesterol (Dyets #101511, Dyets Inc., Bethlehem, Pa.) for maximum period of 40 weeks. At each of the time-points, including 0 (baseline), 4, 10, 24 and 40 weeks, for each of the conditions (strain-diet combination), 15 mice (3 pools of 5) were harvested for RNA isolation (total of 405 mice). Additional mice were used for histology for quantification of atherosclerotic lesions as described above. A separate cohort of sixteen-week-old apoE-deficient mice on high fat diet for two weeks (4 pools of 3 aortas) was also used for classification purposes.

After perfusion of mice with saline, the aortas were carefully dissected in their entireties from the aortic root to the common iliac and subsequently were flash frozen in liquid nitrogen. Total RNA was isolated as described previously (Tabibiazar et al. (2003) Circ Res 93:1192-1201) using a modified two-step purification protocol. RNA integrity was also assessed using the Agilent 2100 Bioanalyzer System with RNA 6000 Pico LabChip Kit (Agilent).

First strand cDNA was synthesized from 10 μg of total RNA from each pool and from a whole 17.5-day embryo for reference RNA in the presence of Cy5 or Cy3 dCTP, respectively. Hybridization to a mouse 60mer oligo microarray (G4120A, Agilent Technologies, Palo Alto, Calif.) (Carter et al. (2003) Genome Res 13:1011-1021) was performed following manufacture's instructions, generating three biological replicates for each of the time points. The RNA from the group of sixteen-week-old mice was linearly amplified and hybridized to a different array (G4121A, Agilent Technologies). Technical validation of the microarray has been performed previously using quantitative real-time reverse transcriptase polymerase chain reaction (results reported in Tabibiazar et al. (2005), supra). Primers and probes for 10 representative differentially expressed genes were obtained from Applied Biosystems Assays-on-Demand. A total of 90 reactions, including triplicate assays on three pools of five aortas, was performed from representative RNA samples used for microarray experiments, demonstrating a high correlation between the two platforms (Pearson correlation of 0.82).

Data Processing

Image acquisition of the mouse oligo microarrays was performed on an Agilent G2565AA Microarray Scanner System and feature extraction was performed with Agilent feature extraction software (version A.6. 1.1, Agilent Technologies). Normalization was carried out using a LOWESS algorithm. Dye-normalized signals of Cy3 and Cy5 channels were used in calculating log ratios. Features with reference values of <2.5 standard deviation for the negative control features were regarded as missing values. Those features with values in at least ⅔ of the experiments and present in at least one of the replicates were retained for further analysis. Reproducibility of microarray results, as measured by the variation between arrays for signal intensities, was assessed using box plots (GeneData,Inc., South San Francisco, Calif.). For further statistical analysis of the data, a K-nearest-neighbor (KNN) algorithm was applied to impute missing values (Troyansakaya et al. (2001) Bioinformatics 17:520-525). Numerical raw data were then migrated into an Oracle relational database (CoBi) that has been designed specifically for microarray data analysis (GeneData, Inc.). Heat maps were generated using “HeatMap Builder” software (Blake and Ridker (2002) J Intern Med 252:283-294). All microarray data were submitted to the National Center for Biotechnology information's Gene Expression Omnibus (GEO GSE1560; www.ncbi.nlm.nih.gov/geo/).

Data Analysis

i) Principal components analysis

For each gene the average log expression values were computed at the four post-baseline observation times, 4, 10, 24, and 40 weeks. This was done separately for the six different (diet, strain) combinations, for example ApoE on high fat, presumably the most atherogenic combination. Differences of these vectors were taken for various interesting contrasts, e.g., for ApoE, high-fat minus C3H, normal chow, giving N=20280 vectors of length 4, one for each gene. Principal components analysis of the N vectors showed a consistent pattern, with the first principal vector indicating a roughly linear increase with observation time.
ii) Time course regression analysis

A standard ANACOVA model was fit separately to the log expression values for each gene, using a model incorporating strain, diet, and time period effects. A single important “z value” was extracted from each ANACOVA analysis, for example corresponding to the significance of the time slope difference between the ApoE, high-fat combination and the average of the other five combinations. The N z-values were then analyzed simultaneously, using empirical Bayes false discovery rate methods described previously (Efron (2004) J Amer Stat Assoc 99:82-95; Efron and Tibshirani (2002) Genetic Epidemiology 23:70-86; Efron et al. (2001) J Amer Stat Assoc 96:1151-1160. These analyses identified a set of several hundred genes clearly associated with atherosclerosis progression.
iii) Time course area under the curve analysis

Area under the curve (AUC) analysis was employed as described previously (Tabibiazar et al. (2005), supra). For each sequence of 4 triplicate gene expression measurements over time, the measurement at time 0 was subtracted from all values. The signed area under the curve was then computed. The area is a natural measure of change over time. These areas were then used to compute an F-statistic for the 6 groups (3 mouse strains and 2 diets) and 3 replicates (between sum of squares/within sum of squares). A permutation analysis, similar to that employed in Significance Analysis of Microarrays (SAM) (Tusher et al. Proc Natl Acad Sci 98:5116-5121), was carried out to estimate the false discovery rate (q-value or “FDR”) for different levels of the F-statistic.
iv) Enrichment analysis

For enrichment analysis, the Expressionist software (GeneData, Inc.), which employs the Fisher exact test to derive biological themes within particular gene sets defined by functional annotation with Gene Ontology (GO) terms (www.geneontology.org) and Biocarta pathways (www.biocarta.com/genes/allpathways.asp), was used. In this way, over-representation of a particular annotation term corresponding to a group of genes was quantified.
v) Support vector machine for gene selection

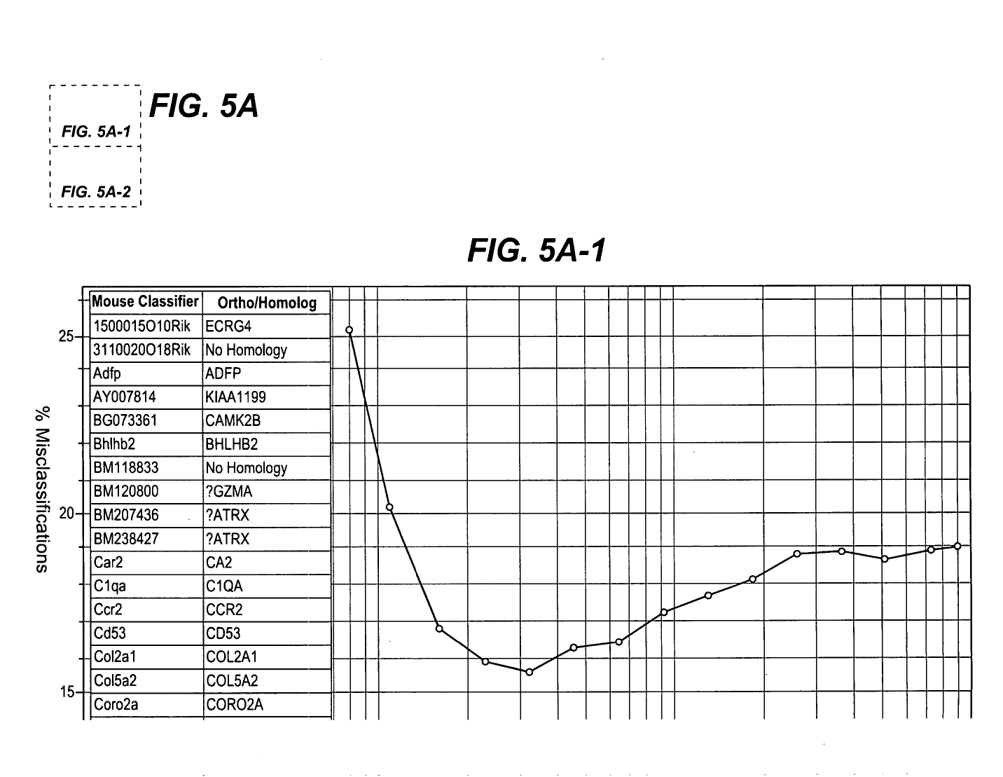

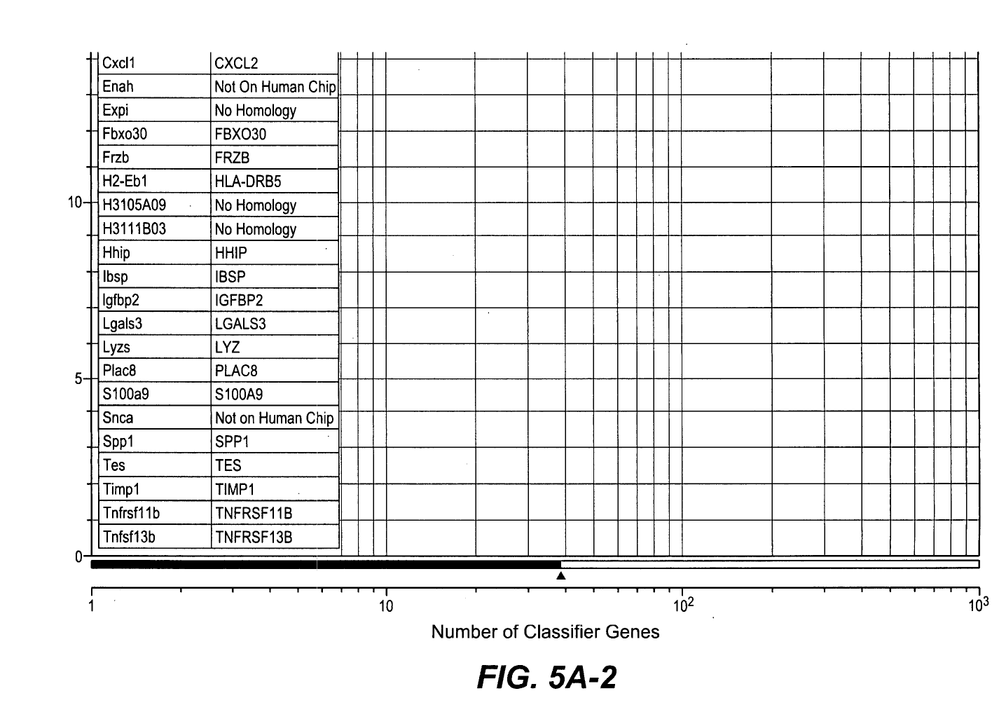

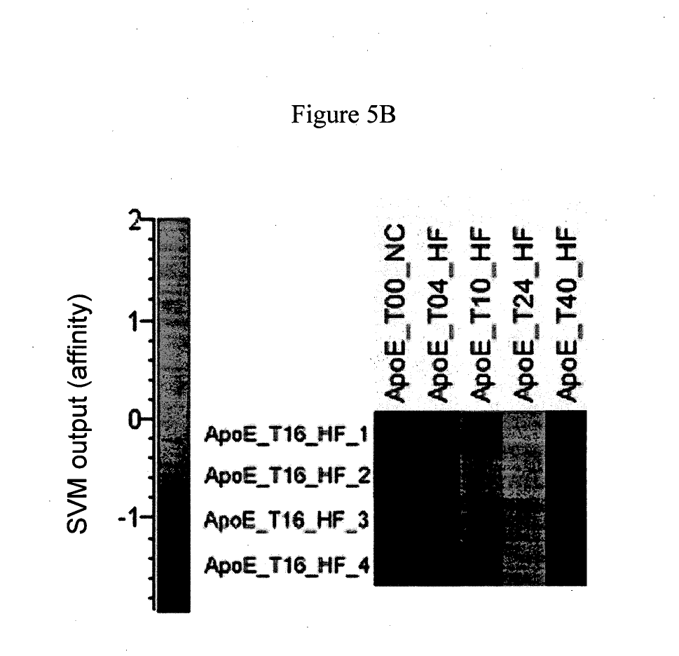

For supervised analyses, the Expressionist software (GeneData USA), which employs Support Vector Machine (SVM) algorithm (Burges (1998) Data Mining and Knowledge Discovery 2:121-167),was used to rank genes based on their utility for class discrimination between time points 0, 4, 10, 24, and 40 weeks in apoE mice on high-fat diet. SVM is a binary classifier, so in order to classify multiple categories, N classifiers were created that classify one group vs. a combination of the rest of the groups (“one vs. all” classifiers) (Ramaswamy et al. (2001) Proc Natl Acad Sci 98:15149-15154). The larger set of genes identified by the time-course analysis was used for this analysis. This method was then used to determine the optimal number of ranked genes to classify the experiments into their correct groups at minimal error rate. The optimal error rate or misclassification is calculated by cross-validation with 25% of the experiments as the test group and the rest as the training group. This is reiterated 1000 times (FIG. 5A). In this study, a linear Kernel was used, since a nonlinear Gaussian kernel yielded similar results. This minimal subset of classifier genes was then used for cross-validation as well as classification of other independent gene expression profiling datasets.
vi) Analysis of independent datasets.

The SVM algorithm was utilized for classification of independent groups of experiments (Yeang et al. (2001) Bioinformatics 17 Suppl 1:S316-322). In this analysis, the primary time-course experiments were used (corresponding to 5 time points mentioned above) as the training set and the independent set of experiments (different array and labeling methodology) as the test set. SVM output for each experiment based on one-versus-all comparisons was represented graphically in a heatmap format (FIG. 5B), which is the normalized margin value for each of the 5 SVM classifiers mentioned above. The SVM output permits classification of a new experiment according to the 5 SVM hyperplane. The SVM algorithm (Linear Kernel) was also utilized for external validation by classifying different sets of human expression data. In these analyses, a confusion matrix was generated using cross validation with repeated splits into 75% training and 25% test sets to determine the accuracy of classification based on the small subset of genes identified earlier. Results are represented in tabular fashion (Table 3).

Transcriptional Profiling of Human Atherosclerotic Tissue and Atherectomy Samples

For one set of samples, coronary arteries were dissected from explanted hearts of patients undergoing orthotopic heart transplantation. Arteries were divided into 1.5 cm segments, classified as lesion or non-lesion after inspection of the luminal surface under a dissecting microscope. RNA was isolated from each individual sample and hybridized to a microarray. A central portion (1-2mm) of each segment was removed and stored in OCT for later histological staining (hematoxylin and eosin, Masson's trichrome). Samples (n=40) were derived from 17 patients (male 13, female 4, mean age 43 years). Six patients had a diagnosis of ischemic cardiomyopathy, while 11 were classified as non-ischemic, although some vessel segments from the latter had microscopic evidence of coronary artery disease. Of 21 diseased segments, 7 were classified as grade I, 4 grade III and 9 grade V, according to the modified American Heart Association criteria (Virmani et al. (2000) Arterioscler Thromb Vasc Biol 20:1262-1275), and one sample had only macroscopic information available. For a second set of tissues, coronary atherectomy samples were obtained with a cutting atherectomy catheter system (Fox Hollow Inc., Redwood City, Calif.), for chronic atherosclerosis lesions (n=28) and in-stent restonsis lesions (n=14). Patient characteristics in both groups were similar (male 78% vs. 71%, mean age 64 vs. 67). RNA was isolated from each individual sample, labeled by direct or linear amplification methods, and hybridized as described above to a 22k feature custom cardiovascular oligonucleotide microarray designed in conjunction with Agilent Technologies (G2509A, Agilent Inc., Palo Alto, Calif.). Common reference RNA for all human hybridizations was a mixture of 80% HeLa cell RNA and 20% human umbilical vein endothelial cell RNA. Data processing and analysis were performed as described above. For 2-class comparison of gene expression, Significance Analysis of Microarrays (SAM) was used (www-stat.stanford.edu/tibs/SAM/; Tabibiazar et al. (2003), supra; Tusher et al. (2002), supra).

Results and Discussion

Atherosclerosis in the Genetic Models

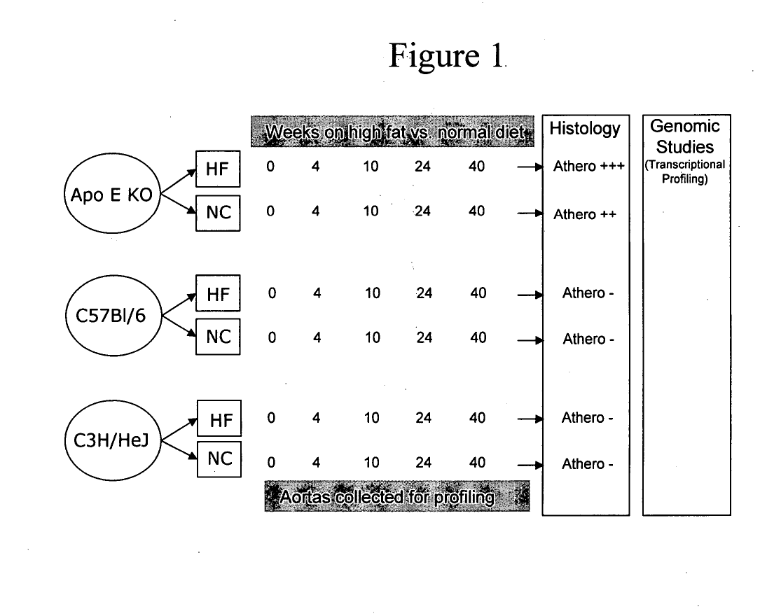

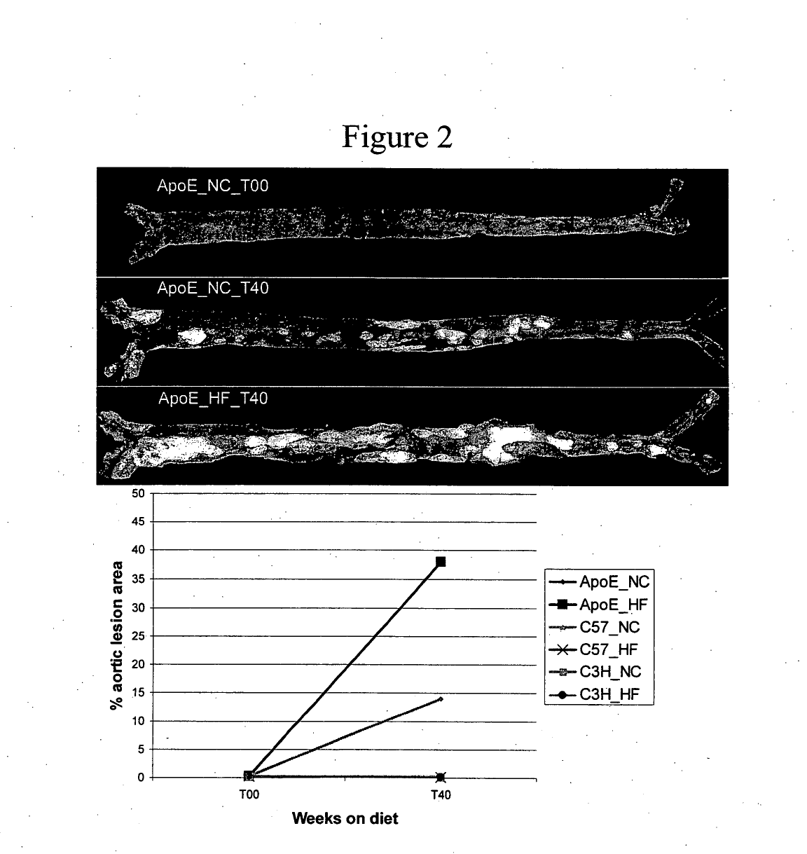

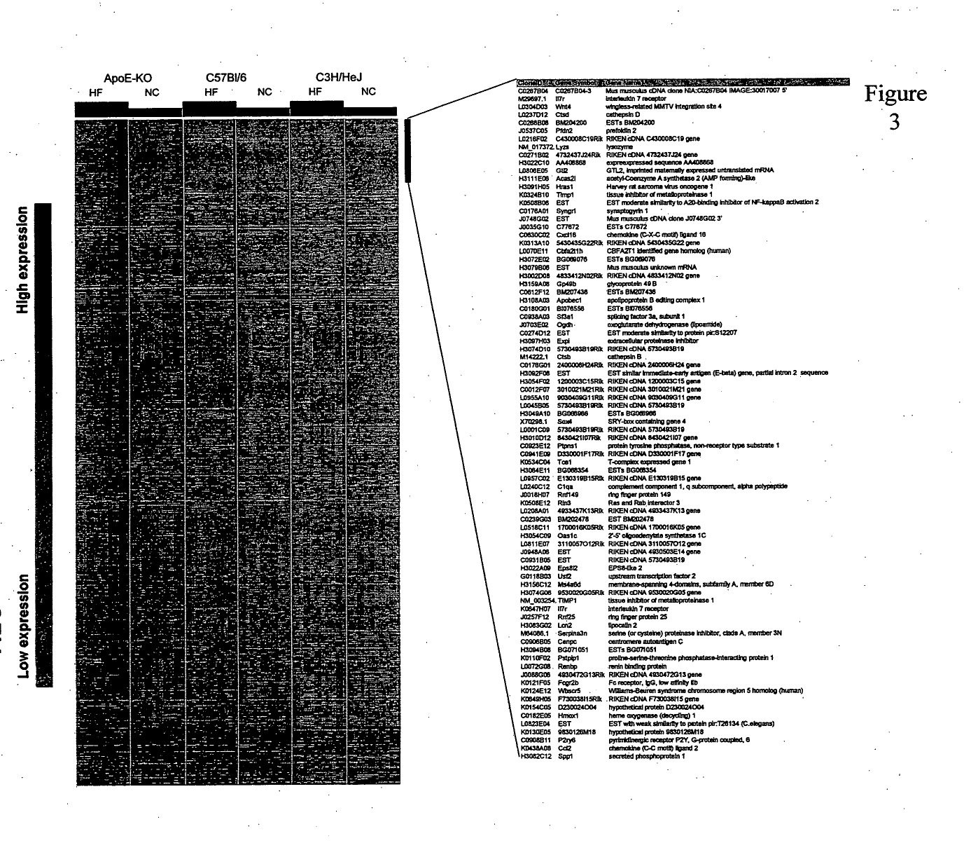

To correlate the gene expression results with the extent of disease in each experimental group, the total atherosclerotic plaque burden in the aorta was determined by calculating a percent lesion area from the ratio of atherosclerotic area to total surface area. ApoE-deficient mice (C57BL/6J-Apoe^{tmlUnc}) (n=7) on high-fat diet were compared to other control mice (n=5-7 for each mouse-diet combination). Representative time-intervals were used for analysis, including baseline measurements in mice prior to initiation of high-fat diet at 4 weeks and end-point measurements corresponding to 40 weeks on either high-fat or normal diet (FIGS. 1, 2). Gross histological evaluation of these mice demonstrated increased atherosclerotic lesions in ApoE-deficient mice on high-fat diet involving about 50% of the entire aorta, and lesser area involved in ApoE-deficient mice on normal diet (FIG. 2). As expected, the control mice on either diet did not demonstrate evidence of atherosclerosis throughout the course of the experiment (Jawien et al. (2004) J Physiol Pharmacol 55:503-517; Nishina et al. (1990) J Lipid Res 31:859-869). Although some fatty infiltrates were noted on histological evaluation of the aortic root in C57 mice on high-fat diet, there were no obvious changes in inflammatory cell infiltrate (Tabibiazar et al. (2005), supra). The metabolic and lipid profiles of these mice were not obtained in this study, since they are well described in the literature (Grimsditch et al., supra; Nishina et al. (1990), supra; Nishina et al. (1993) Lipids 28:599-605).

Temporal Patterns of Gene Expression

Employing a number of mouse models with different propensity to develop atherosclerosis, two different diets, and a longitudinal experimental design, it was possible to factor out differentially regulated genes that are unlikely to be related to the vascular disease process in the apoE deficient model. For instance, age-related and diet-related gene expression patterns that are not linked to vascular disease were eliminated by virtue of their expression in the genetic models that did not develop atherosclerosis. However, the complexity of the experimental design provided significant difficulties related to statistical analysis. Although analytic methods have been proposed to address a single set of time-course microarray data (Luan and Li (2003) Bioinformatics 19:474-482; Park et al. (2003) Bioinformatics 19:694-703; Peddada et al. (2003) Bioinformatics 19:834-841; Xu and Li (2003) Bioinformatics 19:1284-1289), there was no accepted algorithm for comparing differences in patterns of gene expression across multiple longitudinal datasets.

Using principle component analysis, it was determined that the greatest variation in the data was between time points, correlating with the progression of disease described previously for the apoE knockout mouse on high fat diet (Nakashima et al. (1994) Arterioscler Thromb 14:133-140; Reddick et al. (1994) Arterioscler Thromb 14:141-147). Given this finding, a linear regression model was utilized to identify genes that were differentially expressed in ApoE-deficient mice on high-fat diet, compared with all other experimental groups across time. This comparison across strains and dietary groups was employed to focus the analysis on atherosclerosis-specific genes, taking into account gene expression changes in the vessel wall associated with aging, diet, and genetic background. Empirical Bayes and permutation methods were employed to derive a false discovery rate (FDR) and minimize false detection due to multiple testing. With high stringency limits, global FDR<0.05 and local FDR<0.3, 667 genes demonstrated a linear increase with time, whereas only 64 genes showed the opposite profile (FIG. 3).

Genes with Increased Expression in the Atherosclerotic Vessel Wall

The identification of known genes previously linked to atherosclerosis validated the methodology and analysis algorithm. Most striking in this regard were inflammatory genes, including chemokines and chemokine receptors, such as Ccl2, Ccl9, CCr2, CCr5, Cklfsf7, Cxcl1, Cxcl12, Cxcl16, and Cxcr4 (FIG. 3). Also upregulated were interleukin receptor genes, including IL1r, IL2rg, IL4ra, IL7r, IL10ra, IL13ra, and IL15ra, and major histocompatibility complex (MHC) molecules such as H2-EB1 and H2-Ab. The value of transcriptional profiling in this disease was demonstrated by the identification of numerous inflammatory genes not previously linked to atherosclerosis, including CD38, Fcer1g, oncostatin M (Osm) and its receptor (Osmr).

Oncostatin M (Osm) and its cognate receptor (Osmr) are likely to have significant roles in atherosclerosis, based on number of studies that suggest several important related functions for these genes (Mirshahi et al. (2002) Blood Coagul Fibrinolysis 13:449-455. Osm is a member of a cytokine family that regulates production of other cytokines by endothelial cells, including Il6, G-CSF and GM-CSF. Osm also induces Mmp3 and Timp3 gene expression via JAK/STAT signaling (Li et al. (2001) J Immunol 166:3491-3498). It induces cyclooxygenase-2 expression in human vascular smooth muscle cells (Bernard et al. (1999) Circ Res 85:1124-1131), as well as Abcal in HepG2 cells (Langmann et al. (2002) J Biol Chem 277:14443-14450). Interestingly, Stat1, Jak3, Cox2, and Abca1 were among the disease-associated upregulated genes. Additionally, Osm produced by macrophages may contribute to development of vascular calcification (Shioi et al. (2002) Circ Res91:9-16). This may occur via regulation of osteopontin or osteoprotegerin (Palmqvist et al. (2002) J Immunol 169:3353-3362, both of which have demonstrated significant changes in the dataset described herein. Osteopontin (Spp1) is thought to mediate type-1 immune responses (Ashkar et al. (2000) Science 287:860-864. While Spp1 has been extensively studied in atherosclerosis and other immune diseases, some of the osteopontin-related genes identified through these studies are novel and provide additional links between inflammation and calcification. Some of these include Cd44, Hgf; osteoprotegerin, Mglap, Il10ra, Infgr, Runx2, and Ccnd1. Ibsp, (sialoprotein II), was also noted to be upregulated in these studies. Despite its similar expression profile to Spp1 in various cancer types and its binding to the same alpha-v/beta-3 integrin, the role of Ibsp in atherosclerosis has not been elucidated.

Known and novel genes were identified for many other protein classes that have been studied in atherosclerosis. Genes encoding endothelial cell adhesion molecules were among these groups, including Alcam and Vcam1. Extracellular matrix and matrix remodeling proteins were found to be upregulated, including fibronectin, Col8al, Ibsp, Igsf4, Itga6, and thrombospondin-1. Matrix metalloproteinase genes such as Mmp2 and Mmp14 as well as those encoding tissue inhibitors of metalloproteinases, including Timp1, were also among the upregulated genes. Many transcription factors, lipid metabolism and vascular calcification genes, as well as macrophage and smooth muscle cell specific genes, were among those found to be upregulated. New genes were identified in each of these classes, for example, members of the ATP-binding-cassette family that were not previously associated with atherosclerosis were identified through these studies, including Abcc3 and Abcb1b.

Interesting genes linked to atherosclerosis for the first time through these studies encode a variety of functional classes of proteins. For example, genes encoding transcription factors Runx2 and Runx3 were linked to atherosclerosis in these studies. Cytoplasmic signaling molecules Vav1, Hras1, and Kras2 are factors that are well known to have critical signaling functions, but their role in atherosclerosis has not yet been defined. Wispl is a secreted wnt-stimulated cysteine-rich protein that is a member of a family of factors with oncogenic and angiogenic activity. Rgs10 is a member of a family of cytoplasmic factors that regulate signaling through Toll-like receptors and chemokine receptors in immune cells. Among the new classes of genes identified through these studies to be upregulated in atherosclerosis were those encoding histone deacetylases. Among those genes identified were Hdac7and Hdac2. Although there is significant evidence that HDACs have important functions regulating growth, differentiation and inflammation, these molecules have not been well studied in the context of atherosclerosis (Dressel et al. (2001) J Biol Chem 276:17007-17013); Ito et al. (2002) Proc Natl Acad Sci 99:8921-8926). Histone deacetylase inhibitors have been postulated to modulate inflammatory responses (Suuronen et al. (2003) Neurochem 87:407-416).

The data from the experiments described herein has also yielded numerous ESTs and uncharacterized genes. These genes may be attractive candidates for further characterization. One example of such ESTs is 2510004L01Rik, a gene termed “viral hemorrhagic septicemia virus induced gene” (VHSV), which was originally cloned from interferon-stimulated macrophages. This gene is enriched in bone marrow macrophages, is upregulated by CMV infection and is similar to human inflammatory response protein 6 (Chin and Cresswell (2001) Proc Natl Acad Sci 98:15125-15130). Several ESTs such as 5930412E23Rik and 2700094L05Rik have been cloned from hematopoietic stem cells (genome-www5.stanford.edu/cgi-bin/source/sourceSearch), consistent with data suggesting cells in the diseased vessel wall may emanate from the bone marrow (Rauscher et al. (2003) Circulation 108:457-463.

Genes with Decreased Expression in the Atherosclerotic Vessel Wall

The 64 genes that showed decreased expression during progression of atherosclerosis were of interest, given the lack of previous attention to such genes. Sparcl1 (Hevin) is an extracellular matrix protein which is downregulated in the dataset described herein, and may have antiadhesive (Girard and Springer (1996) J Biol Chem 271:4511-4517) and antiproliferative (Claeskens et al. (2000) Br J Cancer 82:1123-1130) properties. It has been shown to be downregulated in neointimal formation and suggested to have a possible protective effect in the vessel wall (Geary et al. (2002) Arterioscler Thromb Vasc Biol 22:2010-2016). Another gene with decreased expression, Tgfb3, may also have a protective effect. The factor encoded by this gene has been shown to decrease scar formation, and to exert an inhibitory effect on G-CSF, suggesting an anti-inflammatory role that would counter pro-inflammatory factors in the vascular wall (Hosokawa et al. (2003) J Dent Res 82:558-564); Jacobsen et al. (1993) JImmunol 151:4534-4544).

Interestingly, numerous genes characteristic of various muscle lineages were shown to be downregulated. For smooth muscle cells, this might reflect decreased expression of differentiation markers. For example, the smooth muscle cell gene caldesmon encodes a marker of differentiated smooth muscle cells (Sobue et al. (1999) Mol Cell Biochem 190:105-118), and previous studies have noted that the population of differentiated contractile smooth muscle cells that express caldesmon is relatively lower in atherosclerotic plaque (Glukhova et al. (1988) Proc Natl Acad Sci 85:9542-9546). Other potential smooth muscle cell marker genes with decreased expression included Csrp1 and Mylk. Other downregulated skeletal and cardiac muscle genes included calsequesterin, which is expressed in fast-twitch skeletal muscle, Usmg4, which is upregulated during skeletal muscle growth, Xin, which is related to cardiac and skeletal muscle development, and Sgcg, that is strongly expressed in skeletal and heart muscle as well as proliferating myoblasts. The possible association of these and other myocyte related genes identified in this study to normal vascular function is not known.

Pathways Analysis

To identify important biological themes represented by genes differentially expressed in the atherosclerotic lesions, the genes were functionally annotated using Gene Ontology (GO) terms (www.geneontology.org) and curated pathway information. Enrichment analysis with the Fisher Exact Test demonstrated several statistically significant ontologies (Table 3), including several associated with inflammation. Inflammatory processes such as immune response, chemotaxis, defense response, antigen processing, inflammatory response, as well as molecular functions such as interleukin receptor activity, cytokine activity, cytokine binding, chemokine and chemokine receptor activity, Tnf-receptor, and MHC I and II receptor activity were noted to be significantly over-represented in the group of genes upregulated with atherosclerosis. Subanalysis of the inflammatory response pathways revealed genes characteristic of the macrophage lineage, as well as both the TH-1 and TH-2 T-cell populations, to be over-represented. Biocarta terms further delineated novel genes that were associated with pathways within the inflammation category, including classical complement, Rac-CyclinD, Egf, and Mrp pathways, as well as those known to be differentially regulated in atherosclerosis, such as Il2, Il7, Il22, Cxcr4, CCr3, Ccr5, Fcer1, and Infg pathways.

In addition to inflammation, other biological processes and molecular functions were over-represented in the group of differentially upregulated genes. These included expected pathways such as wound healing, ossification, proteo- and peptidolysis, apoptosis, nitric oxide mediated signal transduction, cell adhesion and migration, and scavenger receptor activity. However, several pathways that are less known for their role in atherosclerosis were also identified, including carbohydrate metabolism, complement activation, calcium ion hemostasis, collagen catabolism, glycosyl bonds and hydrolase activity, taurine transporter activity, heparin activity, etc. The lack of oxygen radical metabolism among the significant processes was surprising, but consistent with up-regulation of genes related to oxygen radical metabolism in all groups with aging.

Taken together, these pathway analyses support prior observations regarding the importance of inflammatory molecular pathways in atherosclerosis, but additionally, expand the repertoire of molecular pathways that are involved in this disease process.

Identification of Other Time-related Patterns of Gene Expression in Atherosclerosis

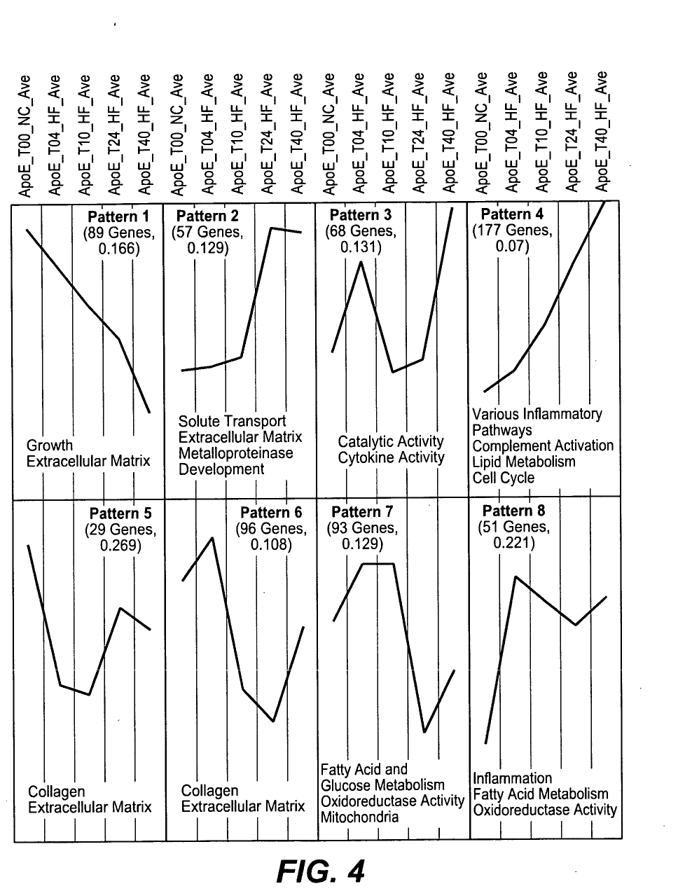

The above analysis examined in detail genes with increased expression levels which correlate with atherosclerotic plaque development. However, additional patterns of gene expression were also identified in these longitudinal studies, to identify classes of genes and pathways not previously identified. For these analyses, the AUC algorithm was employed, which measured expression changes over time, made comparisons between the different strain/diet longitudinal datasets to identify gene expression changes specific for the apoE knockout model, and employed permutation to estimate the FDR (Tabibiazar et al. (2005), supra). Using this methodology several distinct gene expression patterns and pathways that reflect particular biological processes were identified (FIG. 4). For instance, some disease-related pathways were upregulated very early in the disease process and downregulated thereafter (Pattern 6). Others were upregulated early and maintained at relative high expression throughout the time course of the disease (Pattern 8). Whereas the earlier pattern is enriched in pathways representing biological processes such as extracellular matrix and collagen metabolism, as well as DNA replication and response to stress, the later pattern is enriched in pathways representing biological processes such as fatty acid metabolism, oxidoreductase activity and heat-shock protein activity. Some disease related pathways were upregulated in both early and late phases of disease development (Pattern 3), including those associated with metabolism, such as glycolysis and gluconeogenesis. Other patterns (Pattern 4) are represented by key pathways regulating plaque development, including growth factor, cytokine, and cell adhesion activity. Interestingly, inflammation is represented in almost all of the patterns described herein.

Identification of Stage Specific Gene Expression Signature Patterns

Classification approaches to human cancer have provided significant insights regarding the clinical features of the tumor, including propensity to metastasis, drug responsiveness, and long term prognosis (Golub et al. (1999) Science 286:531-537; Lapointe et al. (2004) Proc Natl Acad Sci 101:811-816; Paik et al. (2004) N Engl JMed (“Multigene Assay to Predict Recurrence of Tamoxifen-Treated, Node-Negative Breast Cancer”); Sorlie et al. (2001) Proc Natl Acad Sci 98:10869-10874). For atherosclerosis, the clinical utility of classification algorithms will include prediction of future events. To establish a panel of genes whose expression in the vessel wall can accurately classify disease stage, and which may thus be useful for clinical genomic and biomarker applications, the support vector machines algorithm was employed on this comprehensive mouse model disease data set. Employing the SVM classification algorithm, 38 genes were identified that were able to accurately classify each experiment with one of five defined stages of atherosclerosis in mice (FIG. 5A). The results demonstrated that these genes can distinguish normal from severe lesions with 100% accuracy. The intermediate stages of the disease are also distinguished from the other stages with a high degree of accuracy (88-97%) (Table 3).

To validate the classifier genes, their ability to accurately categorize an independent group of 16 week old apoE knockout mice, which were evaluated with a different array and labeling methodology, was evaluated. The microarray utilized different probes for some of the same genes. Moreover, the labeling methodology used a linear amplification step which may introduce further variability in the data. Using the SVM classification algorithm, each of the 4 replicate experiments was accurately classified with the correct stage of the disease process (FIG. 5B). As indicated by the greater correlation between gene expression in this independent group of mice and gene expression patterns in the original experimental group aged 24 weeks, the classifier genes accurately matched this validation dataset to the closest timepoint in the database.

Identification of Mouse Disease Gene Expression Patterns in Human Coronary Atherosclerosis

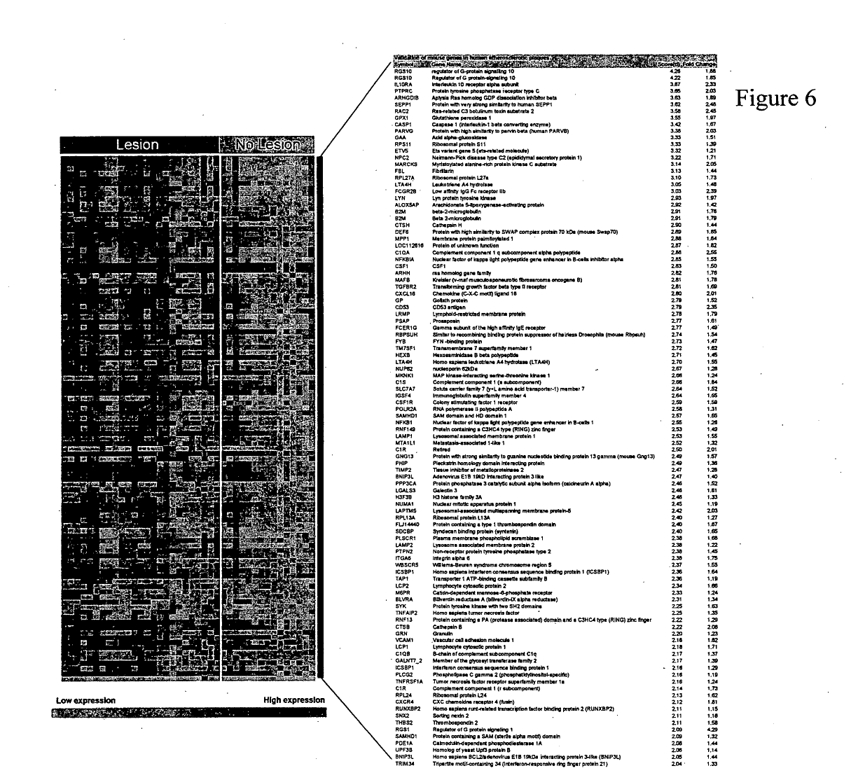

The expression profile of differentially regulated mouse genes was investigated in human coronary artery atherosclerosis. For transcriptional profiling of human atherosclerotic plaque, 40 coronary artery samples, dissected from explanted hearts of 17 patients undergoing orthotopic heart transplantation, were used. Of the 21 diseased segments, lesions ranged in severity from grade I to V (modified American Heart Association criteria based on morphological description (Virmani et al., supra)). For the purpose of this analysis, human artery segments were classified as non-lesion or lesion (combined all grades). Atherosclerosis related mouse genes were matched to human orthologs by gene symbol or by known homology (www.ncbi.nlm.nih.gov/entrez/query.fcgi?DB=homologene). Comparison of expression of the mouse genes between lesion and non-lesion human samples using the significance analysis of microarrays algorithm (FDR<0.025) revealed more than 100 mouse genes with higher expression in the diseased human tissue (FIG. 6). In view of the differences between the tissue samples used in these gene expression experiments, these constitute an important common set of disease relevant genes.

To further test the relevance of our findings in mouse atherosclerosis, the accuracy of the mouse classifier genes was assessed in human atherosclerotic disease, employing established statistical methods. The mouse classifier genes were first used to predict various stages of coronary artery disease in the human arterial samples. The results demonstrated a high degree of accuracy in predicting atherosclerotic disease severity (71.2 to 84.7% accuracy) (Table 3).

Additionally, the mouse classifier genes were used to categorize human atherectomy tissue obtained from coronary vessels treated for chronic atherosclerosis or in-stent restenosis. The pathophysiological basis of restenosis is quite distinct from that of chronic coronary atherosclerosis, and it was of interest to demonstrate that the classifier genes could distinguish the disease processes (Rajagopal and Rockson (2003) Am J Med 115:547-553). The results (Table 3) demonstrated significant accuracy in distinguishing the two types of lesions (85.4 to 93.7% accuracy), further validating the significance of the mouse atherosclerosis gene expression patterns in human disease. The greater accuracy of classification with these samples compared to the arterial segments likely reflects less variation in the clinical profile of the patients, which have much less complex medication and comorbid features than the pre-cardiac transplant patients in the above analysis.

TABLE 2
Biological themes in atherosclerosis. Enrichment analysis of atherosclerosis-related genes
annotated with Gene Ontology and Biocarta terms demonstrates involvement of multiple
molecular pathways and biological processes. Probabilities (p-values) were derived using
Fisher exact test. 8478 of the entire microarray and 513 of genes in our set (including
additional 183 genes which demonstrated Pearson correlation >0.8 with the upregulated
pattern) were annotated with GO, Biocarta, or other terms.

| | List gene # | Total gene # | p-value |
|---|---|---|---|
| Biological Process (GO annotation) | | | |
| immune response | 19 | 78 | <0.0001 |
| chemotaxis | 10 | 23 | <0.0001 |
| cell surface receptor linked signal transduction | 12 | 38 | <0.0001 |
| defense response | 15 | 60 | <0.0001 |
| carbohydrate metabolism | 14 | 67 | <0.0001 |
| antigen processing | 5 | 9 | <0.0001 |
| locomotory behavior | 4 | 6 | <0.0001 |
| inflammatory response | 8 | 30 | <0.0001 |
| complement activation | 5 | 12 | <0.0001 |
| proteolysis and peptidolysis | 25 | 204 | 0.001 |
| antigen presentation | 4 | 10 | 0.002 |
| intracellular signaling cascade | 28 | 269 | 0.003 |
| zinc ion homeostasis | 2 | 2 | 0.004 |
| transmembrane receptor protein | 2 | 2 | 0.004 |
| tyrosine kinase activatio [symbol] | | | |
| hormone metabolism | 2 | 2 | 0.004 |
| hair cell differentiation | 2 | 2 | 0.004 |
| cell death | 2 | 2 | 0.004 |
| exogenous antigen via MHC class II | 3 | 7 | 0.006 |
| ossification | 4 | 14 | 0.008 |
| collagen catabolism | 3 | 8 | 0.010 |
| classical pathway | 3 | 8 | 0.010 |
| vesicle transport along actin filament | 2 | 3 | 0.011 |
| taurine transport | 2 | 3 | 0.011 |
| nitric oxide mediated signal transduction | 2 | 3 | 0.011 |
| negative regulation of angiogenesis | 2 | 3 | 0.011 |
| endogenous antigen via MHC class I | 2 | 3 | 0.011 |
| endogenous antigen | 2 | 3 | 0.011 |
| cellular defense response (sensu Vertebrsta) | 2 | 3 | 0.011 |
| beta-alanine transport | 2 | 3 | 0.011 |
| lymph gland development | 4 | 17 | 0.017 |
| perception of pain | 2 | 4 | 0.020 |
| myeloid blood cell differentiation | 2 | 4 | 0.020 |
| female gamete generation | 2 | 4 | 0.020 |
| cytolysis | 2 | 4 | 0.020 |
| ATP biosynthesis | 4 | 19 | 0.025 |
| regulation of peptidyl-tyrosine phosphorylation | 3 | 11 | 0.025 |
| neurotransmitter transport | 3 | 12 | 0.032 |
| sex differentiation | 2 | 5 | 0.032 |
| exogenous antigen | 2 | 5 | 0.032 |
| call adhesion | 20 | 217 | 0.039 |
| regulation of cell migration | 3 | 13 | 0.040 |
| wound healing | 2 | 6 | 0.047 |
| ureteric bud branching | 2 | 6 | 0.047 |
| cellular defense response | 2 | 6 | 0.047 |
| acute-phase response | 2 | 6 | 0.047 |
| regulation of transcription from Pot II promoter | 6 | 44 | 0.048 |
| hydrogen transport | 3 | 14 | 0.049 |
| calcium ion homeostesis | 3 | 14 | 0.049 |
| Molecular Function (GO annotation) | | | |
| acting on glycosyl bonds | 12 | 31 | <0.0001 |
| interleukin receptor activity | 8 | 13 | <0.0001 |
| hydrolase activity | 67 | 641 | <0.0001 |
| cytokine activity | 13 | 57 | <0.0001 |
| hematopoietin | 9 | 32 | <0.0001 |
| complement activity | 5 | 9 | <0.0001 |
| cytokine binding | 3 | 3 | <0.0001 |
| C-C chemokine receptor activity | 3 | 3 | <0.0001 |
| chemokine activity | 4 | 7 | <0.0001 |
| cysteine-type endopeptidase activity | 11 | 63 | 0.001 |
| tumor necrosis factor receptor activity | 3 | 5 | 0.002 |
| platelet-derived growth factor receptor binding | 2 | 2 | 0.004 |
| cathepsin D activity | 2 | 2 | 0.004 |
| beta-N-acetylhexosaminidase activity | 2 | 2 | 0.004 |
| antimicrobial peptide activity | 2 | 2 | 0.004 |
| scavenger receptor activity | 3 | 6 | 0.004 |
| cysteine-type peptidase activity | 9 | 56 | 0.006 |
| mannosyl-oligosaccharide | 3 | 7 | 0.006 |
| 1,2-alpha-mannosidase activi [symbol] | | | |
| recepter activity | 42 | 479 | 0.009 |
| taurine:sodium symporter activity | 2 | 3 | 0.011 |
| taurine transporter activity | 2 | 3 | 0.011 |
| myosin ATPase activity | 2 | 3 | 0.011 |
| MHC class I receptor activity | 2 | 3 | 0.011 |
| cathepsin B activity | 2 | 3 | 0.011 |
| calcium channel regulator activity | 2 | 3 | 0.011 |
| beta-alanine transporter activity | 2 | 3 | 0.011 |
| catalytic activity | 23 | 230 | 0.012 |
| solute:hydrogen antiporter activity | 2 | 4 | 0.020 |
| protein kinase C activity | 2 | 4 | 0.020 |
| tumor necrosis factor receptor binding | 3 | 11 | 0.025 |
| hydrogen-exporting ATPase activity | 5 | 29 | 0.028 |
| neurotransmitter:sodium symporter activity | 2 | 5 | 0.032 |
| MHC class II receptor activity | 2 | 5 | 0.32 |
| heparin binding | 5 | 31 | 0.037 |
| endopeptidase inhibitor activity | 4 | 22 | 0.041 |
| protein-tyrosine-phosphatase activity | 7 | 54 | 0.043 |
| hydrogen ion transporter activity | 5 | 33 | 0.046 |
| sulfuric ester hydrolase activity | 2 | 6 | 0.047 |
| Cellular Component (GO annotation) | | | |
| extracellular space | 139 | 1148 | <0.0001 |
| lysosome | 26 | 66 | <0.0001 |
| extracellular | 23 | 117 | <0.0001 |
| integral to membrane | 138 | 1637 | <0.0001 |
| membrane | 77 | 862 | <0.0001 |
| integral to plasma membrane | 22 | 205 | 0.006 |
| extracellular matrix | 14 | 114 | 0.009 |
| external side of plasma membrane | 3 | 9 | 0.014 |
| Biocarta Pathways | | | |
| classicPathway | 3 | 3 | <0.0001 |
| il22bppathway | 4 | 7 | <0.0001 |
| nktPathway | 5 | 12 | <0.0001 |
| Ccr5Pathway | 5 | 13 | 0.001 |
| reckPathway | 4 | 8 | 0.001 |
| compPathway | 3 | 4 | 0.001 |
| il7Pathway | 4 | 10 | 0.002 |
| TPOPathway | 5 | 17 | 0.003 |
| cxcr4Pathway | 5 | 17 | 0.003 |
| blymphocytePathway | 2 | 2 | 0.004 |
| il10Pathway | 3 | 7 | 0.006 |
| pdgfPathway | 5 | 22 | 0.009 |
| ionPathway | 2 | 3 | 0.011 |
| egfPathway | 5 | 23 | 0.011 |
| biopeptidesPathway | 5 | 23 | 0.011 |
| bcrPathway | 5 | 25 | 0.015 |
| ghPathway | 4 | 17 | 0.017 |
| fcer1Pathway | 5 | 26 | 0.018 |
| spryPathway | 3 | 10 | 0.019 |
| neutrophilPathway | 2 | 4 | 0.020 |
| mrpPathway | 2 | 4 | 0.020 |
| trkaPathway | 3 | 11 | 0.025 |
| pmlPathway | 3 | 11 | 0.025 |
| srcRPTPPathway | 3 | 12 | 0.032 |
| plcdPathway | 2 | 5 | 0.032 |
| itngPathway | 2 | 5 | 0.032 |
| il2Pathway | 3 | 13 | 0.040 |
| RacCycDPathway | 4 | 22 | 0.041 |
| lymphocytePathway | 2 | 6 | 0.047 |
| nuclearRsPathway | 3 | 14 | 0.049 |
| cdMacPathway | 3 | 14 | 0.049 |
| CCR3Pathway | 3 | 14 | 0.049 |
| Summary annotation for Inflammatory genes | | | |
| defense | 15 | 54 | <0.0001 |
| chemokine | 9 | 22 | <0.0001 |
| interleukin | 9 | 38 | <0.0001 |
| cytokine | 18 | 144 | 0.003 |
| TNF | 4 | 13 | 0.006 |
| TH2 | 4 | 15 | 0.011 |
| TH1 | 4 | 16 | 0.013 |
| macrophage | 3 | 13 | 0.040 |

TABLE 3
Classification of mouse and human atherosclerotic tissues employing mouse classifier genes.
To validate the accuracy of mouse classifier genes in predicting disease severity we utilized
various mouse and human expression datasets. The SVM algorithm was utilized for cross
validation of mouse experiments grouped on the basis of (A) stage of disease (no disease-
apoE time 0, mild disease-apoE at 4 and 10 weeks on normal diet, mild-moderate disease-
apoE at 4 and 10 weeks on highfat diet, moderate disease-apoE at 24 and 40 weeks on normal
diet, and severe disease-apoE at 24 and 40 weeks on high fat diet); (B) 3 different time points
(apoE at 0 vs. 10, vs. 40 weeks); (C) Human coronary artery with lesion vs. no lesion; and (D)
atherectomy samples derived from in-stent restenosis vs. native atherosclerotic lesions.
For each analysis, the accuracy of classification is represented in tabular fashion with the
confusion matrix generated using N-fold cross validation methods.

| A | TRUE | TRUE | TRUE | TRUE | TRUE | |
|---|---|---|---|---|---|---|
| PREDICTED | No dz | Mild_dz | Mild_mod dz | Mod_dz | Severe_dz | Correct [%] |
| No dz | 64 | 0 | 1 | 0 | 0 | 98.5 |
| Mild_dz | 2 | 140 | 0 | 0 | 0 | 98.6 |
| Mild_mod dz | 0 | 0 | 148 | 20 | 0 | 88.1 |
| Mod_dz | 0 | 0 | 3 | 149 | 0 | 98.0 |
| Severe_dz | 0 | 0 | 0 | 0 | 173 | 100.0 |
| Correct [%] | 97.0 | 100.0 | 97.4 | 88.2 | 100.0 | |

| B | TRUE | TRUE | TRUE | |
|---|---|---|---|---|
| PREDICTED | ApoE_T00_NC | ApoE_T10_HF | ApoE_T40_HF | Correct [%] |
| ApoE_T00_NC | 68 | 0 | 0 | 100 |
| ApoE_T10_HF | 0 | 56 | 0 | 100 |
| ApoE_T40_HF | 0 | 0 | 76 | 100 |
| Correct [%] | 100 | 100 | 100 | |

| C | TRUE | TRUE | |
|---|---|---|---|
| PREDICTED | Lesion | No lesion | Correct [%] |
| Lesion | 183 | 33 | 84.7 |
| No lesion | 53 | 131 | 71.2 |
| Correct [%] | 77.5 | 79.9 | |
| D | TRUE | TRUE | |
| PREDICTED | ISR | De novo | Correct [%] |
| ISR | 345 | 44 | 88.7 |
| De novo | 59 | 652 | 91.7 |
| Correct [%] | 85.4 | 93.7 | |

### Example 2

### Mouse Strain—Specific Differences in Vascular Wall Gene Expression and Their Relationship to Vascular Disease

Methods

RNA Preparation and Hybridization to the Microarray

Three-week old female C3H/HeJ, C57B1/6J, and apoE knock-out mice (C57BL/6J-Apoe^{tmlUnc}) were purchased from Jackson Labs (JAX® Mice and Services, Bar Harbor, Me.). At four weeks of age the mice were either continued on normal chow or switched to non-cholate containing high-fat diet which included 21% anhydrous milkfat and 0.15% cholesterol (Dyets #101511, Dyets Inc., Bethlehem, Pa.) for a maximum period of 40 weeks. At each of the time-points, including 0 (baseline), 4, 10, 24 and 40 weeks, for each of the conditions (strain-diet combination), 15 mice were harvested for RNA isolation, for a total of 450 mice. Following Stanford University animal care guidelines, the mice were anesthetized with Avertin and perfused with normal saline. The aortas from the root to the common iliacs were carefully dissected, flash frozen in liquid nitrogen, and divided into three pools of five aortas for further RNA isolation. Total RNA was isolated as described in Tabibiazar et al. (2003) Circ Res 93:1193-1201. First strand cDNA was synthesized from 10 μg of total RNA from each pool and from whole 17.5-day embryo for reference RNA in the presence of Cy5 or Cy3 dCTP, respectively, and hybridized to a mouse 60mer oligo microarray (G4120A, Agilent Technologies, Palo Alto, Calif.), generating three biological replicates for each time point.

Data Processing

Array image acquisition and feature extraction was performed using the Agilent G2565AA Microarray Scanner and feature extraction software version A.6.1.1. Normalization was carried out using a LOWESS algorithm, and Dye-normalized signals were used in calculating log ratios. Features with reference values of<2.5 standard deviations above background for the negative control features were regarded as missing values. Those features with values in at least ⅔ of the experiments and present in at least one of the replicates were retained for further analysis. For SAM analyses, a K-nearest-neighbor (KNN) algorithm was applied to impute for missing values. (Tabibiazar et al. (2003), supra.)

Data Analysis

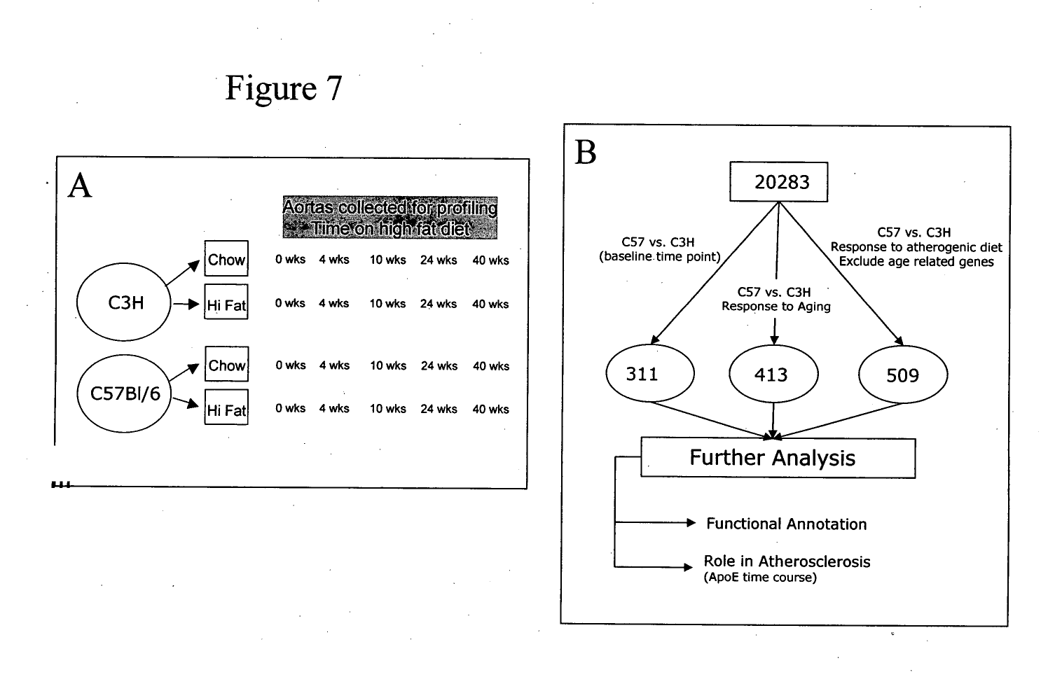

Experimental design and analysis flow chart is depicted in FIG. 7. Significance Analysis of Microarrays (SAM) was employed to identify genes with statistically different expression between the C3H and C57 mice at baseline. (Tabibiazar et al. (2003), supra; Tusher et al. (2001) PNAS 98:5116-5121; Chen et al. (2003) Circulation 108:1432-1439.) For partitioning clustering of the genes with K-Means and self-organizing-maps (SOM), we used positive correlation for distance determination and required complete linkage, which uses the greatest distance between genes to ascribe similarity. SOM and K-Means analyses were performed using Expressionist software (GeneData, Inc., USA). Heatmaps were generated using HeatMap Builder. For enrichment analysis we used the EASE analysis software which employs Gene Ontology (GO) annotation and the Fisher's exact test to derive biological themes within particular gene sets. (Hosack et al. (2003) Genome Biol. 4:R70.) For time-course study, a new statistical algorithm, the Area-Under-Curve (AUC) analysis was devised. For each sequence of 4 triplicate gene expression measurements over time, we first subtracted the measurement at time 0 from all values. We then computed the signed area under the curve. The area is a natural measure of change over time. These areas were then used to compute an F-statistic for comparing C57 and C3H mice across the different diets. A permutation analysis, similar to that employed in SAM, was carried out to estimate the false discovery rate (q-value or “FDR”) for different levels of the F-statistic. For ease of presentation, genes which meet our FDR cutoffs will be referred to as “significant” throughout the remainder of the article. All microarray data were submitted to the NCBI Gene Expression Omnibus (GEO GSE1560; http://www.ncbi.nlm.nih.gov/geo/).

Aortic Lesion Analysis

For select time points within various experimental groups, 5 to 7 female mice were used for histological lesion analysis. Atherosclerosis lesion area was determined as described in Tangirala et al. (1995) 36:2320-2328.

Quantitative Real-Time Reverse Transcriptase-Polymerase Chain Reaction

Primers and probes for 10 representative differentially expressed genes were obtained from Applied Biosystems Assays-on-Demand. A Total of 90 reactions were performed from representative RNA samples used for microarray experiments. These included triplicate assay on three pools of five aortas. cDNA was synthesized and Taqman was performed as described in Tabibiazar et al. (2003), supra.

Results

Baseline Differences in Gene Expression Patterns between the Mouse Strains

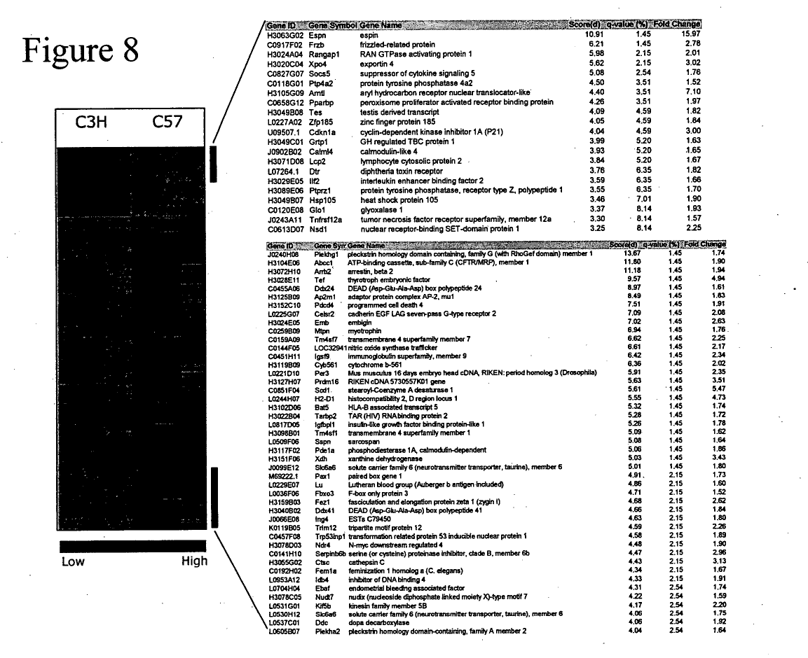

Differences in gene expression levels between the two strains at baseline, before effects of aging or diet become apparent, may identify genes that play a role in determining vascular wall disease susceptibility. To identify such genes SAM was used to compare the vascular wall gene expression of C3H vs. C57 mice at 4 weeks of age, with all animals on normal chow diet. SAM identified 311 genes as being significantly differentially expressed (FDR<0.1 with>1.5 fold difference), and expression patterns of these genes provided a clear partition between C3H and C57 mice (FIG. 8). A separate 2-class comparison (SAM, FDR<0.1) between C57 and apoE-deficient mice with a C57B1/6 genetic background revealed only a few genes, including Apo-E, which were differentially expressed in the 2 groups of mice (data not shown).

Comparison of C3H and C57 vascular wall gene expression at baseline provided a list of compelling candidate genes which reflected differences in biological processes such as growth, differentiation, and inflammation as well as molecular functions such as cathecholamine synthesis, phosphatase activity, peroxisome function, insulin like growth factor activity, and antigen presentation (FIG. 8). These processes were exemplified by higher expression of genes such as Cdknla, Pparbp, protein tyrosine phosphatase-4a2, and Socs5 in C3H mice, compared with genes such as ABCC1, H2-D1, Bat5, IGFBP1, SCD1, and Serpine6b which demonstrated higher expression in C57 mice. These fundamental baseline gene expression differences may determine disease susceptibility as the mice are exposed to age-related stimuli or dietary challenges.

Age-related Differences in Gene Expression Patterns between the Mouse Strains

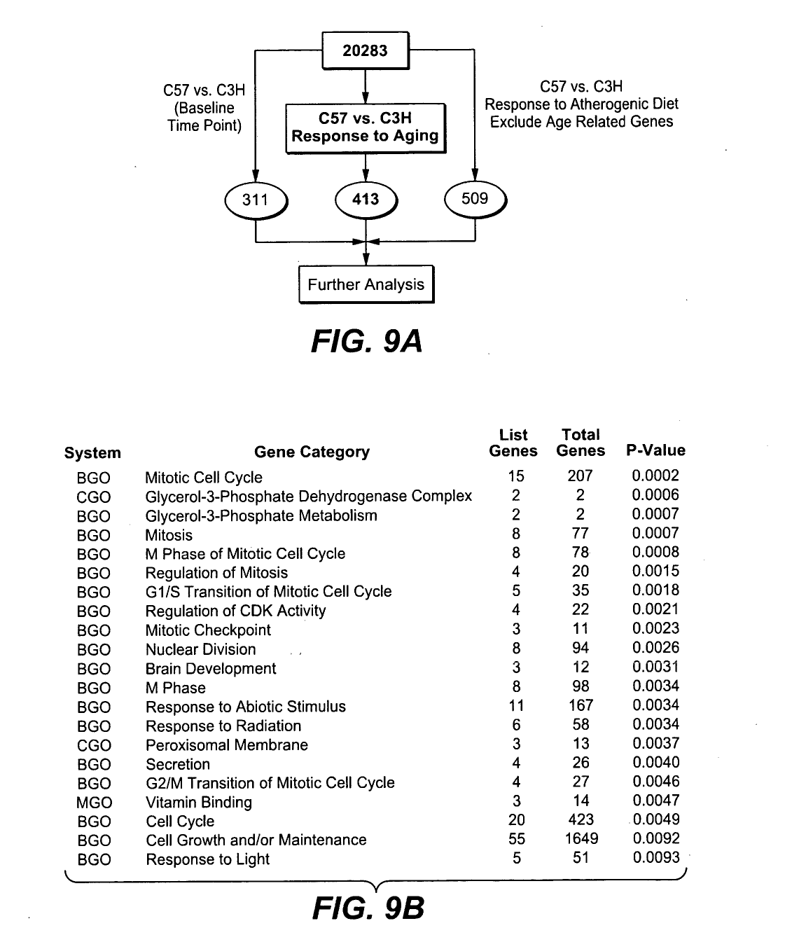

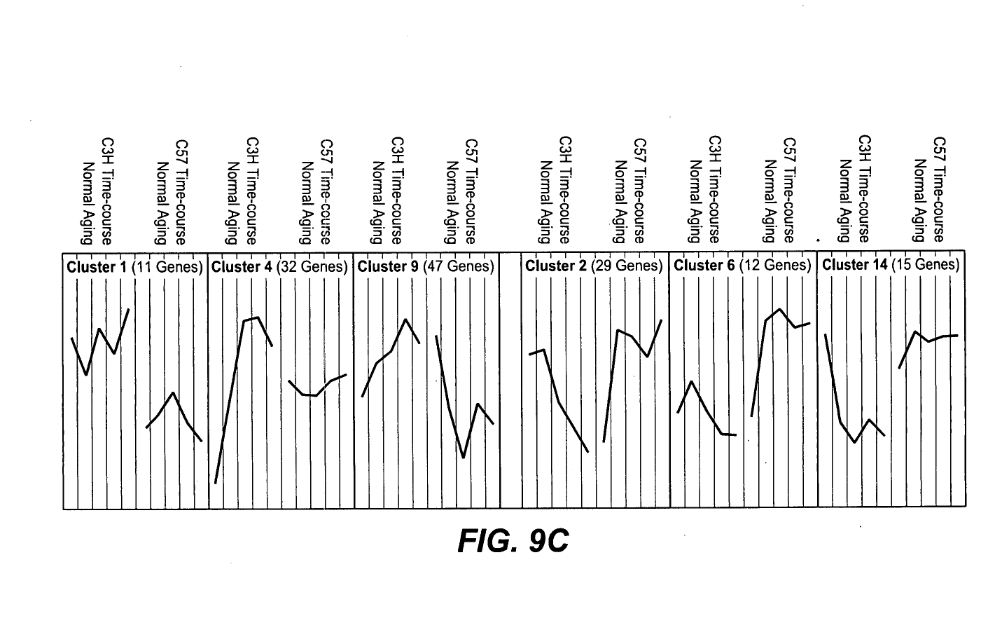

To further examine the vascular wall gene expression differences between C57 and C3H mice, an analysis was performed to identify genes differentially expressed in response to aging (FIG. 9). Data was collected at five time points over a 40 week period. To identify such genes, we developed the Area Under the Curve (AUC) analysis. The AUC analysis relies on a permutation procedure to reduce the number of potential false positives generated due to multiple testing, but still utilizes the increase in statistical power of time-course experimental design. Comparing C57 vs. C3H time-course differences on normal diet with a rigid cutoff (FDR<0.05) did not identify any genes. However, relaxing the AUC stringency (f-statistic>10, FDR <0.45) allowed a large number of genes (413) to be included for pathway over-representation analysis using GO annotation. Functional annotation and group over-representation analysis (Fisher test p-value <0.02) of the resultant differentially expressed genes revealed differences in a number of biological processes, including growth and development, as well as a number of molecular fimctions such as cell cycle control, regulation of mitosis, and metabolism (FIG. 9b). Some of these processes are exemplified by genes with higher expression in C57 mice, such as Aocl (pro-oxidative stress), Bub1 (cell cycle check point), Cyclin B2, as well as genes with higher expression in C3H, including INHBA and INHBB.

Temporally variable genes identified by AUC analysis were further characterized with K-Means clustering to identify dynamic patterns of expression during the aging process (FIG. 3c). Clusters 1, 4, and 9 revealed either higher overall expression or temporally increasing levels of expression in C3H mice compared with C57 mice. In contrast, clusters 2, 6, and 14 revealed the opposite pattern. Of the genes which were noted to be differentially expressed in the two strains during aging, 51 genes were also differentially expressed at baseline, suggesting that baseline differences of certain genes can further be affected with aging.

Diet-related Differences in Gene Expression Patterns between the Mouse Strains

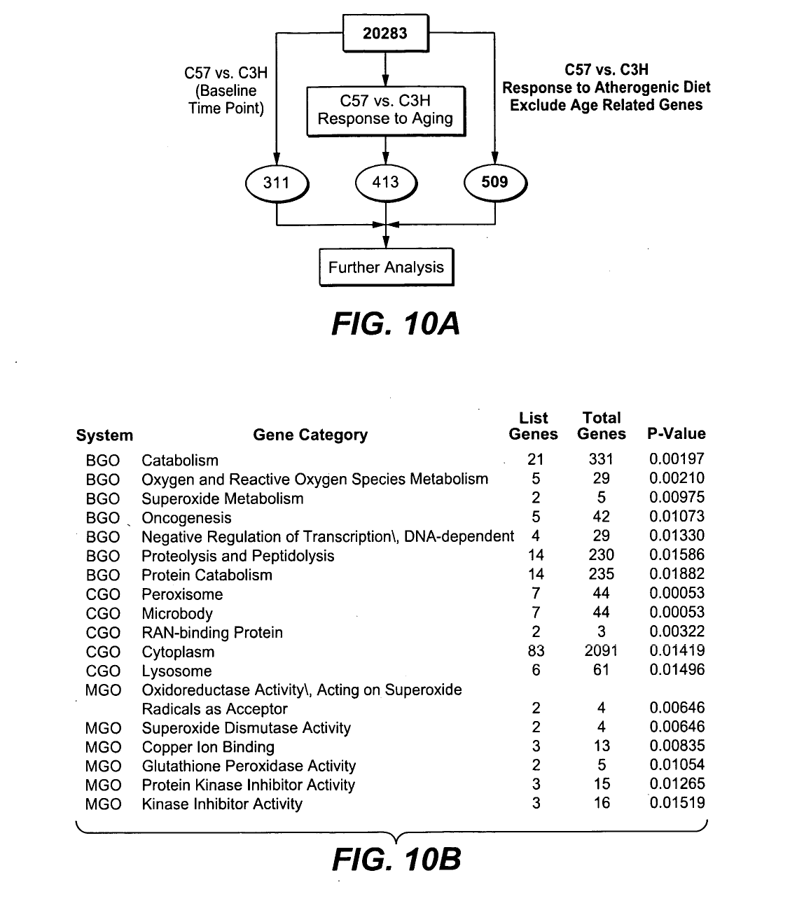

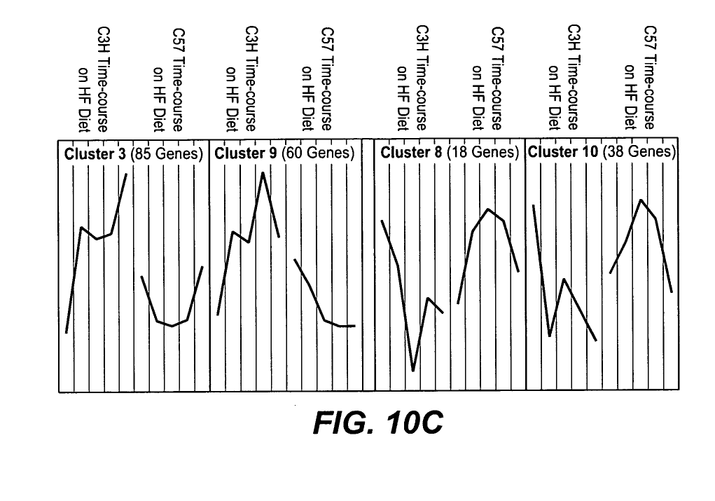

Differential vascular wall response to atherogenic stimuli was determined by comparing temporal gene expression patterns in C57 vs. C3H mice on high-fat diet (FIG. 10A). Comparing C57 vs. C3H time-course differences on high-fat diet with a rigid cutoff (FDR<0.05) identified 35 genes, including Hgfl and Tgf4, which were down regulated in C57 on high-fat diet. Additional known genes, as well as a number of ESTs were also identified. Employing a less stringent AUC cutoff allowed identification of a larger number of genes, which could be evaluated with pathway over-representation analysis using GO annotation. At this level of stringency (f-statistic>10, FDR<0.35), a total of 650 genes with temporally variable expression were identified. Genes that were also differentially regulated by the aging process (141 of 650 genes) were excluded from further analysis of this group. 38 of the remaining 509 genes were among those differentially expressed at baseline. Functional annotation and group over-representation analysis (Fisher test p-value<0.02) of these differentially expressed genes revealed differences in biological processes such as catabolism, oxygen reactive species and superoxide metabolism, and proteo- and peptidolysis as well as molecular functions such as fatty acid metabolism, oxidoreductase and methyltransferase activities (FIG. 10B). Interestingly, this analysis suggested important differences between the two mouse strains with respect to the activity of the peroxisome, microbody and lysosome. Some of these processes were exemplified by genes with higher expression in C3H mice, such as Ccs, Ephx2, Gpx4, Prdx6 (anti-oxidants), Sirt3 (transcriptional repressor), PPARa, and Mcd, as well as genes with higher expression in C57 mice, such as Lysyl oxidase and Cdkn1a. K-means clustering of these genes identified a small number of distinct expression patterns (FIG. 10C), with clusters 3 and 9 revealing increased gene expression in C3H mice and clusters 8 and 10 showing the opposite pattern.

Evaluation of Strain-specific Differentially Regulated Genes in the ApoE Model

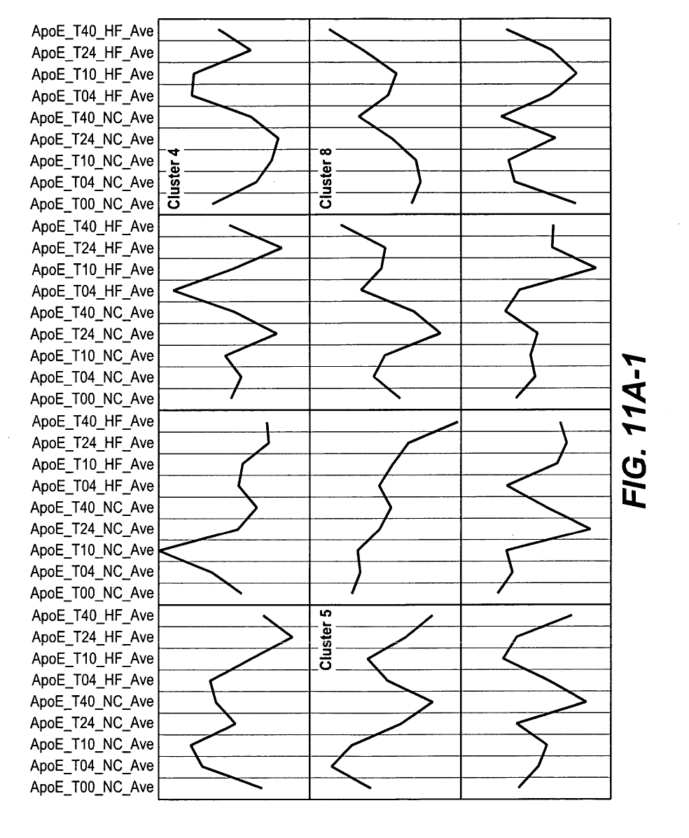

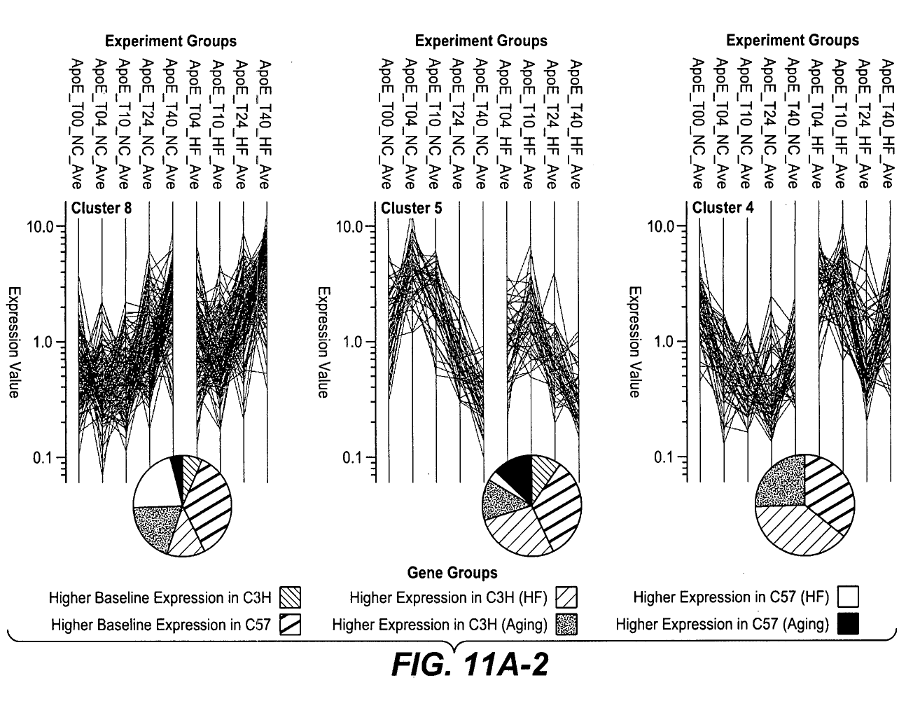

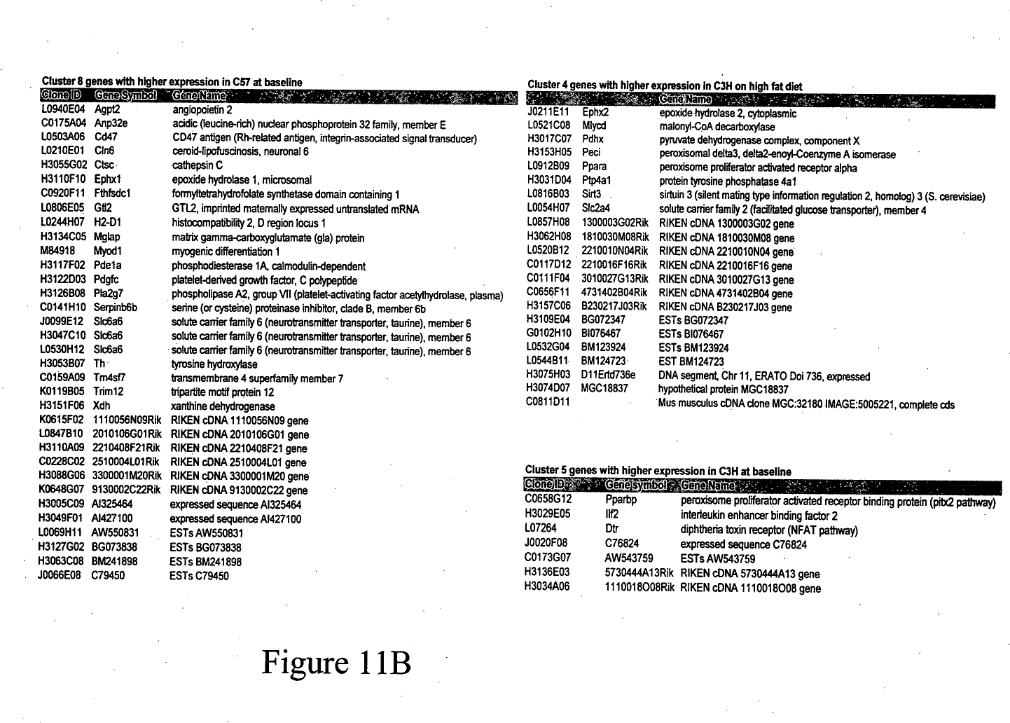

Using these techniques, a significant number of genes have been identified that are differentially expressed in the atherosclerosis resistant C3H and susceptible C57 mice, some of which are likely involved in atherogenesis and some of which are likely irrelevant to the process. To further select genes most likely to be involved in atherogenesis, expression in apoE-deficient mice fed normal or high-fat diet over a period of 40 weeks was investigated (FIG. 1 1). We utilized SOM analysis to visualize the expression profiles of these subsets of genes throughout the development and progression of atherosclerosis in the ApoE-deficient mice. The analysis revealed several patterns of gene expression. For example, SOM cluster 8 demonstrated a consistently increasing pattern of expression which correlated with disease progression in the apoE-deficient mice (FIG. 11). As evidenced by the pie chart, this cluster is enriched with genes that were identified as more highly expressed in C57 versus C3H mice at baseline (i.e., potentially atherogenic). In contrast, clusters 4, 5, and 6 showed decreasing expression with disease progression. The decreased expression of genes in cluster 4 was somewhat attenuated with high-fat challenge of the ApoE-deficient mice. This cluster is particularly enriched with genes that had revealed a higher expression in C3H mice (i.e., potentially atheroprotective) with atherogenic stimuli and with aging.

Given C3H resistance and C57 susceptibility to atherosclerosis, as an initial hypothesis it was postulated that genes with higher expression in C3H mice confer resistance, whereas genes with higher expression in C57 mice may have a pro-atherogenic role. With this point of reference, gene clusters were further examined. For example, limiting the list of genes in SOM cluster 8 (genes with increased expression with atherosclerosis) to those that also had higher baseline expression in C57 mice yielded an interesting set of genes that may be atherogenic. This group included inflammation related genes such as H2-D1, Pdgfc, Paf, and Cd47. Other compelling genes included Agpt2, Mglap, Xdh, Th, and Ctsc. Conversely, limiting the list of genes in clusters 4 and 5 to those with higher expression in C3H mice identified a group of genes with potential athero-protective function. Some of those genes included Pparα, Pparbp, as well as Ptp4a1, and Mcd.

Lesion Analysis in the Genetic Models

To address whether some of the gene expression differences are related to presence of atherosclerotic lesion in C57 mice, the total atherosclerotic burden was determined in the aorta by calculating a percent lesion area in aortas of C57 (n=5) and C3H (n=5) mice. Comparisons were made at time 0 and 40 weeks on normal or high-fat diet. Non-cholate containing high-fat diet was used to prevent caustic effects on the vascular wall. As expected, C57 and C3H mice on either diet did not demonstrate evidence of atherosclerosis throughout the course of the experiment, suggesting that observed gene expression changes cannot be explained by different cellular composition of the vessel wall. Although minimal fatty infiltrates were noted on histological evaluation of the aortic root in C57 mice on high-fat diet, there were no obvious changes in inflammatory cell infiltrate.

Quantitative RT-PCR Validation of Expression Differences

To validate the array results with quantitative RT-PCR and assure that the statistical analyses were identifying truly differentially expressed genes, ten representative genes were assayed by quantitative RT-PCR. Several genes were used from each group of significant genes. There is high degree of correlation between the two methodologies (Pearson correlation of 0.86), validating the results of the microarray analyses.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents and patent applications cited herein are hereby incorporated by reference in their entirety for all purposes to the same extent as if each individual publication, patent or patent application were specifically and individually indicated to be so incorporated by reference.

We claim:

1. A system for detecting gene expression, comprising at least two isolated polynucleotide molecules, wherein each of said at least two isolated polynucleotide molecules detects an expressed gene product from a gene that is differentially expressed in atherosclerotic disease in a mammal, wherein said gene is selected from the group of genes corresponding to the polynucleotide sequences depicted in SEQ ID NOs: 1-927.

2. A system for detecting gene expression, comprising at least two isolated polynucleotide sequences, wherein each of said at least two isolated polynucleotide molecules detects an expressed gene product from a gene that is differentially expressed in atherosclerotic disease in a mammal, wherein said gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

3. A system for detecting gene expression according to claim 1, wherein at least one of said isolated polynucleotide molecules detects a expressed gene product from a gene selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

4. A system according to claim 1, wherein the isolated polynucleotide molecules are immobilized on an array.

5. A system according to claim 4, wherein the array is selected from the group consisting of a chip array, a plate array, a bead array, a pin array, a membrane array, a solid surface array, a liquid array, an oligonucleotide array, polynucleotide array or a cDNA array, a microtiter plate, a membrane, and a chip.

6. A system according to claim 1, wherein the isolated polynucleotides are selected from the group consisting of synthetic DNA, genomic DNA, cDNA, RNA, or PNA.

7. A kit comprising the system of claim 1.

8. A kit comprising the system of claim 4.

9. A method of monitoring atherosclerotic disease in an individual, comprising detecting the expression level of at least one gene selected from the group of genes corresponding to the polynucleotide sequences depicted in SEQ ID NOs: 1-927.

10. The method of claim 9, wherein said at least one gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

11. The method of claim 9, comprising detecting the expression level of at least two of said genes.

12. The method of claim 11, wherein at least one of said at least two genes is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

13. The method of claim 9, comprising detecting the expression level of at least ten of said genes.

14. The method of claim 13, wherein at least one of said at least ten genes is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

15. The method of claim 9, comprising detecting the expression level of at least one hundred of said genes.

16. The method of claim 15, wherein at least one of said at least one hundred genes is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

17. The method of claim 9, wherein said atherosclerotic disease comprises coronary artery disease.

18. The method of claim 9, wherein said atherosclerotic disease comprises carotid atherosclerosis.

19. The method of claim 9, wherein said atherosclerotic disease comprises peripheral vascular disease.

20. The method of claim 9, wherein said expression level is detected by measuring the RNA level expressed by said one or more genes.

21. The method of claim 20, comprising isolating RNA from said individual prior to detecting the RNA expression level.

22. The method of claim 20, wherein said detection of said RNA expression level comprises amplifying RNA from said individual.

23. The method of claim 22, wherein amplification of RNA comprises polymerase chain reaction (PCR).

24. The method of claim 20, wherein detection of said RNA expression level comprises hybridization of RNA from said individual to a polynucleotide corresponding to said at least one gene selected from the group of genes corresponding to the polynucleotide sequences depicted in SEQ ID NOs: 1-927.

25. The method of claim 20, wherein said expression level is detected by measuring the protein level expressed by said one or more genes.

26. The method of claim 9, further comprising selecting an appropriate therapy for said atherosclerotic disease.

27. The method of claim 9, comprising detecting the expression of said at least one gene in serum from said individual.

28. The method of claim 20, comprising measuring said RNA level in serum from said individual.

29. The method of claim 25, comprising measuring said protein level in serum from said individual.

30. A method of monitoring atherosclerotic disease in an individual, comprising detecting RNA expressed from at least one gene selected from the group of genes corresponding to at least one polynucleotide sequence depicted in SEQ ID NOs: 1-927.

31. The method of claim 30, wherein said at least one gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

32. The method of claim 30, comprising measuring said RNA in serum from said individual.

33. A method of monitoring atherosclerotic disease in an individual, comprising detecting protein expressed from at least one gene selected from the group of genes corresponding to at least one polynucleotide sequence depicted in SEQ ID NOs: 1-927.

34. The method of claim 33, wherein said at least one gene is selected from the group of genes corresponding to the polynucleotide sequences depicted in SEQ ID NOs: 8, 14, 26, 32, 50, 64, 83, 99, 142, 154, 159, 161, 177, 181, 200, 390, 430, 434, 439, 440, 476, 491, 508, 530, 534, 565, 567, 572, 624, 647, 657, 690, 733, 745, 806, 824, 886, 882, 901, 905, 913, and 927.

35. The method of claim 33, comprising measuring said protein in serum from said individual.
